(19) United States
(12) Patent Application Publication
(10) Pub. No.: US 2026/0148848 A1
(43) Pub. Date: May 28, 2026

(54) BIOMARKERS FOR DIAGNOSIS AND TREATMENT OF ENDOCRINE HYPERTENSION, AND METHODS OF IDENTIFICATION THEREOF

(71) Applicants: INSERM (INSTITUT NATIONAL DE LA SANTE ET DE LA RECHERCHE MEDICALE), Paris (FR); UNIVERSITE PARIS CITE, Paris (FR); UNIVERSITY OF DUNDEE, Dundee (GB); RADBOUD UMC, Nijmegen (NL); UNIVERSITY OF BIRMINGHAM, Birmingham (GB); UNIVERSITY OF GLASGOW, Glasgow (GB); UNIVERSITÄT ZÜRICH, Zürich (CH); ASSISTANCE PUBLIQUE - HÔPITAUX DE PARIS, Paris (FR)

(72) Inventors: Maria-Christina ZENNARO, Paris (FR); Laurence AMAR, Paris (FR); Arlt WIEBKE, Birmingham (GB); Felix BEUSCHLEIN, Zürich (CH); Eleanor DAVIES, Glasgow (GB); Smarti REEL, Dundee (GB); Parminder Singh REEL, Dundee (GB); Emily JEFFERSON, Dundee (GB); Graeme EISENHOFER, Dresden (DE); Scott MACKENZIE, Glasgow (GB); Alessandro PRETE, Birmingham (GB); Angela TAYLOR, Birmingham (GB); Jaap DEINUM, Nijmegen (NL)

(21) Appl. No.: 18/998,204

(22) PCT Filed: Jul. 28, 2023
(86) PCT No.: PCT/EP2023/071052

§ 371 Date: Jan. 24, 2025

(30) Foreign Application Priority Data
Jul. 29, 2022 (EP) 22306151.6

(51) Int. Cl. G16H 50/20; C12Q 1/6883; G01N 33/74; G01N 33/94; G06N 20/00; G16H 20/10; G16H 50/70

(52) U.S. Cl. CPC G16H 50/20; C12Q 1/6883; G01N 33/743; G01N 33/9433; G06N 20/00; G16H 20/10; G16H 50/70; C12Q 2600/112; C12Q 2600/178; G01N 2333/575; G01N 2800/56; G01N 2800/60

(57) ABSTRACT

The disclosure relates to a combination of biomarkers comprising at least: (i-a) one biomarker selected in each of the following group of biomarkers: Patient's age, Plasma steroids, and Urinary steroids, and at least one biomarker selected in at least one of the group of biomarkers: O-methylated catecholamines, Small metabolites, and miRNA; or (i-b) one biomarker selected in each of the following group of biomarkers: Plasma steroids, Urinary steroids, and Small metabolites, and at least one biomarker selected in at least one of the group of biomarkers: Patient's age, O-methylated catecholamines, and miRNA. The combinations of biomarkers may be used for stratifying a hypertensive patient among different hypertensive diseases comprising Endocrine Hypertension (EHT), Primary Aldosteronism (PA), Pheochromocytoma/Functional Paraganglioma (PPGL), Cushing's Syndrome (CS), and Primary Hypertension (PHT).

### TECHNICAL FIELD

The present disclosure relates to the field of hypertension, in particular Endocrine, or secondary, Hypertension (EHT). More particularly, the disclosure relates to a combination of biomarkers, or molecular signature, for identifying a hypertensive disease, in particular for stratifying a hypertensive patient in a EHT or Primary Hypertension (PHT).

### TECHNICAL BACKGROUND

Arterial hypertension affects up to 25% of the general population and is responsible for 10.4 million deaths per year worldwide (Lancet, 2017; 389 (10064): 37-55). Although a large therapeutic arsenal exists, blood pressure control is sub-optimal in up to two-thirds of patients. Even small increments in blood pressure are associated with increased cardiovascular risk, with 62% of cerebrovascular disease and 49% of ischemic heart disease being attributable to hypertension.

Although the determination of blood pressure and detection of hypertension in an individual may be easily carried out with a sphygmomanometer, specific diagnostic of the type of hypertension proves to be far more difficult.

Detection and identification of secondary forms of hypertension, also known as endocrine hypertension (EHT), are keys to targeted management of the underlying disease and prevention of cardiovascular complications. Endocrine forms of hypertension include a group of adrenal disorders resulting in increased production of hormones affecting blood pressure regulation: primary aldosteronism (PA), pheochromocytoma/functional paraganglioma (PPGL) and Cushing's syndrome (CS). These diseases are associated with increased cardiovascular and metabolic risk and with diminished quality of life (https://cordis.europa.eu/project/id/633983).

Diagnostic procedures for EHT are complex and require referral to specialized centers. Due to the complexity of the work-up, the diagnosis of adrenal forms of hypertension is frequently overlooked and consequently, treatment of the conditions is either not instituted or delayed by 3-5 years after hypertension onset, when cardiovascular and metabolic complications are established. Consequently, patients with EHT remain exposed to an increased risk of renal and cardiovascular complications, including stroke, coronary artery disease, fatal or debilitating cardiac and cerebrovascular events and also to life-long, costly, often futile, antihypertensive treatment, as well as reduced quality of life (QoL) (Savard et al., Hypertension. 2013; 62 (2): 331-336; Mulatero et al., J Clin Endocrinol Metab. 2013; 98 (12): 4826-4833; Rossi et al., Hypertension. 2013; 62 (1): 62-69; Prejbisz et al., J Hypertens. 2011; 29 (11): 2049-2060; Dekkers et al., J Clin Endocrinol Metab. 2013; 98 (6): 2277-2284; Kunzel et al., J Psychiatr Res 2012; 46:1650-1654).

Practical approaches to the diagnosis of endocrine causes of hypertension have been recently reviewed in Yang et al (Nephrology, 22 (2017) 663-677).

Without clear-cut symptoms or signs other than hypertension, it is necessary to carry out biochemical screening to diagnose primary aldosteronism. Patients have to be adequately prepared to ensure accuracy of the results and to remove possible bias caused by medication or diet (Funder et al., J Clin Endocrinol Metab. 2016; 101 (5): 1889-1916). If confirmatory tests are needed, they will involve specialist units. General practitioners are the first entry port for early detection of PA. However, they usually do not have a good knowledge of PA and its diagnostic guidelines. As a result, PA is often under-diagnosed. The aldosterone to renin ratio (ARR) is the recommended test for PA. However, the accuracy and repeatability of this test are such that there is a range of recommended cutoffs. In addition, this test comes with various units for measurement making the comparison between different results difficult. Furthermore, this test reveals itself particularly sensitive to multiple factors such as diet, posture and medications which may affect plasma aldosterone and renin.

Diagnosis of Cushing's syndrome relies upon a combination of tests including 24-hour urine free cortisol (UFC) excretion, overnight 1 mg dexamethasone suppression test (DST) and midnight salivary cortisol. Abnormal results yielded with two of those tests give a positive diagnosis. Nonetheless, there are some limitations, as UFC test may give false negative results in patients with moderate to severe chronic renal impairment or in patients with mild cases of Cushing's syndrome and may give false positive results in patients with pseudo-Cushing's syndrome. It is therefore recommended to repeat at least twice.

Diagnosis of PPGL include measurements of plasma free O-methylated catecholamines or urinary fractionated O-methylated catecholamines. Measurements of plasma O-methylated catecholamines may consist of dosing plasma metabolites of catecholamines taken in supine position (Lenders et al., J Clin Endocrinol Metab, 2014; 99 (6): 1915-1942). Notwithstanding, false positive results are common, with a rate of 19-21% for both plasma free and urine fractionated O-methylated catecholamines. False positive results can be caused by some medications, stress, illnesses, or inappropriate sampling.

Several tests are available to diagnose EHT and differentiate EHT from PHT, or even to differentiate between the different EHTs, PA, CS and PPGGL. Unfortunately, as exposed herein, those tests are complex, lengthy and present limitations which may affect their reliability.

Therefore, there is a need to have a method for stratifying a hypertensive patient among different types of hypertensive patients, such as EHT patient and PHT patient, or PA patient, CS patient and PPGL patient.

Recent technological and methodological developments have given rise to what is now known as omics—a domain of study that includes genomics, as well as epigenomics, transcriptomics, proteomics, and metabolomics. Omics as a whole holds the promise to provide a completest and more accurate picture of an organism's or species' molecular structure; a clearer understanding of the structure and function of molecules downstream from genomic processes may prove pivotal in our understanding of those processes. Rather than relying simply on specific genetic variants in candidate genes in known hypertension pathways, omic methods offer a global picture of all heritable factors influencing hypertension. For complex traits like hypertension, which involve multiple pathways and organs, omic approaches offer the advantage of allowing identification of novel hypertensive mechanisms to further dissect and characterize hypertension pathophysiology (Arnett et al., Circ Res, 2018; 122:1409-1419).

An integrative approach for combining these different omics, in particular thanks to the availability of recent high-throughput computational technologies, would however be advantageous for improving prognostics and predictive accuracy of disease phenotypes. Machine learning provides an efficient way to extract valuable biological knowledge from heterogeneous data with underlying mechanisms (Reel et al., 2021).

Recently, steroid profiling combined with machine learning has been used for identification and subtype classification of patients with primary aldosteronism (Eisenhofer et al., JAMA Netw Open. 2020).

There is a need to have methods for stratifying hypertensive patients among Endocrine Hypertension (EHT), Primary Aldosteronism (PA), Pheochromocytoma/Functional Paraganglioma (PPGL), Cushing's Syndrome (CS) and Primary Hypertension (PHT).

There is a need to have methods for stratifying hypertensive patients with improved accuracy.

There is a need to have methods for stratifying hypertensive patients with improved sensitivity and specificity.

There is a need to have biomarkers for stratifying hypertensive patients among Endocrine Hypertension (EHT), Primary Aldosteronism (PA), Pheochromocytoma/Functional Paraganglioma (PPGL), Cushing's Syndrome (CS) and Primary Hypertension (PHT).

There is a need to have biomarkers for use in methods for stratifying hypertensive patients with improved accuracy.

There is a need to have kits for stratifying a hypertensive patient among a plurality of hypertensive diseases.

There is a need to have computer program product for stratifying a hypertensive patient among a plurality of hypertensive diseases.

There is a need to have a combination of biomarkers for use in a method for treating a hypertensive disease in a patient in need thereof.

There is a need to have a method for stratifying and treating a hypertensive patient.

There is a need to develop a sensitive and specific omics-based method for stratifying hypertensive patients using machine-learning based technologies.

There is a need to develop a sensitive and specific omics-based method for stratifying hypertensive patients among Endocrine Hypertension (EHT) and Primary Hypertension (PHT) using machine-learning based technologies.

There is a need to develop a sensitive and specific omics-based method for stratifying hypertensive patients among Endocrine Hypertension (EHT) using machine-learning based technologies.

There is a need to develop a sensitive and specific omics-based method for stratifying hypertensive patients among Endocrine Hypertension (EHT), Primary Aldosteronism (PA), Pheochromocytoma/Functional Paraganglioma (PPGL), Cushing's Syndrome (CS) and Primary Hypertension (PHT) using machine-learning based technologies.

There is a need to have combinations of biomarkers for differentiating EHT from PHT.

There is a need to have combinations of biomarkers for differentiating Primary Aldosteronism (PA) vs Pheochromocytoma/Functional Paraganglioma (PPGL) vs Cushing's Syndrome (CS).

There is a need to have combinations of biomarkers for differentiating Primary Aldosteronism (PA) vs Pheochromocytoma/Functional Paraganglioma (PPGL) vs Cushing's Syndrome (CS) vs Primary Hypertension (PHT).

The present disclosure aims at satisfying all or part of those needs.

### SUMMARY

According to one of its objects, the present disclosure relates to a combination of biomarkers comprising at least:
(i-a) one biomarker selected in each of the following group of biomarkers: Patient's age, Plasma steroids, and Urinary steroids, and at least one biomarker selected in at least one of the group of biomarkers: O-methylated catecholamines, Small metabolites, and miRNA; or
(i-b) one biomarker selected in each of the following group of biomarkers: Plasma steroids, Urinary steroids, and Small metabolites, and at least one biomarker selected in at least one of the group of biomarkers: Patient's age, O-methylated catecholamines, and miRNA,
(i-c) one biomarker selected in each of the following group of biomarkers: Urinary steroids, and at least one biomarker selected in at least one of the group of biomarkers: Plasma steroids, Small metabolites, and miRNA,
(i-d) one biomarker selected in each of the following group of biomarkers: Patient's age, Plasma steroids, Urinary steroids, and Small metabolites, and at least one biomarker selected in at least one of the group of biomarkers: O-methylated catecholamines, and miRNA, or
(i-e) one biomarker selected in each of the following group of biomarkers: O-methylated catecholamines, and at least one biomarker selected in at least one of the group of biomarkers: Patient's age, Urinary steroids, and Small metabolites.

In some embodiments, the combination of biomarkers may comprise at least:
(i-a) age, plasma 11-deoxycorticosterone, plasma 11-deoxycortisol, plasma 18OH-corticosterone, plasma 18OH-cortisol, plasma 18oxo-Cortisol, plasma 21-deoxycortisol, plasma aldosterone, plasma corticosterone, plasma dehydroepiandrosterone sulfate (DHEAS), urinary 18-hydroxycortisol (18-OHF), urinary α-cortol (acortol), urinary pregnanediol (PD), 3α,5β-tetrahydroaldosterone (THAldo), urinary tetrahydrodeoxycorticosterone (THDOC), and urinary tetrahydro-11-deoxycortisol (THS);
and at least one additional biomarker selected in the group comprising, or consisting in, plasma hsa-let-7g-5p, plasma hsa-miR-106b-3p, plasma hsa-miR-301a-3p, plasma hsa-miR-485-3p, plasma 3-methoxytyramine, plasma metanephrine, plasma normetanephrine, plasma 11-dehydrocorticosterone, plasma cortisol, plasma cortisone, urinary 11-β-hydroxy-androsterone (11-β-OHAn), urinary 17-OH-pregnanolone (17-HP), urinary 5-pregnanediol (PD), urinary 5-pregnenetriol (5-PT), urinary 5α-tetrahydrocortisol (5αTHF), urinary androsterone (An), urinary cortisol, urinary cortisone, urinary dehydroepiandrosterone (DHEA), urinary etiocholanolone (Etio), urinary pregnenetriol (PT), urinary tetrahydro-11-dehydrocorticosterone (THAs), urinary tetrahydrocorticosterone (THB), urinary tetrahydrocortisol (THF), urinary tetrahydrocortisone (THE), urinary α-cortolone (Acortolone), urinary β-cortol (Bcortol), urinary β-cortolone (Bcortolone), plasma acetylcarnitine (C2), plasma C6 (C4:1-DC) (hexanoylcarnitine (fumarylcarnitine)), plasma creatinine, plasma decanoylcarnitine (C10), plasma dodecanoylcarnitine (C12), plasma glutamic acid, plasma H1 (sum of hexoses), plasma lysoPC a C16:0, plasma lysoPC a C17:0, plasma octadecadienylcarnitine (C18:2), plasma octadecenoylcarnitine (C18:1), plasma PC aa C32:1, plasma PC aa C32:2, plasma PC aa C32:3, plasma PC aa C34:1, plasma PC aa C34:2, plasma PC aa C34:3, plasma PC aa C34:4, plasma PC aa C36:1, plasma PC aa C36:2, plasma PC aa C36:3, plasma PC ae C36:3, plasma serotonin, plasma serotonin 1 tryptophan ratio (Serotonin/Trp), plasma spermidine, plasma tetradecenoylcarnitine (C14:1), plasma total dimethylarginine/arginine ratio (Total DMA/Arg), and combinations thereof;
(i-b) plasma 11-deoxycorticosterone, plasma 11-deoxycortisol, plasma 18OH-corticosterone, plasma 18OH-cortisol, plasma 18oxo-cortisol, plasma 21-deoxycortisol, plasma aldosterone, urinary 18-hydroxycortisol (18-OHF), urinary 3α,5β-tetrahydroaldosterone (THAldo), urinary tetrahydrodeoxycorticosterone (THDOC), urinary tetrahydro-11-deoxycortisol (THS), plasma decanoylcarnitine (C10), plasma dodecanoylcarnitine (C12), plasma octadecadienylcarnitine (C18:2), plasma H1 (sum of hexoses), plasma PC aa C32:1, plasma PC aa C34:3, and plasma serotonin, plasma serotonin/tryptophan ratio (Serotonin/Trp),
and at least one additional biomarker selected in the group comprising, or consisting in, age, plasma hsa-let-7g-5p, plasma lysoPC a C16:0, plasma lysoPC a C17:0, plasma normetanephrine, plasma corticosterone, plasma dehydroepiandrosterone sulfate (DHEAS), urinary pregnanediol (PD), plasma acetylcarnitine (C2), plasma C6 (C4:1-DC) (hexanoylcarnitine (fumarylcarnitine)), plasma citrulline/arginine ratio (Cit/Arg), plasma creatinine, plasma glutamic acid (Glu), plasma octadecenoylcarnitine (C18:1), plasma PC aa C32:2, plasma PC aa C32:3, plasma PC aa C34:1, plasma PC aa C34:2, plasma PC aa C36:2, plasma PC aa C36:3, plasma PC ae C36:3, plasma taurine, plasma tetradecenoylcarnitine (C14:1), plasma total dimethylarginine/arginine ratio (Total DMA/Arg), and combinations thereof,
(i-c) urinary α-cortol (acortol), and urinary tetrahydro-11-deoxycortisol (THS),
and at least one of plasma 11-deoxycorticosterone, plasma 11-deoxycortisol, plasma dehydroepiandrosterone (DHEA), plasma dehydroepiandrosterone sulfate (DHEAS), plasma hsa-miR-19a-3p, plasma methioninesulfoxide/methionine ratio (Met-SO/Met), plasma tryptophan, urinary 5-pregnenetriol (5-PT), urinary androsterone (An), urinary cortisol, urinary etiocholanolone (Etio), and combinations thereof
(i-d) age, plasma 11-deoxycorticosterone, plasma 11-deoxycortisol, plasma 18OH-corticosterone, plasma 18OH-cortisol, plasma 18oxo-cortisol, plasma 21-deoxycortisol, plasma a 18-hydroxycortisol (18-OHF), urinary pregnanediol (PD), urinary 3α,5β-tetrahydroaldosterone (THAldo), urinary tetrahydrodeoxycorticosterone (THDOC), urinary tetrahydro-11-deoxycortisol (THS), plasma decanoylcarnitine (C10), plasma tetradecenoylcarnitine (C14:1), plasma octadecenoylcarnitine (C18:1), plasma octadecadienylcarnitine (C18:2), plasma acetylcarnitine (C2), plasma C6 (C4:1-DC) (hexanoylcarnitine), plasma lysoPC a C17:0, plasma PC aa C32:1, plasma PC aa C32:2, plasma PC aa C32:3, plasma PC aa C34:2, plasma PC aa C34:3, plasma PC aa C36:3, plasma serotonin, and plasma serotonin/tryptophan ratio (Serotonin/Trp),
and at least one of plasma creatinine, plasma dehydroepiandrosterone sulfate (DHEAS), plasma dodecanoylcarnitine (C12), plasma H1 (sum of hexoses), plasma lysoPC a C16:0, plasma PC aa C34:1, plasma PC aa C34:4, plasma PC aa C36:1, plasma PC aa C36:2, plasma taurine, urinary dehydroepiandrosterone (DHEA), and combinations thereof, or
(i-e) methoxytyramine, plasma metanephrine, and plasma normetanephrine,
and at least one of age, plasma acetylornithine (Ac-Orn), urinary androsterone (An), urinary etiocholanolone (Etio), and combinations thereof.

As shown in the Examples section, the inventors have observed that it was possible to define specific combinations of biomarkers, including omic-type biomarkers, for stratifying hypertensive patients among PHT and EHT. Further, it was possible to define specific combinations of biomarkers for stratifying hypertensive patients among PA vs PPGL vs CS vs PHT (also named in the description or in the example ALL-ALL or ALL vs ALL). Also, it was possible to define specific combinations of biomarkers for stratifying hypertensive patients among PA vs PPGL. Also, it was possible to define specific combinations of biomarkers for stratifying hypertensive patients among PA vs CS. Also, it was possible to define specific combinations of biomarkers for stratifying hypertensive patients among CS vs PPGL.

Advantageously, the combinations of biomarkers allow developing specific and sensitive methods for stratifying hypertensive patients among hypertension diseases. The combinations of biomarkers can be used with trained classifiers to improve the sensitivity and the specificity of methods for stratifying a hypertensive patient among hypertensive diseases.

In embodiments, the at least one additional biomarker in (i-a) may be selected from plasma metanephrine, plasma normetanephrine, urinary cortisol, urinary dehydroepiandrosterone (DHEA), urinary tetrahydro-11-dehydrocorticosterone (THAs), urinary tetrahydrocortisone (THE), urinary tetrahydrocortisol (THF), and combinations thereof.

In embodiments, the at least one additional biomarker in (i-a) is a combination comprising at least, or consisting in, plasma metanephrine, plasma normetanephrine, urinary cortisol, urinary dehydroepiandrosterone (DHEA), urinary tetrahydro-11-dehydrocorticosterone (THAs), urinary tetrahydrocortisone (THE), and urinary tetrahydrocortisol (THF).

In embodiments, the at least one additional biomarker in (i-b) is plasma normetanephrine.

In embodiments, the at least one additional biomarker in (i-c) is urinary androsterone (An).

The combinations of biomarkers may further be used in methods for stratifying a hypertensive patient among a plurality of hypertensive diseases, for screening antihypertensive treatments or for selecting an antihypertensive treatment for a given hypertensive patient.

Another object of the disclosure relates to a use of a combination of biomarkers for stratifying a hypertensive patient among a plurality of hypertensive diseases.
(i-a) When the plurality of hypertensive diseases is comprising Primary Aldosteronism (PA), Pheochromocytoma/Functional Paraganglioma (PPGL), Cushing's Syndrome (CS) and Primary Hypertension (PHT), then the combination of biomarkers may be a combination of biomarkers (i-a) as above defined or defined elsewhere in the specification.
(i-b) When the plurality of hypertensive diseases is comprising Endocrine Hypertension (EHT) and Primary Hypertension (PHT), then the combination of biomarkers may be a combination of biomarkers (i-b) as above defined or defined elsewhere in the specification.
(i-c) When the plurality of hypertensive diseases is comprising Cushing's Syndrome (CS) and Primary Hypertension (PHT), then the combination of biomarkers may be a combination of biomarkers (i-c) as above defined or defined elsewhere in the specification.
(i-d) When the plurality of hypertensive diseases is Primary Aldosteronism (PA) and Primary Hypertension (PHT), then the combination of biomarkers may be a combination of biomarkers (i-d) as above defined or defined elsewhere in the specification.
(i-e) When the plurality of hypertensive diseases is Pheochromocytoma/Functional Paraganglioma (PPGL) and Primary Hypertension (PHT), then the combination of biomarkers may be a combination of biomarkers (i-e) as above defined or defined elsewhere in the specification.

Another object of the disclosure relates to a use of a combination of biomarkers for stratifying a hypertensive patient among a plurality of hypertensive diseases,
(i-a) when the plurality of hypertensive diseases is comprising Primary Aldosteronism (PA), Pheochromocytoma/Functional Paraganglioma (PPGL), Cushing's Syndrome (CS) and Primary Hypertension (PHT), then the combination of biomarkers may be a combination of biomarkers (i-a) as above defined or defined elsewhere in the specification, or
(i-b) when the plurality of hypertensive diseases is comprising Endocrine Hypertension (EHT) and Primary Hypertension (PHT), then the combination of biomarkers may be a combination of biomarkers (i-b) as above defined or defined elsewhere in the specification, or
(i-c) when the plurality of hypertensive diseases is comprising Cushing's Syndrome (CS) and Primary Hypertension (PHT), then the combination of biomarkers may be a combination of biomarkers (i-c) as above defined or defined elsewhere in the specification, or
(i-d) when the plurality of hypertensive diseases is Primary Aldosteronism (PA) and Primary Hypertension (PHT), then the combination of biomarkers may be a combination of biomarkers (i-d) as above defined or defined elsewhere in the specification, or
(i-e) when the plurality of hypertensive diseases is Pheochromocytoma/Functional Paraganglioma (PPGL) and Primary Hypertension (PHT), then the combination of biomarkers may be a combination of biomarkers (i-e) as above defined or defined elsewhere in the specification,
wherein the use comprises:
   measuring, ex vivo, said combination of biomarkers, and
   operating a trained classifier on said measured combination of biomarkers for obtaining a probability, for each hypertensive disease, of associating the hypertensive patient with a hypertensive disease in order to stratify said hypertensive patient among said plurality of types of hypertensive diseases according to (i-a) or according to (i-b) or according to (i-c) or according to (i-d) or according to (i-e),
said trained classifier being a classifier trained beforehand to learn a plurality of combinations of biomarkers previously selected based on at least one computed evaluation parameter and for several comparisons of at least two types of hypertensive patients.

According to another object, the disclosure relates to a method for stratifying a hypertensive patient among a plurality of types of hypertensive diseases.
(i-a) The plurality of types of hypertensive diseases may comprise Primary Aldosteronism (PA), Pheochromocytoma/Functional Paraganglioma (PPGL), Cushing's Syndrome (CS) and Primary Hypertension (PHT), or (i-b) the plurality of types of hypertensive diseases comprising Endocrine Hypertension (EHT) and Primary Hypertension (PHT), or (i-c) the plurality of hypertensive diseases comprising Cushing's Syndrome (CS) and Primary Hypertension (PHT), or (i-d) the plurality of hypertensive diseases comprising Primary Aldosteronism (PA) and Primary Hypertension (PHT), or (i-e) the plurality of hypertensive diseases comprising Pheochromocytoma/Functional Paraganglioma (PPGL) and Primary Hypertension (PHT).

The method may comprise the use at least one classifier trained beforehand to learn a plurality of combinations of biomarkers previously selected based on at least one computed evaluation parameter and for several comparisons of at least two types of hypertensive patients, and
the method may comprise at least the steps of:
a) measuring, in suitable biological samples previously isolated from said patient, a combination of biomarkers,
wherein for the plurality of hypertensive diseases according to (i-a), the combination of biomarkers may be a combination of biomarkers (i-a) as above defined or defined elsewhere in the specification, or
wherein for the plurality of hypertensive diseases according to (i-b), the combination of biomarkers may be a combination of biomarkers (i-b) as above defined or defined elsewhere in the specification, or
wherein for the plurality of hypertensive diseases according to (i-c), the combination of biomarkers is a combination of biomarkers (i-c) as above defined or defined elsewhere in the specification, or
wherein for the plurality of hypertensive diseases according to (i-d), the combination of biomarkers is a combination of biomarkers (i-d) as above defined or defined elsewhere in the specification, or
wherein for the plurality of hypertensive diseases according to (i-e), the combination of biomarkers is a combination of biomarkers (i-e) as above defined or defined elsewhere in the specification,
b) operating said trained classifier on the combination of biomarkers obtained at step a) from said hypertensive patient to stratify said hypertensive patient among said plurality of types of hypertensive diseases according to (i-a) or according to (i-b).

The method further comprises a step of obtaining for each type of hypertensive disease a probability associating the hypertensive patient to said hypertensive disease.

According to another object, the disclosure relates to a method for stratifying a hypertensive patient among a plurality of types of hypertensive diseases,
(i-a) the plurality of types of hypertensive diseases may comprise Primary Aldosteronism (PA), Pheochromocytoma/Functional Paraganglioma (PPGL), Cushing's Syndrome (CS) and Primary Hypertension (PHT), or
(i-b) the plurality of types of hypertensive diseases comprising Endocrine Hypertension (EHT) and Primary Hypertension (PHT), or
(i-c) the plurality of hypertensive diseases comprising Cushing's Syndrome (CS) and Primary Hypertension (PHT), or
(i-d) the plurality of hypertensive diseases comprising Primary Aldosteronism (PA) and Primary Hypertension (PHT), or
(i-e) the plurality of hypertensive diseases comprising Pheochromocytoma/Functional Paraganglioma (PPGL) and Primary Hypertension (PHT)
the method comprising the use at least one classifier trained beforehand to learn a plurality of combinations of biomarkers previously selected based on at least one computed evaluation parameter and for several comparisons of at least two types of hypertensive patients, and
the method comprising at least the steps of:
a) measuring, ex vivo, a combination of biomarkers,
wherein for the plurality of hypertensive diseases according to (i-a), the combination of biomarkers is a combination of biomarkers (i-a) as above defined or defined elsewhere in the specification, or
wherein for the plurality of hypertensive diseases according to (i-b), the combination of biomarkers is a combination of biomarkers (i-b) as above defined or defined elsewhere in the specification, or
wherein for the plurality of hypertensive diseases according to (i-c), the combination of biomarkers is a combination of biomarkers (i-c) as above defined or defined elsewhere in the specification, or
wherein for the plurality of hypertensive diseases according to (i-d), the combination of biomarkers is a combination of biomarkers (i-d) as above defined or defined elsewhere in the specification, or
wherein for the plurality of hypertensive diseases according to (i-e), the combination of biomarkers is a combination of biomarkers (i-e) as above defined or defined elsewhere in the specification,
b) operating said trained classifier on the combination of biomarkers obtained at step a) from said hypertensive patient for obtaining a probability, for each hypertensive disease, of associating the hypertensive patient with a hypertensive disease in order to stratify said hypertensive patient among said plurality of types of hypertensive diseases according to (i-a) or according to (i-b), or according to (i-c), or according to (i-d), or according to (i-e).

The trained classifier may be selected from Decision Trees (J48), Naïve Bayes (NB), K-nearest neighbours (IBk), LogitBoost (LB), support vector machine (SVM), Logistic Model Tree (LMT), Bagging, Simple Logistic (SL), Random Forest (RF) and Sequential Minimal Optimisation (SMO).

The trained classifier may be selected from LogitBoost (LB), Simple Logistic (SL), and Random Forest (RF).

The classifier may have been trained with at least one predefined input dataset according to a method comprising at least the steps of:
a) for said at least one predefined input dataset and for at least one given comparison between at least a first and a second hypertensive patient, each patient having a first and a second type of hypertensive disease selected in said group of hypertensive diseases, the first and second type hypertensive disease being different, using said classifier to rank several combinations of biomarkers based on a computation of at least one evaluation parameter, and
b) based on said computed evaluation parameter(s), selecting a combination of biomarkers in order to stratify a hypertensive patient among said plurality of types of hypertensive diseases.

The evaluation parameter may be chosen among accuracy, sensitivity, specificity, AUC, F1, Kappa score, and combinations thereof.

According to another object, the disclosure relates to a kit for stratifying a hypertensive patient among a plurality of hypertensive diseases.

(i-a) The plurality of hypertensive diseases may comprise Primary Aldosteronism (PA), Pheochromocytoma/Functional Paraganglioma (PPGL), Cushing's Syndrome (CS) and Primary Hypertension (PHT), and the kit may comprise at least means for measuring a combination of biomarkers (i-a) as above defined or defined elsewhere in the specification.

Or, (i-b) the plurality of hypertensive diseases may comprise Endocrine Hypertension (EHT) and Primary Hypertension (PHT), and the kit may comprise at least means for measuring a combination of biomarkers (i-b) as above defined or defined elsewhere in the specification.

Or, (i-c) the plurality of hypertensive diseases comprising Cushing's Syndrome (CS) and Primary Hypertension (PHT), and the kit comprising at least means for measuring a combination of biomarkers (i-c) as above defined or defined elsewhere in the specification.

Or, (i-d) the plurality of hypertensive diseases comprising Primary Aldosteronism (PA) and Primary Hypertension (PHT), and the kit comprising at least means for measuring a combination of biomarkers (i-d) as above defined or defined elsewhere in the specification.

Or, (i-e) the plurality of hypertensive diseases comprising Pheochromocytoma/Functional Paraganglioma (PPGL) and Primary Hypertension (PHT), and the kit comprising at least means for measuring a combination of biomarkers (i-e) as above defined or defined elsewhere in the specification.

According to another object, the disclosure relates to a computer program product for stratifying a hypertensive patient among a plurality of hypertensive diseases.
(i-a) The plurality of hypertensive diseases comprising Primary Aldosteronism (PA), Pheochromocytoma/Functional Paraganglioma (PPGL), Cushing's Syndrome (CS) and Primary Hypertension (PHT), or (i-b) the plurality of hypertensive diseases comprising Endocrine Hypertension (EHT) and Primary Hypertension (PHT), or (i-c) the plurality of hypertensive diseases comprising Cushing's Syndrome (CS) and Primary Hypertension (PHT), or (i-d) the plurality of hypertensive diseases comprising Primary Aldosteronism (PA) and Primary Hypertension (PHT), or (i-e) the plurality of hypertensive diseases comprising Pheochromocytoma/Functional Paraganglioma (PPGL) and Primary Hypertension (PHT).

The computer program may use
(1) at least one classifier trained beforehand to learn a plurality of combinations of biomarkers previously selected based on at least one computed evaluation parameter and for several comparisons of at least a first and a second type of hypertensive disease selected in a group (i-a), (i-b), (i-c), (i-d) and (i-e), of hypertensive diseases comprising
(i-a) Primary Aldosteronism (PA), Pheochromocytoma/Functional Paraganglioma (PPGL), Cushing's Syndrome (CS) and Primary Hypertension (PHT), or
(i-b) Endocrine Hypertension (EHT) and Primary Hypertension (PHT), or
(i-c) Cushing's Syndrome (CS) and Primary Hypertension (PHT), or
(i-d) Primary Aldosteronism (PA) and Primary Hypertension (PHT), or
(i-e) Pheochromocytoma/Functional Paraganglioma (PPGL) and Primary Hypertension (PHT),
the first and second type hypertensive disease being different, wherein said classifier has been trained with at least one predefined input dataset according to a method comprising at least the steps of:
a) for said at least one predefined input dataset and for at least one given comparison between at least a first and a second hypertensive patient, each patient having a first and a second type of hypertensive disease selected in said group of hypertensive diseases (i-a), (i-b), (i-c), (i-d) and (i-e), the first and second type hypertensive disease being different, using said classifier to rank several combinations of biomarkers based on a computation of at least one evaluation parameter, and
b) based on said computed evaluation parameter(s), selecting a combination of biomarkers in order to stratify a hypertensive patient among said plurality of hypertensive diseases (i-a), (i-b), (i-c), (i-d) and (i-e),
(2) with at least one input of measured biomarkers, said input of measured biomarkers being obtained by measuring, in suitable biological samples previously isolated from said patient, a combination of biomarkers (i-a) as above defined or defined elsewhere in the specification wherein the plurality of hypertensive diseases is according to (i-a) or a combination of biomarkers (i-b) as above defined or defined elsewhere in the description wherein the plurality of hypertensive diseases is according to (i-b), or a combination of biomarkers (i-c) wherein the plurality of hypertensive diseases is according to (i-c), or a combination of biomarkers (i-d) wherein the plurality of hypertensive diseases is according to (i-d), or a combination of biomarkers (i-e) wherein the plurality of hypertensive diseases is according to (i-e),
the computer program product comprising instructions which, when the program is executed by a computer, cause the computer to, for said at least one input of measured biomarkers, use said trained classifier for associating each hypertensive disease of said plurality of hypertensive diseases (i-a), (i-b), (i-c), (i-d) or (i-e), with a probability of associating the hypertensive patient with said hypertensive disease in order to stratify said hypertensive patient among said plurality of hypertensive diseases (i-a), (i-b), (i-c), (i-d) and (i-e).

According to another object, the disclosure relates to a use of a combination of biomarkers in a method for stratifying and treating a hypertensive disease in a patient in need thereof,
the hypertensive disease being selected among
(i-a) Primary Aldosteronism (PA), Pheochromocytoma/Functional Paraganglioma (PPGL), Cushing's Syndrome (CS) and Primary Hypertension (PHT), and the combination of biomarkers being a combination of biomarkers (i-a) as above defined or defined elsewhere in the description, or
(i-b) Endocrine Hypertension (EHT) and Primary Hypertension (PHT), and the combination of biomarkers being a combination of biomarkers (i-b) as above defined or defined elsewhere in the description, or
(i-c) the plurality of hypertensive diseases comprising Cushing's Syndrome (CS) and Primary Hypertension (PHT), and the combination of biomarkers being a combination of biomarkers (i-c) as above defined or defined elsewhere in the description, or
(i-d) the plurality of hypertensive diseases comprising Primary Aldosteronism (PA) and Primary Hypertension (PHT), and the combination of biomarkers being a combination of biomarkers (i-d) as above defined or defined elsewhere in the description, or
(i-e) the plurality of hypertensive diseases comprising Pheochromocytoma/Functional Paraganglioma (PPGL) and Primary Hypertension (PHT), and the combination of biomarkers being a combination of biomarkers (i-e) as above defined or defined elsewhere in the description.

According to another object, the disclosure relates to a combination of biomarkers for use in a method for treating a hypertensive disease in a patient in need thereof,
the hypertensive disease being selected among
(i-a) Primary (PA), Pheochromocytoma/Functional Paraganglioma (PPGL), Cushing's Syndrome (CS) and Primary Hypertension (PHT), and the combination of biomarkers being a combination of biomarkers (i-a) as above defined or defined elsewhere in the specification, or
(i-b) Endocrine Hypertension (EHT) and Primary Hypertension (PHT), and the combination of biomarkers being a combination of biomarkers (i-b) as above defined or defined elsewhere in the specification, or
(i-c) Cushing's Syndrome (CS) and Primary Hypertension (PHT), and the combination of biomarkers being a combination of biomarkers (i-c) as above defined or defined elsewhere in the specification, or
(i-d) Primary Aldosteronism (PA) and Primary Hypertension (PHT), and the combination of biomarkers being a combination of biomarkers (i-d) as above defined or defined elsewhere in the specification, or
(i-e) Pheochromocytoma/Functional Paraganglioma (PPGL) and Primary Hypertension (PHT), and the combination of biomarkers being a combination of biomarkers (i-e) as above defined or defined elsewhere in the specification,
the method of treating comprising a step of stratifying the hypertensive patient among the hypertensive diseases of (i-a) or (i-b), said step of stratifying comprising:
measuring, ex vivo, said combination of biomarkers, and
operating a trained classifier on said measured combination of biomarkers for obtaining a probability, for each hypertensive disease, of associating the hypertensive patient with a hypertensive disease in order to stratify said hypertensive patient among said plurality of types of hypertensive diseases according to (i-a) or according to (i-b),
said trained classifier being a classifier trained beforehand to learn a plurality of combinations of biomarkers previously selected based on at least one computed evaluation parameter and for several comparisons of at least two types of hypertensive patients.

According to another object, the disclosure relates to a method for stratifying and treating a hypertensive patient, said method comprising stratifying said hypertensive patient among a plurality of hypertensive diseases,
(i-a) the plurality of hypertensive diseases comprising Primary Aldosteronism (PA), Pheochromocytoma/Functional Paraganglioma (PPGL), Cushing's Syndrome (CS) and Primary Hypertension (PHT), and the combination of biomarkers being a combination of biomarkers (i-a) as above defined or defined elsewhere in the description, or
(i-b) the plurality of hypertensive diseases comprising Endocrine Hypertension (EHT) and Primary Hypertension (PHT), and the combination of biomarkers being a combination of biomarkers (i-b) as above defined or defined elsewhere in the description, or
(i-c) the plurality of hypertensive diseases comprising Cushing's Syndrome (CS) and Primary Hypertension (PHT), and the combination of biomarkers being a combination of biomarkers (i-c) as above defined or defined elsewhere in the description, or
(i-d) the plurality of hypertensive diseases comprising Primary Aldosteronism (PA) and Primary Hypertension (PHT), and the combination of biomarkers being a combination of biomarkers (i-d) as above defined or defined elsewhere in the description, or
(i-e) the plurality of hypertensive diseases comprising Pheochromocytoma/Functional Paraganglioma (PPGL) and Primary Hypertension (PHT), and the combination of biomarkers being a combination of biomarkers (i-e) as above defined or defined elsewhere in the description.
and treating said patient with a therapeutic treatment presumed to treat or to relieve at least one symptom of the hypertensive disease, the method comprising at least the steps of:
a) stratifying said hypertensive patient among said plurality of hypertensive diseases (i-a), (i-b), (i-c), (i-d) and (i-e), according to the method of the disclosure, for associating a hypertensive disease to said patient,
b) selecting a therapeutic treatment presumed to treat or to relieve at least one symptom of the hypertensive disease associated to said patient, and
c) administering to said patient said therapeutic treatment selected at step b).

According to another object, the disclosure relates to a method for treating a hypertensive patient, said method comprising:
a) stratifying said hypertensive patient among a plurality of hypertensive diseases,
(i-a) the plurality of hypertensive diseases comprising Primary Aldosteronism (PA), Pheochromocytoma/Functional Paraganglioma (PPGL), Cushing's Syndrome (CS) and Primary Hypertension (PHT), and the combination of biomarkers being a combination of biomarkers (i-a) as above defined or defined elsewhere in the specification, or
(i-b) the plurality of hypertensive diseases comprising Endocrine Hypertension (EHT) and Primary Hypertension (PHT), and the combination of biomarkers being a combination of biomarkers (i-b) as above defined or defined elsewhere in the specification, or
(i-c) the plurality of hypertensive diseases comprising Cushing's Syndrome (CS) and Primary Hypertension (PHT), and the combination of biomarkers being a combination of biomarkers (i-c) as above defined or defined elsewhere in the description, or
(i-d) the plurality of hypertensive diseases comprising Primary Aldosteronism (PA) and Primary Hypertension (PHT), and the combination of biomarkers being a combination of biomarkers (i-d) as above defined or defined elsewhere in the description, or
(i-e) the plurality of hypertensive diseases comprising Pheochromocytoma/Functional Paraganglioma (PPGL) and Primary Hypertension (PHT), and the combination of biomarkers being a combination of biomarkers (i-e) as above defined or defined elsewhere in the description,
the stratification of said hypertensive patient among said plurality of hypertensive diseases (i-a), (i-b), (i-c), (i-d) and (i-e), being carried out according to the method as above defined or defined elsewhere in the specification, for associating a hypertensive disease to said patient,
b) selecting a therapeutic treatment presumed to treat or to relieve at least one symptom of the hypertensive disease associated to said patient, and
c) treating said patient by administering to said patient said therapeutic treatment selected at step b).

### DESCRIPTION OF THE FIGURES

FIG. 1: shows summary of samples and omics availability.

FIG. 2: shows the table of the tested plasma metanephrines.

FIG. 3: shows the table of the tested plasma steroids.

FIG. 4: shows the table of the tested urinary steroids.

FIG. 5: shows the table of the tested plasma small metabolites, ratio and combination thereof.

FIG. 6: shows the table of the tested plasma miRNA.

FIG. 7: shows block diagrams of some steps of a second example of the method according to the invention.

FIG. 8: shows details of randomly partitioned training and testing datasets with Cushing's syndrome (CS), primary aldosteronism (PA), pheochromocytoma or paraganglioma (PPGL) and primary hypertension (PHT).

FIG. 9: shows classification results for evaluating best classifier and best feature selection method on ALL-ALL disease combination using training set of multi-omics data.

FIG. 10: shows the classification metrics of top-performing classifiers on the test set of 5 disease combinations trained using multi-omics and 5 mono-omics.

FIG. 11: shows the prediction performance of top-performing classifiers (on test set) for ALL-ALL, EHT-PHT, PA-PHT, PPGL-PHT and CS-PHT combinations.

FIG. 12: shows confusion matrices of top performing classifiers on test set for ALL-ALL, EHT-PHT, PA-PHT, PPGL-PHT and CS-PHT disease combinations,

FIG. 13: shows ROC curves for EHT-PHT, PA-PHT, PPGL-PHT and CS-PHT with the top-performing classifiers and their respective AUC values.

FIG. 14: shows classification results for training and testing set using top performing classifiers trained with and without balanced data for ALL-ALL disease combination.

FIG. 15: shows classification results for training and testing set using top performing classifiers trained with and without balanced data for EHT-PHT disease combination.

FIG. 16: shows classification results for training and testing set using top performing classifiers trained for PA-PHT disease combination. The training dataset was balanced, therefore no synthetic samples or down-sampling approach was used.

FIG. 17: shows classification results for training and testing set using top performing classifiers trained with and without balanced data for PPGL-PHT disease combination.

FIG. 18: shows classification results for training and testing set using top performing classifiers trained with and without balanced data for CS-PHT disease combination.

FIG. 19: shows the count and percentage contribution of different omics in the whole multi-omics dataset.

FIG. 20: shows count and percentage contribution of selected features for multi-omics classification within each of 5 disease combinations.

FIG. 21: shows top common features amongst disease combinations for multi-omics as Venn diagram.

FIG. 22: shows a table listing the details of unique biomarkers with overlapping disease combinations.

FIG. 23: shows top common features amongst multi-omics and mono-omics for ALL-ALL disease combinations.

FIG. 24: shows top common features amongst multi-omics and mono-omics for EHT-PHT disease combinations.

FIG. 25: shows top common features amongst multi-omics and mono-omics for PA-PHT disease combinations.

FIG. 26: shows top common features amongst multi-omics and mono-omics for PPGL-PHT disease combinations.

FIG. 27: shows top common features amongst multi-omics and mono-omics for CS-PHT disease combinations.

FIG. 28: shows violin plots of most discriminating PmiRNA, PMetas, PSteroids and USteroids features selected for ALL-ALL disease combination in multi-omics classifier and corresponding values for NV.

FIG. 29: shows principal component analysis using top features of training data for ALL-ALL, EHT-PHT, PA-PHT, PPGL-PHT and CS-PHT disease combination along with NV samples.

FIG. 30: shows heatmap with mean classification performance metrics (over 100 random repeats) using multi-omics and 5 individual omics with 3 best classifiers for 5 disease combinations in Scenario 1 (Set A & B), 2 (Set C & D) and 3 (Set E & F).

FIG. 31: shows joint heatmap for 5 disease combinations showing the list of top features truncated with repeat frequency cutoff value of 50 (over 100 random repeats) selected during the classification using PMetas, PSteroids, USteroids, PmiRNA and PSmallMB data for Set A-F.

FIG. 32: shows Joint heatmap for 5 disease combinations showing the list of top features truncated with repeat frequency cutoff value of 50 (over 100 random repeats) selected during the classification using MOmics data for Set A-F.

FIG. 33: Schematic showing ML pipeline within the WP1 and the outcome probabilities for the validation set.

FIG. 34: Map showing the multi-omics features used by top-performing models for the (5) ALL-ALL, EHT-PHT, PA-PHT, PPGL-PHT and CS-PHT disease combinations.

### DETAILED DESCRIPTION

### Definitions

Unless otherwise defined herein, scientific and technical terms used in connection with the present disclosure shall have the meanings that are commonly understood by those of ordinary skill in the art. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, may provide one of skill with a general dictionary of many of the terms used in this disclosure. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Methods are performed according to kits manufacturer's specifications, as commonly accomplished in the art or as described herein. Further, unless otherwise required by context, singular terms shall include pluralities and plural terms shall include the singular.

Units, prefixes, and symbols are denoted in their Système International des Unités (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

All publications and other references mentioned herein are incorporated by reference in their entirety. Although a number of documents are cited herein, this citation does not constitute an admission that any of these documents forms part of the common general knowledge in the art.

It is to be noted that as used herein and in the appended claims, the singular forms “a”, “an”, and “the” include plural referents unless the context clearly dictates otherwise. Thus, for example, reference to “a biomarker” includes a plurality of such biomarkers, and so forth.

It is understood that aspects and embodiments of the present disclosure described herein include “having,” “comprising,” “consisting in,” and “consisting essentially of” aspects and embodiments. The words “have” and “comprise,” or variations such as “has,” “having,” “comprises,” or “comprising,” will be understood to imply the inclusion of the stated element(s) (such as a composition of matter or a method step) but not the exclusion of any other elements. The term “consisting in” implies the inclusion of the stated element(s), to the exclusion of any additional elements. The term “consisting essentially of” implies the inclusion of the stated elements, and possibly other element(s) where the other element(s) do not materially affect the basic and novel characteristic(s) of the disclosure. It is understood that the different embodiments of the disclosure using the term “comprising” or equivalent cover the embodiments where this term is replaced with “consisting in” or “consisting essentially of”.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%.

Within the disclosure, the term ‘significantly” used with respect to change intends to mean that the observed change is noticeable and/or it has a statistic meaning.

Within the disclosure, the term “substantially” used in conjunction with a feature of the disclosure intends to define a set of embodiments related to this feature which are largely but not wholly similar to this feature.

As used herein, the terms “stratifying” or “stratification” used with regard to a patient intends to refer to a process by which the patient is assigned a defined status or condition, as a specific hypertensive disease.

As used herein, the terms “patient”, “individual” or “subject” are used interchangeably and intends to refer to a human.

Within the meaning of the disclosure, “endocrine hypertension” (EHT) refers to a secondary-type of hypertension which is caused by an excessive hormone production from the adrenal gland. Endocrine hypertension includes primary aldosteronism (PA), due to autonomous production of aldosterone from an aldosterone-producing adenoma or bilateral adrenal hyperplasia, pheochromocytoma/functional paraganglioma (PPGL) due to excess production of catecholamines from the adrenal gland or a functional paraganglioma, and Cushing's syndrome (CS), due to autonomous production of cortisol due to an adrenal or pituitary tumor.

Within the meaning of the disclosure, “primary hypertension” (PHT) refers to a form of hypertension that by definition has no identifiable cause (not secondary to a phenomenon). It is the most common type of hypertension, affecting about 85-95% of hypertensive patients. It is likely to be the consequence of an interaction between genetic and environmental factors.

Within the meaning of the disclosure, “biomarker” intends to refer to a quantifiable biological characteristic that is objectively measured and evaluated as an indicator of normal or pathogenic biological processes, or of pharmacologic responses to a therapeutic intervention. It can be any substance, structure, or process that can be measured in the body and influence or predict the incidence of an outcome or a disease, the effect of a treatment or an intervention. A biomarker can be a biological molecule, such as, for example, a nucleic acid, a peptide, a protein, an hormone, and the like or can be physical parameter of the patient such as the age, height, weight, BMI, or sex.

A biomarker may be chosen at least among O-methylated catecholamines, miRNA, steroids, Small metabolites, patient's status, such as age, or gender. O-methylated catecholamines, miRNA and Small metabolites may be determined in plasma, steroids may be determined in plasma or urine.

As used herein, a “combination of biomarkers” intends to refer to a set of biomarkers measured in suitable biological samples previously taken from a patient and which, taken together, may be used as a molecular signature of the hypertensive disease affecting the patient.

The terms “measure”, “measuring”, “measured”, or any equivalent thereof used within the disclosure in relation with the biomarkers intend to mean the quantification or qualification of the biomarkers.

The quantification is a measure of a quantity of a biomarker which may be expressed in volume, in mole, in weight, in weight by weight or by volume of the matrix containing the biomarker, such as a concentration, in particular a molar concentration. For example, a quantification of a biomarker may be expressed in ng/ml or pg/ml. Also, the quantification of a biomarker may be expressed relatively to the quantification of another biomarker or to a reference (or standard). In such case, the quantification of the concerned biomarkers may be expressed as a ratio, such as a weight:weight ratio or a molar ratio. The numerical value of the age of a patient is a quantification of the biomarker “age” taken as a biomarker of a patient's status.

The qualification of a biomarker is the determination of the presence or absence of the concerned biomarker. It may also be a non-numerical value of the status of a patient, such as sex (male/female) or menopause status (pre/post-menopause).

The determination, such as quantification or qualification, of a biomarker may be carried out by any known techniques in the art applicable to the concerned biomarker.

Measuring ex vivo a combination of biomarkers. Within the disclosure, the expression “measuring ex vivo a combination of biomarkers” intends to refer to a step carried outside the body of the patient, for instance on suitable biological samples previously isolated from the patient. In case of patient's age, the measure may be determined on the basis of the birthdate of the patient or of a previously obtained measure of the bone density. “Measuring ex vivo a combination of biomarkers” is used interchangeably with the expression “measuring, in suitable biological samples previously isolated from said patient, a combination of biomarkers”. For sake of conciseness, “measuring, in suitable biological samples previously isolated from said patient, a combination of biomarkers” may, depending on the context, include the determination of the age of the patient even if this later is per se carried out on an isolated biological sample but is carried out on the basis of the birthdate of the patient or of a previously obtained measure of the bone density.

Within the meaning of the disclosure, the terms “omic” or “omics” refer to the characterization and quantification of pools of biological molecules that translate into the structure, function, and dynamics of an organism or organisms. That covers fields such as genomics, transcriptomic, proteomics or metabolomics.

“Small metabolite” as used herein intends to refer to a wide range of low molecular weight organic compounds, of molecular weight ranging from about 50 to about 1500 daltons (Da), involved in a biological process as a substrate or product. Metabolites are the products and intermediates of cellular metabolism. A Human Metabolome Database is accessible at https://hmdb.ca/. Metabolites can have a multitude of functions, including energy conversion, signaling, epigenetic influence, and cofactor activity. “Metabolites” or “Small metabolites” are subject-matter of studies by metabolomics. Metabolomics is the study of metabolite profiles. It utilizes mass spectrometry methods or nuclear magnetic resonance spectrometry to analyze many different metabolites in a biologic sample. Metabolites or Small metabolites include, for example, uric acid, lipids and derivatives, such as palmitoleic acid (c16:1), palmitic acid (c16:0), 1,2-diglyceride (c36:2), amino acids and derivatives such as proline, isoleucine, or as 4-hydroxyproline, sugars and derivatives such as arabitol, ribitol, or xylitol, mannose or galactose, etc. Small metabolites are typically used as biomarkers of biological processes. “Small metabolites” are well known in the art as illustrated by Qiu S, Cai Y, Yao H, et al. Small molecule metabolites: discovery of biomarkers and therapeutic targets. Signal Transduct Target Ther. 2023; 8 (1): 132. Published 2023 Mar. 20. doi: 10.1038/s41392-023-01399-3 or Töpfer N, Kleessen S, Nikoloski Z. Integration of metabolomics data into metabolic networks. Front Plant Sci. 2015; 6:49. Published 2015 Feb. 17. Doi: 10.3389/fpls.2015.00049.

As used herein “metabolites” or “Small metabolites” do not include O-methylated catecholamines and steroids (or even miRNA) which are determined elsewhere.

Within the meaning of the disclosure, a “type of hypertensive patient” intends to refer to a patient suffering from EHT or PHT, or from PA, CS or PPGL. Hereafter, for example, a hypertensive patient suffering from EHT will be referred to a EHT patient.

A “type of hypertensive disease” refers to a hypertensive selected among EHT or PHT, or from PA, CS or PPGL.

As used herein, the terms “prevent”, “preventing” or “delay progression of” (and grammatical variants thereof) with respect to a disease or disorder relate to prophylactic treatment of the disease or the disorder, e.g., in a patient suspected to have the disease, or at risk for developing the disease. Prevention may include, but is not limited to, preventing or delaying onset or progression of the disease and/or maintaining one or more symptoms of the disease or disorder at a desired or sub-pathological level. The term “prevent” does not require the 100% elimination of the possibility or likelihood of occurrence of the event. Rather, it denotes that the likelihood of the occurrence of the event has been reduced in the presence of a composition or method as described herein.

As used herein, in the context of an immune response elicitation, the terms “treat”, “treatment”, “therapy” and the like refer to the administration or consumption of a composition as disclosed herein with the purpose to cure, heal, alleviate, relieve, alter, remedy, ameliorate, improve, or affect a disease or a disorder, the symptoms of the condition, or to prevent or delay the onset of the symptoms, complications, or otherwise arrest or inhibit further development of the disorder in a statistically significant manner. Also, as used herein, in the context of the present disclosure, the terms “treat”, “treatment” and the like refer to relief from or alleviation of pathological processes of a disorder. In the context of the present disclosure, insofar as it relates to any of the other conditions recited herein, the terms “treat”, “treatment”, and the like refer to relieving or alleviating one or more symptoms associated with such condition.

Within the meaning of the disclosure, “anti-hypertensive therapeutic treatment” intends to refer to any drug or intervention intended to provide a therapeutic effect, and which can be used to prevent and/or treat a hypertensive disease in a patient in need thereof. Those agents and interventions and their use according to the specifics of the patient are well within the common practice and general knowledge of the skilled person in the art. The nature of the anti-hypertensive agent or intervention may be selected according to the type of hypertensive patient to be treated. Hence, an anti-hypertensive agent or intervention may not be used similarly for the treatment of a EHT patient or a PHT patient, or for a PA patient, a PPGL patient or a CS patient.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure. All publications mentioned herein are incorporated herein by reference to disclose and describe the methods and/or materials in connection with which the publications are cited.

The list of sources, ingredients, and components as described hereinafter are listed such that combinations and mixtures thereof are also contemplated and within the scope herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All lists of items, such as, for example, lists of ingredients, are intended to and should be interpreted as Markush groups. Thus, all lists can be read and interpreted as items “selected from the group consisting of’ the list of items “and combinations and mixtures thereof.”

Referenced herein may be trade names for components including various ingredients utilized in the present disclosure. The inventors herein do not intend to be limited by materials under any particular trade name. Equivalent materials (e.g., those obtained from a different source under a different name or reference number) to those referenced by trade name may be substituted and utilized in the descriptions herein.

In the description of the various embodiments of the present disclosure, various embodiments or individual features are disclosed. As will be apparent to the ordinarily skilled practitioner, all combinations of such embodiments and features are possible and can result in preferred executions of the present disclosure. While various embodiments and individual features of the present invention have been illustrated and described, various other changes and modifications can be made without departing from the spirit and scope of the invention. As will also be apparent, all combinations of the embodiments and features taught in the present disclosure are possible and can result in preferred executions of the invention.

### Biomarkers and Combinations Thereof.

The disclosure relates to combinations of biomarkers. The combinations of biomarkers may be used for stratifying a hypertensive patient among different types of hypertensive diseases, such as EHT, PHT, PA, CS and PPGL.

The combinations of biomarkers may comprise at least one biomarker selected in each group of biomarkers of a set of at least two groups of biomarkers. The at least two groups of biomarkers are selected among: Patient's age; miRNAs; O-methylated catecholamines; Steroids; and Small metabolites.

The biomarkers miRNAs, O-methylated catecholamines, steroids, and Small metabolites may be determined in one or more isolated biological samples taken from the patient. The determination of the biomarkers is made in vitro.

The uses and methods disclosed herein may be in vitro uses and methods.

Suitable biological samples obtained from a patient may be a blood, urine, fecal, sweat, saliva, or tissue samples, such as sample of skin. When a blood sample is taken for analysis, whole blood, serum or plasma sample may be used.

Suitable isolated biological samples may be plasma and/or urinary samples.

Depending on the combination of biomarkers, more than one biological sample may be used. For example, a plurality of samples of same nature may be used, such as a plurality of blood samples. Also, a plurality of samples of different nature may be used, such as a blood sample and a urinary sample, or a plurality of blood samples and a plurality of urinary samples.

Determination of a biomarker or a combination of biomarker in a sample may be made by several possible analytical methodologies. The choice of the analytical method depends on the nature of the biomarker(s) to be analyzed and may be selected by a skilled person upon his or her general knowledge.

Suitable analytical methods may be, for example, mass spectrometry linked to a pre-separation step such as chromatography, immuno-detection, in particular a quantitative immunoassay such as a Western blot or ELISA, multi-analyte biochip, Biochip Array Technology system (BAT), PCRs, multiplexed-PCRs, RT-PCRs, nucleic acids-based chips or micro-arrays, HPLC coupled with coulometric detection or Liquid chromatography-tandem mass spectrometry, as chromatography/mass spectrometry, UHPLC-ESI-QTOF-MS/MS, or LC-MS/MS, NMR, LC/GC-FID, Direct Flow Injection MS/MS, LC ESI-MS/MS, MS/MS, Isothermal amplification, Next-generation sequencing, Hybridization chain reaction, or Near-infrared technology.

For example, steroids profiling may be carried out with ultra-high performance liquid chromatography-tandem mass spectrometry (uHPLC-MS/MS, or for short LC-MS/MS), gas chromatography-mass spectrometry (GC-MS), or supercritical fluid chromatography-tandem mass spectrometry (SFC-MS/MS).

### Age

A biomarker usable in a combination of biomarkers disclosed herein may be the age of the patient.

The age of a patient is a clinical parameter of a patient. This parameter may be determined based on the birthdate of the patient or bone density.

A combination of biomarkers for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may comprise at least Patient's age.

A combination of biomarkers for stratifying a hypertensive patient among a plurality of types of hypertensive diseases, the plurality of types of hypertensive diseases comprising Endocrine Hypertension (EHT) and Primary Hypertension (PHT), may not comprise Patient's age. Alternatively, a combination of biomarkers for stratifying a hypertensive patient among a plurality of types of hypertensive diseases, the plurality of types of hypertensive diseases comprising Endocrine Hypertension (EHT) and Primary Hypertension (PHT), may comprise Patient's age.

A combination of biomarkers for stratifying a hypertensive patient among a plurality of types of hypertensive diseases, the plurality of types of hypertensive diseases comprising Cushing's Syndrome (CS) and Primary Hypertension (PHT), may not comprise Patient's age.

A combination of biomarkers for stratifying a hypertensive patient among a plurality of types of hypertensive diseases, the plurality of types of hypertensive diseases comprising Primary Aldosteronism (PA) and Primary Hypertension (PHT), may comprise at least Patient's age.

A combination of biomarkers for stratifying a hypertensive patient among a plurality of types of hypertensive diseases, the plurality of types of hypertensive diseases comprising Pheochromocytoma/Functional Paraganglioma (PPGL) and Primary Hypertension (PHT), may not comprise Patient's age. Alternatively, a combination of biomarkers for stratifying a hypertensive patient among a plurality of types of hypertensive diseases, the plurality of types of hypertensive diseases comprising Pheochromocytoma/Functional Paraganglioma (PPGL) and Primary Hypertension (PHT), may comprise Patient's age.

### Omics Biomarkers

A biomarker to be used in a combination of biomarkers as disclosed herein may be an omic biomarker. An omic biomarker may be a metabolomic or a genomic-based biomarker. An omic biomarker may be a O-methylated catecholamine, a steroid, a small metabolite, or a miRNA.

In embodiments, an omic biomarker may be determined in a plasma or in a urinary sample.

O-methylated catecholamines, Small metabolites, miRNA may be determined in plasma samples.

Steroids may be determined in plasma and/or urinary samples.

O-methylated catecholamines, steroids, and Small metabolites are metabolomics-based biomarkers. Metabolomics is the study of endogenous and exogenous small (typically 50-1500 Da) molecules comprising the substrates and products of metabolic processes. The metabolome is the aggregate of all metabolites in a biological system. Examples of such metabolites include amino and fatty acids, lipids, sugars, and phenolic compounds. The Human Metabolome Database 46 currently contains >114 100 entries. Like the transcriptome and proteome, the metabolome is cell and tissue-specific. Several analytic laboratory approaches are used to characterize the metabolome, including mass spectrometry and nuclear magnetic resonance spectroscopy. Some methods affect chemical separation (e.g., by gas chromatography or high-performance liquid chromatography) before detection, whereas others do not (shotgun metabolomics). Global (or untargeted) metabolomics methods can provide data on 1000+ metabolites, whereas targeted methods typically assay a particular class of molecules (e.g., lipids) (Arnett et al., Circ Res. 2018; 122 (10): 1409-1419.).

miRNAs

A biomarker to be used within a combination of biomarkers of the disclosure may be a miRNA biomarker.

Plasma miRNAs are transcriptomic-based biomarkers. The transcriptomics seeks to identify and quantify all RNA transcripts (potentially including messenger, transfer, ribosomal, and noncoding regulatory RNAs) produced by a cell or organism under specific conditions (Arnett et al., 2018). Transcriptomics provides a snapshot of which genes are actively being expressed by a cell or tissue at a given time. The two commonly used transcriptomic laboratory methods are microarrays and RNA-Seq.

The transcriptome varies among tissues from the same organism. Choice of sample matrix depends on factors such as accessibility and study objectives. Typically, transcriptomic studies compare gene expression profiles under ≥2 different experimental conditions, such as different environmental exposures or different disease states. There are several ways to analyze transcriptomic data. Heat maps offer a simple way to visually display differences in expression between experimental conditions. Gene co-expression network analysis can characterize regulatory programs and associate genes of unknown function with metabolic processes. Pathway analysis uses information cataloged in functional gene annotation databases to identify metabolic, signaling, and gene regulatory pathways that may be in play with a given gene expression pattern.

One way to analyze transcriptomic biomarkers, and in particular miRNA, is heat map.

The plasma miRNAs may be determined or quantified in a plasma sample isolated from a patient.

Before determination, miRNA may be extracted from biological samples, and in particular from plasma samples according to the method described in Sourvinou et al. (Journal of Molecular Diagnostics, Vol. 15, No. 6, November 2013 or Moody et al. Clin Epigenetics. 2017 Oct. 24; 9:119)

Determination or quantification of miRNA, in particular plasma miRNA, may be carried out according to any known techniques in the art. For example, a useful analytical method may be digital PCR, quantitative RT-PCR, Microarray, Isothermal amplification, Next-generation sequencing, Hybridization chain reaction, or Near-infrared technology (Sourvinou et al., Journal of Molecular Diagnostics, Vol. 15, No. 6, November 2013; Ma, Jie et al., Biomarker insights vol. 8 127-36. 14 Nov. 2013; Moody et al. Clin Epigenetics. 2017 Oct. 24; 9:119; Wright et al. Sci Rep 10, 825 (2020)).

In some embodiments, the miRNAs may be determined using real-time RT-PCR methodology, which can generate a cycle threshold (Ct) value for each miRNA which can been calibrated and normalised. That allows cross-plate and cross-sample comparison. The Ct is a relative value inversely proportional to transcript quantity and is influenced by various factors including the detection chemistry and the combination of normalising/calibrating miRNAs employed. One way to normalize the Ct relies upon the number of spikes-in used in the controls and upon endogenous miRNAs.

Amounts of miRNA may be expressed in numbers of copies of miRNA per volume unit of sample, usually μL, or in weight of miRNA per volume unit of sample, such as ng/μL.

miRNA biomarkers considered in the methods disclosed herein are known in the art, and further information may be obtained from miRBase: the microRNA database (http://www.mirbase.org/index.shtml).

miRNA biomarkers suitable for the disclosure may be selected from hsa-let-7b-5p, hsa-let-7d-3p, hsa-let-7d-5p, hsa-let-7g-5p, hsa-miR-103a-3p, hsa-miR-106b-3p, hsa-miR-107, hsa-miR-130a-3p, hsa-miR-130b-3p, hsa-miR-140-5p, hsa-miR-144-3p, hsa-miR-146a-5p, hsa-miR-148b-3p, hsa-miR-150-5p, hsa-miR-151a-3p, hsa-miR-152-3p, hsa-miR-155-5p, hsa-miR-15a-5p, hsa-miR-15b-3p, hsa-miR-16-2-3p, hsa-miR-16-5p, hsa-miR-181a-5p, hsa-miR-195-5p, hsa-miR-199a-5p, hsa-miR-19a-3p, hsa-miR-19b-3p, hsa-miR-210-3p, hsa-miR-222-3p, hsa-miR-22-3p, hsa-miR-23a-3p, hsa-miR-23b-3p, hsa-miR-24-3p, hsa-miR-25-3p, hsa-miR-26b-5p, hsa-miR-27a-3p, hsa-miR-27b-3p, hsa-miR-301a-3p, hsa-miR-30c-5p, hsa-miR-30d-5p, hsa-miR-324-5p, hsa-miR-32-5p, hsa-miR-328-3p, hsa-miR-335-5p, hsa-miR-33a-5p, hsa-miR-342-3p, hsa-miR-363-3p, hsa-miR-423-5p, hsa-miR-451a, hsa-miR-485-3p, hsa-miR-486-5p, hsa-miR-495-3p, hsa-miR-497-5p, hsa-miR-502-3p, hsa-miR-629-5p, and hsa-miR-92a-3p.

A miRNA biomarker suitable for the disclosure may be selected from plasma hsa-let-7g-5p, plasma hsa-miR-106b-3p, plasma hsa-miR-301a-3p, plasma hsa-miR-485-3p, plasma hsa-miR-19a-3p, and combinations thereof.

A miRNA biomarker suitable for the disclosure may be selected from plasma hsa-let-7g-5p, plasma hsa-miR-106b-3p, plasma hsa-miR-301a-3p, plasma hsa-miR-485-3p, and combinations thereof.

A miRNA biomarker suitable for the disclosure may be plasma hsa-miR-106b-3p.

A miRNA biomarker suitable for the disclosure may be plasma hsa-let-7g-5p.

A miRNA biomarker suitable for the disclosure may be plasma hsa-miR-19a-3p.

A combination of miRNA biomarkers suitable for the disclosure may comprise at least or consist in plasma hsa-miR-106b-3p. The combination may further comprise plasma hsa-let-7g-5p. The combination may also further comprise plasma hsa-miR-301a-3p, and plasma hsa-miR-485-3p.

A combination of miRNA biomarkers suitable for the disclosure may comprise at least or consist in a combination of plasma hsa-let-7g-5p, plasma hsa-miR-106b-3p, plasma hsa-miR-301a-3p, and plasma hsa-miR-485-3p.

A combination of miRNA biomarkers for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may comprise at least, or may consist in, plasma hsa-miR-106b-3p. The combination may further comprise plasma hsa-let-7g-5p. The combination may also further comprise plasma hsa-miR-301a-3p, and plasma hsa-miR-485-3p.

A combination of miRNA biomarkers for stratifying a hypertensive patient among a plurality of types of hypertensive diseases, the plurality of types of hypertensive diseases comprising Endocrine Hypertension (EHT) and Primary Hypertension (PHT), may comprise at least, or may consist in, plasma hsa-let-7g-5p.

A combination of miRNA biomarkers for stratifying a hypertensive patient among a plurality of types of hypertensive diseases, the plurality of types of hypertensive diseases comprising Cushing's Syndrome (CS) and Primary Hypertension (PHT), may comprise at least, or may consist in, plasma hsa-miR-19a-3p.

A combination of biomarkers for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may not comprise any miRNA biomarkers.

A combination of biomarkers for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may comprise, as miRNA biomarker, at least plasma hsa-miR-106b-3p. The combination may further comprise plasma hsa-let-7g-5p. The combination may also further comprise plasma hsa-miR-301a-3p, and plasma hsa-miR-485-3p.

A combination of biomarkers for stratifying a hypertensive patient among a plurality of types of hypertensive diseases, the plurality of types of hypertensive diseases comprising Endocrine Hypertension (EHT) and Primary Hypertension (PHT), may not comprise any miRNA.

A combination of biomarkers for stratifying a hypertensive patient among a plurality of types of hypertensive diseases, the plurality of types of hypertensive diseases comprising Endocrine Hypertension (EHT) and Primary Hypertension (PHT), may comprise, as miRNA biomarker, at least plasma hsa-let-7g-5p.

A combination of biomarkers for stratifying a hypertensive patient among a plurality of types of hypertensive diseases, the plurality of types of hypertensive diseases comprising Cushing's Syndrome (CS) and Primary Hypertension (PHT), may not comprise any miRNA.

A combination of biomarkers for stratifying a hypertensive patient among a plurality of types of hypertensive diseases, the plurality of types of hypertensive diseases comprising Cushing's Syndrome (CS) and Primary Hypertension (PHT), may comprise, as miRNA biomarker, at least plasma hsa-miR-19a-3p.

A combination of biomarkers for stratifying a hypertensive patient among a plurality of types of hypertensive diseases, the plurality of types of hypertensive diseases comprising Primary Aldosteronism (PA) and Primary Hypertension (PHT), may not comprise any miRNA.

A combination of biomarkers for stratifying a hypertensive patient among a plurality of types of hypertensive diseases, the plurality of types of hypertensive diseases comprising Pheochromocytoma/Functional Paraganglioma (PPGL) and Primary Hypertension (PHT), may not comprise any miRNA.

### O-Methylated Catecholamines

A biomarker to be used within the disclosure may be a O-methylated catecholamine.

A O-methylated catecholamine is a metabolite resulting from the O-methylation of a catecholamine, e.g., dopamine, epinephrine, norepinephrine, by a catechol O-methyltransferase (COMT). As used herein O-methylated catecholamine intends also to cover 3-methyldopa.

A combination of biomarkers of the disclosure may comprise a O-methylated catecholamine or a combination of O-methylated catecholamines.

O-methylated catecholamines may be determined, and in particular may be quantified, in a plasma sample isolated from a patient.

Determination or quantification of O-methylated catecholamines may be carried out according to any known techniques in the art. For example, a useful analytical method may be HPLC coupled with coulometric detection or Liquid chromatography-tandem mass spectrometry (LC-MS/MS), which can be used for quantifying plasma O-methylated catecholamines (Niec et al., J Chromatogr B Analyt Technol Biomed Life Sci., 2015; Lee et al., Ann Lab Med. 2015 September; 35 (5): 519-522; Osinga et al., Clin Biochem. 2016 September; 49 (13-14): 983-988).

In some embodiments, determination or quantification of O-methylated catecholamines may be determined by ultraperformance liquid chromatography-tandem mass spectrometry as disclosed in Peitzsch et al. (Ann Clin Biochem. 2013 March; 50 (Pt 2): 147-55).

Amounts of a O-methylated catecholamine may be expressed in weight/volume unit of sample, such as ng/ml or pg/ml of plasma.

Interval for reference ranges of plasma O-methylated catecholamines may vary according to age and gender, as well as according to the used analytical method. Nonetheless, reference ranges are known in the art, as disclosed, for example, by Peitzsch et al., Ann Clin Biochem. 2013 March; 50 (Pt 2): 147-55 or by Eisenhofer et al., Ann Clin Biochem. 2013; 50 (Pt 1): 62-69.

A O-methylated catecholamine suitable for the disclosure may be a plasma O-methylated catecholamine selected from a group comprising at least or consisting in normetanephrine, metanephrine, 3-methoxytyramine, 3-O-methyldopa, and combinations thereof.

A plasma O-methylated catecholamine may be selected from a group comprising at least or consisting in normetanephrine, metanephrine, 3-methoxytyramine, and combinations thereof.

A plasma O-methylated catecholamine may be selected from a group comprising at least or consisting in normetanephrine, metanephrine, and combinations thereof.

A plasma O-methylated catecholamine may be normetanephrine.

A combination of plasma O-methylated catecholamines may comprise at least or consist in a combination of normetanephrine and metanephrine. The combination may further comprise at least 3-methoxytyramine.

A combination of plasma O-methylated catecholamines for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may comprise at least or consist in normetanephrine and metanephrine. The combination may further comprise 3-methoxytyramine.

A plasma O-methylated catecholamine for stratifying a hypertensive patient among a plurality of types of hypertensive diseases, the plurality of types of hypertensive diseases comprising Endocrine Hypertension (EHT) and Primary Hypertension (PHT), may be normetanephrine

A combination of plasma O-methylated catecholamines for stratifying a hypertensive patient among a plurality of types of hypertensive diseases, the plurality of types of hypertensive diseases comprising Pheochromocytoma/Functional Paraganglioma (PPGL) and Primary Hypertension (PHT), may comprise at least or consist in 3-methoxytyramine, normetanephrine and metanephrine.

A combination of biomarkers for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may not comprise any plasma O-methylated catecholamines.

A combination of biomarkers for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may comprise, as plasma O-methylated catecholamines, at least plasma normetanephrine and plasma metanephrine. The combination may further comprise plasma 3-methoxytyramine

A combination of biomarkers for stratifying a hypertensive patient among a plurality of types of hypertensive diseases, the plurality of types of hypertensive diseases comprising Endocrine Hypertension (EHT) and Primary Hypertension (PHT), may not comprise any plasma O-methylated catecholamines.

A combination of biomarkers for stratifying a hypertensive patient among a plurality of types of hypertensive diseases, the plurality of types of hypertensive diseases comprising Endocrine Hypertension (EHT) and Primary Hypertension (PHT), may comprise, as plasma O-methylated catecholamine, at least plasma normetanephrine.

A combination of biomarkers for stratifying a hypertensive patient among a plurality of types of hypertensive diseases, the plurality of types of hypertensive diseases comprising Cushing's Syndrome (CS) and Primary Hypertension (PHT), may not comprise any plasma O-methylated catecholamines.

A combination of biomarkers for stratifying a hypertensive patient among a plurality of types of hypertensive diseases, the plurality of types of hypertensive diseases comprising Primary Aldosteronism (PA) and Primary Hypertension (PHT), may not comprise any plasma O-methylated catecholamines.

A combination of biomarkers for stratifying a hypertensive patient among a plurality of types of hypertensive diseases, the plurality of types of hypertensive diseases comprising Pheochromocytoma/Functional Paraganglioma (PPGL) and Primary Hypertension (PHT), may comprise, as plasma O-methylated catecholamines, at least plasma 3-methoxytyramine, plasma metanephrine, and plasma normetanephrine.

### Steroids

A biomarker to be used within the disclosure may be a steroid. A steroid may be a plasma and/or a urinary steroid.

Steroids may be determined, and in particular may be quantified, in a plasma sample and/or a urinary sample isolated from a patient.

Determination or quantification of steroids may be carried out according to any known techniques in the art. For example, a useful analytical method may be ELISA, liquid column chromatography, gas chromatography/mass spectrometry, UHPLC-ESI-QTOF-MS/MS, or LC-MS/MS (Allende et al., Chromatographia 77, 637-642 (2014); van der Veenet et al., Clin Biochem. 2019; 68:15-23; and Renterghem et al., Journal of Chromatography B, Volume 1141, 2020, 122026; Andrew et al., Best Pract Res Clin Endocrinol Metab. 2001; 15 (1): 1-16).

In some embodiments, determination or quantification of steroids may be carried out by liquid chromatography coupled with tandem mass spectrometry (LC-MS/MS) as disclosed in (Eisenhofer et al., JAMA Netw Open. 2020 Sep. 1; 3 (9); Peitzsch et al., J Steroid Biochem Mol Biol. 2014 January; 145:75-84; or Bancos et al., Lancet Diabetes Endocrinol. 2020 September; 8 (9): 773-781).

In some embodiments, urinary steroids may be quantified in urine samples collected over 24 hours, timed collections for shorter periods than 24 hours, first morning urine or spontaneous urine collection.

Amounts of a steroid may be expressed in weight/volume unit of sample, such as mol/L, pmol/L, nmol/L, ng/ml, or pg/ml of plasma or urine, and in particular in ng/ml or mol/L.

Interval for reference ranges of plasma and urinary steroids may vary according to age and gender, as well as according to the used analytical method. Nonetheless, reference ranges are known in the art, as disclosed, for example, by Eisenhofer et al. (Clin Chim Acta. 2017 July; 470:115-124) or by Van Renterghem et al. (Steroids. 2010 February; 75 (2): 154-63).

A steroid may be determined in a plasma sample.

A steroid may be determined in a urinary sample.

A steroid suitable for the disclosure may be selected from plasma aldosterone, plasma androstenedione, plasma corticosterone, plasma cortisol, plasma cortisone, plasma dehydroepiandrosterone (DHEA), plasma dehydroepiandrosterone sulfate (DHEAS), plasma 11-deoxycorticosterone, plasma 11-deoxycortisol, plasma 17OH-progesterone, plasma 18OH-cortisol, plasma 18oxo-cortisol, plasma 21-deoxycortisol, plasma 18OH-corticosterone, plasma 11-dehydrocorticosterone, urinary α-cortol (Acortol), urinary α-cortolone (Acortolone), urinary androsterone (An), urinary β-cortol (Bcortol), urinary β-cortolone, urinary cortisol, urinary cortisone, urinary dehydroepiandrosterone (DHEA), urinary etiocholanolone (Etio), urinary pregnanediol (PD), urinary pregnenetriol (PT), urinary 3α,5β-tetrahydroaldosterone (THAldo), urinary tetrahydro-11-dehydrocorticosterone (THAs), urinary tetrahydrocorticosterone (THB), urinary tetrahydrodeoxycorticosterone (THDOC), urinary tetrahydrocortisone (THE), urinary tetrahydrocortisol (THF), urinary tetrahydro-11-deoxycortisol (THS), urinary 11-β-hydroxy-androsterone (11-β-OHAn), urinary 11-β-hydroxy-etiocholanolone (11-βOHEt), urinary 11-oxo-etiocholanolone (11OxoEt), urinary 17-OH-pregnanolone (17-HP), urinary 18-hydroxycortisol (18-OHF), urinary 5α-tetrahydrocorticosterone (5-αTHB), urinary 5α-tetrahydrocortisol (5αTHF), urinary 5-pregnanediol (PD), urinary 5-pregnenetriol (5-PT), and combinations thereof.

A steroid suitable for the disclosure may be selected from plasma 11-dehydrocorticosterone, plasma 11-deoxycorticosterone, plasma 11-deoxycortisol, plasma 18OH-corticosterone, plasma 18OH-cortisol, plasma 18oxo-cortisol, plasma 21-deoxycortisol, plasma aldosterone, plasma corticosterone, plasma cortisol, plasma cortisone, plasma dehydroepiandrosterone (DHEA), plasma dehydroepiandrosterone sulfate (DHEAS), urinary 11-β-hydroxy-androsterone (11-β-OHAn), urinary 17-OH-pregnanolone (17-HP), urinary 18-hydroxycortisol (18-OHF), urinary 5α-tetrahydrocortisol (5αTHF), urinary 5-pregnanediol (PD), urinary 5-pregnenetriol (5-PT), urinary α-cortol (Acortol), urinary α-cortolone (Acortolone), urinary androsterone (An), urinary β-cortol (Bcortol), urinary β-cortolone (Bcortolone), urinary cortisol, urinary cortisone, urinary dehydroepiandrosterone (DHEA), urinary etiocholanolone (Etio), urinary pregnanediol (PD), urinary pregnenetriol (PT), urinary 3α,5β-tetrahydroaldosterone (THAldo), urinary tetrahydro-11-dehydrocorticosterone (THAs), urinary tetrahydrocorticosterone (THB), urinary tetrahydrodeoxycorticosterone (THDOC), urinary tetrahydrocortisone (THE), urinary tetrahydrocortisol (THF), urinary tetrahydro-11-deoxycortisol (THS), and combinations thereof.

### Plasma Steroids

A plasma steroid may be selected from a group comprising at least or consisting in aldosterone, androstenedione, corticosterone, cortisol, cortisone, dehydroepiandrosterone (DHEA), dehydroepiandrosterone sulfate (DHEAS), 11-deoxycorticosterone, 11-deoxycortisol, 17OH-progesterone, 18OH-cortisol, 18oxo-cortisol, 21-deoxycortisol, 18OH-corticosterone, and combinations thereof.

A plasma steroid suitable for the disclosure may be selected from 11-dehydrocorticosterone, 11-deoxycorticosterone, 11-deoxycortisol, 18OH-corticosterone, 18OH-cortisol, 18oxo-cortisol, 21-deoxycortisol, aldosterone, corticosterone, cortisol, cortisone, dehydroepiandrosterone (DHEA), dehydroepiandrosterone sulfate (DHEAS).

A plasma steroid may be selected from a group comprising at least or consisting in 11-deoxycorticosterone, 11-deoxycortisol, 18OH-corticosterone, 18OH-cortisol, 18oxo-cortisol, 21-deoxycortisol, aldosterone, and combinations thereof. The group may further comprise corticosterone. The group may also further comprise dehydroepiandrosterone sulfate (DHEAS). The group may also further comprise 11-dehydrocorticosterone. The group may also further comprise cortisone. The group may also further comprise cortisol.

A plasma steroid may be selected from a group comprising at least or consisting in 11-deoxycorticosterone, 11-deoxycortisol, 18OH-corticosterone, 18OH-cortisol, 18oxo-cortisol, 21-deoxycortisol, aldosterone, corticosterone, dehydroepiandrosterone sulfate (DHEAS), and combinations thereof. The group may further comprise cortisone. The group may also further comprise cortisol. The group may also further comprise 11-dehydrocorticosterone.

A plasma steroid may be selected from a group comprising at least or consisting in 11-deoxycorticosterone, 11-deoxycortisol, 18OH-corticosterone, 18OH-cortisol, 18oxo-cortisol, 21-deoxycortisol, aldosterone, and combinations thereof. The group may further comprise corticosterone. The group may also further comprise dehydroepiandrosterone sulfate (DHEAS).

A plasma steroid may be selected from a group comprising at least or consisting in 11-deoxycortisol, dehydroepiandrosterone (DHEA), dehydroepiandrosterone sulfate (DHEAS), and combinations thereof. The group may further comprise 11-deoxycorticosterone.

A plasma steroid may be selected from a group comprising at least or consisting in 11-deoxycorticosterone, 11-deoxycortisol, 18OH-corticosterone, 18OH-cortisol, 18oxo-cortisol, 21-deoxycortisol, aldosterone, and combinations thereof. The group may further comprise dehydroepiandrosterone sulfate (DHEAS).

A combination of plasma steroid may comprise at least or consist in a combination of 11-deoxycorticosterone, 11-deoxycortisol, 18OH-corticosterone, 18OH-cortisol, 18oxo-cortisol, 21-deoxycortisol, aldosterone, and combinations thereof. The combination may further comprise corticosterone. The combination may also further comprise dehydroepiandrosterone sulfate (DHEAS). The combination may also further comprise 11-dehydrocorticosterone. The combination may also further comprise cortisone. The combination may also further comprise cortisol.

A combination of plasma steroids may comprise at least or consist in a combination of 11-deoxycorticosterone, 11-deoxycortisol, 18OH-corticosterone, 18OH-cortisol, 18oxo-cortisol, 21-deoxycortisol, aldosterone, corticosterone, and dehydroepiandrosterone sulfate (DHEAS). The combination may further comprise 11-dehydrocorticosterone. The combination may also further comprise cortisone. The combination may also further comprise cortisol.

A combination of plasma steroids may comprise at least or consist in a combination of 11-deoxycorticosterone, 11-deoxycortisol, 18OH-corticosterone, 18OH-cortisol, 18oxo-cortisol, 21-deoxycortisol, and aldosterone. The combination may further comprise corticosterone. The combination also may further comprise dehydroepiandrosterone sulfate (DHEAS).

A combination of plasma steroids may comprise at least or consist in a combination of 11-deoxycortisol, dehydroepiandrosterone (DHEA), dehydroepiandrosterone sulfate (DHEAS). The combination may further comprise 11-deoxycorticosterone.

A combination of plasma steroids may comprise at least or consist in a combination of 11-deoxycorticosterone, 11-deoxycortisol, 18OH-corticosterone, 18OH-cortisol, 18oxo-cortisol, 21-deoxycortisol, and aldosterone. The combination may further comprise dehydroepiandrosterone sulfate (DHEAS).

A combination of biomarkers for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may comprise, as plasma steroids, at least plasma 11-deoxycorticosterone, plasma 11-deoxycortisol, plasma 18OH-corticosterone, plasma 18OH-cortisol, plasma 18oxo-cortisol, plasma 21-deoxycortisol, plasma aldosterone, plasma corticosterone, and plasma dehydroepiandrosterone sulfate (DHEAS). The combination of biomarkers may further comprise, as plasma steroids, at least one plasma steroid selected from the group comprising or consisting in plasma 11-dehydrocorticosterone, plasma cortisol, and plasma cortisone.

A combination of biomarkers for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may comprise, as plasma steroids, at least plasma 11-dehydrocorticosterone, plasma 11-deoxycorticosterone, plasma 11-deoxycortisol, plasma 18OH-corticosterone, plasma 18OH-cortisol, plasma 18oxo-cortisol, plasma 21-deoxycortisol, plasma aldosterone, plasma corticosterone, plasma cortisol, plasma cortisone, and plasma dehydroepiandrosterone sulfate (DHEAS).

A combination of biomarkers for stratifying a hypertensive patient among a plurality of types of hypertensive diseases, the plurality of types of hypertensive diseases comprising Endocrine Hypertension (EHT) and Primary Hypertension (PHT), may comprise, as plasma steroids, at least plasma 11-deoxycorticosterone, plasma 11-deoxycortisol, plasma 18OH-corticosterone, plasma 18OH-cortisol, plasma 18oxo-cortisol, plasma 21-deoxycortisol, and plasma aldosterone. The combination of biomarkers may further comprise, as plasma steroids, plasma corticosterone. The combination of biomarkers may further comprise, as plasma steroids, plasma dehydroepiandrosterone sulfate (DHEAS). The combination of biomarkers may further comprise, as plasma steroids, plasma corticosterone and plasma dehydroepiandrosterone sulfate (DHEAS).

A combination of biomarkers for stratifying a hypertensive patient among a plurality of types of hypertensive diseases, the plurality of types of hypertensive diseases comprising Endocrine Hypertension (EHT) and Primary Hypertension (PHT), may comprise, as plasma steroids, at least plasma 11-deoxycorticosterone, plasma 11-deoxycortisol, plasma 18OH-corticosterone, plasma 18OH-cortisol, plasma 18oxo-cortisol, plasma 21-deoxycortisol, plasma aldosterone, plasma corticosterone, and plasma dehydroepiandrosterone sulfate (DHEAS).

A combination of biomarkers for stratifying a hypertensive patient among a plurality of types of hypertensive diseases, the plurality of types of hypertensive diseases comprising Cushing's Syndrome (CS) and Primary Hypertension (PHT), may not comprise any plasma steroids.

A combination of biomarkers for stratifying a hypertensive patient among a plurality of types of hypertensive diseases, the plurality of types of hypertensive diseases comprising Cushing's Syndrome (CS) and Primary Hypertension (PHT), may comprise, as plasma steroids, at least plasma 11-deoxycortisol, plasma dehydroepiandrosterone (DHEA), and plasma dehydroepiandrosterone sulfate (DHEAS). The combination of biomarkers may further comprise, as plasma steroids, plasma 11-deoxycorticosterone.

A combination of biomarkers for stratifying a hypertensive patient among a plurality of types of hypertensive diseases, the plurality of types of hypertensive diseases comprising Primary Aldosteronism (PA) and Primary Hypertension (PHT), may comprise, as plasma steroids, at least plasma 11-deoxycorticosterone, plasma 11-deoxycortisol, plasma 18OH-corticosterone, plasma 18OH-Cortisol, plasma 18oxo-Cortisol, plasma 21-deoxycortisol, and plasma aldosterone. The combination of biomarkers may further comprise, as plasma steroids, dehydroepiandrosterone sulfate (DHEAS).

A combination of biomarkers for stratifying a hypertensive patient among a plurality of types of hypertensive diseases, the plurality of types of hypertensive diseases comprising Pheochromocytoma/Functional Paraganglioma (PPGL) and Primary Hypertension (PHT), may not comprise any plasma steroids.

### Urinary Steroids

A urinary steroid may be selected from a group comprising at least or consisting in α-cortol (Acortol), α-cortolone (Acortolone), androsterone (An), β-cortol (Bcortol), β-cortolone, cortisol, cortisone, dehydroepiandrosterone (DHEA), etiocholanolone (Etio), pregnanediol (PD), pregnenetriol (PT), 3α,5β-tetrahydroaldosterone (THAldo), tetrahydro-11-dehydrocorticosterone (THAs), tetrahydrocorticosterone (THB), tetrahydrodeoxycorticosterone (THDOC), tetrahydrocortisone (THE), tetrahydrocortisol (THF), tetrahydro-11-deoxycortisol (THS), 11-β-hydroxy-androsterone (11-β-OHAn), 11-β-hydroxy-etiocholanolone (11-βOHEt), urinary 11-oxo-etiocholanolone (11OxoEt), 17-OH-pregnanolone (17-HP), 18-hydroxycortisol (18-OHF), 5α-tetrahydrocorticosterone (5-αTHB), 5α-tetrahydrocortisol (5αTHF), 5-pregnanediol (PD), 5-pregnenetriol (5-PT), plasma 11-dehydrocorticosterone, and combinations thereof.

A urinary steroid may be selected from a group comprising at least or consisting in 11-β-hydroxy-androsterone (11-β-OHAn), 17-OH-pregnanolone (17-HP), 18-hydroxycortisol (18-OHF), 5α-tetrahydrocortisol (5αTHF), 5-pregnanediol (PD), 5-pregnenetriol (5-PT), α-cortol (Acortol), α-cortolone (Acortolone), androsterone (An), β-cortol (Bcortol), β-cortolone (Bcortolone), cortisol, cortisone, dehydroepiandrosterone (DHEA), etiocholanolone (Etio), pregnanediol (PD), pregnenetriol (PT), 3α,5β-tetrahydroaldosterone (THAldo), tetrahydro-11-dehydrocorticosterone (THAs), tetrahydrocorticosterone (THB), tetrahydrodeoxycorticosterone (THDOC), tetrahydrocortisone (THE), tetrahydrocortisol (THF), tetrahydro-11-deoxycortisol (THS), and combinations thereof.

A urinary steroid may be selected from a group comprising at least or consisting in 18-hydroxycortisol (18-OHF), 3α,5β-tetrahydroaldosterone (THAldo), tetrahydrodeoxycorticosterone (THDOC), tetrahydro-11-deoxycortisol (THS) and combinations thereof. The group may also further comprise α-cortol (acortol). The group may also further comprise pregnanediol (PD).

A urinary steroid may be selected from a group comprising at least or consisting in 18-hydroxycortisol (18-OHF), 3α,5β-tetrahydroaldosterone (THAldo), tetrahydrodeoxycorticosterone (THDOC), tetrahydro-11-deoxycortisol (THS), α-cortol (acortol), pregnanediol (PD), and combinations thereof. The group may further comprise at least one of 11-β-hydroxy-androsterone (11-β-OHAn), 17-OH-pregnanolone (17-HP), 5-pregnanediol (PD), 5-pregnenetriol (5-PT), 5α-tetrahydrocortisol (5αTHF), androsterone (An), cortisol, cortisone, dehydroepiandrosterone (DHEA), etiocholanolone (Etio), pregnenetriol (PT), tetrahydro-11-dehydrocorticosterone (THAs), tetrahydrocorticosterone (THB), tetrahydrocortisol (THF), tetrahydrocortisone (THE), α-cortolone (Acortolone), β-cortol (Bcortol), β-cortolone (Bcortolone), and combinations thereof.

A urinary steroid may be selected from a group comprising at least or consisting in 18-hydroxycortisol (18-OHF), 3α,5β-tetrahydroaldosterone (THAldo), tetrahydrodeoxycorticosterone (THDOC), tetrahydro-11-deoxycortisol (THS), and combinations thereof. The group may also further comprise pregnanediol (PD).

A urinary steroid may be selected from a group comprising at least or consisting in α-cortol (acortol), tetrahydro-11-deoxycortisol (THS) and combinations thereof. The group may further comprise 5-pregnenetriol (5-PT). The group may also further comprise androsterone (An). The group may also further comprise cortisol. The group may also further comprise etiocholanolone (Etio).

A urinary steroid may be selected from a group comprising at least or consisting in 18-hydroxycortisol (18-OHF), pregnanediol (PD), 3α,5β-tetrahydroaldosterone (THAldo), tetrahydrodeoxycorticosterone (THDOC), tetrahydro-11-deoxycortisol (THS), and combinations thereof. The group may also further comprise dehydroepiandrosterone (DHEA).

A urinary steroid may be selected from a group comprising at least or consisting in androsterone (An), etiocholanolone (Etio), and combinations thereof.

A combination of urinary steroid may comprise at least or consist in a combination of 18-hydroxycortisol (18-OHF), 3α,5β-tetrahydroaldosterone (THAldo), tetrahydrodeoxycorticosterone (THDOC), and tetrahydro-11-deoxycortisol (THS). The combination may also further comprise α-cortol (acortol). The combination may also further comprise pregnanediol (PD).

A combination of urinary steroid may comprise at least or consist in a combination of 18-hydroxycortisol (18-OHF), 3α,5β-tetrahydroaldosterone (THAldo), tetrahydrodeoxycorticosterone (THDOC), tetrahydro-11-deoxycortisol (THS), α-cortol (acortol), and pregnanediol (PD). The combination may further comprise at least one of 11-β-hydroxy-androsterone (11-β-OHAn), 17-OH-pregnanolone (17-HP), 5-pregnanediol (PD), 5-pregnenetriol (5-PT), 5α-tetrahydrocortisol (5αTHF), androsterone (An), cortisol, cortisone, dehydroepiandrosterone (DHEA), etiocholanolone (Etio), pregnenetriol (PT), tetrahydro-11-dehydrocorticosterone (THAs), tetrahydrocorticosterone (THB), tetrahydrocortisol (THF), tetrahydrocortisone (THE), α-cortolone (Acortolone), β-cortol (Bcortol), β-cortolone (Bcortolone), and combinations thereof.

A combination of urinary steroid may comprise at least or consist in a combination of 18-hydroxycortisol (18-OHF), 3α,5β-tetrahydroaldosterone (THAldo), tetrahydrodeoxycorticosterone (THDOC), and tetrahydro-11-deoxycortisol (THS). The combination may also further comprise pregnanediol (PD).

A combination of urinary steroid may comprise at least or consist in a combination of α-cortol (acortol), and tetrahydro-11-deoxycortisol (THS). The group may further comprise 5-pregnenetriol (5-PT). The combination may also further comprise androsterone (An). The combination may also further comprise cortisol. The combination may also further comprise etiocholanolone (Etio).

A combination of urinary steroid may comprise at least or consist in a combination of 18-hydroxycortisol (18-OHF), pregnanediol (PD), 3α,5β-tetrahydroaldosterone (THAldo), tetrahydrodeoxycorticosterone (THDOC), and tetrahydro-11-deoxycortisol (THS). The combination may also further comprise dehydroepiandrosterone (DHEA).

A combination of urinary steroid may comprise at least or consist in a combination of androsterone (An), and etiocholanolone (Etio).

A combination of biomarkers for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may comprise, as urinary steroids, at least urinary 18-hydroxycortisol (18-OHF), urinary 3α,5β-tetrahydroaldosterone (THAldo), urinary tetrahydrodeoxycorticosterone (THDOC), urinary tetrahydro-11-deoxycortisol (THS), urinary α-cortol (acortol), and urinary pregnanediol (PD). The combination of biomarkers may further comprise, as urinary steroids, at least one of urinary 11-β-hydroxy-androsterone (11-β-OHAn), urinary 17-OH-pregnanolone (17-HP), urinary 5-pregnanediol (PD), urinary 5-pregnenetriol (5-PT), urinary 5α-tetrahydrocortisol (5αTHF), urinary androsterone (An), urinary cortisol, urinary cortisone, urinary dehydroepiandrosterone (DHEA), urinary etiocholanolone (Etio), urinary pregnenetriol (PT), urinary tetrahydro-11-dehydrocorticosterone (THAs), urinary tetrahydrocorticosterone (THB), urinary tetrahydrocortisol (THF), urinary tetrahydrocortisone (THE), urinary α-cortolone (Acortolone), urinary β-cortol (Bcortol), urinary β-cortolone (Bcortolone), and combinations thereof.

A combination of biomarkers for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may comprise, as urinary steroids, at least urinary 18-hydroxycortisol (18-OHF), urinary α-cortol (acortol), urinary cortisol, urinary dehydroepiandrosterone (DHEA), urinary pregnanediol (PD), urinary 3α,5β-tetrahydroaldosterone (THAldo), urinary tetrahydro-11-dehydrocorticosterone (THAs), urinary tetrahydrodeoxycorticosterone (THDOC), urinary tetrahydrocortisone (THE), urinary tetrahydrocortisol (THF), and urinary tetrahydro-11-deoxycortisol (THS).

A combination of biomarkers for stratifying a hypertensive patient among a plurality of types of hypertensive diseases, the plurality of types of hypertensive diseases comprising Endocrine Hypertension (EHT) and Primary Hypertension (PHT), may comprise, as urinary steroids, at least urinary 18-hydroxycortisol (18-OHF), urinary 3α,5β-tetrahydroaldosterone (THAldo), urinary tetrahydrodeoxycorticosterone (THDOC), and urinary tetrahydro-11-deoxycortisol (THS). The combination of biomarkers may also further comprise, as urinary steroids, urinary pregnanediol (PD).

A combination of biomarkers for stratifying a hypertensive patient among a plurality of types of hypertensive diseases, the plurality of types of hypertensive diseases comprising Endocrine Hypertension (EHT) and Primary Hypertension (PHT), may comprise, as urinary steroids, at least urinary 18-hydroxycortisol (18-OHF), urinary 3α,5β-tetrahydroaldosterone (THAldo), urinary tetrahydrodeoxycorticosterone (THDOC), and urinary tetrahydro-11-deoxycortisol (THS).

A combination of biomarkers for stratifying a hypertensive patient among a plurality of types of hypertensive diseases, the plurality of types of hypertensive diseases comprising Cushing's Syndrome (CS) and Primary Hypertension (PHT), may comprise, as urinary steroids, urinary α-cortol (acortol), and urinary tetrahydro-11-deoxycortisol (THS). The combination may further comprise, as urinary steroids, urinary 5-pregnenetriol (5-PT). The combination may also further comprise, as urinary steroids, urinary androsterone (An). The combination may also further comprise, as urinary steroids, urinary cortisol. The combination may also further comprise urinary etiocholanolone (Etio).

A combination of biomarkers for stratifying a hypertensive patient among a plurality of types of hypertensive diseases, the plurality of types of hypertensive diseases comprising Cushing's Syndrome (CS) and Primary Hypertension (PHT), may comprise, as urinary steroids, urinary α-cortol (acortol), urinary androsterone (An), and urinary tetrahydro-11-deoxycortisol (THS).

A combination of biomarkers for stratifying a hypertensive patient among a plurality of types of hypertensive diseases, the plurality of types of hypertensive diseases comprising Primary Aldosteronism (PA) and Primary Hypertension (PHT), may comprise, as urinary steroids, at least urinary 18-hydroxycortisol (18-OHF), urinary pregnanediol (PD), urinary 3α,5β-tetrahydroaldosterone (THAldo), urinary tetrahydrodeoxycorticosterone (THDOC), and urinary tetrahydro-11-deoxycortisol (THS). The combination may also further comprise, as urinary steroids, urinary dehydroepiandrosterone (DHEA).

A combination of biomarkers for stratifying a hypertensive patient among a plurality of types of hypertensive diseases, the plurality of types of hypertensive diseases comprising Pheochromocytoma/Functional Paraganglioma (PPGL) and Primary Hypertension (PHT), may not comprise any urinary steroids.

A combination of biomarkers for stratifying a hypertensive patient among a plurality of types of hypertensive diseases, the plurality of types of hypertensive diseases comprising Pheochromocytoma/Functional Paraganglioma (PPGL) and Primary Hypertension (PHT), may comprise, as urinary steroids, at least urinary etiocholanolone (Etio). The combination may further comprise urinary androsterone (An).

A combination of biomarkers for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may comprise, as plasma steroids, at least plasma 11-deoxycorticosterone, plasma 11-deoxycortisol, plasma 18OH-corticosterone, plasma 18OH-cortisol, plasma 18oxo-cortisol, plasma 21-deoxycortisol, plasma aldosterone, plasma corticosterone, and plasma dehydroepiandrosterone sulfate (DHEAS) and as urinary steroids, at least urinary 18-hydroxycortisol (18-OHF), urinary 3α,5β-tetrahydroaldosterone (THAldo), urinary tetrahydrodeoxycorticosterone (THDOC), urinary tetrahydro-11-deoxycortisol (THS), urinary α-cortol (acortol), and urinary pregnanediol (PD). The combination of biomarkers may further comprise, as plasma steroids, at least one plasma steroid selected from the group comprising or consisting in plasma 11-dehydrocorticosterone, plasma cortisol, and plasma cortisone. The combination of biomarkers may further comprise, as urinary steroids, at least one of urinary 11-β-hydroxy-androsterone (11-β-OHAn), urinary 17-OH-pregnanolone (17-HP), urinary 5-pregnanediol (PD), urinary 5-pregnenetriol (5-PT), urinary 5α-tetrahydrocortisol (5αTHF), urinary androsterone (An), urinary cortisol, urinary cortisone, urinary dehydroepiandrosterone (DHEA), urinary etiocholanolone (Etio), urinary pregnenetriol (PT), urinary tetrahydro-11-dehydrocorticosterone (THAs), urinary tetrahydrocorticosterone (THB), urinary tetrahydrocortisol (THF), urinary tetrahydrocortisone (THE), urinary α-cortolone (Acortolone), urinary β-cortol (Bcortol), urinary β-cortolone (Bcortolone), and combinations thereof.

A combination of biomarkers for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may comprise, as plasma steroids, at least plasma 11-dehydrocorticosterone, plasma 11-deoxycorticosterone, plasma 11-deoxycortisol, plasma 18OH-corticosterone, plasma 18OH-cortisol, plasma 18oxo-cortisol, plasma 21-deoxycortisol, plasma aldosterone, plasma corticosterone, plasma cortisol, plasma cortisone, and plasma dehydroepiandrosterone sulfate (DHEAS) and as urinary steroids, at least urinary 18-hydroxycortisol (18-OHF), urinary α-cortol (acortol), urinary cortisol, urinary dehydroepiandrosterone (DHEA), urinary pregnanediol (PD), urinary 3α,5β-tetrahydroaldosterone (THAldo), urinary tetrahydro-11-dehydrocorticosterone (THAs), urinary tetrahydrodeoxycorticosterone (THDOC), urinary tetrahydrocortisone (THE), urinary tetrahydrocortisol (THF), and urinary tetrahydro-11-deoxycortisol (THS).

A combination of biomarkers for stratifying a hypertensive patient among a plurality of types of hypertensive diseases, the plurality of types of hypertensive diseases comprising Endocrine Hypertension (EHT) and Primary Hypertension (PHT), may comprise, as plasma steroids, at least plasma 11-deoxycorticosterone, plasma 11-deoxycortisol, plasma 18OH-corticosterone, plasma 18OH-cortisol, plasma 18oxo-cortisol, plasma 21-deoxycortisol, and plasma aldosterone, and as urinary steroids, at least urinary 18-hydroxycortisol (18-OHF), urinary 3α,5β-tetrahydroaldosterone (THAldo), urinary tetrahydrodeoxycorticosterone (THDOC), and urinary tetrahydro-11-deoxycortisol (THS). The combination of biomarkers may further comprise, as plasma steroids, plasma corticosterone. The combination of biomarkers may further comprise, as plasma steroids, plasma dehydroepiandrosterone sulfate (DHEAS). The combination of biomarkers may further comprise, as plasma steroids, plasma corticosterone and plasma dehydroepiandrosterone sulfate (DHEAS). The combination of biomarkers may also further comprise, as urinary steroids, urinary pregnanediol (PD).

A combination of biomarkers for stratifying a hypertensive patient among a plurality of types of hypertensive diseases, the plurality of types of hypertensive diseases comprising Endocrine Hypertension (EHT) and Primary Hypertension (PHT), may comprise, as plasma steroids, at least plasma 11-deoxycorticosterone, plasma 11-deoxycortisol, plasma 18OH-corticosterone, plasma 18OH-cortisol, plasma 18oxo-cortisol, plasma 21-deoxycortisol, plasma aldosterone, plasma corticosterone, and plasma dehydroepiandrosterone sulfate (DHEAS), and as urinary steroids, at least urinary 18-hydroxycortisol (18-OHF), urinary 3α,5β-tetrahydroaldosterone (THAldo), urinary tetrahydrodeoxycorticosterone (THDOC), and urinary tetrahydro-11-deoxycortisol (THS).

A combination of biomarkers for stratifying a hypertensive patient among a plurality of types of hypertensive diseases, the plurality of types of hypertensive diseases comprising Cushing's Syndrome (CS) and Primary Hypertension (PHT), may not comprise any plasma steroids and may comprise as urinary steroids, urinary α-cortol (acortol), and urinary tetrahydro-11-deoxycortisol (THS). The combination may further comprise, as urinary steroids, urinary 5-pregnenetriol (5-PT). The combination may also further comprise, as urinary steroids, urinary androsterone (An). The combination may also further comprise, as urinary steroids, urinary cortisol. The combination may also further comprise urinary etiocholanolone (Etio).

A combination of biomarkers for stratifying a hypertensive patient among a plurality of types of hypertensive diseases, the plurality of types of hypertensive diseases comprising Cushing's Syndrome (CS) and Primary Hypertension (PHT), may comprise, as plasma steroids, at least plasma 11-deoxycortisol, plasma dehydroepiandrosterone (DHEA), and plasma dehydroepiandrosterone sulfate (DHEAS) and as urinary steroids, urinary α-cortol (acortol), urinary androsterone (An), and urinary tetrahydro-11-deoxycortisol (THS). The combination of biomarkers may further comprise, as plasma steroids, plasma 11-deoxycorticosterone.

A combination of biomarkers for stratifying a hypertensive patient among a plurality of types of hypertensive diseases, the plurality of types of hypertensive diseases comprising Primary Aldosteronism (PA) and Primary Hypertension (PHT), may comprise, as plasma steroids, at least plasma 11-deoxycorticosterone, plasma 11-deoxycortisol, plasma 18OH-corticosterone, plasma 18OH-Cortisol, plasma 18oxo-Cortisol, plasma 21-deoxycortisol, and plasma aldosterone, and as urinary steroids, at least urinary 18-hydroxycortisol (18-OHF), urinary pregnanediol (PD), urinary 3α,5β-tetrahydroaldosterone (THAldo), urinary tetrahydrodeoxycorticosterone (THDOC), and urinary tetrahydro-11-deoxycortisol (THS). The combination of biomarkers may further comprise, as plasma steroids, dehydroepiandrosterone sulfate (DHEAS). The combination may also further comprise, as urinary steroids, urinary dehydroepiandrosterone (DHEA).

A combination of biomarkers for stratifying a hypertensive patient among a plurality of types of hypertensive diseases, the plurality of types of hypertensive diseases comprising Pheochromocytoma/Functional Paraganglioma (PPGL) and Primary Hypertension (PHT), may not comprise any plasma steroids and may not comprise any urinary steroids.

A combination of biomarkers for stratifying a hypertensive patient among a plurality of types of hypertensive diseases, the plurality of types of hypertensive diseases comprising Pheochromocytoma/Functional Paraganglioma (PPGL) and Primary Hypertension (PHT), may not comprise any plasma steroids and may comprise, as urinary steroids, at least urinary etiocholanolone (Etio). The combination may further comprise urinary androsterone (An).

### Small Metabolites

A biomarker to be used within the disclosure may be a small metabolite. A small metabolite is an organic compound, of molecular weight ranging from about 50 to about 1500 daltons (Da) and identified as a product and/or intermediate of cellular metabolism. Small metabolites do not include O-methylated catecholamines, steroids, or miRNA. A Human Metabolome Database is accessible at https://hmdb.ca/.

Small metabolites considered in the methods disclosed herein are known in the art, and information such as detailed structure or reference ranges may be obtained from The Human Metabolome Database (https://hmdb.ca/) and in particular from the metaP-server (http://metap.helmholtz-muenchen.de/metap2/).

Small metabolites disclosed herein are further described in the Human Metabolome Database (https://hmdb.ca/).

Small metabolites may be determined, and in particular may be quantified, in a plasma sample isolated from a patient.

Determination or quantification of Small metabolites may be carried out according to any known techniques in the art. For example, a useful analytical method may be gas chromatography-mass spectrometry [GC-MS], liquid chromatography-MS, NMR, LC/GC-FID, Direct Flow Injection MS/MS, LC ESI-MS/MS, MS/MS, which can be used to profile and quantify Small metabolites (Fiehn et al. Methods Mol Biol. 2007; 358:3-17; Psychogios et al. (2011) The Human Serum Metabolome. PLOS ONE 6 (2): e16957; Ando et al., Magn Reson Med Sci. 2013; 12 (2): 129-135).

In one exemplary embodiment, the measure of presence and quantification of Small metabolites may be carried out by LC ESI-MS/MS as disclosed in Romisch-Margl et al. (Metabolomics 2012; 8:133-142) or in Zukunft et al. (Chromatographia 2013; 76:1295-1305).

Depending on the type of Small metabolites to be determined, such as lipids, amino acids, different methods may be used. For example, NMR may be used in particular for amino acids, while GC-MS may be used in particular for fatty acids. A person skilled in the art may select the appropriate methods of determination depending on the small metabolite or set of Small metabolites to be determined, in particular to be quantified.

Amounts of Small metabolites may be expressed in weight/volume unit of sample, such as ng/ml or pg/ml of plasma. Alternatively, some metabolites, for example, amino acids, such as citrulline and arginine, or spermidine and putrescine, may be expressed in weight or molar ratio with other Small metabolites. Therefore, the ratio spermidine/putrescine or citrulline/arginine may be used as Small metabolites biomarkers instead of the individual Small metabolites.

Within the context of the disclosure, the acronym “PC” used in connection with Small metabolites intends to mean phosphatidylcholine. The following upper letter C with the figures, e.g., 17:0 or 16:1, such as in C17:0 and C16:1, intends to mean the total number of carbon atoms of the fatty chains and the total number of unsaturated bonds in the fatty chains of the phosphatidylcholine.

SM intends to refer to sphingomyelin. MUFA intends to refer to monounsaturated fatty acids. PUFA intends to refer to polyunsaturated fatty acid. SFA intends to refer to saturated fatty acids. Here after, indicated amino acids are mentioned using the standard 3-letters code, e.g. Trp for tryptophan, Met for methionine, Tyr for tyrosine, Arg for arginine, and so on. LysoPC stands for lysophosphatidylcholine.

The full names of the abbreviated metabolites disclosed herein are given in the following table.

A small metabolite suitable for the methods as disclosed herein may be any Small metabolites, or any combinations thereof, selected from the following Table 1:

TABLE 1
Small metabolites

| Plasma Small metabolites | Full Name | KEGG Code | HMBD Code |
|---|---|---|---|
| C0 | Carnitine (free) | C00487 | |
| C2 | Acetylcarnitine | | HMDB00201 |
| C3 | Propionylcarnitine | C03017 | HMDB00824 |
| C3-DC (C4OH) | Hydroxypropionylcarnitine | C02301 | |
| | (Malonylcarnitine) | | |
| C4 | Butyrylcarnitine | C02862 | HMDB02013 |
| | Isobutyrylcarnitine | | |
| C4:1 | Butenylcarnitine | | HMDB02095 |
| C5 | Isovalerylcarnitine | | HMDB00378 |
| | 2-Methylbutyrylcarnitine | | |
| | Valerylcarnitine | | |
| C6 (C4:1-DC) | Hexanoylcarnitine | | |
| | (Fumarylcarnitine) | | |
| C6:1 | Hexenoylcarnitine | | |
| C10 | Decanoylcarnitine | C03299 | |
| C10:1 | Decenoylcarnitine | C02301 | |
| C10:2 | Decadienoylcarnitine | C02301 | |
| C12 | Dodecanoylcarnitine | | HMDB02250 |
| | [Laurylcarnitine] | | |
| C12-DC | Dodecanedioylcarnitine | | |
| C14 | Tetradecanoylcarnitine | | HMDB05066 |
| | [=Myristylcarnitine] | | |
| C14:1 | Tetradecenoylcarnitine | | HMDB02014 |
| | [Myristoleylcarnitine] | | |
| C14:2 | Tetradecadienoylcarnitine | C02301 | |
| C16 | Hexadecanoylcarnitine | C02990 | HMDB00222 |
| | [Palmitoylcarnitine] | | |
| C16:1 | Hexadecenoylcarnitine | | HMDB06317 |
| | [=Palmitoleylcarnitine] | | |
| C16:2 | Hexadecadienylcarnitine | | |
| C18 | Octadecanoylcarnitine | | HMDB00848 |
| | [Stearylcarnitine] | | |
| C18.1 | Octenoylcarnitine | | HMDB06464 |
| C18:1-OH | Hydroxyoctadecenoylcarnitine | | |
| C18.2 | Octadecadienoylcarnitine | | HMDB06461 |
| | [Linoleylcarnitine] | | |
| Ala | Alanine | C01401 | |
| Arg | Arginine | C02385 | |
| Asn | Asparagine | C16438 | |
| Asp | Aspartic acid | C16433 | |
| Cit | Citrulline | C00327 | |
| Gln | Glutamine | C00303 | |
| Glu | Glutamic acid | C00302 | |
| Gly | Glycine | C00037 | |
| His | Histidine | C00768 | |
| Ile | Isoleucine | C16434 | |
| Leu | Leucine | C16439 | |
| Lys | Lysine | C16440 | |
| Met | Methionine | C01733 | |
| Orn | Ornithine | C01602 | |
| Phe | Phenylalanine | C02057 | |
| Pro | Proline | C16435 | |
| Ser | Serine | C00716 | |
| Thr | Threonine | C00188 | |
| Trp | Tryptophan | C00806 | |
| Tyr | Tyrosine | C01536 | |
| Val | Valine | C16436 | |
| Ac-Orn | Acetylornithine | C00437 | |
| Serotonin | Serotonin | C00780 | |
| Serotonin/Trp | serotonin/tryptophan ratio | | |
| alphaAAA | alpha-Aminoadipid acid | C00956 | |
| Creatinine | Creatinine | C00791 | |
| Met-SO | Methionine-Sulfoxide | C02989 | |
| Putrescine | Putrescine | C00134 | |
| Spermidine | Spermidine | C00315 | |
| t4-OH-Pro | Hydroxyproline | C01015 | |
| Taurine | Taurine | C00245 | |
| totalDMA | Total dimethylarginine | C03626 | |
| lysoPCaC140 | lysoPC a C14:0 | C04230 | |
| lysoPCaC160 | lysoPC a C16:0 | C04230 | |
| lysoPCaC161 | lysoPC a C16:1 | C04230 | |
| lysoPCaC170 | lysoPC a C17:0 | C04230 | |
| lysoPCaC180 | lysoPC a C18:0 | C04230 | |
| lysoPCaC181 | lysoPC a C18:1 | C03916 | |
| lysoPCaC182 | lysoPC a C18:2 | C04230 | |
| lysoPCaC203 | lysoPC a C20:3 | C04230 | |
| lysoPCaC204 | lysoPC a C20:4 | C04230 | |
| lysoPCaC280 | lysoPC a C28:0 | | |
| lysoPCaC281 | lysoPC a C28:1 | | |
| PCaaC240 | PC aa C24:0 | | |
| PCaaC260 | PC aa C26:0 | | |
| PCaaC281 | PC aa C28:1 | C00157 | |
| PCaaC300 | PC aa C30:0 | C00157 | |
| PCaaC320 | PC aa C32:0 | C00157 | |
| PCaaC321 | PC aa C32:1 | C00157 | |
| PCaaC322 | PC aa C32:2 | | |
| PCaaC323 | PC aa C32:3 | C00157 | |
| PCaaC341 | PC aa C34:1 | C00157 | |
| PCaaC342 | PC aa C34:2 | C00157 | |
| PCaaC343 | PC aa C34:3 | C00157 | |
| PCaaC344 | PC aa C34:4 | C00157 | |
| PCaaC360 | PC aa C36:0 | C00157 | |
| PCaaC361 | PC aa C36:1 | C00157 | |
| PCaaC362 | PC aa C36:2 | C00157 | |
| PCaaC363 | PC aa C36:3 | C00157 | |
| PCaaC364 | PC aa C36:4 | C00157 | |
| PCaaC365 | PC aa C36:5 | C00157 | |
| PCaaC366 | PC aa C36:6 | C00157 | |
| PCaaC380 | PC aa C38:0 | C00157 | |
| PCaaC381 | PC aa C38:1 | | |
| PCaaC383 | PC aa C38:3 | C00157 | |
| PCaaC384 | PC aa C38:4 | C00157 | |
| PCaaC385 | PC aa C38:5 | C00157 | |
| PCaaC386 | PC aa C38:6 | C00157 | |
| PCaaC402 | PC aa C40:2 | C00157 | |
| PCaaC403 | PC aa C40:3 | C00157 | |
| PCaaC404 | PC aa C40:4 | C00157 | |
| PCaaC405 | PC aa C40:5 | C00157 | |
| PCaaC406 | PC aa C40:6 | C00157 | |
| PCaaC420 | PC aa C42:0 | C00157 | |
| PCaaC421 | PC aa C42:1 | C00157 | |
| PCaaC422 | PC aa C42:2 | C00157 | |
| PCaaC424 | PC aa C42:4 | C00157 | |
| PCaaC425 | PC aa C42:5 | C00157 | |
| PCaaC426 | PC aa C42:6 | C00157 | |
| PCaeC300 | PC ae C30:0 | | |
| PCaeC321 | PC ae C32:1 | | |
| PCaeC322 | PC ae C32:2 | | |
| PCaeC340 | PC ae C34:0 | | |
| PCaeC341 | PC ae C34:1 | | |
| PCaeC342 | PC ae C34:2 | | |
| PCaeC343 | PC ae C34:3 | | |
| PCaeC360 | PC ae C36:0 | | |
| PCaeC361 | PC ae C36:1 | | |
| PCaeC362 | PC ae C36:2 | | |
| PCaeC363 | PC ae C36:3 | | |
| PCaeC364 | PC ae C36:4 | | |
| PCaeC365 | PC ae C36:5 | | |
| PCaeC380 | PC ae C38:0 | | |
| PCaeC381 | PC ae C38:1 | | |
| PCaeC382 | PC ae C38:2 | | |
| PCaeC383 | PC ae C38:3 | | |
| PCaeC384 | PC ae C38:4 | | |
| PCaeC385 | PC ae C38:5 | | |
| PCaeC386 | PC ae C38:6 | | |
| PCaeC401 | PC ae C40:1 | | |
| PCaeC402 | PC ae C40:2 | | |
| PCaeC403 | PC ae C40:3 | | |
| PCaeC404 | PC ae C40:4 | | |
| PCaeC405 | PC ae C40:5 | | |
| PCaeC406 | PC ae C40:6 | | |
| PCaeC420 | PC ae C42:0 | | |
| PCaeC421 | PC ae C42:1 | | |
| PCaeC422 | PC ae C42:2 | | |
| PCaeC423 | PC ae C42:3 | | |
| PCaeC424 | PC ae C42:4 | | |
| PCaeC425 | PC ae C42:5 | | |
| PCaeC443 | PC ae C44:3 | | |
| PCaeC444 | PC ae C44:4 | | |
| PCaeC445 | PC ae C44:5 | | |
| PCaeC446 | PC ae C44:6 | | |
| SMOHC141 | SM (OH) C14:1 | | |
| SMOHC161 | SM (OH) C16:1 | C00550 | |
| SMOHC221 | SM (OH) C22:1 | C00550 | |
| SMOHC222 | SM (OH) C22:2 | C00550 | |
| SMOHC241 | SM (OH) C24:1 | C00550 | |
| SMC160 | SM C16:0 | C00550 | |
| SMC161 | SM C16:1 | C00550 | |
| SMC180 | SM C18:0 | C00550 | |
| SMC181 | SM C18:1 | C00550 | |
| SMC202 | SM C20:2 | C00550 | |
| SMC240 | SM C24:0 | C00550 | |
| SMC241 | SM C24:1 | C00550 | |
| SMC260 | SM C26:0 | | |
| SMC261 | SM C26:1 | | |
| H1 | Sum of Hexoses (including | | |
| | Glucose) | | |
| (C2 + C3):C0 | (C2 + C3):C0 Ratio | | |
| AAA | alpha-Aminoadipid acid | C00956 | |
| ADMA | Asymmetric dimethylarginine | | |
| ADMA/Arg | Asymmetric dimethylarginine/ | | |
| | Arg Ratio | | |
| BCAA | Ile + Leu + Val | | |
| C2C0 | C2:C0 Ratio | | |
| Cit/Arg | Citrulline/Arg Ratio | | |
| Cit/Orn | Citrulline/Ornithine Ratio | | |
| CPT-I ratio | (C16 + C18)/C0 Ratio | | |
| Essential AA | Ile + Leu + Lys + Phe + Thr + | | |
| | Trp + Val + Met | | |
| Fisher ratio | BCAA/AAA Ratio | | |
| Glucogenic AA | Alanine + Glycine + Serine | | |
| Kynurenine | | | |
| Kynurenine/Trp | Kynurenine/tryptophan Ratio | | |
| Met-SO/Met | Methionine sulfoxide/ | | |
| | Methionine Ratio | | |
| Non-essential AA | Total AA − essential AA | | |
| Orn/Arg | Ornithine/Arginine Ratio | | |
| Putrescine/Orn | Putrescine/Ornithine Ratio | | |
| SDMA | Symmetric dimethylarginine | | |
| SDMA/Arg | Symmetric dimethylarginine/ | | |
| | Arg Ratio | | |
| Total AA | Ala + Arg + Asn + Asp + Cit + | | |
| | Gln + Glu + Gly + His + Ile + | | |
| | Leu + Lys + Met + Orn + Phe + | | |
| | Pro + Ser + Thr + Trp + Tyr + | | |
| | Val | | |
| Total DMA/Arg | Total dimethylarginine/Arg | | |
| | Ratio | | |
| Total SM-OH | SM (OH) C14:1 + SM (OH) | | |
| | C16:1 + SM (OH) C22:1 + SM | | |
| | (OH) C22:2 + SM (OH) C24:1 | | |
| Tyr/Phe | Tyrosine/Phenylalanine Ratio | | |

In some embodiments, a small metabolite suitable for the disclosure may selected from a group comprising or consisting in plasma acetylcarnitine (C2), plasma acetylornithine (Ac-Orn), plasma C6 (C4:1-DC) (hexanoylcarnitine (fumarylcarnitine)), plasma citrulline/arginine ratio (Cit/Arg), plasma creatinine, plasma decanoylcarnitine (C10), plasma dodecanoylcarnitine (C12), plasma glutamic acid (Glu), plasma H1 (sum of hexoses), plasma lysoPC a C16:0, plasma lysoPC a C17:0, plasma methioninesulfoxide/methionine ratio (Met-SO/Met), plasma octadecadienylcarnitine (C18:2), plasma octadecenoylcarnitine (C18:1), plasma PC aa C32:1, plasma PC aa C32:2, plasma PC aa C32:3, plasma PC aa C34:1, plasma PC aa C34:2, plasma PC aa C34:3, plasma PC aa C34:4, plasma PC aa C36:1, plasma PC aa C36:2, plasma PC aa C36:3, plasma PC ae C36:3, plasma serotonin, plasma serotonin/tryptophan ratio (Serotonin/Trp), plasma spermidine, plasma taurine, plasma tetradecenoylcarnitine (C14:1), plasma total dimethylarginine/arginine ratio (Total DMA/Arg), plasma tryptophan, and combinations thereof.

In some embodiments, a small metabolite suitable for the disclosure may selected from a group comprising or consisting in, plasma acetylcarnitine (C2), plasma C6 (C4:1-DC) (hexanoylcarnitine (fumarylcarnitine)), plasma creatinine, plasma decanoylcarnitine (C10), plasma dodecanoylcarnitine (C12), plasma H1 (sum of hexoses), plasma lysoPC a C16:0, plasma lysoPC a C17:0, plasma octadecadienylcarnitine (C18:2), plasma octadecenoylcarnitine (C18:1), plasma PC aa C32:1, plasma PC aa C32:2, plasma PC aa C32:3, plasma PC aa C34:1, plasma PC aa C34:2, plasma PC aa C34:3, plasma PC aa C36:2, plasma PC aa C36:3, plasma serotonin, plasma serotonin/tryptophan ratio (Serotonin/Trp), plasma tetradecenoylcarnitine (C14:1), and combinations thereof. The group may further comprise at least plasma acetylornithine (Ac-Orn). The group may further comprise at least plasma citrulline/arginine ratio (Cit/Arg). The group may further comprise at least plasma glutamic acid (Glu). The group may further comprise at least plasma methioninesulfoxide/methionine ratio (Met-SO/Met). The group may further comprise at least plasma PC aa C34:4. The group may further comprise at least plasma PC aa C36:1. The group may further comprise at least plasma PC ae C36:3. The group may further comprise at least plasma spermidine. The group may further comprise at least plasma taurine. The group may further comprise at least plasma total dimethylarginine/arginine ratio (Total DMA/Arg). The group may further comprise at least plasma tryptophan.

A small metabolite suitable for the disclosure may be plasma acetylornithine (Ac-Orn).

In some embodiments, a small metabolite suitable for the disclosure may be selected from a group comprising or consisting in plasma decanoylcarnitine (C10), plasma dodecanoylcarnitine (C12), plasma tetradecenoylcarnitine (C14:1), plasma octadecadienylcarnitine (C18:2), plasma acetylcarnitine (C2), plasma C6 (C4:1-DC) (hexanoylcarnitine), plasma glutamic acid, plasma H1 (sum of hexoses), plasma lysoPC a C17:0, plasma PC aa C32:1, plasma PC aa C32:2, plasma PC aa C32:3, plasma PC aa C34:2, plasma PC aa C34:3, plasma PC aa C36:3, plasma serotonin, plasma serotonin/tryptophan ratio (Serotonin/Trp), and combinations thereof. The group may further comprise at least plasma octadecenoylcarnitine (C18:1). The group may further comprise at least plasma creatinine. The group may further comprise at least plasma lysoPC a C16:0. The group may further comprise at least plasma PC aa C34:1. The group may further comprise at least plasma PC aa C34:4. The group may further comprise at least plasma PC aa C36:1. The group may further comprise at least plasma PC aa C36:2. The group may further comprise at least plasma PC ae C36:3. The group may further comprise at least plasma spermidine. The group may further comprise at least plasma total dimethylarginine/arginine ratio (Total DMA/Arg).

In some embodiments, a small metabolite suitable for the disclosure may be selected from a group comprising or consisting in plasma decanoylcarnitine (C10), plasma dodecanoylcarnitine (C12), plasma octadecadienylcarnitine (C18:2), plasma H1 (sum of hexoses), plasma PC aa C32:1, plasma PC aa C34:3, plasma serotonin, plasma serotonin/tryptophan ratio (Serotonin/Trp), and combinations thereof. The group may further comprise at least plasma lysoPC a C16:0. The group may further comprise at least plasma lysoPC a C17:0. The group may further comprise at least plasma acetylcarnitine (C2). The group may further comprise at least plasma C6 (C4:1-DC) (hexanoylcarnitine (fumarylcarnitine)). The group may further comprise at least plasma citrulline/arginine ratio (Cit/Arg). The group may further comprise at least plasma creatinine. The group may further comprise at least plasma glutamic acid (Glu). The group may further comprise at least plasma octadecenoylcarnitine (C18:1). The group may further comprise at least plasma PC aa C32:2. The group may further comprise at least plasma PC aa C32:3. The group may further comprise at least plasma PC aa C34:1. The group may further comprise at least plasma PC aa C34:2. The group may further comprise at least plasma PC aa C36:2. The group may further comprise at least plasma PC aa C36:3. The group may further comprise at least plasma PC ae C36:3. The group may further comprise at least plasma taurine. The group may further comprise at least plasma tetradecenoylcarnitine (C14:1). The group may further comprise at least plasma total dimethylarginine/arginine ratio (Total DMA/Arg).

In some embodiments, a small metabolite suitable for the disclosure may be selected from a group comprising or consisting in plasma methioninesulfoxide/methionine ratio (Met-SO/Met), plasma tryptophan, and combinations thereof.

In some embodiments, a small metabolite suitable for the disclosure may be selected from a group comprising or consisting in plasma decanoylcarnitine (C10), plasma tetradecenoylcarnitine (C14:1), plasma octadecenoylcarnitine (C18:1), plasma octadecadienylcarnitine (C18:2), plasma acetylcarnitine (C2), plasma C6 (C4:1-DC) (hexanoylcarnitine), plasma lysoPC a C17:0, plasma PC aa C32:1, plasma PC aa C32:2, plasma PC aa C32:3, plasma PC aa C34:2, plasma PC aa C34:3, plasma PC aa C36:3, plasma serotonin, plasma serotonin/tryptophan ratio (Serotonin/Trp), and combinations thereof. The group may further comprise at least plasma creatinine. The group may further comprise at least plasma dodecanoylcarnitine (C12). The group may further comprise at least plasma H1 (sum of hexoses). The group may further comprise at least plasma lysoPC a C16:0. The group may further comprise at least plasma octadecenoylcarnitine (C18:1). The group may further comprise at least plasma PC aa C34:1. The group may further comprise at least plasma PC aa C34:4. The group may further comprise at least plasma PC aa C36:1. The group may further comprise at least plasma PC aa C36:2. The group may further comprise at least plasma taurine.

In some embodiments, a small metabolite suitable for the disclosure may be plasma acetylornithine (Ac-Orn).

A combination of Small metabolites suitable for the disclosure may comprise at least or consist in a combination of plasma acetylcarnitine (C2), plasma C6 (C4:1-DC) (hexanoylcarnitine (fumarylcarnitine)), plasma creatinine, plasma decanoylcarnitine (C10), plasma dodecanoylcarnitine (C12), plasma H1 (sum of hexoses), plasma lysoPC a C16:0, plasma lysoPC a C17:0, plasma octadecadienylcarnitine (C18:2), plasma octadecenoylcarnitine (C18:1), plasma PC aa C32:1, plasma PC aa C32:2, plasma PC aa C32:3, plasma PC aa C34:1, plasma PC aa C34:2, plasma PC aa C34:3, plasma PC aa C36:2, plasma PC aa C36:3, plasma serotonin, plasma serotonin/tryptophan ratio (Serotonin/Trp), and plasma tetradecenoylcarnitine (C14:1). The combination may further comprise at least plasma acetylornithine (Ac-Orn). The combination may further comprise at least plasma citrulline/arginine ratio (Cit/Arg). The combination may further comprise at least plasma glutamic acid (Glu). The combination may further comprise at least plasma methioninesulfoxide/methionine ratio (Met-SO/Met). The combination may further comprise at least plasma PC aa C34:4. The combination may further comprise at least plasma PC aa C36:1. The combination may further comprise at least plasma PC ae C36:3. The combination may further comprise at least plasma spermidine. The combination may further comprise at least plasma taurine. The combination may further comprise at least plasma total dimethylarginine/arginine ratio (Total DMA/Arg). The combination may further comprise at least plasma tryptophan.

A combination of Small metabolites suitable for the disclosure may comprise at least or consist in a combination of plasma decanoylcarnitine (C10), plasma dodecanoylcarnitine (C12), plasma tetradecenoylcarnitine (C14:1), plasma octadecadienylcarnitine (C18:2), plasma acetylcarnitine (C2), plasma C6 (C4:1-DC) (hexanoylcarnitine), plasma glutamic acid, plasma H1 (sum of hexoses), plasma lysoPC a C17:0, plasma PC aa C32:1, plasma PC aa C32:2, plasma PC aa C32:3, plasma PC aa C34:2, plasma PC aa C34:3, plasma PC aa C36:3, plasma serotonin, and plasma serotonin/tryptophan ratio (Serotonin/Trp). The combination may further comprise at least plasma octadecenoylcarnitine (C18:1). The combination may further comprise at least plasma creatinine. The combination may further comprise at least plasma lysoPC a C16:0. The combination may further comprise at least plasma PC aa C34:1. The combination may further comprise at least plasma PC aa C34:4. The combination may further comprise at least plasma PC aa C36:1. The combination may further comprise at least plasma PC aa C36:2. The combination may further comprise at least plasma PC ae C36:3. The combination may further comprise at least plasma spermidine. The combination may further comprise at least plasma total dimethylarginine/arginine ratio (Total DMA/Arg).

A combination of Small metabolites suitable for the disclosure may comprise at least or consist in a combination of plasma decanoylcarnitine (C10), plasma dodecanoylcarnitine (C12), plasma octadecadienylcarnitine (C18:2), plasma H1 (sum of hexoses), plasma PC aa C32:1, plasma PC aa C34:3, plasma serotonin, and plasma serotonin/tryptophan ratio (Serotonin/Trp). The combination may further comprise at least plasma lysoPC a C16:0. The combination may further comprise at least plasma lysoPC a C17:0. The combination may further comprise at least plasma acetylcarnitine (C2). The combination may further comprise at least plasma C6 (C4:1-DC) (hexanoylcarnitine (fumarylcarnitine)). The combination may further comprise at least plasma citrulline/arginine ratio (Cit/Arg). The combination may further comprise at least plasma creatinine. The combination may further comprise at least plasma glutamic acid (Glu). The combination may further comprise at least plasma octadecenoylcarnitine (C18:1). The combination may further comprise at least plasma PC aa C32:2. The combination may further comprise at least plasma PC aa C32:3. The combination may further comprise at least plasma PC aa C34:1. The combination may further comprise at least plasma PC aa C34:2. The combination may further comprise at least plasma PC aa C36:2. The combination may further comprise at least plasma PC aa C36:3. The combination may further comprise at least plasma PC ae C36:3. The combination may further comprise at least plasma taurine. The combination may further comprise at least plasma tetradecenoylcarnitine (C14:1). The combination may further comprise at least plasma total dimethylarginine/arginine ratio (Total DMA/Arg).

A combination of Small metabolites suitable for the disclosure may comprise at least or consist in a combination of plasma methioninesulfoxide/methionine ratio (Met-SO/Met) and plasma tryptophan.

A combination of Small metabolites suitable for the disclosure may comprise at least or consist in a combination of plasma decanoylcarnitine (C10), plasma tetradecenoylcarnitine (C14:1), plasma octadecenoylcarnitine (C18:1), plasma octadecadienylcarnitine (C18:2), plasma acetylcarnitine (C2), plasma C6 (C4:1-DC) (hexanoylcarnitine), plasma lysoPC a C17:0, plasma PC aa C32:1, plasma PC aa C32:2, plasma PC aa C32:3, plasma PC aa C34:2, plasma PC aa C34:3, plasma PC aa C36:3, plasma serotonin, and plasma serotonin/tryptophan ratio (Serotonin/Trp). The combination may further comprise at least plasma creatinine. The combination may further comprise at least plasma dodecanoylcarnitine (C12). The combination may further comprise at least plasma H1 (sum of hexoses). The combination may further comprise at least plasma lysoPC a C16:0. The combination may further comprise at least plasma octadecenoylcarnitine (C18:1). The combination may further comprise at least plasma PC aa C34:1. The combination may further comprise at least plasma PC aa C34:4. The combination may further comprise at least plasma PC aa C36:1. The combination may further comprise at least plasma PC aa C36:2. The combination may further comprise at least plasma taurine.

A combination of Small metabolites suitable for the disclosure may comprise at least or consist in acetylornithine (Ac-Orn).

A combination of biomarkers for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may not comprise any Small metabolites.

A combination of biomarkers for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may comprise, as Small metabolites, at least plasma decanoylcarnitine (C10), plasma dodecanoylcarnitine (C12), plasma tetradecenoylcarnitine (C14:1), plasma octadecadienylcarnitine (C18:2), plasma acetylcarnitine (C2), plasma C6 (C4:1-DC) (hexanoylcarnitine), plasma glutamic acid, plasma H1 (sum of hexoses), plasma lysoPC a C17:0, plasma PC aa C32:1, plasma PC aa C32:2, plasma PC aa C32:3, plasma PC aa C34:2, plasma PC aa C34:3, plasma PC aa C36:3, plasma serotonin, and plasma serotonin/tryptophan ratio (Serotonin/Trp). The combination may further comprise at least plasma octadecenoylcarnitine (C18:1). The combination may further comprise at least plasma creatinine. The combination may further comprise at least plasma lysoPC a C16:0. The combination may further comprise at least plasma PC aa C34:1. The combination may further comprise at least plasma PC aa C34:4. The combination may further comprise at least plasma PC aa C36:1. The combination may further comprise at least plasma PC aa C36:2. The combination may further comprise at least plasma PC ae C36:3. The combination may further comprise at least plasma spermidine. The combination may further comprise at least plasma total dimethylarginine/arginine ratio (Total DMA/Arg).

A combination of biomarkers for stratifying a hypertensive patient among a plurality of types of hypertensive diseases, the plurality of types of hypertensive diseases comprising Endocrine Hypertension (EHT) and Primary Hypertension (PHT), may comprise, as Small metabolites, at least plasma decanoylcarnitine (C10), plasma dodecanoylcarnitine (C12), plasma octadecadienylcarnitine (C18:2), plasma H1 (sum of hexoses), plasma PC aa C32:1, plasma PC aa C34:3, plasma serotonin, and plasma serotonin/tryptophan ratio (Serotonin/Trp). The combination may further comprise at least plasma lysoPC a C16:0. The combination may further comprise at least plasma lysoPC a C17:0. The combination may further comprise at least plasma acetylcarnitine (C2). The combination may further comprise at least plasma C6 (C4:1-DC) (hexanoylcarnitine (fumarylcarnitine)). The combination may further comprise at least plasma citrulline/arginine ratio (Cit/Arg). The combination may further comprise at least plasma creatinine. The combination may further comprise at least plasma glutamic acid (Glu). The combination may further comprise at least plasma octadecenoylcarnitine (C18:1). The combination may further comprise at least plasma PC aa C32:2. The combination may further comprise at least plasma PC aa C32:3. The combination may further comprise at least plasma PC aa C34:1. The combination may further comprise at least plasma PC aa C34:2. The combination may further comprise at least plasma PC aa C36:2. The combination may further comprise at least plasma PC aa C36:3. The combination may further comprise at least plasma PC ae C36:3. The combination may further comprise at least plasma taurine. The combination may further comprise at least plasma tetradecenoylcarnitine (C14:1). The combination may further comprise at least plasma total dimethylarginine/arginine ratio (Total DMA/Arg).

A combination of biomarkers for stratifying a hypertensive patient among a plurality of types of hypertensive diseases, the plurality of types of hypertensive diseases comprising Cushing's Syndrome (CS) and Primary Hypertension (PHT), may not comprise any Small metabolites.

A combination of biomarkers for stratifying a hypertensive patient among a plurality of types of hypertensive diseases, the plurality of types of hypertensive diseases comprising Cushing's Syndrome (CS) and Primary Hypertension (PHT), may comprise, as Small metabolites, at least plasma methioninesulfoxide/methionine ratio (Met-SO/Met) and plasma tryptophan.

A combination of biomarkers for stratifying a hypertensive patient among a plurality of types of hypertensive diseases, the plurality of types of hypertensive diseases comprising Primary Aldosteronism (PA) and Primary Hypertension (PHT), may comprise, as Small metabolites, at least plasma decanoylcarnitine (C10), plasma tetradecenoylcarnitine (C14:1), plasma octadecenoylcarnitine (C18:1), plasma octadecadienylcarnitine (C18:2), plasma acetylcarnitine (C2), plasma C6 (C4:1-DC) (hexanoylcarnitine), plasma lysoPC a C17:0, plasma PC aa C32:1, plasma PC aa C32:2, plasma PC aa C32:3, plasma PC aa C34:2, plasma PC aa C34:3, plasma PC aa C36:3, plasma serotonin, and plasma serotonin/tryptophan ratio (Serotonin/Trp). The combination may further comprise at least plasma creatinine. The combination may further comprise at least plasma dodecanoylcarnitine (C12). The combination may further comprise at least plasma H1 (sum of hexoses). The combination may further comprise at least plasma lysoPC a C16:0. The combination may further comprise at least plasma octadecenoylcarnitine (C18:1). The combination may further comprise at least plasma PC aa C34:1. The combination may further comprise at least plasma PC aa C34:4. The combination may further comprise at least plasma PC aa C36:1. The combination may further comprise at least plasma PC aa C36:2. The combination may further comprise at least plasma taurine.

A combination of biomarkers for stratifying a hypertensive patient among a plurality of types of hypertensive diseases, the plurality of types of hypertensive diseases comprising Pheochromocytoma/Functional Paraganglioma (PPGL) and Primary Hypertension (PHT), may not comprise any Small metabolites.

A combination of biomarkers for stratifying a hypertensive patient among a plurality of types of hypertensive diseases, the plurality of types of hypertensive diseases comprising Pheochromocytoma/Functional Paraganglioma (PPGL) and Primary Hypertension (PHT), may comprise, as Small metabolites, at least acetylornithine (Ac-Orn).

### Combinations of Biomarkers

The disclosure relates to various combinations of different type of biomarkers as above indicated. The combinations of biomarkers may be for use for stratifying a hypertensive patient among a plurality of types of hypertensive diseases.

A plurality of types of hypertensive diseases may comprise at least two of Primary Aldosteronism (PA), Pheochromocytoma/Functional Paraganglioma (PPGL), Cushing's Syndrome (CS) and Primary Hypertension (PHT).

A plurality of types of hypertensive diseases may comprise Endocrine Hypertension (EHT) and Primary Hypertension (PHT).

A plurality of types of hypertensive diseases may comprise Cushing's Syndrome (CS) and Primary Hypertension (PHT).

A plurality of types of hypertensive diseases may comprise Primary Aldosteronism (PA) and Primary Hypertension (PHT).

A plurality of types of hypertensive diseases may comprise Pheochromocytoma/Functional Paraganglioma (PPGL) and Primary Hypertension (PHT).

A plurality of types of hypertensive diseases may comprise Primary Aldosteronism (PA), Pheochromocytoma/Functional Paraganglioma (PPGL), Cushing's Syndrome (CS) and Primary Hypertension (PHT).

A plurality of types of hypertensive diseases may comprise Endocrine Hypertension (EHT), Primary Aldosteronism (PA), Pheochromocytoma/Functional Paraganglioma (PPGL), Cushing's Syndrome (CS) and Primary Hypertension (PHT).

A combination of biomarkers suitable for the disclosure may comprise at least one biomarker selected in at least one, two, three, four, five or six of the following groups of biomarkers: Patient's age, O-methylated catecholamines, Plasma steroids, Urinary steroids, Small metabolites, and miRNAs.

The groups of biomarkers are as above detailed.

A combination of biomarkers suitable for the disclosure may comprise at least one biomarker selected in at least 1, 2, 3, 4, or 5 of the groups of biomarkers.

A combination of biomarkers selected in a single group of biomarkers may comprise at least three biomarkers selected in said group of biomarkers.

A combination of biomarkers selected in two groups of biomarkers may comprise at least three biomarkers selected in one of said two group of biomarkers.

A combination of biomarkers selected in three groups of biomarkers may comprise at least three biomarkers selected in one of said three group of biomarkers.

At least one group of biomarkers among the 1, 2, 3, 4, 5 or 6 groups used for a combination of biomarkers of the disclosure provides at least 3 biomarkers.

A combination of biomarkers suitable for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may comprise at least one biomarker selected in each of the following group of biomarkers: Patient's age, Plasma steroids, and Urinary steroids, and at least one biomarker selected in at least one of the group of biomarkers: O-methylated catecholamines, Small metabolites, and miRNA.

A combination of biomarkers suitable for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may comprise at least one biomarker selected in each of the following group of biomarkers: Patient's age, O-methylated catecholamines, Plasma steroids, and Urinary steroids.

A combination of biomarkers suitable for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may comprise at least one biomarker selected in each of the following group of biomarkers: Patient's age, O-methylated catecholamines, Plasma steroids, Urinary steroids, and at least one biomarker selected in at least one of the group of biomarkers Small metabolites, and miRNA.

A combination of biomarkers suitable for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may comprise at least one biomarker selected in each of the following group of biomarkers: Patient's age, O-methylated catecholamines, Plasma steroids, Urinary steroids, and Small metabolites.

A combination of biomarkers suitable for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may comprise at least one biomarker selected in each of the following group of biomarkers: Patient's age, O-methylated catecholamines, Plasma steroids, Urinary steroids, and miRNA.

A combination of biomarkers suitable for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may comprise at least one biomarker selected in each of the following group of biomarkers: Patient's age, O-methylated catecholamines, Plasma steroids, Urinary steroids, Small metabolites, and miRNA.

The combinations of O-methylated catecholamines, Plasma steroids, Urinary steroids, Small metabolites, and miRNA which can be used may be as above defined.

A combination of biomarkers suitable for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), may comprise at least age, plasma 11-deoxycorticosterone, plasma 11-deoxycortisol, plasma 18OH-corticosterone, plasma 18OH-cortisol, plasma 18oxo-cortisol, plasma 21-deoxycortisol, plasma aldosterone, plasma corticosterone, plasma dehydroepiandrosterone sulfate (DHEAS), urinary 18-hydroxycortisol (18-OHF), urinary α-cortol (acortol), urinary pregnanediol (PD), urinary 3α,5β-tetrahydroaldosterone (THAldo), urinary tetrahydrodeoxycorticosterone (THDOC), and urinary tetrahydro-11-deoxycortisol (THS).

The combination may further comprise at least one of plasma 3-methoxytyramine, plasma metanephrine, plasma normetanephrine, plasma 11-dehydrocorticosterone, plasma cortisol, plasma cortisone, plasma hsa-let-7g-5p, plasma hsa-miR-106b-3p, plasma hsa-miR-301a-3p, plasma hsa-miR-485-3p, plasma acetylcarnitine (C2), plasma C6 (C4:1-DC) (hexanoylcarnitine (fumarylcarnitine)), plasma creatinine, plasma decanoylcarnitine (C10), plasma dodecanoylcarnitine (C12), plasma glutamic acid, plasma H1 (sum of hexoses), plasma lysoPC a C16:0, plasma lysoPC a C17:0, plasma octadecadienylcarnitine (C18:2), plasma octadecenoylcarnitine (C18:1), plasma PC aa C32:1, plasma PC aa C32:2, plasma PC aa C32:3, plasma PC aa C34:1, plasma PC aa C34:2, plasma PC aa C34:3, plasma PC aa C34:4, plasma PC aa C36:1, plasma PC aa C36:2, plasma PC aa C36:3, plasma PC ae C36:3, plasma serotonin, plasma serotonin ratio/tryptophan (Serotonin/Trp), plasma spermidine, plasma tetradecenoylcarnitine (C14:1), plasma total dimethylarginine/arginine ratio (Total DMA/Arg), urinary 11-β-hydroxy-androsterone (11-β-OHAn), urinary 17-OH-pregnanolone (17-HP), urinary 5-pregnanediol (PD), urinary 5-pregnenetriol (5-PT), urinary 5α-tetrahydrocortisol (5αTHF), urinary androsterone (An), urinary cortisol, urinary cortisone, urinary dehydroepiandrosterone (DHEA), urinary etiocholanolone (Etio), urinary pregnenetriol (PT), urinary tetrahydro-11-dehydrocorticosterone (THAs), urinary tetrahydrocorticosterone (THB), urinary tetrahydrocortisol (THF), urinary tetrahydrocortisone (THE), urinary α-cortolone (Acortolone), urinary β-cortol (Bcortol), urinary β-cortolone (Bcortolone), and combinations thereof.

The combination may further comprise at least one of plasma metanephrine, plasma normetanephrine, urinary cortisol, urinary dehydroepiandrosterone (DHEA), urinary tetrahydro-11-dehydrocorticosterone (THAs), urinary tetrahydrocortisol (THF), urinary tetrahydrocortisone (THE), and combinations thereof.

The combination may further comprise a combination comprising at least plasma metanephrine, plasma normetanephrine, urinary cortisol, urinary dehydroepiandrosterone (DHEA), urinary tetrahydro-11-dehydrocorticosterone (THAs), urinary tetrahydrocortisol (THF), urinary tetrahydrocortisone (THE), and combinations thereof.

The combination of biomarkers may comprise or consist in age, plasma metanephrine, plasma normetanephrine, plasma 11-deoxycorticosterone, plasma 11-deoxycortisol, plasma 18OH-corticosterone, plasma 18OH-cortisol, plasma 18oxo-cortisol, plasma 21-deoxycortisol, plasma aldosterone, plasma corticosterone, plasma dehydroepiandrosterone sulfate (DHEAS), urinary 18-hydroxycortisol (18-OHF), urinary α-cortol (acortol), urinary cortisol, urinary dehydroepiandrosterone (DHEA), urinary pregnanediol (PD), urinary 3α,5β-tetrahydroaldosterone (THAldo), urinary tetrahydro-11-dehydrocorticosterone (THAs), urinary tetrahydrodeoxycorticosterone (THDOC), urinary tetrahydrocortisone (THE), urinary tetrahydrocortisol (THF), and urinary tetrahydro-11-deoxycortisol (THS).

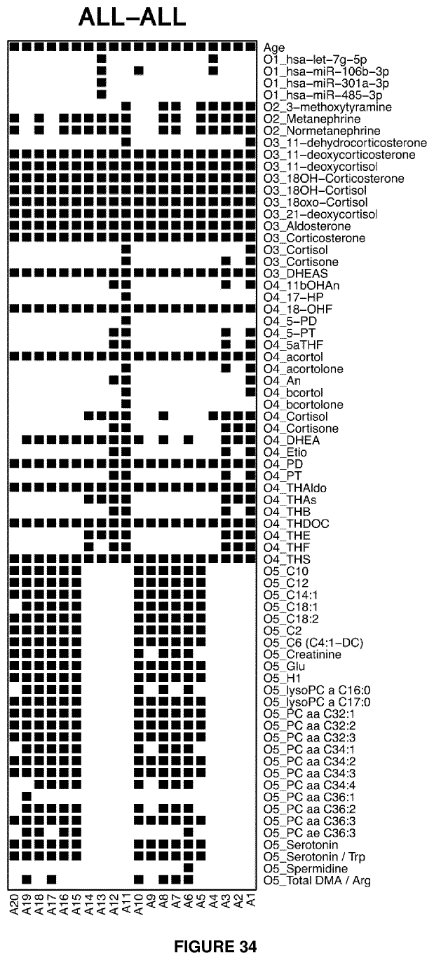

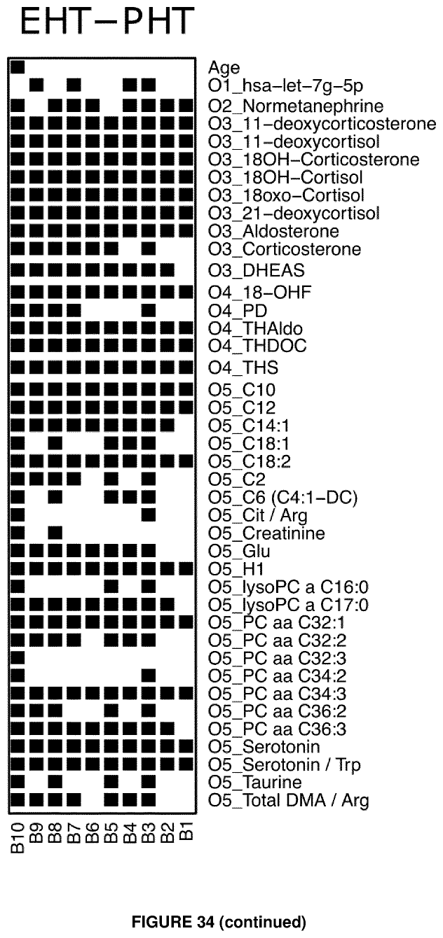

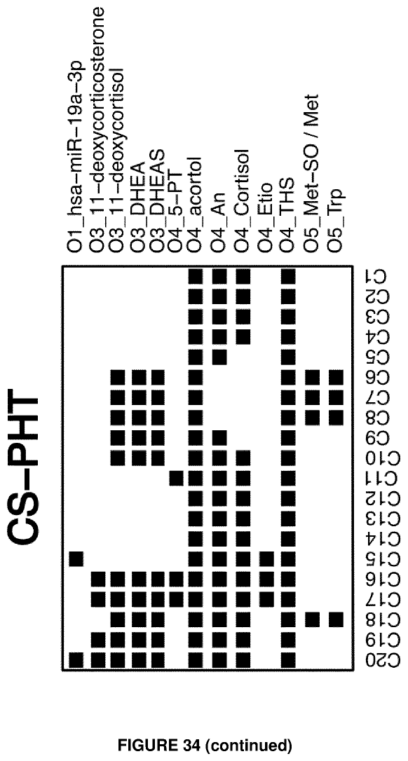

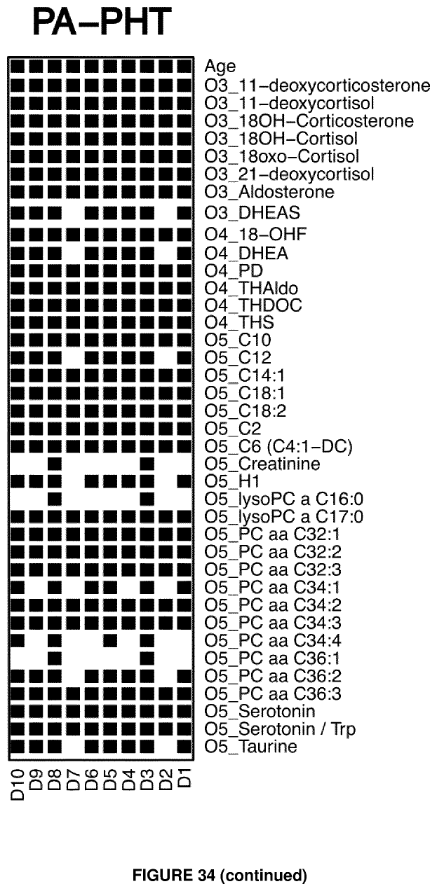

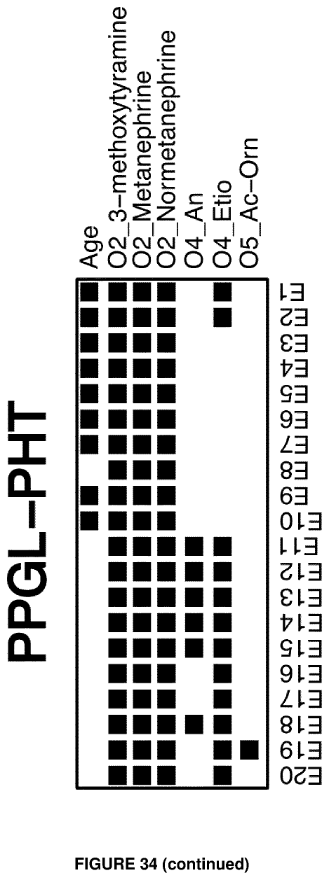

The combination of biomarkers may comprise or consist in any of the combinations represented on FIG. 34 and identified by the references A1 to A20.

A combination of biomarkers for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT) may be selected among the combinations A1, A2, A3, A4, A5, A6, A7, A8, A9, A10, A11, A12, A13, A14, A15, A16, A17, A18, A19 and A20 as set out on FIG. 34 (ALL vs ALL).

In some embodiments, a combination of biomarkers for stratifying a hypertensive patient among a plurality of types of hypertensive diseases, the plurality of types of hypertensive diseases comprising Primary Aldosteronism n (PA), Pheochromocytoma/Functional Paraganglioma (PPGL), Cushing's Syndrome (CS) and Primary Hypertension (PHT) may be selected among the combinations A13, A14 and A15 as set out on FIG. 34 (ALL vs ALL). In some embodiments, the combination of biomarkers may the combination A13 as set out on FIG. 34 (ALL vs ALL). In some embodiments, the combination of biomarkers may the combination A14 as set out on FIG. 34 (ALL vs ALL). In some embodiments, the combination of biomarkers may the combination A15 as set out on FIG. 34 (ALL vs ALL).

In some embodiments, for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT) one may use at least two, or more, of the combinations of biomarkers selected among the combinations A1, A2, A3, A4, A5, A6, A7, A8, A9, A10, A11, A12, A13, A14, A15, A16, A17, A18, A19 and A20 as set out on FIG. 34 (ALL vs ALL).

In some embodiments, for stratifying a hypertensive patient among a plurality of types of hypertensive diseases, the plurality of types of hypertensive diseases comprising Primary Aldosteronism (PA), Pheochromocytoma/Functional Paraganglioma (PPGL), Cushing's Syndrome (CS) and Primary Hypertension (PHT), one may use a set of three combinations of biomarkers, the set comprising the combinations A13, A14, and A15 as set out on FIG. 34 (ALL vs ALL).

A combination of biomarkers suitable for stratifying a hypertensive patient among a plurality of types of hypertensive diseases, the plurality of types of hypertensive diseases comprising Endocrine Hypertension (EHT) and Primary Hypertension (PHT), may comprise at least one biomarker selected in each of the following group of biomarkers: Plasma steroids, Urinary steroids, and Small metabolites, and at least one biomarker selected in at least one of the group of biomarkers: Patient's age, O-methylated catecholamines, and miRNA.

A combination of biomarkers suitable for stratifying a hypertensive patient among a plurality of types of hypertensive diseases, the plurality of types of hypertensive diseases comprising Endocrine Hypertension (EHT) and Primary Hypertension (PHT), may comprise at least one biomarker selected in each of the following group of biomarkers: O-methylated catecholamines, Plasma steroids, Urinary steroids, and Small metabolites.

A combination of biomarkers suitable for stratifying a hypertensive patient among a plurality of types of hypertensive diseases, the plurality of types of hypertensive diseases comprising Endocrine Hypertension (EHT) and Primary Hypertension (PHT), may comprise at least one biomarker selected in each of the following group of biomarkers: Plasma steroids, Urinary steroids, Small metabolites, and miRNA.

A combination of biomarkers suitable for stratifying a hypertensive patient among a plurality of types of hypertensive diseases, the plurality of types of hypertensive diseases comprising Endocrine Hypertension (EHT) and Primary Hypertension (PHT), may comprise at least one biomarker selected in each of the following group of biomarkers: O-methylated catecholamines, Plasma steroids, Urinary steroids, Small metabolites, and miRNA.

A combination of biomarkers suitable for stratifying a hypertensive patient among a plurality of types of hypertensive diseases, the plurality of types of hypertensive diseases comprising Endocrine Hypertension (EHT) and Primary Hypertension (PHT), may comprise at least one biomarker selected in each of the following group of biomarkers: Patient's age, O-methylated catecholamines, Plasma steroids, Urinary steroids, and Small metabolites.

The combinations of O-methylated catecholamines, Plasma steroids, Urinary steroids, Small metabolites, and miRNA which can be used may be as above defined.

A combination of biomarkers suitable for stratifying a hypertensive patient among a plurality of types of hypertensive diseases, the plurality of types of hypertensive diseases comprising Endocrine Hypertension (EHT) and Primary Hypertension (PHT), may comprise at least plasma 11-deoxycorticosterone, plasma 11-deoxycortisol, plasma 18OH-corticosterone, plasma 18OH-cortisol, plasma 18oxo-cortisol, plasma 21-deoxycortisol, plasma 18-hydroxycortisol aldosterone, urinary (18-OHF), urinary 3α,5β-tetrahydroaldosterone (THAldo), urinary tetrahydrodeoxycorticosterone (THDOC), urinary tetrahydro-11-deoxycortisol (THS), plasma decanoylcarnitine (C10), plasma dodecanoylcarnitine (C12), plasma octadecadienylcarnitine (C18:2), plasma H1 (sum of hexoses), plasma PC aa C32:1, plasma PC aa C34:3, plasma serotonin, plasma serotonin/tryptophan ratio (Serotonin/Trp).

The combination may further comprise at least one of age, plasma hsa-let-7g-5p, plasma lysoPC a C16:0, plasma lysoPC a C17:0, plasma normetanephrine, plasma corticosterone, plasma dehydroepiandrosterone sulfate (DHEAS), urinary pregnanediol (PD), plasma acetylcarnitine (C2), plasma C6 (C4:1-DC) (hexanoylcarnitine (fumarylcarnitine)), plasma citrulline/arginine ratio (Cit/Arg), plasma creatinine, plasma glutamic acid (Glu), plasma octadecenoylcarnitine (C18:1), plasma PC aa C32:2, plasma PC aa C32:3, plasma PC aa C34:1, plasma PC aa C34:2, plasma PC aa C36:2, plasma PC aa C36:3, plasma PC ae C36:3, plasma taurine, plasma tetradecenoylcarnitine (C14:1), plasma total dimethylarginine/arginine ratio (Total DMA/Arg), and combinations thereof.

The combination may further comprise at least plasma normetanephrine.

The combination of biomarkers may comprise or consist in plasma normetanephrine, plasma 11-deoxycorticosterone, plasma 11-deoxycortisol, plasma 18OH-corticosterone, plasma 18OH-cortisol, plasma 18oxo-cortisol, plasma 21-deoxycortisol, plasma aldosterone, urinary 18-hydroxycortisol (18-OHF), urinary 3α,5β-tetrahydroaldosterone (THAldo), urinary tetrahydrodeoxycorticosterone (THDOC), urinary tetrahydro-11-deoxycortisol (THS), plasma decanoylcarnitine (C10), plasma dodecanoylcarnitine (C12), plasma octadecadienylcarnitine (C18:2), plasma H1 (sum of hexoses), plasma PC aa C32:1, plasma PC aa C34:3, plasma serotonin, plasma serotonin/tryptophan ratio (Serotonin/Trp).

The combination of biomarkers may comprise or consist in any of the combinations represented on FIG. 34 and identified by the references B1 to B10.

A combination of biomarkers suitable for stratifying a hypertensive patient among a plurality of types of hypertensive diseases, the plurality of types of hypertensive diseases comprising Endocrine Hypertension (EHT) and Primary Hypertension (PHT), may be selected among the combinations B1, B2, B3, B4, B5, B6, B7, B8, B9, and B10 as set out on FIG. 34 (EHT vs PHT).

In some embodiments, for stratifying a hypertensive patient among a plurality of types of hypertensive diseases, the plurality of types of hypertensive diseases comprising Endocrine Hypertension (EHT) and Primary Hypertension (PHT), one may use at least two, or more, of the combinations of biomarkers selected among the combinations B1, B2, B3, B4, B5, B6, B7, B8, B9, and B10 as set out on FIG. 34 (EHT vs PHT).

A combination of biomarkers for stratifying a hypertensive patient among a plurality of types of hypertensive diseases, the plurality of types of hypertensive diseases comprising Cushing's Syndrome (CS) and Primary Hypertension (PHT), may comprise at least three biomarkers selected in the group of biomarkers: Urinary steroids, and at least one biomarker selected in at least one of the group of biomarkers: Plasma steroids, Small metabolites, and miRNA.

A combination of biomarkers for stratifying a hypertensive patient among a plurality of types of hypertensive diseases, the plurality of types of hypertensive diseases comprising Cushing's Syndrome (CS) and Primary Hypertension (PHT), may comprise at least one biomarker selected in each of the following group of biomarkers: Urinary steroids and Plasma steroids.

A combination of biomarkers for stratifying a hypertensive patient among a plurality of types of hypertensive diseases, the plurality of types of hypertensive diseases comprising Cushing's Syndrome (CS) and Primary Hypertension (PHT), may comprise at least one biomarker selected in each of the following group of biomarkers: Urinary steroids and miRNA.

A combination of biomarkers for stratifying a hypertensive patient among a plurality of types of hypertensive diseases, the plurality of types of hypertensive diseases comprising Cushing's Syndrome (CS) and Primary Hypertension (PHT), may comprise at least one biomarker selected in each of the following group of biomarkers: Urinary steroids Plasma steroids, and miRNA.

A combination of biomarkers for stratifying a hypertensive patient among a plurality of types of hypertensive diseases, the plurality of types of hypertensive diseases comprising Cushing's Syndrome (CS) and Primary Hypertension (PHT), may comprise at least one biomarker selected in each of the following group of biomarkers: Urinary steroids, Plasma steroids, and Small metabolites.

The combinations of Plasma steroids, Urinary steroids, Small metabolites, and miRNA which can be used may be as above defined.

A combination of biomarkers for stratifying a hypertensive patient among a plurality of types of hypertensive diseases, the plurality of types of hypertensive diseases comprising Cushing's Syndrome (CS) and Primary Hypertension (PHT), may comprise at least urinary α-cortol (acortol), and urinary tetrahydro-11-deoxycortisol (THS).

The combination may further comprise at least one of plasma 11-deoxycorticosterone, plasma 11-deoxycortisol, plasma dehydroepiandrosterone (DHEA), plasma dehydroepiandrosterone sulfate (DHEAS), plasma hsa-miR-19a-3p, plasma methioninesulfoxide/methionine ratio (Met-SO/Met), plasma tryptophan, urinary 5-pregnenetriol (5-PT), urinary androsterone (An), urinary cortisol, urinary etiocholanolone (Etio), and combinations thereof.

The combination may further comprise urinary androsterone (An).

The combination of biomarkers may comprise or consist in urinary α-cortol (acortol), urinary androsterone (An), and urinary tetrahydro-11-deoxycortisol (THS).

The combination of biomarkers may comprise or consist in any of the combinations represented on FIG. 34 and identified by the references C1 to C20.

A combination of biomarkers for stratifying a hypertensive patient among a plurality of types of hypertensive diseases, the plurality of types of hypertensive diseases comprising Cushing's Syndrome (CS) and Primary Hypertension (PHT), may be selected among the combinations C1, C2, C3, C4, C5, C6, C7, C8, C9, C10, C11, C12, C13, C14, C15, C16, C17, C18, C19 and C20 as set out on FIG. 34 (CS vs PHT).

In some embodiments, for stratifying a hypertensive patient among a plurality of types of hypertensive diseases, the plurality of types of hypertensive diseases comprising Cushing's Syndrome (CS) and Primary Hypertension (PHT), one may use at least two, or more, of the combinations of biomarkers selected among the combinations C1, C2, C3, C4, C5, C6, C7, C8, C9, C10, C11, C12, C13, C14, C15, C16, C17, C18, C19 and C20 as set out on FIG. 34 (CS vs PHT).

A combination of biomarkers for stratifying a hypertensive patient among a plurality of types of hypertensive diseases, the plurality of types of hypertensive diseases comprising Primary Aldosteronism (PA) and Primary Hypertension (PHT), may comprise at least one biomarker selected in each of the following group of biomarkers: Patient's age, Plasma steroids, Urinary steroids, and Small metabolites, and at least one biomarker selected in at least one of the group of biomarkers: O-methylated catecholamines, and miRNA.

A combination of biomarkers for stratifying a hypertensive patient among a plurality of types of hypertensive diseases, the plurality of types of hypertensive diseases comprising Primary Aldosteronism (PA) and Primary Hypertension (PHT), may comprise at least one biomarker selected in each of the following group of biomarkers: Patient's age, Plasma steroids, Urinary steroids, Small metabolites, O-methylated catecholamines, and miRNA.

The combinations of O-methylated catecholamines, Plasma steroids, Urinary steroids, Small metabolites, and miRNA which can be used may be as above defined.

A combination of biomarkers for stratifying a hypertensive patient among a plurality of types of hypertensive diseases, the plurality of types of hypertensive diseases comprising Primary Aldosteronism (PA) and Primary Hypertension (PHT), may comprise at least age, plasma 11-deoxycorticosterone, plasma 11-deoxycortisol, plasma 18OH-corticosterone, plasma 18OH-cortisol, plasma 18oxo-cortisol, plasma 21-deoxycortisol, plasma aldosterone, urinary 18-hydroxycortisol (18-OHF), urinary pregnanediol (PD), urinary 3α,5β-tetrahydroaldosterone (THAldo), urinary tetrahydrodeoxycorticosterone (THDOC), urinary tetrahydro-11-deoxycortisol (THS), plasma decanoylcarnitine (C10), plasma tetradecenoylcarnitine (C14:1), plasma octadecenoylcarnitine (C18:1), plasma octadecadienylcarnitine (C18:2), plasma acetylcarnitine (C2), plasma C6 (C4:1-DC) (hexanoylcarnitine), plasma lysoPC a C17:0, plasma PC aa C32:1, plasma PC aa C32:2, plasma PC aa C32:3, plasma PC aa C34:2, plasma PC aa C34:3, plasma PC aa C36:3, plasma serotonin, and plasma serotonin/tryptophan ratio (Serotonin/Trp).

The combination may further comprise at least one of plasma creatinine, plasma dehydroepiandrosterone sulfate (DHEAS), plasma dodecanoylcarnitine (C12), plasma H1 (sum of hexoses), plasma lysoPC a C16:0, plasma PC aa C34:1, plasma PC aa C34:4, plasma PC aa C36:1, plasma PC aa C36:2, plasma taurine, urinary dehydroepiandrosterone (DHEA), and combinations thereof.

The combination of biomarkers may comprise or consist in age, plasma 11-deoxycorticosterone, plasma 11-deoxycortisol, plasma 18OH-corticosterone, plasma 18OH-cortisol, plasma 18oxo-cortisol, plasma 21-deoxycortisol, plasma aldosterone, urinary 18-hydroxycortisol (18-OHF), urinary pregnanediol (PD), urinary 3α,5β-tetrahydroaldosterone (THAldo), urinary tetrahydrodeoxycorticosterone (THDOC), urinary tetrahydro-11-deoxycortisol (THS), plasma decanoylcarnitine (C10), plasma tetradecenoylcarnitine (C14:1), plasma octadecenoylcarnitine (C18:1), plasma octadecadienylcarnitine (C18:2), plasma acetylcarnitine (C2), plasma C6 (C4:1-DC) (hexanoylcarnitine), plasma lysoPC a C17:0, plasma PC aa C32:1, plasma PC aa C32:2, plasma PC aa C32:3, plasma PC aa C34:2, plasma PC aa C34:3, plasma PC aa C36:3, plasma serotonin, and plasma serotonin/tryptophan ratio (Serotonin/Trp).

The combination of biomarkers may comprise or consist in any of the combinations represented on FIG. 34 and identified by the references D1 to D10.

A combination of biomarkers for stratifying a hypertensive patient among a plurality of types of hypertensive diseases, the plurality of types of hypertensive diseases comprising Primary Aldosteronism (PA) and Primary Hypertension (PHT), may be selected among the combinations D1, D2, D3, D4, D5, D6, D7, D8, D9, and D10 as set out on FIG. 34 (PA vs PHT).

In some embodiments, for stratifying a hypertensive patient among a plurality of types of hypertensive diseases, the plurality of types of hypertensive diseases comprising Primary Aldosteronism (PA) and Primary Hypertension (PHT) one may use at least two, or more, of the combinations of biomarkers selected among the combinations D1, D2, D3, D4, D5, D6, D7, D8, D9, and D10 as set out on FIG. 34 (PA vs PHT).

A combination of biomarkers for stratifying a hypertensive patient among a plurality of types of hypertensive diseases, the plurality of types of hypertensive diseases comprising Pheochromocytoma/Functional Paraganglioma (PPGL) and Primary Hypertension (PHT), may comprise at least three biomarkers selected in the group of biomarkers: O-methylated catecholamines, and at least one biomarker selected in at least one of the group of biomarkers: Patient's age, Urinary steroids, and Small metabolites.

A combination of biomarkers for stratifying a hypertensive patient among a plurality of types of hypertensive diseases, the plurality of types of hypertensive diseases comprising Pheochromocytoma/Functional Paraganglioma (PPGL) and Primary Hypertension (PHT), may comprise at least three biomarkers selected in O-methylated catecholamines, and the Patient's age.

A combination of biomarkers for stratifying a hypertensive patient among a plurality of types of hypertensive diseases, the plurality of types of hypertensive diseases comprising Pheochromocytoma/Functional Paraganglioma (PPGL) and Primary Hypertension (PHT), may comprise at least three biomarkers selected in O-methylated catecholamines, the Patient's age, and at least one biomarker selected in Urinary steroids.

A combination of biomarkers for stratifying a hypertensive patient among a plurality of types of hypertensive diseases, the plurality of types of hypertensive diseases comprising Pheochromocytoma/Functional Paraganglioma (PPGL) and Primary Hypertension (PHT), may comprise at least three biomarkers selected in O-methylated catecholamines, and at least one biomarker selected in Urinary steroids.

A combination of biomarkers for stratifying a hypertensive patient among a plurality of types of hypertensive diseases, the plurality of types of hypertensive diseases comprising Pheochromocytoma/Functional Paraganglioma (PPGL) and Primary Hypertension (PHT), may comprise at least three biomarkers selected in O-methylated catecholamines, and at least one biomarker selected in each of the following group of biomarkers: Urinary steroids and Small metabolites.

The combinations of O-methylated catecholamines, Urinary steroids, and Small metabolites which can be used may be as above defined.

A combination of biomarkers for stratifying a hypertensive patient among a plurality of types of hypertensive diseases, the plurality of types of hypertensive diseases comprising Pheochromocytoma/Functional Paraganglioma (PPGL) and Primary Hypertension (PHT), may comprise at least plasma 3-methoxytyramine, plasma metanephrine, and plasma normetanephrine.

The combination may further comprise at least one of age, plasma acetylornithine (Ac-Orn), urinary androsterone (An), urinary etiocholanolone (Etio), and combinations thereof.

The combination of biomarkers may comprise or consist in plasma 3-methoxytyramine, plasma metanephrine, and plasma normetanephrine.

The combination of biomarkers may comprise or consist in any of the combinations represented on FIG. 34 and identified by the references E1 to E20.

A combination of biomarkers for stratifying a hypertensive patient among a plurality of types of hypertensive diseases, the plurality of types of hypertensive diseases comprising Pheochromocytoma/Functional Paraganglioma (PPGL) and Primary Hypertension (PHT), may be selected among the combinations E1, E2, E3, E4, E5, E6, E7, E8, E9, E10, E11, E12, E13, E14, E15, E16, E17, E18, E19, and E20 as set out on FIG. 34 (PPGL vs PHT).

In some embodiments, for stratifying a hypertensive patient among a plurality of types of hypertensive diseases, the plurality of types of hypertensive diseases comprising Pheochromocytoma/Functional Paraganglioma (PPGL) and Primary Hypertension (PHT) one may use at least two, or more, of the combinations of biomarkers selected among the combinations E1, E2, E3, E4, E5, E6, E7, E8, E9, E10, E11, E12, E13, E14, E15, E16, E17, E18, E19, and E20 as set out on FIG. 34 (PPGL vs PHT).

### Methods Using Biomarkers and Combinations Thereof.

The disclosure relates to methods and uses for stratifying a hypertensive patient among a plurality of hypertensive diseases, such as EHT, PHT, PA, CS and PPGL.

The methods and uses comprises the use of a combination of biomarkers as above described.

The methods and uses may be used for stratifying a hypertensive patient among Primary Aldosteronism (PA) vs Pheochromocytoma/Functional Paraganglioma (PPGL) vs Cushing's Syndrome (CS) vs Primary Hypertension (PHT), or among EHT vs PHT, or among CS vs PHT, or among PA vs PHT, or among PPGL vs PHT.

The methods and uses may be used for diagnosing an EHT, and/or for diagnosing a PHT, and/or for diagnosing a PA, and/or for diagnosing a CS, and/or a PPGL.

Also commonly known as essential hypertension, primary hypertension (PHT) is an elevated blood pressure disorder whose causes are not readily identifiable. Its prevalence raises with age in most populations. Due to fact that the causes are unknown, this type of hypertension is also known as essential hypertension.

Primary aldosteronism (PA) is recognized as a treatable cause of hypertension with a prevalence ranging from 4.6% to 13.0% in patients with hypertension and up to 20% in those with treatment-resistant hypertension (Yang et al., Nephrology, 22 (2017) 663-677). Primary aldosteronism (PA) is a heterogeneous condition, due to aldosterone-producing adenoma (40-50%) or bilateral adrenal hyperplasia (50-60%), also called idiopathic hyperaldosteronism, in the majority of cases. Unilateral adrenal hyperplasia represents <2% of cases and aldosterone-producing adrenocortical carcinoma is extremely rare. Primary aldosteronism may be inherited in familial hyperaldosteronism type I-IV or occur in conjunction with other abnormalities in PASNA (PA, seizures and neurological abnormalities), a rare syndrome featuring PA and neuromuscular abnormalities. In PA, aldosterone production is autonomous, thereby leading to elevated aldosterone levels that are not suppressible by sodium loading or volume expansion, together with low or suppressed renin (Funder et al., J Clin Endocrinol Metab. 2016; 101 (5): 1889-1916). Different genetic abnormalities have been associated with familial forms of the disease and APA (Fernandes-Rosa et al., Trends Mol Med. 2020; Zennaro et al., Nat Rev Endocrinol. 2020 October; 16 (10): 578-589).

Cushing's syndrome (CS) refers to a state of glucocorticoid excess with multiple deleterious manifestations, such as hypertension, obesity and glucose intolerance; all conferring increased cardiovascular risk. The most common cause by far is iatrogenic from exogenous glucocorticoids, which initially needs to be excluded. Cushing's syndrome is considered a rare endocrine disorder with an incidence of 2-3 cases per million per year with 80% attributed to ACTH-dependent causes and 20% due to ACTH-independent causes (Nieman et al., J Clin Endocrinol Metab. 2008; 93 (5): 1526-1540). The disease may be associated to different genetic abnormalities, such as USP8 gene mutations in Cushing's disease and mutations in PRKAR1A, ARMC5, MEN1, APC, FH as well as PRKACA in adrenal Cushing syndrome (Vaduva et al., J Endocr Soc 2020).

Phaeochromocytomas and functional paragangliomas (PPGL) are rare neuroendocrine tumours associated with hypertension due to the autonomous production of catecholamines such as adrenaline and noradrenaline. Phaeochromocytomas are derived from the chromaffin cells of the adrenal medulla, whereas paragangliomas arise from the sympathetic ganglia. PPGL have an estimated annual incidence of 0.5-0.8 per 100 000 person-years and probably account for 0.2-0.6% of hypertensive individuals. While most cases are sporadic disease presenting in midlife, approximately 30% are of all patients with PPGLs carry disease-causing germline mutations (Lenders et al., J Clin Endocrinol Metab. 2014; 99 (6): 1915-1942).

Methods and uses may be carried ex vivo or in vitro. Methods and uses may be carried on biological samples isolated from a patient.

The different types of hypertensive patient considered herein are: EHT patients, PHT patients, PA patients, CS patients and PPGL patients.

The disclosure relates to a use of a combination of biomarkers for stratifying a hypertensive patient among a plurality of hypertensive diseases.

In some embodiments, the disclosure relates to a use of a combination of biomarkers for stratifying a hypertensive patient among a plurality of hypertensive diseases, the plurality of hypertensive diseases comprising Primary Aldosteronism (PA), Pheochromocytoma/Functional Paraganglioma (PPGL), Cushing's Syndrome (CS) and Primary Hypertension (PHT), and the combination of biomarkers being a combination of biomarkers as above defined.

In some embodiments, the disclosure relates to a use of a combination of biomarkers for stratifying a hypertensive patient among a plurality of hypertensive diseases, the plurality of hypertensive diseases comprising Endocrine Hypertension (EHT) and Primary Hypertension (PHT), and the combination of biomarkers being a combination of biomarkers as above defined.

In some embodiments, the disclosure relates to a use of a combination of biomarkers for stratifying a hypertensive patient among a plurality of hypertensive diseases, the plurality of hypertensive diseases comprising Cushing's Syndrome (CS) and Primary Hypertension (PHT), and the combination of biomarkers being a combination of biomarkers as above defined.

In some embodiments, the disclosure relates to a use of a combination of biomarkers for stratifying a hypertensive patient among a plurality of hypertensive diseases, the plurality of hypertensive diseases comprising Primary Aldosteronism (PA) and Primary Hypertension (PHT), and the combination of biomarkers being a combination of biomarkers as above defined.

In some embodiments, the disclosure relates to a use of a combination of biomarkers for stratifying a hypertensive patient among a plurality of hypertensive diseases, the plurality of hypertensive diseases comprising Pheochromocytoma/Functional Paraganglioma (PPGL) and Primary Hypertension (PHT), and the combination of biomarkers being a combination of biomarkers as above defined.

A use of a combination of biomarkers for stratifying a hypertensive patient among a plurality of hypertensive diseases.
wherein the use comprises:
   measuring, ex vivo, said combination of biomarkers, and
   operating a trained classifier on said measured combination of biomarkers for obtaining a probability, for each hypertensive disease, of associating the hypertensive patient with a hypertensive disease in order to stratify said hypertensive patient among said plurality of types of hypertensive diseases,
said trained classifier being a classifier trained beforehand to learn a plurality of combinations of biomarkers previously selected based on at least one computed evaluation parameter and for several comparisons of at least two types of hypertensive patients.

A use of a combination of biomarkers for stratifying a hypertensive patient among a plurality of hypertensive diseases:
(i-a) when the plurality of hypertensive diseases is comprising Primary Aldosteronism (PA), Pheochromocytoma/Functional Paraganglioma (PPGL), Cushing's Syndrome (CS) and Primary Hypertension (PHT), then the combination of biomarkers may be a combination of biomarkers (i-a) as above defined or defined elsewhere in the specification.
(i-b) when the plurality of hypertensive diseases is comprising Endocrine Hypertension (EHT) and Primary Hypertension (PHT), then the combination of biomarkers may be a combination of biomarkers (i-b) as above defined or defined elsewhere in the specification,
wherein the use comprises:
   measuring, ex vivo, said combination of biomarkers, and
   operating a trained classifier on said measured combination of biomarkers for obtaining a probability, for each hypertensive disease, of associating the hypertensive patient with a hypertensive disease in order to stratify said hypertensive patient among said plurality of types of hypertensive diseases according to (i-a) or according to (i-b),
said trained classifier being a classifier trained beforehand to learn a plurality of combinations of biomarkers previously selected based on at least one computed evaluation parameter and for several comparisons of at least two types of hypertensive patients.

In some embodiments, a use may implement a set of at least two, for example 3, or more, combinations of biomarkers, as well as, in some cases, several different classifiers. In such embodiments, a set of probabilities is obtained, each corresponding to a combination of biomarkers and/or a classifier and/or a disease prediction. The hypertensive patient may be associated with the hypertensive disease obtaining the highest predicted probability of being associated with said patient. In a variant, a majority voting scheme may be used: each individual classifier may provide probability for each disease prediction which can be then transformed into a label, said labels from each independent different classifiers may be used to run a majority voting scheme to determine a single final label.

The disclosure relates to a combination of biomarkers as above defined for use in a method for stratifying a hypertensive patient among a plurality of hypertensive diseases.

In some embodiments, the disclosure relates to a method for stratifying a hypertensive patient among a plurality of types of hypertensive diseases,
the plurality of types of hypertensive diseases comprising Primary Aldosteronism (PA), Pheochromocytoma/Functional Paraganglioma (PPGL), Cushing's Syndrome (CS) and Primary Hypertension (PHT),
the method using at least one classifier trained beforehand to learn a plurality of combinations of biomarkers previously selected based on at least one computed evaluation parameter and for several comparisons of at least two types of hypertensive patients, and
the method comprising at least the steps of:
a) measuring, ex vivo, e.g., in suitable biological samples previously isolated from said patient, a combination of biomarkers,
wherein for said plurality of hypertensive diseases, the combination of biomarkers is a combination of biomarkers as above defined,
b) operating said trained classifier on the combination of biomarkers obtained at step a) from said hypertensive patient to stratify said hypertensive patient among said plurality of types of hypertensive diseases.

The step b) may comprise operating the trained classifier on the combination of biomarkers obtained at step a) from said hypertensive patient for obtaining a probability, for each hypertensive disease, of associating the hypertensive patient with a hypertensive disease in order to stratify said hypertensive patient among said plurality of types of hypertensive diseases.

In some embodiments, the disclosure relates to a method for stratifying a hypertensive patient among a plurality of types of hypertensive diseases,
the plurality of types of hypertensive diseases comprising Endocrine Hypertension (EHT) and Primary Hypertension (PHT),
the method using at least one classifier trained beforehand to learn a plurality of combinations of biomarkers previously selected based on at least one computed evaluation parameter and for several comparisons of at least two types of hypertensive patients, and
the method comprising at least the steps of:
a) measuring, ex vivo, e.g., in suitable biological samples previously isolated from said patient, a combination of biomarkers,
wherein for said plurality of hypertensive diseases, the combination of biomarkers is a combination of biomarkers as previously defined,
b) operating said trained classifier on the combination of biomarkers obtained at step a) from said hypertensive patient to stratify said hypertensive patient among said plurality of types of hypertensive diseases.

The step b) may comprise operating the trained classifier on the combination of biomarkers obtained at step a) from said hypertensive patient for obtaining a probability, for each hypertensive disease, of associating the hypertensive patient with a hypertensive disease in order to stratify said hypertensive patient among said plurality of types of hypertensive diseases.

In some embodiments, the disclosure relates to a method for stratifying a hypertensive patient among a plurality of types of hypertensive diseases,
the plurality of types of hypertensive diseases comprising Cushing's Syndrome (CS) and Primary Hypertension (PHT),
the method using at least one classifier trained beforehand to learn a plurality of combinations of biomarkers previously selected based on at least one computed evaluation parameter and for several comparisons of at least two types of hypertensive patients, and
the method comprising at least the steps of:
a) measuring, ex vivo, e.g., in suitable biological samples previously isolated from said patient, a combination of biomarkers,
wherein for said plurality of hypertensive diseases, the combination of biomarkers is a combination of biomarkers as previously defined,
b) operating said trained classifier on the combination of biomarkers obtained at step a) from said hypertensive patient to stratify said hypertensive patient among said plurality of types of hypertensive diseases.

The step b) may comprise operating the trained classifier on the combination of biomarkers obtained at step a) from said hypertensive patient for obtaining a probability, for each hypertensive disease, of associating the hypertensive patient with a hypertensive disease in order to stratify said hypertensive patient among said plurality of types of hypertensive diseases.

In some embodiments, the disclosure relates to a method for stratifying a hypertensive patient among a plurality of types of hypertensive diseases,
the plurality of types of hypertensive diseases comprising Primary Aldosteronism (PA) and Primary Hypertension (PHT),
the method using at least one classifier trained beforehand to learn a plurality of combinations of biomarkers previously selected based on at least one computed evaluation parameter and for several comparisons of at least two types of hypertensive patients, and
the method comprising at least the steps of:
a) measuring, ex vivo, e.g., in suitable biological samples previously isolated from said patient, a combination of biomarkers,
wherein for said plurality of hypertensive diseases, the combination of biomarkers is a combination of biomarkers as previously defined,
b) operating said trained classifier on the combination of biomarkers obtained at step a) from said hypertensive patient to stratify said hypertensive patient among said plurality of types of hypertensive diseases.

The step b) may comprise operating the trained classifier on the combination of biomarkers obtained at step a) from said hypertensive patient for obtaining a probability, for each hypertensive disease, of associating the hypertensive patient with a hypertensive disease in order to stratify said hypertensive patient among said plurality of types of hypertensive diseases.

In some embodiments, the disclosure relates to a method for stratifying a hypertensive patient among a plurality of types of hypertensive diseases,
the plurality of types of hypertensive diseases comprising Pheochromocytoma/Functional Paraganglioma (PPGL) and Primary Hypertension (PHT),
the method using at least one classifier trained beforehand to learn a plurality of combinations of biomarkers previously selected based on at least one computed evaluation parameter and for several comparisons of at least two types of hypertensive patients, and
the method comprising at least the steps of:
a) measuring, ex vivo, e.g., in suitable biological samples previously isolated from said patient, a combination of biomarkers,
wherein for said plurality of hypertensive diseases, the combination of biomarkers is a combination of biomarkers as previously defined,
b) operating said trained classifier on the combination of biomarkers obtained at step a) from said hypertensive patient to stratify said hypertensive patient among said plurality of types of hypertensive diseases.

The step b) may comprise operating the trained classifier on the combination of biomarkers obtained at step a) from said hypertensive patient for obtaining a probability, for each hypertensive disease, of associating the hypertensive patient with a hypertensive disease in order to stratify said hypertensive patient among said plurality of types of hypertensive diseases.

The methods may comprise a step of quantifying or detecting the presence of the biomarkers of the considered combination of biomarkers. The quantification or detection of the biomarkers may be carried out according to any suitable known techniques in the art.

A method of the disclosure may further comprise a step of obtaining for each type of hypertensive disease a probability associating the hypertensive patient to said hypertensive disease.

The trained classifier may be selected from Decision Trees (J48), Naïve Bayes (NB), K-nearest neighbours (IBk), LogitBoost (LB), support vector machine (SVM), Logistic Model Tree (LMT), Bagging, Simple Logistic (SL), Random Forest (RF) and Sequential Minimal Optimisation (SMO).

In some embodiments, the trained classifier may be selected from LogitBoost (LB), Simple Logistic (SL), and Random Forest (RF).

The classifier may have been trained with at least one predefined input dataset according to a method comprising at least the steps of:
a) for said at least one predefined input dataset and for at least one given comparison between at least a first and a second hypertensive patient, each patient having a first and a second type of hypertensive disease selected in said group of hypertensive diseases, the first and second type hypertensive disease being different, using said classifier to rank several combinations of biomarkers based on a computation of at least one evaluation parameter, and
b) based on said computed evaluation parameter(s), selecting a combination of biomarkers in order to stratify a hypertensive patient among said plurality of types of hypertensive diseases.

The evaluation parameter may be chosen among accuracy, sensitivity, specificity, AUC, F1, Kappa score, and combinations thereof.

The method may comprise the transmission, to the patient or a medical expert, of an output of the trained classifier, for example a probability estimating which type of hypertensive patients said patient is, or several probabilities, each corresponding to one type of hypertensive patients.

The probability(ies) is/are in the form of a numerical value, for example a value comprised between 0 and 1. In a variant the probability(ies) is/are in the form of a letter, especially showing that a patient is thought to belong to a type of hypertensive patients, for example group A, group B, group C, and so on.

The probabilities attribute a risk to a patient of being stratified in one of the hypertensive diseases: “EHT”, “PHT”, “PPGL”, “CS”, or “PA”.

The probability(ies) may be transmitted to a user by any suitable mean, for example by being displayed on a screen of an electronic device, printed, or by vocal synthesis.

The probability(ies) may be used as entry value in another program, and/or may be combined to other information, for example clinical and/or biological data.

In some embodiments, a method may implement a set of at least two, for example 3, or more, combinations of biomarkers, as well as, in some cases, several different classifiers. In such embodiments, a set of probabilities is obtained, each corresponding to a combination of biomarkers and/or a classifier and/or a disease prediction. The hypertensive patient may be associated with the hypertensive disease obtaining the highest predicted probability of being associated with said patient. In a variant, a majority voting scheme may be used: each individual classifier may provide probability for each disease prediction which can be then transformed into a label, said labels from independent different classifiers may be used to run a majority voting scheme to determine a single final label.

Each step of the methods according to the invention may be carried out on an electronic system, in particular a personal computer, a calculation server or a medical imaging device, preferably comprising at least a microcontroller and a memory.

In some embodiments, in the methods, the classifier may be trained beforehand to learn a plurality of combinations of biomarkers previously selected based on at least one computed evaluation parameter and for several comparisons of at least a first and a second type of hypertensive disease selected in a group of hypertensive diseases comprising Primary Aldosteronism (PA), Pheochromocytoma/Functional Paraganglioma (PPGL), Cushing's Syndrome (CS), Primary Hypertension (PHT), and Endocrine Hypertension (EHT).

A method for training a classifier to learn a plurality of combinations of biomarkers in order to stratify hypertensive patients suspected to have a hypertension among a plurality of hypertension diseases, may use at least one computed evaluation parameter, several comparisons of at least two types of hypertensive patients and several predefined input datasets. The method may comprise at least the following steps:
for each predefined input dataset and for each comparison between at least two types of hypertensive patients, selecting at least one combination of biomarkers based on a computation of said at least one evaluation parameter, and
training the classifier to learn said selected combinations of biomarkers associated with the comparisons between said types of hypertensive patients.

A method for learning a plurality of combinations of biomarkers for stratifying a hypertensive patient suspected to have a hypertension among a plurality of hypertensive diseases may use at least one classifier with at least one predefined input dataset and may comprise:
for said at least one predefined input dataset and for at least one given comparison between at least two types of hypertensive patients, a use of said classifier to rank several combinations of biomarkers based on a computation of at least one evaluation parameter, and
based on said computed evaluation parameter(s), a selection of a combination of biomarkers in order to stratify said hypertensive patient among said plurality of hypertensive diseases.

In the methods for learning a plurality of combinations of biomarkers, the at least one given comparison is preferably chosen among all the types of hypertensive patients versus all the types (ALL-ALL), EHT versus PHT, PPGL versus PHT, CS versus PHT and PA versus PHT. The at least one given comparison may also be chosen among PPGL versus CS, PPGL versus PA and CS versus PA.

The methods for learning a plurality of combinations of biomarkers are advantageously computer-implemented methods.

The methods for stratifying a hypertensive patient according to the disclosure are advantageously computer-implemented methods.

At least a part of the at least one predefined input dataset is advantageously extracted from at least one biological sample previously isolated from said patient.

The input dataset may comprise at least one omic determination in a biological sample obtained from a patient, and/or a patient's age. An omic to be determined may be chosen from a O-methylated catecholamine, a steroid, a small metabolite, or a miRNA. A steroid may plasma and/or urinary steroids.

An evaluation parameter may be chosen among accuracy, sensitivity, specificity, AUC, F1, and Kappa score, and a combination thereof.

By “classifier”, it has to be understood a learning model with associated learning algorithms that analyze data, used for classification and regression analysis.

The at least one classifier may be chosen among Decision Trees (J48), Naïve Bayes (NB), K-nearest neighbours (IBk), LogitBoost (LB), support vector machine (SVM), Logistic Model Tree (LMT), Bagging, Simple Logistic (SL), Random Forest (RF) and Sequential Minimal Optimisation (SMO). In a variant, the classifier is a neuronal network. However, this list is not exhaustive, and the invention is not limited to a specific type of classifier. The combinations of biomarkers may be split into a training set and a test set.

In some embodiments, the classifier may be selected from LogitBoost (LB), Simple Logistic (SL), and Random Forest (RF).

The predefined input dataset includes a parameter for choosing a comparison between at least two types of hypertensive patients and/or at least one biomarker or at least one combination of biomarkers.

At least one feature selection method may be used during the step of selecting the combinations of biomarkers, in particular wrapper-based and filter-based methods. However, this list is not exhaustive, and the invention is not limited to a specific type of feature selection method.

In a variant, no feature selection method is used. All combinations of biomarkers are thus ranked, no feature reduction being applied.

The predefined input dataset may comprise or not outlier biomarkers. Predefined input datasets may thus include or exclude outlier values. Extreme outliers may be removed by applying the quartile method. Excluding outliers may provide a better biomarkers identification and then better performances for stratifying a patient suspected to have a hypertension disease among several types of hypertensive diseases.

Several classifiers may be used independently, and at least two classifiers, especially three, are selected based on said computed evaluation parameter(s).

In the case where feature reduction is applied, several feature selection methods may be successively used to perform the step of selecting the combinations of biomarkers, and at least one feature selection method is selected based on said computed evaluation parameter(s). The classifier and/or the feature selection method with the best evaluation may thus be selected, which allows having an efficient and reliable identification method.

In some embodiments, the disclosure relates to a combination of biomarkers for use in a method for treating a hypertensive disease in a patient in need thereof, the hypertensive disease being selected among a plurality of hypertensive diseases,
the method of treating comprising a step of stratifying the hypertensive patient among the plurality of hypertensive diseases, said step of stratifying comprising:
   measuring, ex vivo, said combination of biomarkers, and
   operating a trained classifier on said measured combination of biomarkers for obtaining a probability, for each hypertensive disease, of associating the hypertensive patient with a hypertensive disease in order to stratify said hypertensive patient among said plurality of types of hypertensive diseases,
said trained classifier being a classifier trained beforehand to learn a plurality of combinations of biomarkers previously selected based on at least one computed evaluation parameter and for several comparisons of at least two types of hypertensive patients.

In some embodiments, the disclosure relates to a use of a combination of biomarkers in a method for stratifying and treating a hypertensive disease in a patient in need thereof, the hypertensive disease being selected among Primary Aldosteronism (PA), Pheochromocytoma/Functional Paraganglioma (PPGL), Cushing's Syndrome (CS) and Primary Hypertension (PHT), and the combination of biomarkers being a combination of biomarkers as above defined.

In some embodiments, the disclosure relates to a use of a combination of biomarkers in a method for stratifying and treating a hypertensive disease in a patient in need thereof, the hypertensive disease being selected among Endocrine Hypertension (EHT) and Primary Hypertension (PHT), and the combination of biomarkers being a combination of biomarkers as above defined.

In some embodiments, the disclosure relates to a use of a combination of biomarkers in a method for stratifying and treating a hypertensive disease in a patient in need thereof, the hypertensive disease being selected among Cushing's Syndrome (CS) and Primary Hypertension (PHT), and the combination of biomarkers being a combination of biomarkers as above defined.

In some embodiments, the disclosure relates to a use of a combination of biomarkers in a method for stratifying and treating a hypertensive disease in a patient in need thereof, the hypertensive disease being selected among Primary Aldosteronism (PA) and Primary Hypertension (PHT), and the combination of biomarkers being a combination of biomarkers as above defined.

In some embodiments, the disclosure relates to a use of a combination of biomarkers in a method for stratifying and treating a hypertensive disease in a patient in need thereof, the hypertensive disease being selected among Pheochromocytoma/Functional Paraganglioma (PPGL) and Primary Hypertension (PHT), and the combination of biomarkers being a combination of biomarkers as above defined.

In some embodiments, the disclosure relates to a method for treating a hypertensive patient, said method comprising:
a) stratifying said hypertensive patient among a plurality of hypertensive diseases,
the stratification of said hypertensive patient among said plurality of hypertensive diseases being carried out according to a method comprising the steps of:
   measuring, ex vivo, said combination of biomarkers, and
   operating a trained classifier on said measured combination of biomarkers for obtaining a probability, for each hypertensive disease, of associating the hypertensive patient with a hypertensive disease in order to stratify said hypertensive patient among said plurality of types of hypertensive diseases,
said trained classifier being a classifier trained beforehand to learn a plurality of combinations of biomarkers previously selected based on at least one computed evaluation parameter and for several comparisons of at least two types of hypertensive patients,
b) selecting a therapeutic treatment presumed to treat or to relieve at least one symptom of the hypertensive disease associated to said patient, and
c) treating said patient by administering to said patient said therapeutic treatment selected at step b).

In some embodiments, the disclosure relates to a method for stratifying and treating a hypertensive patient, said method comprising stratifying said hypertensive patient among a plurality of hypertensive diseases,
the plurality of hypertensive diseases comprising Primary Aldosteronism (PA), Pheochromocytoma/Functional Paraganglioma (PPGL), Cushing's Syndrome (CS) and Primary Hypertension (PHT), and the combination of biomarkers being a combination of biomarkers as above defined,
and treating said patient with a therapeutic treatment presumed to treat or to relieve at least one symptom of the hypertensive disease, the method comprising at least the steps of:
a) stratifying said hypertensive patient among said plurality of hypertensive diseases according to a method of the disclosure, for associating a hypertensive disease to said patient,
b) selecting a therapeutic treatment presumed to treat or to relieve at least one symptom of the hypertensive disease associated to said patient, and
c) administering to said patient said therapeutic treatment selected at step b).

In some embodiments, the disclosure relates to a method for stratifying and treating a hypertensive patient, said method comprising stratifying said hypertensive patient among a plurality of hypertensive diseases,
the plurality of hypertensive diseases comprising Endocrine Hypertension (EHT) and Primary Hypertension (PHT), and the combination of biomarkers being a combination of biomarkers as above defined,
and treating said patient with a therapeutic treatment presumed to treat or to relieve at least one symptom of the hypertensive disease, the method comprising at least the steps of:
a) stratifying said hypertensive patient among said plurality of hypertensive diseases according to a method of the disclosure, for associating a hypertensive disease to said patient,
b) selecting a therapeutic treatment presumed to treat or to relieve at least one symptom of the hypertensive disease associated to said patient, and
c) administering to said patient said therapeutic treatment selected at step b).

In some embodiments, the disclosure relates to a method for stratifying and treating a hypertensive patient, said method comprising stratifying said hypertensive patient among a plurality of hypertensive diseases,
the plurality of hypertensive diseases comprising Cushing's Syndrome (CS) and Primary Hypertension (PHT), and the combination of biomarkers being a combination of biomarkers as above defined,
and treating said patient with a therapeutic treatment presumed to treat or to relieve at least one symptom of the hypertensive disease, the method comprising at least the steps of:
a) stratifying said hypertensive patient among said plurality of hypertensive diseases according to a method of the disclosure, for associating a hypertensive disease to said patient,
b) selecting a therapeutic treatment presumed to treat or to relieve at least one symptom of the hypertensive disease associated to said patient, and
c) administering to said patient said therapeutic treatment selected at step b).

In some embodiments, the disclosure relates to a method for stratifying and treating a hypertensive patient, said method comprising stratifying said hypertensive patient among a plurality of hypertensive diseases,
the plurality of hypertensive diseases comprising Primary Aldosteronism (PA) and Primary Hypertension (PHT), and the combination of biomarkers being a combination of biomarkers as above defined,
and treating said patient with a therapeutic treatment presumed to treat or to relieve at least one symptom of the hypertensive disease, the method comprising at least the steps of:
a) stratifying said hypertensive patient among said plurality of hypertensive diseases according to a method of the disclosure, for associating a hypertensive disease to said patient,
b) selecting a therapeutic treatment presumed to treat or to relieve at least one symptom of the hypertensive disease associated to said patient, and
c) administering to said patient said therapeutic treatment selected at step b).

In some embodiments, the disclosure relates to a method for stratifying and treating a hypertensive patient, said method comprising stratifying said hypertensive patient among a plurality of hypertensive diseases,
the plurality of hypertensive diseases comprising Pheochromocytoma/Functional Paraganglioma (PPGL) and Primary Hypertension (PHT), and the combination of biomarkers being a combination of biomarkers as above defined,
and treating said patient with a therapeutic treatment presumed to treat or to relieve at least one symptom of the hypertensive disease, the method comprising at least the steps of:
a) stratifying said hypertensive patient among said plurality of hypertensive diseases according to a method of the disclosure, for associating a hypertensive disease to said patient,
b) selecting a therapeutic treatment presumed to treat or to relieve at least one symptom of the hypertensive disease associated to said patient, and
c) administering to said patient said therapeutic treatment selected at step b).

An anti-hypertensive agent is an active agent which, according to the case, is acknowledged for the treatment of PHT, PA, CS or PPGL.

The method of treatment as disclosed herein may also comprise a step of observing the relieving, reduction, amelioration, improvement or cure of symptoms or signs of the EHT, in particular blood pressure.

PHT, PA, CS and PPGL treatments are well known in the art (Williams et al., J Hypertens. 2018; 36 (10): 1953-2041; Funder et al., J Clin Endocrinol Metab. 2016; 101 (5): 1889-1916; Nieman et al., J Clin Endocrinol Metab. 2008; 93 (5): 1526-1540; Lenders et al., J Clin Endocrinol Metab. 2014; 99 (6): 1915-1942; Feelders et al., J Clin Endocrinol Metab. 2013; 98 (2): 425-438). They may comprise surgery and/or administration of therapeutically active compounds.

As possible anti-hypertensive agent useful for the treatment of PHT, one may cite the ACE inhibitors, the angiotensin receptor blockers, the beta-blockers, the calcium channel blockers, and the diuretics (thiazides and thiazide-like diuretics such as chlorthalidone and indapamide).

As a matter of example, treatment of PA may comprise administration to the patient in need thereof, as an active agent, of at least one mineralocorticoid receptor (MR) antagonist, such as spironolactone or eplerenone. Also, one may consider the use of the epithelial sodium channel antagonists, such as amiloride, ACE inhibitors, angiotensin receptor blockers, or calcium channel blockers.

PPGL treatment may comprise administering to the patient in need thereof at least one α-adrenergic receptor blocker or one calcium channel blocker.

CS treatment may comprise administration to the patient in need thereof at least one steroidogenesis inhibitor or a glucocorticoid antagonist. As an active agent useful for the treatment of CS, one may mention ketoconazole, mitotane, etomidate, metyrapone, cabergoline, pasireotide, or mifepristone.

Dosage and schedule of administration of a therapeutic composition intended to treat or relieve PA, CS or PPGL, or associated symptoms, are adapted to the patient in need thereof according to age, body weight, blood pressure, and/or gender. Adjustment of a treatment to the specifics of a patient is within the common knowledge of the skilled person.

### Kits and Uses Thereof

In some embodiments, the disclosure relates to a kit for stratifying a hypertensive patient among a plurality of hypertensive diseases,
the plurality of hypertensive diseases comprising Primary Aldosteronism (PA), Pheochromocytoma/Functional Paraganglioma (PPGL), Cushing's Syndrome (CS) and Primary Hypertension (PHT), and the kit comprising at least means for measuring a combination of biomarkers as above defined.

In some embodiments, the disclosure relates to a kit for stratifying a hypertensive patient among a plurality of hypertensive diseases,
the plurality of hypertensive diseases comprising Endocrine Hypertension (EHT) and Primary Hypertension (PHT), and the kit comprising at least means for measuring a combination of biomarkers as above defined.

In some embodiments, the disclosure relates to a kit for stratifying a hypertensive patient among a plurality of hypertensive diseases,
the plurality of hypertensive diseases comprising Cushing's Syndrome (CS) and Primary Hypertension (PHT), and the kit comprising at least means for measuring a combination of biomarkers as above defined.

In some embodiments, the disclosure relates to a kit for stratifying a hypertensive patient among a plurality of hypertensive diseases,
the plurality of hypertensive diseases comprising Primary Aldosteronism (PA) and Primary Hypertension (PHT), and the kit comprising at least means for measuring a combination of biomarkers as above defined.

In some embodiments, the disclosure relates to a kit for stratifying a hypertensive patient among a plurality of hypertensive diseases,
the plurality of hypertensive diseases comprising Pheochromocytoma/Functional Paraganglioma (PPGL) and Primary Hypertension (PHT), and the kit comprising at least means for measuring a combination of biomarkers as above defined.

In some embodiments, the disclosure relates to a kit for stratifying a hypertensive patient among a plurality of hypertensive diseases,

In some embodiments, a kit according to the disclosure may comprise a notice of instructions setting out the steps of the methods for stratifying a hypertensive patient among a plurality of hypertensive diseases, as detailed in the disclosure.

In some embodiments, a kit may comprise means for determining the presence or quantifying miRNA as disclosed herein. Such means may be means for implementing a digital PCR, a quantitative RT-PCR, a Microarray, an Isothermal amplification, a Next-generation sequencing, a Hybridization chain reaction, or a Near-infrared technology.

In some embodiments, a kit may comprise means for determining the presence or quantifying O-methylated catecholamines as disclosed herein. Such means may be means for implementing a HPLC coupled with coulometric detection or a Liquid chromatography-tandem mass spectrometry (LC-MS/MS)

In some embodiments, a kit may comprise means for determining the presence or quantifying steroids as disclosed herein, in particular plasma steroids and/or urinary steroids. Such means may be means for implementing an ELISA, a liquid column chromatography, a gas chromatography/mass spectrometry, an UHPLC-ESI-QTOF-MS/MS, or a LC-MS/MS.

In some embodiments, a kit may comprise means for determining the presence or quantifying Small metabolites as disclosed herein. Such means may be means for implementing a gas chromatography-mass spectrometry [GC-MS], a liquid chromatography-MS, a NMR, a LC/GC-FID, a Direct Flow Injection MS/MS, a LC ESI-MS/MS, or a MS/MS.

### Computer Program Products

Such methods according to the invention are advantageously performed by means of computer programs, automatically on any electronic system comprising a processor, especially a computer.

In some embodiments, the disclosure relates to a computer program product for stratifying a hypertensive patient among a plurality of hypertensive diseases,
the plurality of hypertensive diseases comprising Primary Aldosteronism (PA), Pheochromocytoma/Functional Paraganglioma (PPGL), Cushing's Syndrome (CS) and Primary Hypertension (PHT),
the computer program using
(1) at least one classifier trained beforehand to learn a plurality of combinations of biomarkers previously selected based on at least one computed evaluation parameter and for several comparisons of at least a first and a second type of hypertensive disease selected in a group of hypertensive diseases comprising Primary Aldosteronism (PA), Pheochromocytoma/Functional Paraganglioma (PPGL), Cushing's Syndrome (CS) and Primary Hypertension (PHT),
the first and second type hypertensive disease being different, wherein said classifier has been trained with at least one predefined input dataset according to a method comprising at least the steps of:
a) for said at least one predefined input dataset and for at least one given comparison between at least a first and a second hypertensive patient, each patient having a first and a second type of hypertensive disease selected in said group of hypertensive diseases, the first and second type hypertensive disease being different, using said classifier to rank several combinations of biomarkers based on a computation of at least one evaluation parameter, and
b) based on said computed evaluation parameter(s), selecting a combination of biomarkers in order to stratify a hypertensive patient among said plurality of hypertensive diseases,
(2) with at least one input of measured biomarkers, said input of measured biomarkers being obtained by measuring, in suitable biological samples previously isolated from said patient, a combination of biomarkers as above defined for said plurality of
the computer program product comprising instructions which, when the program is executed by a computer, cause the computer to, for said at least one input of measured biomarkers, use said trained classifier for associating each hypertensive disease of said plurality of hypertensive diseases with a probability of associating the hypertensive patient with said hypertensive disease in order to stratify said hypertensive patient among said plurality of hypertensive diseases.

In some embodiments, the disclosure relates to a computer program product for stratifying a hypertensive patient among a plurality of hypertensive diseases,
the plurality of hypertensive diseases comprising Endocrine Hypertension (EHT) and Primary Hypertension (PHT),
the computer program using
(1) at least one classifier trained beforehand to learn a plurality of combinations of biomarkers previously selected based on at least one computed evaluation parameter and for several comparisons of at least a first and a second type of hypertensive disease selected in a group of hypertensive diseases comprising Endocrine Hypertension (EHT) and Primary Hypertension (PHT),
the first and second type hypertensive disease being different, wherein said classifier has been trained with at least one predefined input dataset according to a method comprising at least the steps of:
a) for said at least one predefined input dataset and for at least one given comparison between at least a first and a second hypertensive patient, each patient having a first and a second type of hypertensive disease selected in said group of hypertensive diseases, the first and second type hypertensive disease being different, using said classifier to rank several combinations of biomarkers based on a computation of at least one evaluation parameter, and
b) based on said computed evaluation parameter(s), selecting a combination of biomarkers in order to stratify a hypertensive patient among said plurality of hypertensive diseases,
(2) with at least one input of measured biomarkers, said input of measured biomarkers being obtained by measuring, in suitable biological samples previously isolated from said patient, a combination of biomarkers as above defined for said plurality of
the computer program product comprising instructions which, when the program is executed by a computer, cause the computer to, for said at least one input of measured biomarkers, use said trained classifier for associating each hypertensive disease of said plurality of hypertensive diseases with a probability of associating the hypertensive patient with said hypertensive disease in order to stratify said hypertensive patient among said plurality of hypertensive diseases.

In some embodiments, the disclosure relates to a computer program product for stratifying a hypertensive patient among a plurality of hypertensive diseases,
the plurality of hypertensive diseases comprising Cushing's Syndrome (CS) and Primary Hypertension (PHT),
the computer program using
(1) at least one classifier trained beforehand to learn a plurality of combinations of biomarkers previously selected based on at least one computed evaluation parameter and for several comparisons of at least a first and a second type of hypertensive disease selected in a group of hypertensive diseases comprising Cushing's Syndrome (CS) and Primary Hypertension (PHT),
the first and second type hypertensive disease being different, wherein said classifier has been trained with at least one predefined input dataset according to a method comprising at least the steps of:
a) for said at least one predefined input dataset and for at least one given comparison between at least a first and a second hypertensive patient, each patient having a first and a second type of hypertensive disease selected in said group of hypertensive diseases, the first and second type hypertensive disease being different, using said classifier to rank several combinations of biomarkers based on a computation of at least one evaluation parameter, and
b) based on said computed evaluation parameter(s), selecting a combination of biomarkers in order to stratify a hypertensive patient among said plurality of hypertensive diseases,
(2) with at least one input of measured biomarkers, said input of measured biomarkers being obtained by measuring, in suitable biological samples previously isolated from said patient, a combination of biomarkers as above defined for said plurality of
the computer program product comprising instructions which, when the program is executed by a computer, cause the computer to, for said at least one input of measured biomarkers, use said trained classifier for associating each hypertensive disease of said plurality of hypertensive diseases with a probability of associating the hypertensive patient with said hypertensive disease in order to stratify said hypertensive patient among said plurality of hypertensive diseases.

In some embodiments, the disclosure relates to a computer program product for stratifying a hypertensive patient among a plurality of hypertensive diseases,
the plurality of hypertensive diseases comprising Primary Aldosteronism (PA) and Primary Hypertension (PHT),
the computer program using
(1) at least one classifier trained beforehand to learn a plurality of combinations of biomarkers previously selected based on at least one computed evaluation parameter and for several comparisons of at least a first and a second type of hypertensive disease selected in a group of hypertensive diseases Primary Aldosteronism (PA) and Primary Hypertension (PHT),
the first and second type hypertensive disease being different, wherein said classifier has been trained with at least one predefined input dataset according to a method comprising at least the steps of:
a) for said at least one predefined input dataset and for at least one given comparison between at least a first and a second hypertensive patient, each patient having a first and a second type of hypertensive disease selected in said group of hypertensive diseases, the first and second type hypertensive disease being different, using said classifier to rank several combinations of biomarkers based on a computation of at least one evaluation parameter, and
b) based on said computed evaluation parameter(s), selecting a combination of biomarkers in order to stratify a hypertensive patient among said plurality of hypertensive diseases,
(2) with at least one input of measured biomarkers, said input of measured biomarkers being obtained by measuring, in suitable biological samples previously isolated from said patient, a combination of biomarkers as above defined for said plurality of
the computer program product comprising instructions which, when the program is executed by a computer, cause the computer to, for said at least one input of measured biomarkers, use said trained classifier for associating each hypertensive disease of said plurality of hypertensive diseases with a probability of associating the hypertensive patient with said hypertensive disease in order to stratify said hypertensive patient among said plurality of hypertensive diseases.

In some embodiments, the disclosure relates to a computer program product for stratifying a hypertensive patient among a plurality of hypertensive diseases,
the plurality of hypertensive diseases comprising Pheochromocytoma/Functional Paraganglioma (PPGL) and Primary Hypertension (PHT),
the computer program using
(1) at least one classifier trained beforehand to learn a plurality of combinations of biomarkers previously selected based on at least one computed evaluation parameter and for several comparisons of at least a first and a second type of hypertensive disease selected a group of in hypertensive diseases comprising Pheochromocytoma/Functional Paraganglioma (PPGL) and Primary Hypertension (PHT),
the first and second type hypertensive disease being different, wherein said classifier has been trained with at least one predefined input dataset according to a method comprising at least the steps of:
a) for said at least one predefined input dataset and for at least one given comparison between at least a first and a second hypertensive patient, each patient having a first and a second type of hypertensive disease selected in said group of hypertensive diseases, the first and second type hypertensive disease being different, using said classifier to rank several combinations of biomarkers based on a computation of at least one evaluation parameter, and
b) based on said computed evaluation parameter(s), selecting a combination of biomarkers in order to stratify a hypertensive patient among said plurality of hypertensive diseases,
(2) with at least one input of measured biomarkers, said input of measured biomarkers being obtained by measuring, in suitable biological samples previously isolated from said patient, a combination of biomarkers as above defined for said plurality of
the computer program product comprising instructions which, when the program is executed by a computer, cause the computer to, for said at least one input of measured biomarkers, use said trained classifier for associating each hypertensive disease of said plurality of hypertensive diseases with a probability of associating the hypertensive patient with said hypertensive disease in order to stratify said hypertensive patient among said plurality of hypertensive diseases.

The features defined above for the methods of the disclosure apply to the computer program products.

### EXAMPLES

The following working examples illustrate the embodiments of the disclosure that are presently best known. However, it is to be understood that the following are only exemplary or illustrative of the application of the principles of the present disclosure. Numerous modifications and alternative compositions, methods, and systems may be devised by those skilled in the art without departing from the spirit and scope of the present disclosure.

### Example 1: Materials and Methods

### Materials and Methods

### Patients Details and Multi-Omics Data

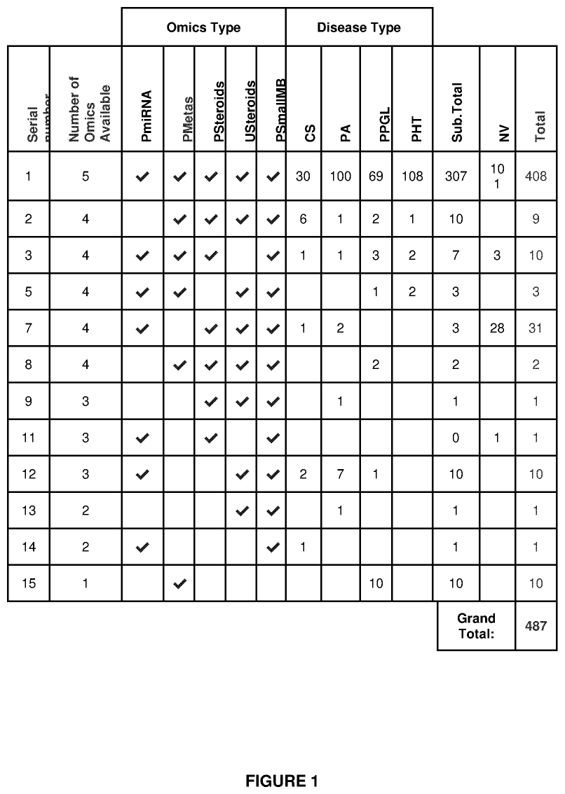

The study included 487 patients with PA, PPGL, CS and PHT as well as normotensive volunteers (NV) (PA=113, PPGL=88, CS=41, PHT=112, and NV=133), who were recruited by reference centres for adrenal disorders of the ENS@T-HT Horizon2020 consortium. (ENSAT-HT Project, n.d.) Diagnosis was based on current guidelines for each disease in each expert centre. Omics studies involved measurements of plasma miRNA (PmiRNA: 173), plasma O-methylated catecholamines (PMetas: 4), plasma steroids (PSteroids: 16), urinary steroid metabolites (USteroids: 27), and plasma Small metabolites (PSmallMB: 189) in biosamples collected from each patient within 24 h. After completion of omics measurements, quality controls and data cleaning, 408 patients had complete omics sets for further analysis (FIG. 1). Patients' demographic characteristics are summarized in Table 2.

Table 2 below shows a repartition of the number of patients for the input datasets 2 and 3 and according to four types of hypertensive patients: Primary Aldosteronism (PA), Pheochromocytoma/Functional Paraganglioma (PPGL), Cushing's Syndrome (CS), and Primary Hypertension (PHT).

TABLE 2

| Type of | | | | Input dataset 3 | |
|---|---|---|---|---|---|
| hypertensive | Patient | Input dataset 2 | | Patient | Patient |
| patients | Count | Male | Female | age >=50 | age <50 |
| CS | 30 | 3 | 27 | 15 | 15 |
| PA | 100 | 57 | 43 | 41 | 59 |
| PPGL | 69 | 30 | 39 | 39 | 30 |
| PHT | 108 | 47 | 61 | 69 | 39 |
| NV | 101 | 61 | 40 | 5 | 96 |
| (Normotensive | | | | | |
| Volunteers | | | | | |

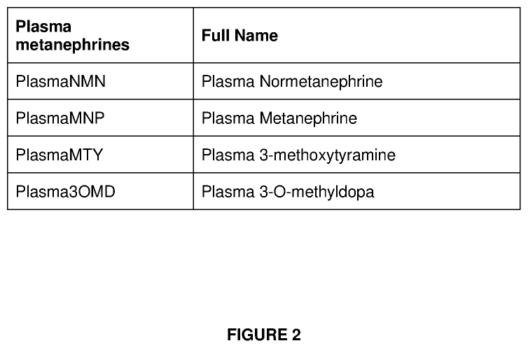

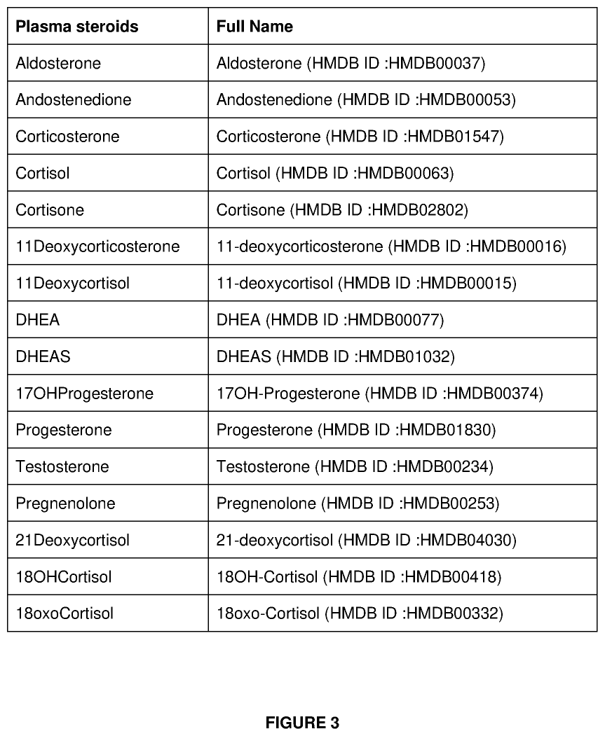

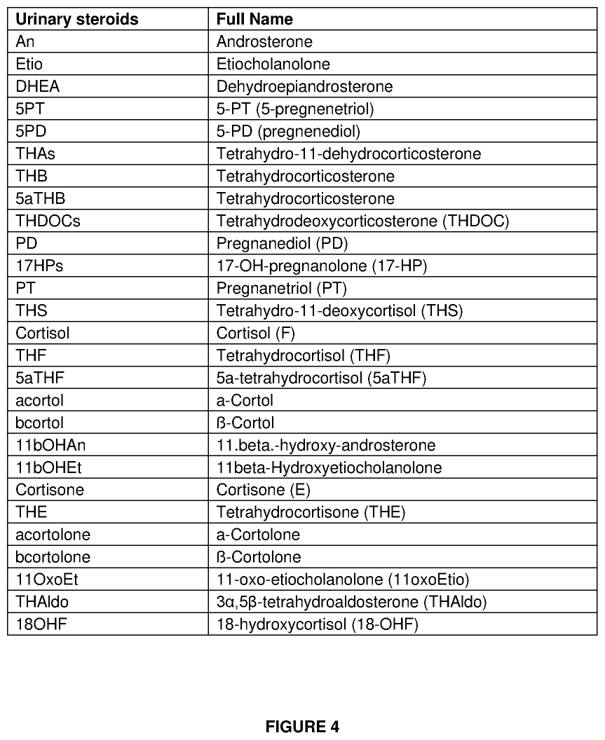

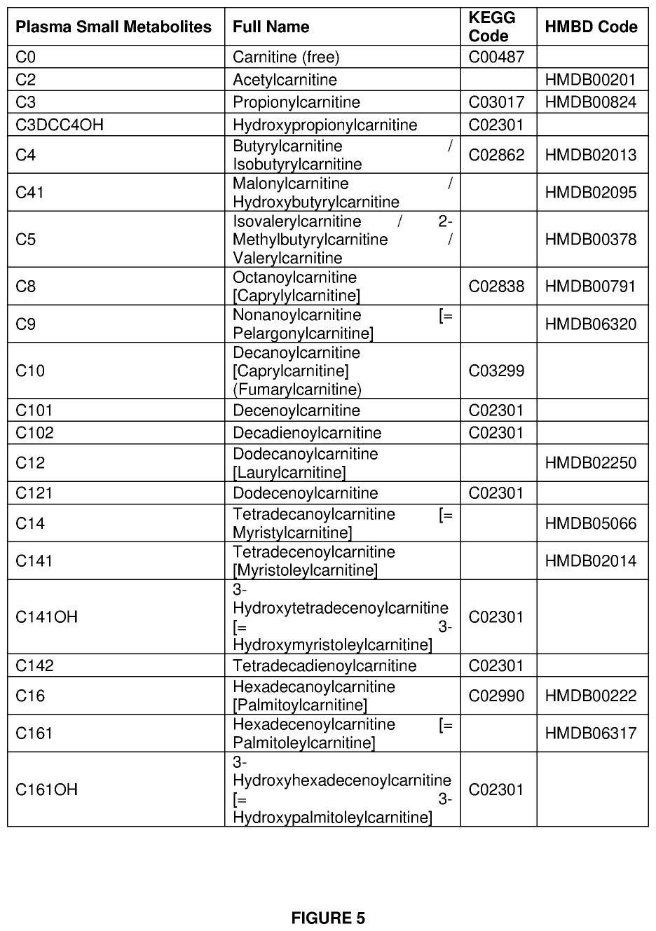

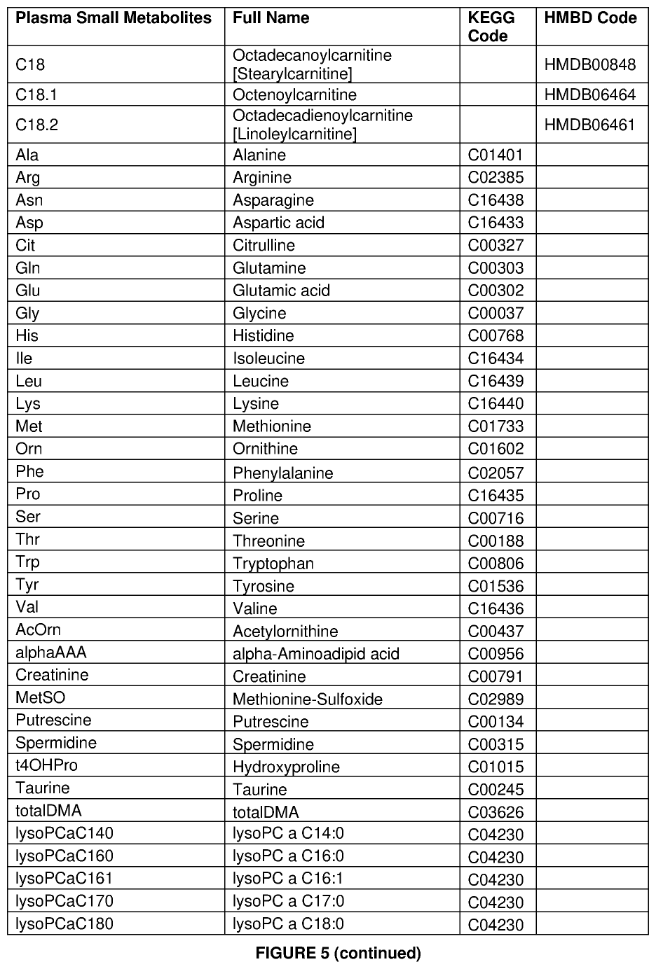

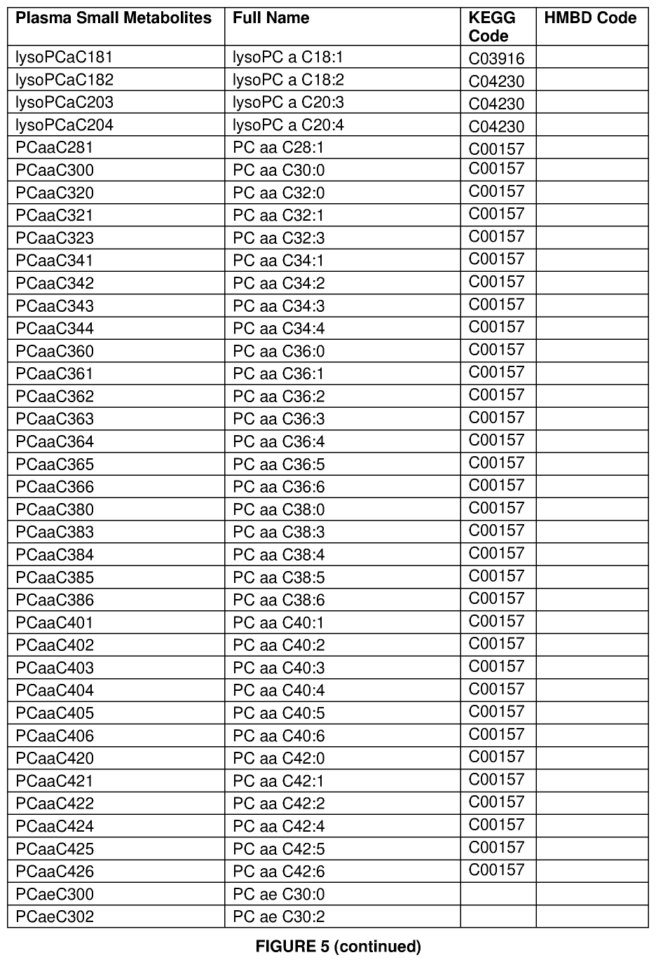

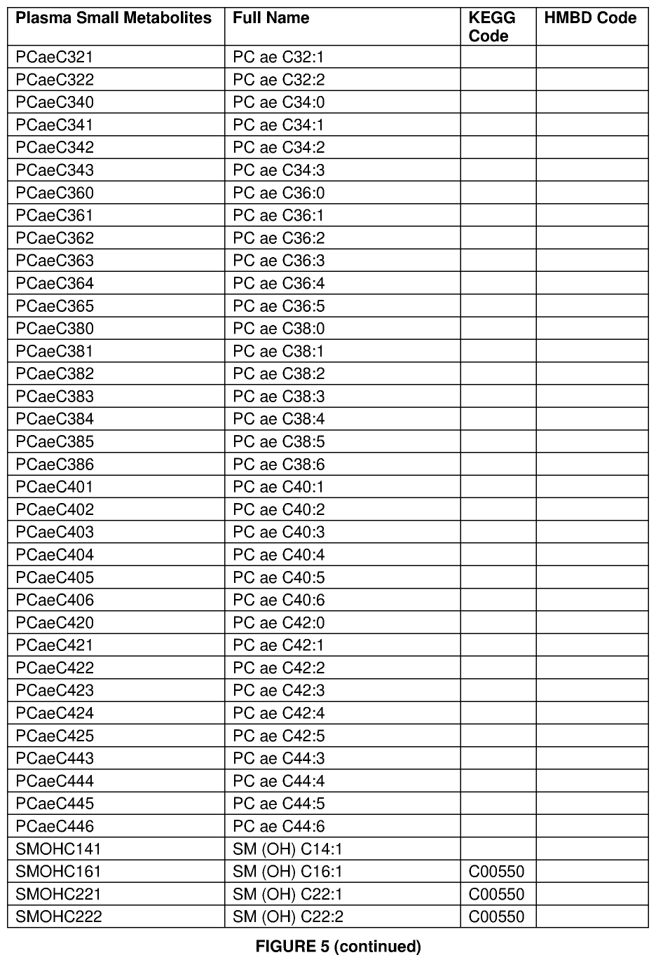

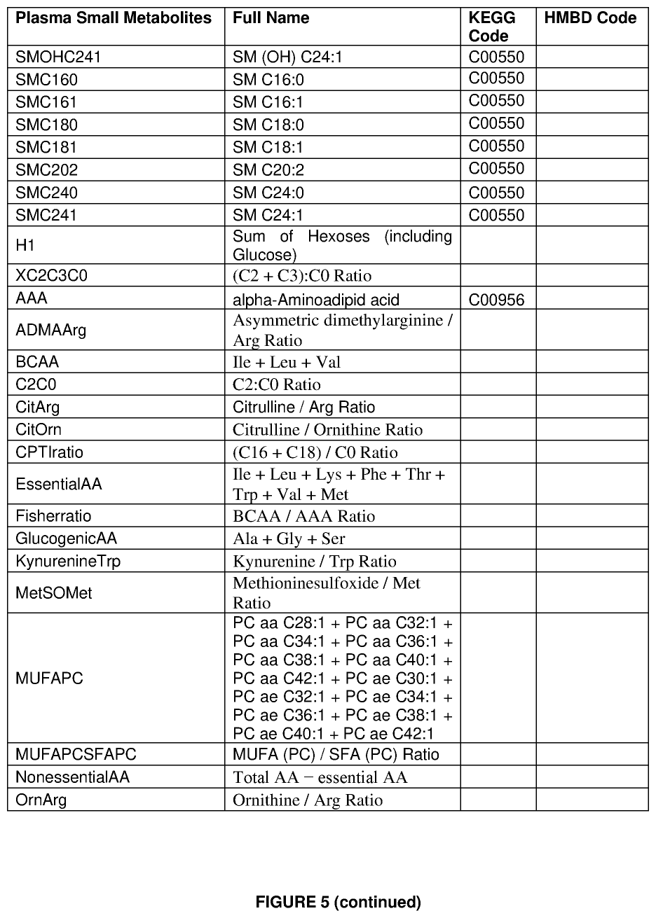

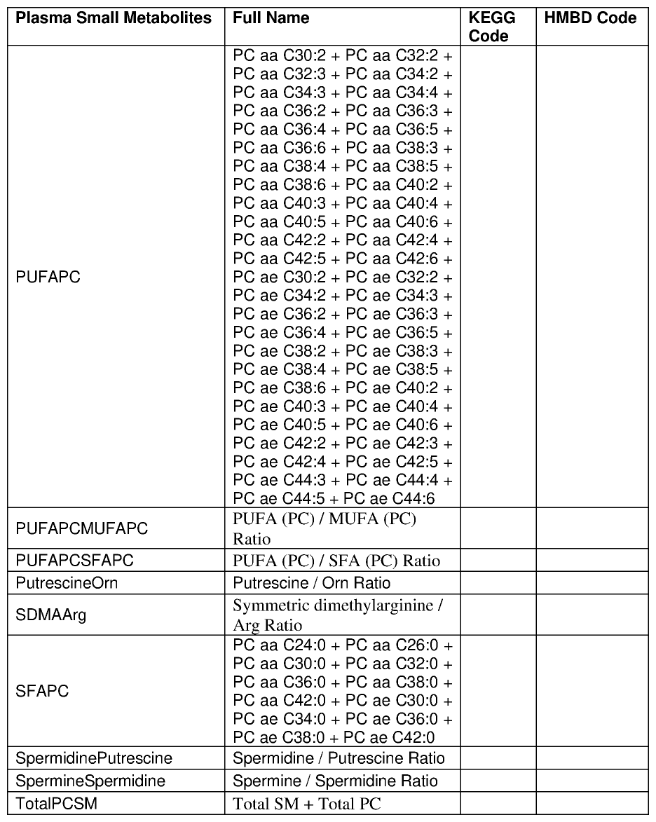

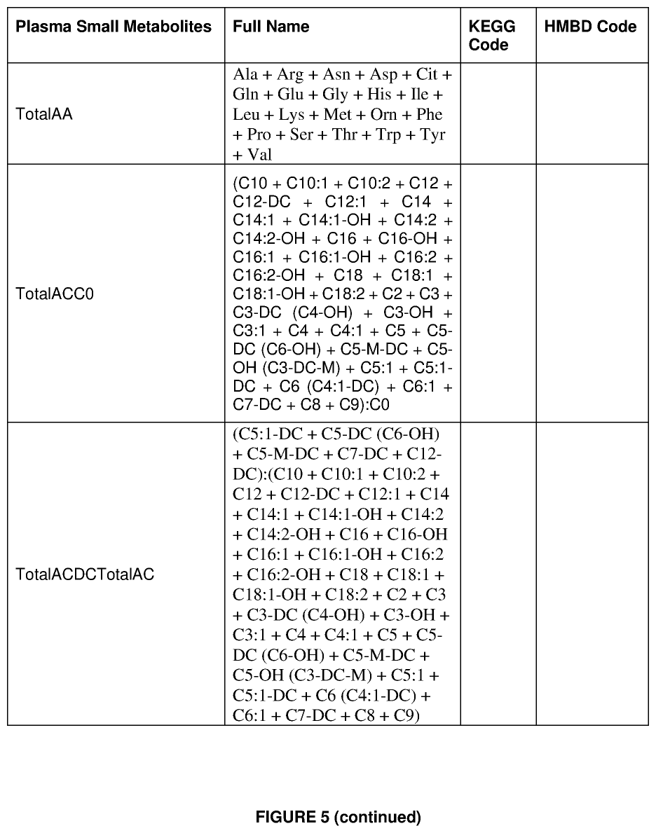

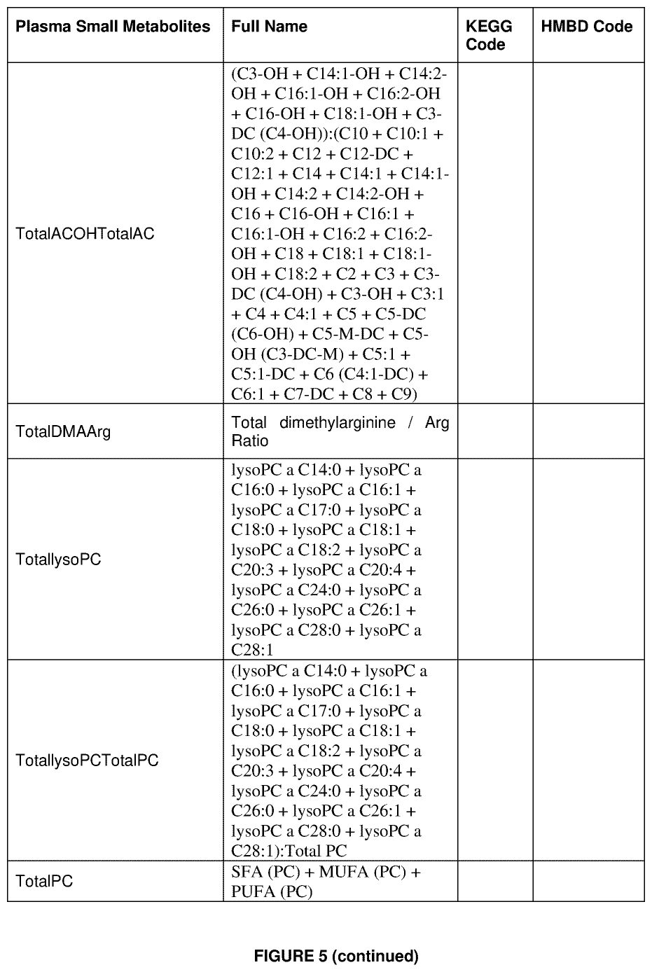

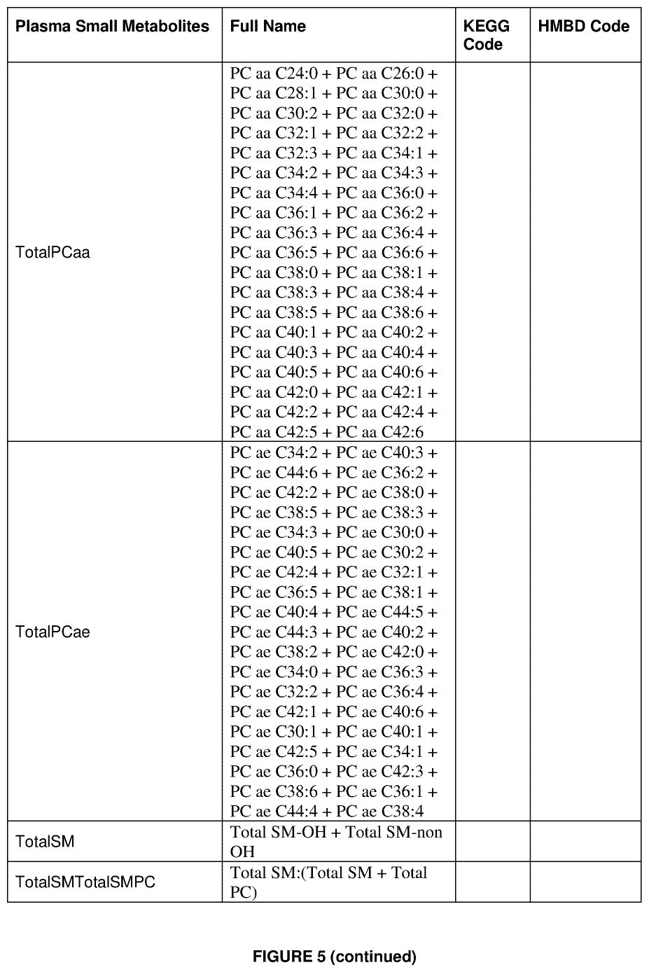

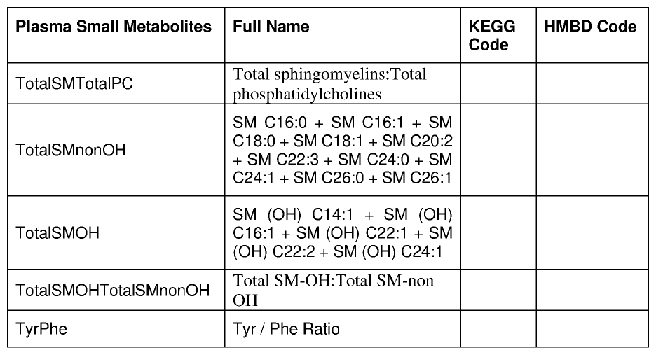

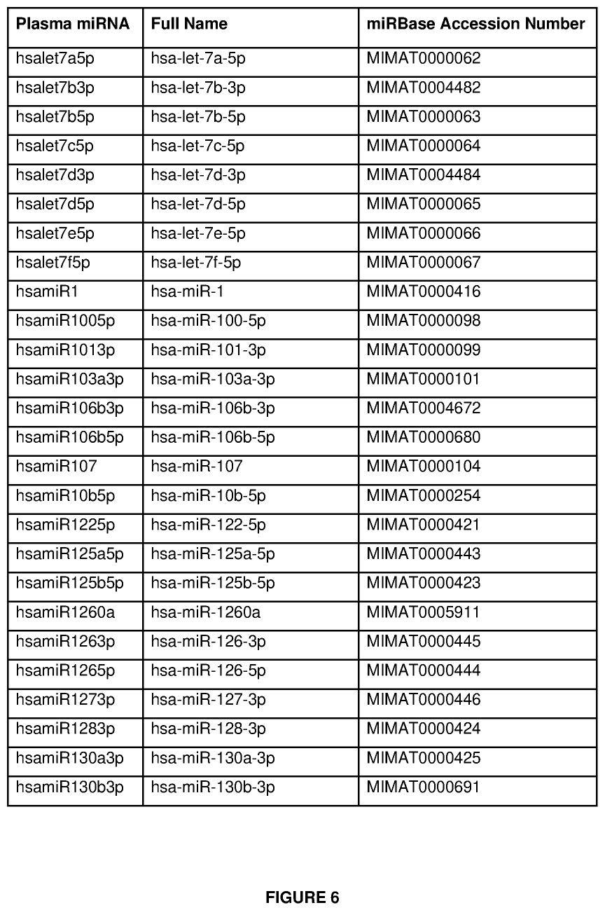

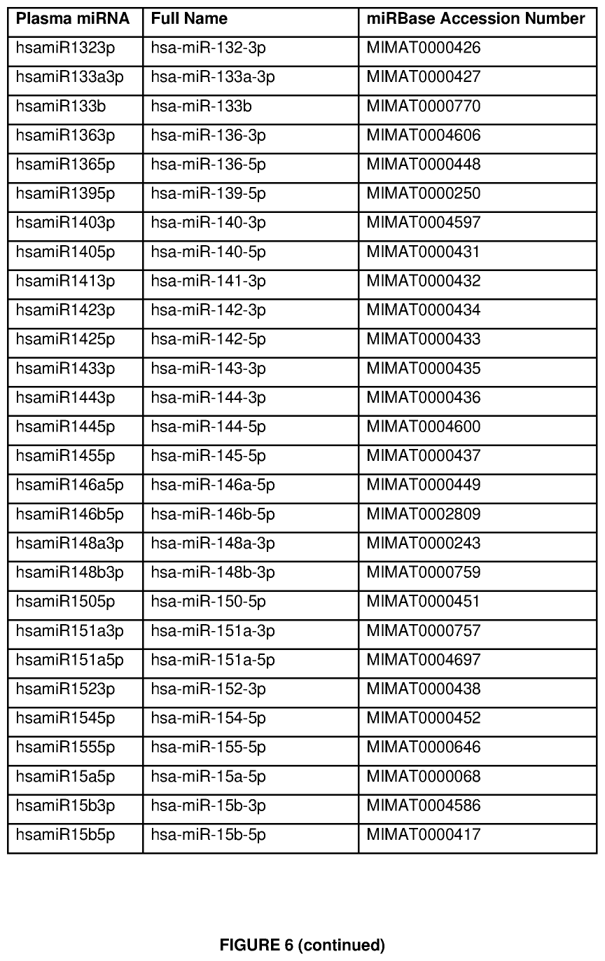

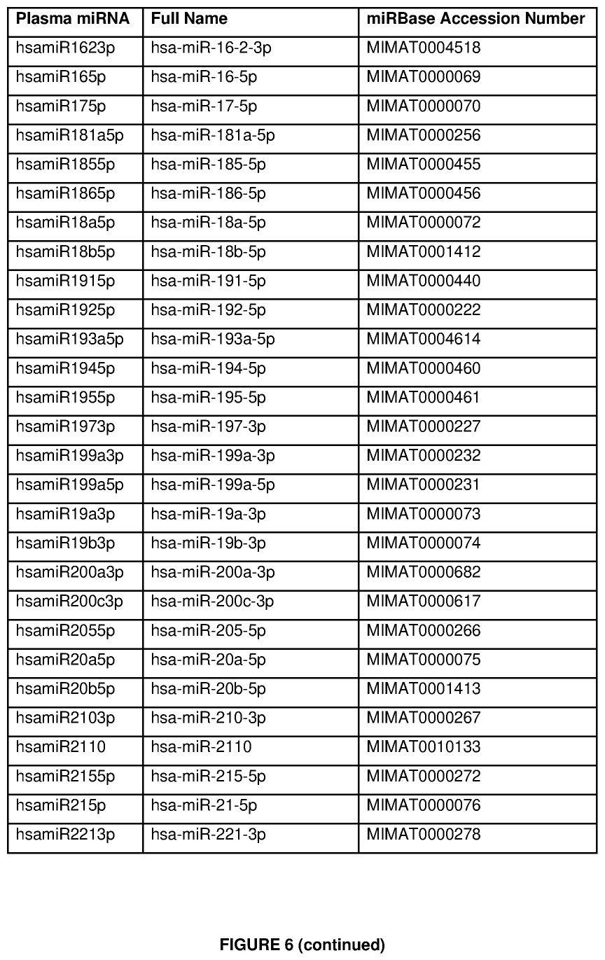

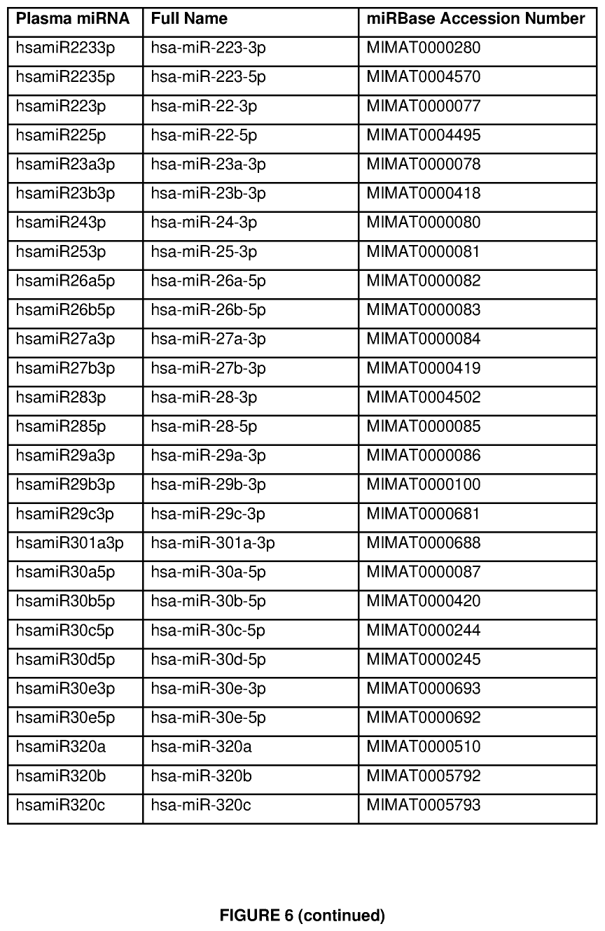

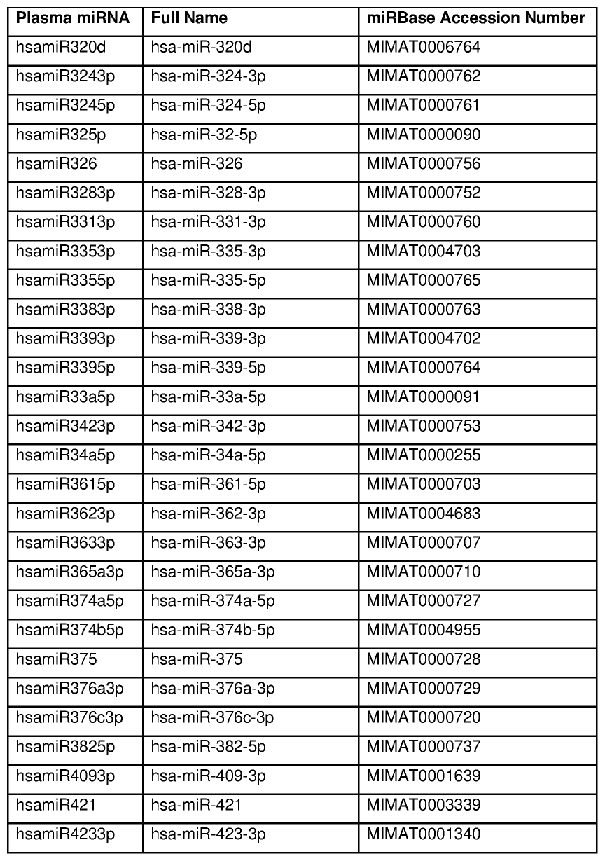

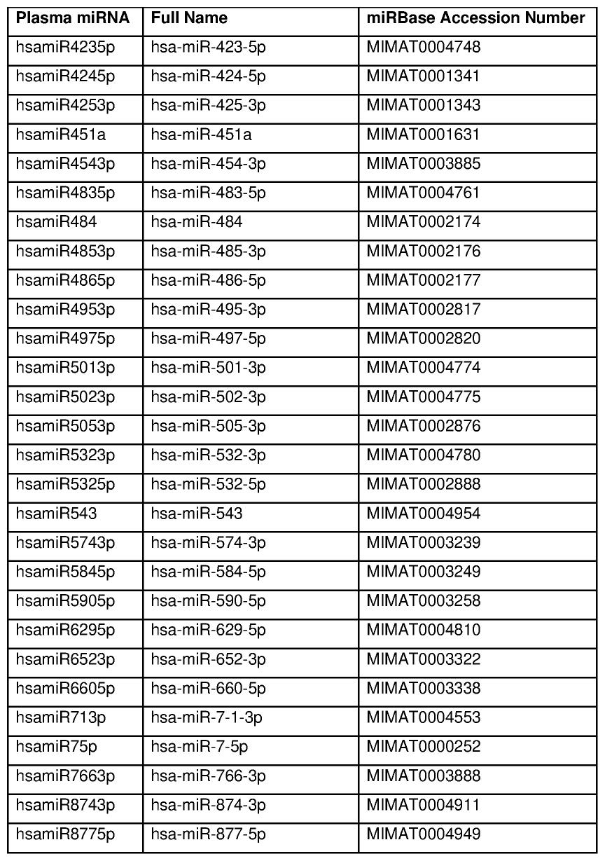

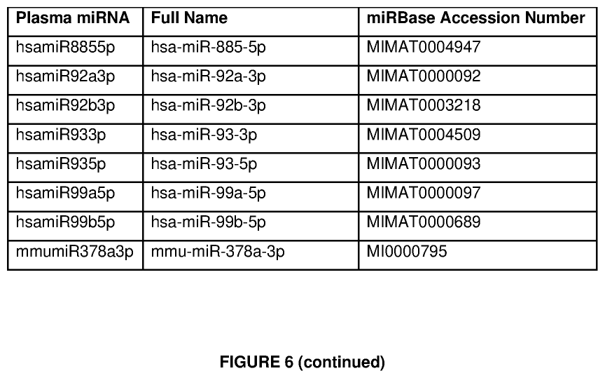

After quality controls omics measurements for all five omics were available for 408 (CS=30, PA=100, PPGL=69, PHT=108, and NV=101) patients. In total 408 MOmics features (PmiRNA: 173, PMetas: 4, PSteroids: 16, USteroids: 27, and PSmallMB: 189) along with age and sex were exported and used to conduct supervised ML experiments (Table 2). The names of features were prefixed with ‘O1_’, ‘O2_’, ‘O3_’, ‘O4_’, and ‘O5_’, for PmiRNA, PMetas, PSteroids, USteroids, and PSmallMB respectively. The MOmics and mono-omics datasets were randomly split into training (˜80%) and testing (˜20%) set. The complete list of all MOmics features is included in FIGS. 2-6.

### Biomarker Discovery Using Supervised Machine Learning

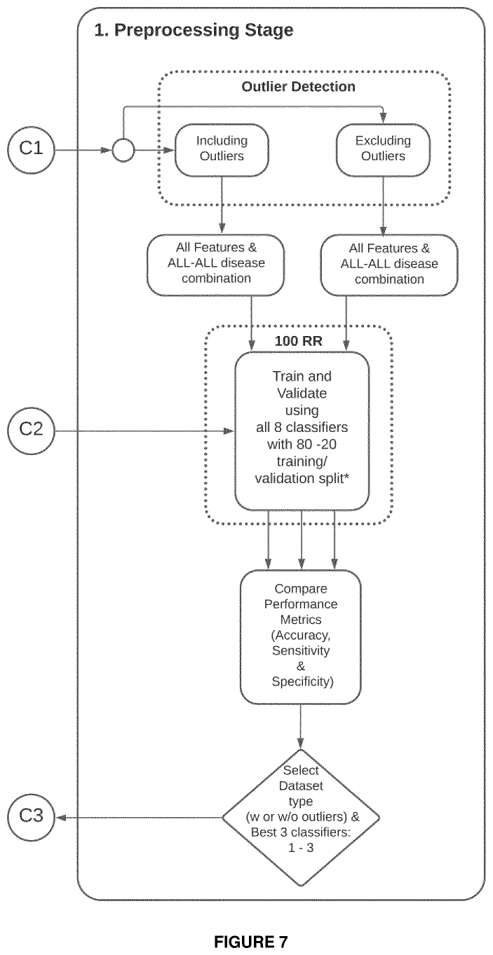

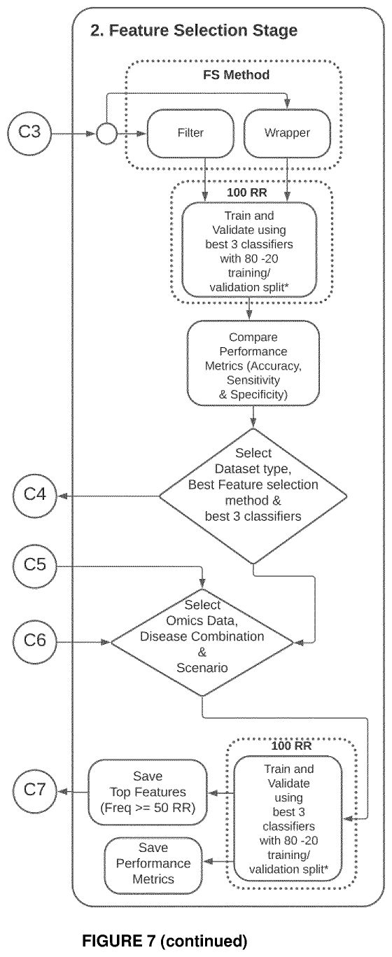

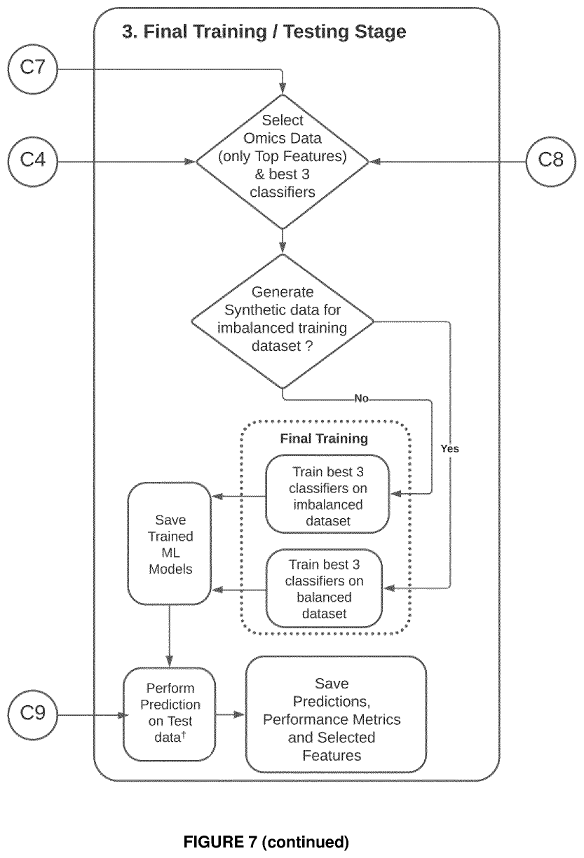

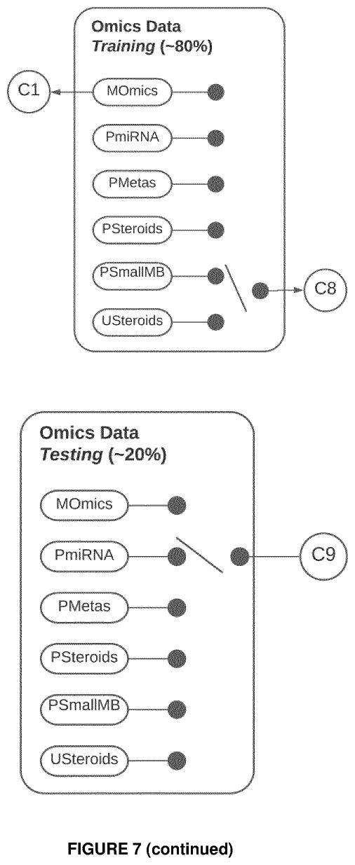

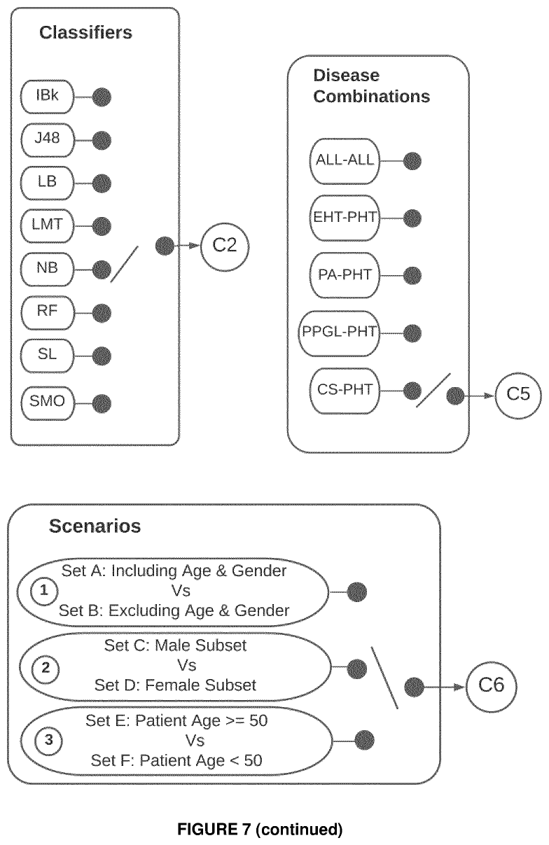

The biomarker discovery involved the selection of disease combinations, outlier detection, choice of supervised ML classifiers, configuration of experiment parameters, and consideration of different evaluation scenarios (FIG. 7).

In this second example, the biomarker discovery comprised three stages (FIG. 7): a pre-processing (outlier detection and choice of supervised ML classifiers) in stage 1, feature selection in stage 2 and final training/testing in stage 3. Classification was performed on ALL-ALL (PPGL vs PA vs CS vs PHT), EHT (PPGL+PA+CS)-PHT, and each individual endocrine hypertension (i.e., PPGL/PA/CS)-PHT.

### Disease Combinations

The five different disease combinations used for classification were: ALL-ALL (PPGL vs PA vs CS vs PHT), EHT (PPGL+PA+CS)-PHT, and each individual endocrine hypertension (i.e., PPGL/PA/CS)-PHT. These combinations did not include NV since the key question addressed was: How can hypertensive patients be stratified amongst themselves? Omics data from NV were used to evaluate how they vary from patients with different forms of hypertension.

### Outlier Detection

To study the impact of outliers on classification, two sets of results were analyzed, as shown in Stage 1 (FIG. 7) i.e., 1) Using data including outliers and 2) applying 3 times 1.5 quartile method to remove extreme outliers (excluding outliers). The outliers were refilled using the maximum value.

### Classifiers, Feature Selection and Classification Performance Metrics

An assorted set of 8 different classifiers: Decision Trees (J48), (Breiman, 1998) Naïve Bayes (NB), (Zhang, 2004) K-nearest neighbours (IBk), (Bentley, 1975) LogitBoost (LB), (Friedman et al., 1998) Logistic Model Tree (LMT), (Landwehr et al., 2005) Simple Logistic (SL), (Sumner et al., 2005) Random Forest (RF), (Breiman, 2001) and Sequential Minimal Optimisation (SMO) (Platt, 1998) were used (Stage 1 of FIG. 7). The classification was implemented using the caret (Kuhn, 2008) and RWeka (Hornik et al., 2009) in R. (R Core Team, 2013) A further training-validation (80-20%) split was performed on the training set for 100 RR using random seed for each iteration to ensure reproducibility. For feature selection, wrapper (Boruta) (Kursa & Rudnicki, 2010) and filter (Correlation-based feature selection—CFS) (Hall, 1999) methods were compared (Stage 2 of FIG. 7). The classifications were evaluated over 100 random repeats (RR) using performance metrics: balanced accuracy, sensitivity, specificity, AUC, F1, and Kappa score. The balanced accuracy allows adjusting for the class imbalance problem.

### Evaluation Scenarios

One of the key objectives of the analysis was to identify the list of most discriminating features for a given disease combination. Possible bias due to the age or sex of the patients was studied with different sets of scenarios. Table 3 summarizes the three scenarios, which were investigated for each disease combination along with their justification and Set combinations.

TABLE 3

| | Input dataset (=scenario) | Comments |
|---|---|---|
| 1 | Set A (using all omics, age & gender) | To study the impact of age and gender as |
| | vs Set B (only using all omics) | discriminating biomarkers. |
| 2 | Set C (Male subset) vs | To study the influence of gender by |
| | Set D (Female subset) | comparing the classification accuracy and to |
| | | find gender-specific discriminating |
| | | biomarkers. |
| 3 | Set E (Patient age >=50) vs | To investigate how omics are affected by |
| | Set F (Patient age <50) | age and hormonal status, based on average |
| | | female menopausal age i.e. 50 years. |
| 4 | Set G (With synthetic samples) vs | To overcome the class imbalance problem |
| | Set A vs | and study the impact of top feature selection |
| | Set A1 (reduced biomarkers from Set | on accuracy. |
| | A) | |

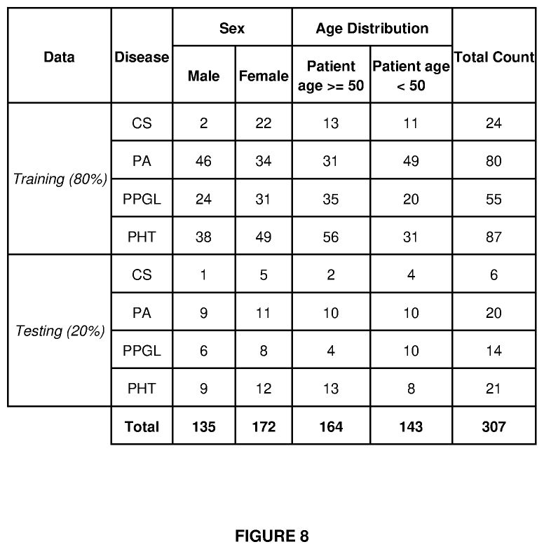

FIG. 8 shows the corresponding patient count. The top features from Set A with a cut-off for the feature frequency of 50 was used for the final training/testing stage (FIG. 7). This value was chosen empirically as a trade-off for finding the optimal number of reduced features without impacting the classification performance. Also, in order to minimise the impact of the class imbalance problem, additional synthetic samples were generated using SMOTE (Chawla et al., 2002) for CS and PPGL. In the case of the EHT-PHT disease combination, a down-sampling approach was used for class balancing instead of synthetic samples.

Stage 3 of the schematic shows the steps for the final/testing stage using test data (FIG. 7). The omic type and disease combination was selected, followed by the training of the best 3 classifiers. The trained model was then used to classify the test data. The prediction outcomes, the various performance metrics and the list of selected features were then saved and compared at the end of each classification. The characteristics of the final set of discriminating features was then evaluated with respect to NV using PCA analysis. All the classifications shown in FIG. 7 were employed on MOmics data and then on all five mono-omics individually.

### Results

### Evaluation of Data-Driven Pre-Processing and Feature Selection Methods

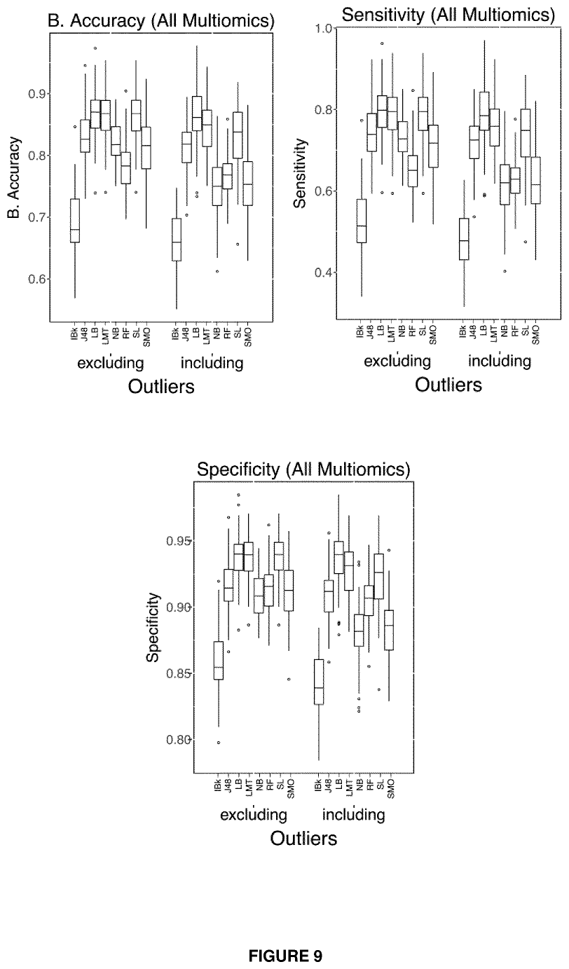

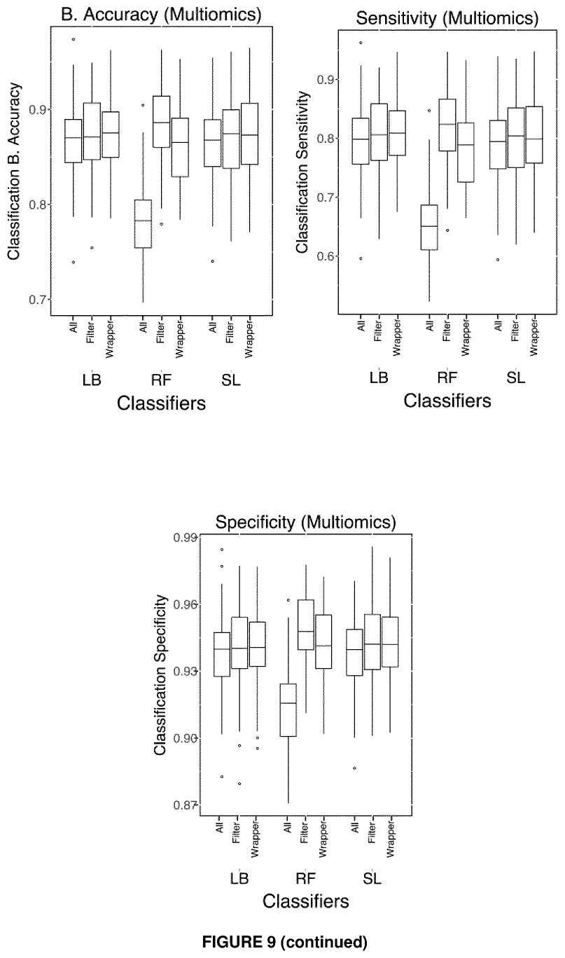

First, the classification performance for ALL-ALL using MOmics data excluding and including outliers was evaluated (FIG. 9). Excluding outliers provided better classification as observed from various performance metrics. SL provided ˜4%, 5%, and 1% increases in balanced accuracy, sensitivity, and specificity when excluding outliers, respectively. Overall, LB, SL, and RF were the best performing classifiers. Next, feature selection methods were compared using LB, RF, and SL classifiers for ALL-ALL disease combination (FIG. 9). Both filter and wrapper methods provided comparable classification performance. However, wrapper method was chosen for next stages of analysis since it evaluates the feature subsets as search problem to find key dependencies amongst features.

### Overall Classification of Primary and Endocrine Hypertension

The top 3 ML models were trained using the reduced training dataset which used top features from 100 RR for each disease combination (See stage 3 in FIG. 7). These trained classifiers were then evaluated on the test set. The corresponding performance metrics and related discriminating features selected by the classifiers were as follows:

### Performance Metrics

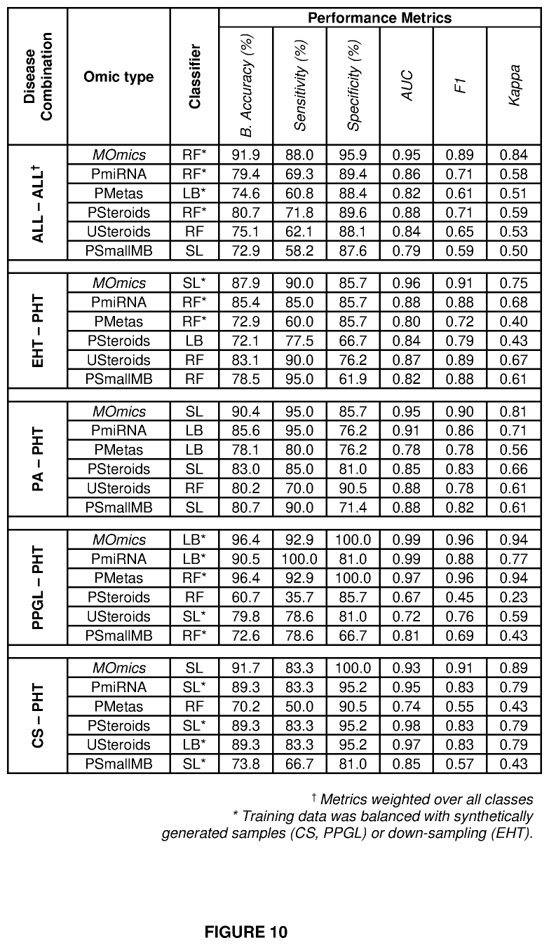

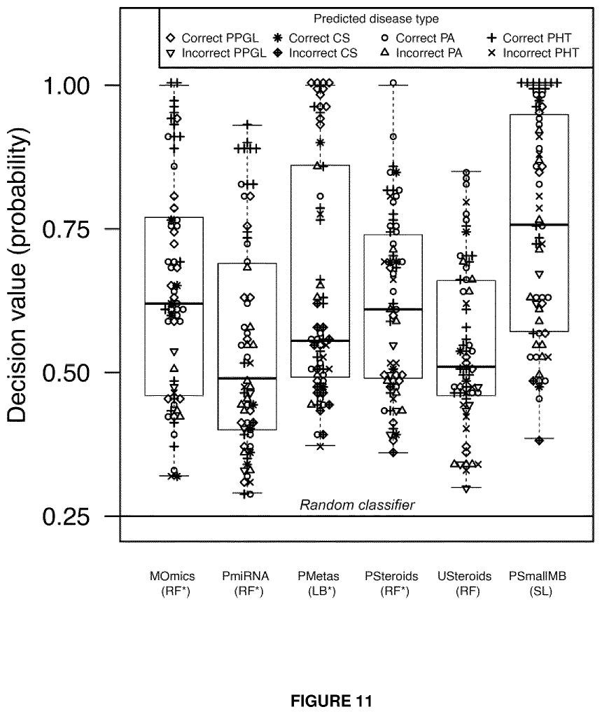

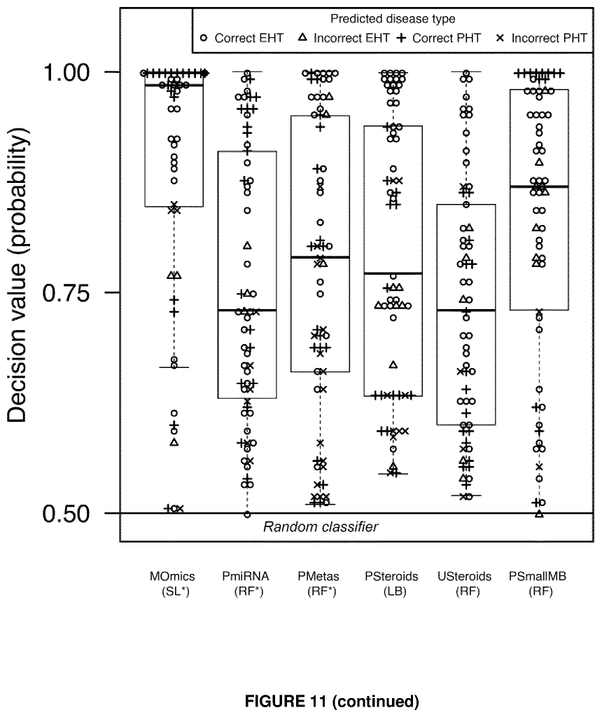

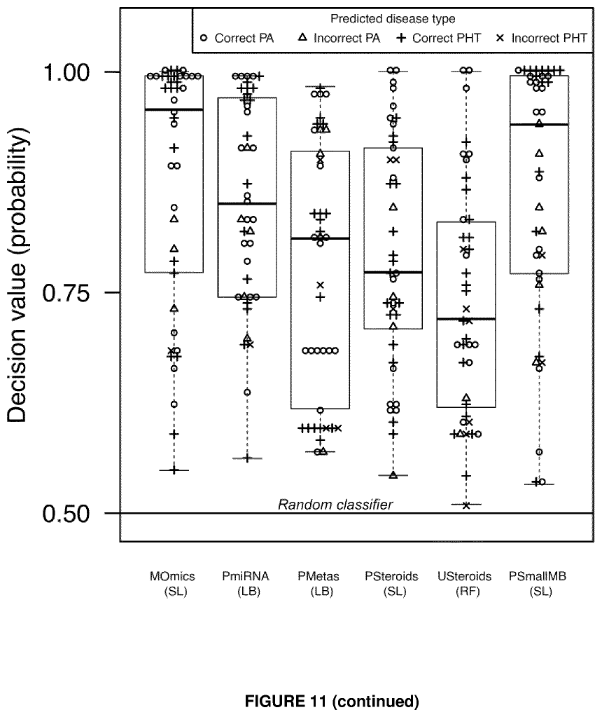

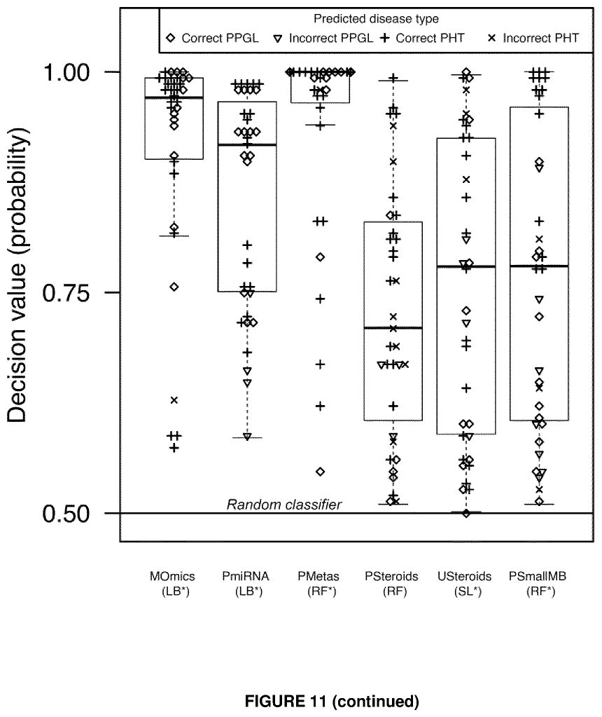

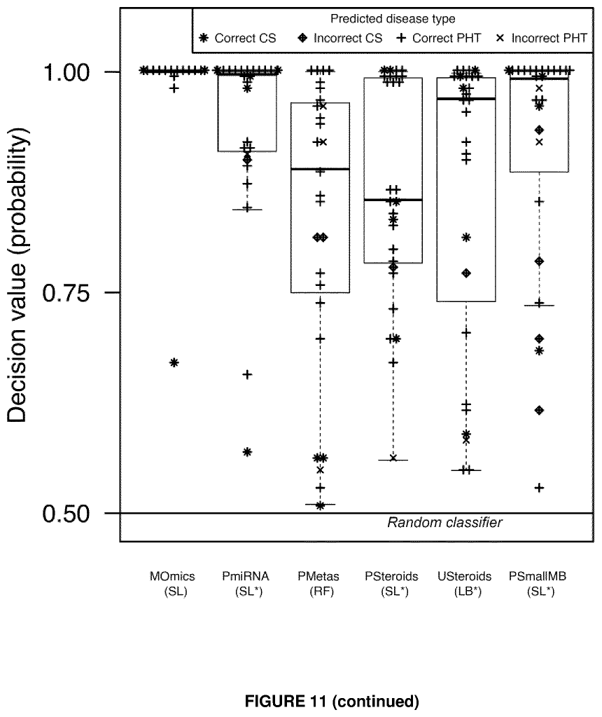

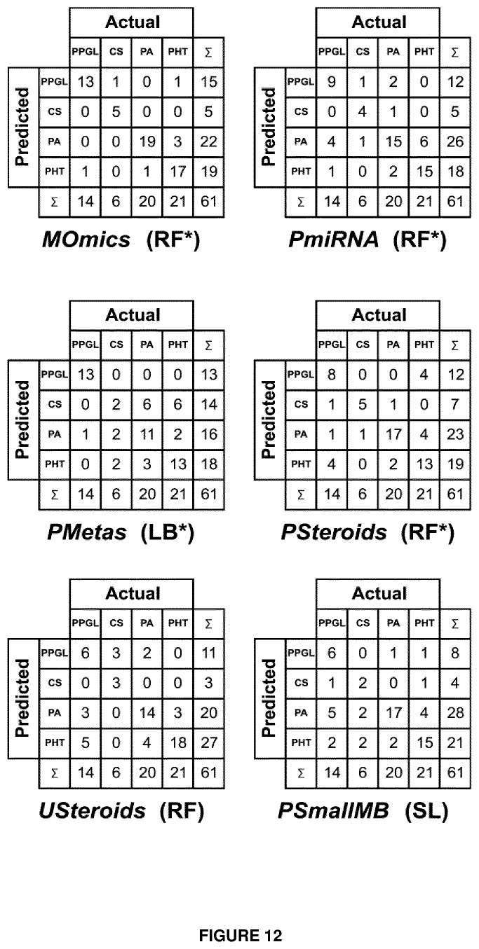

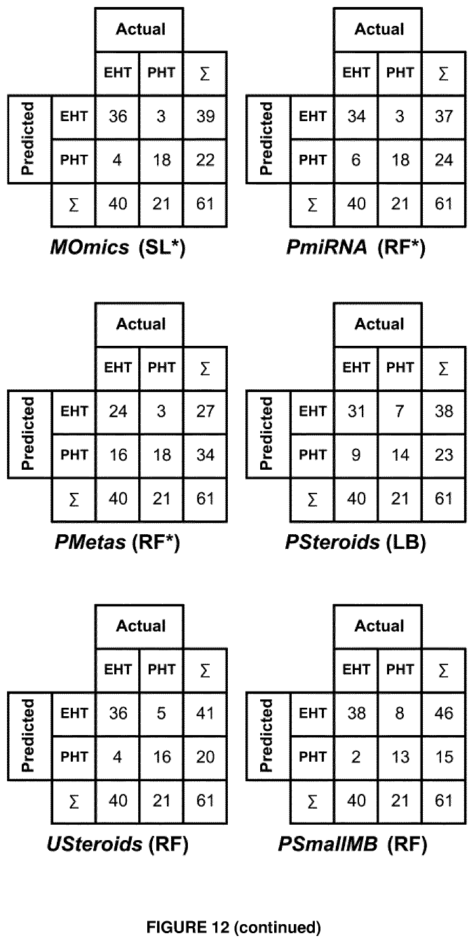

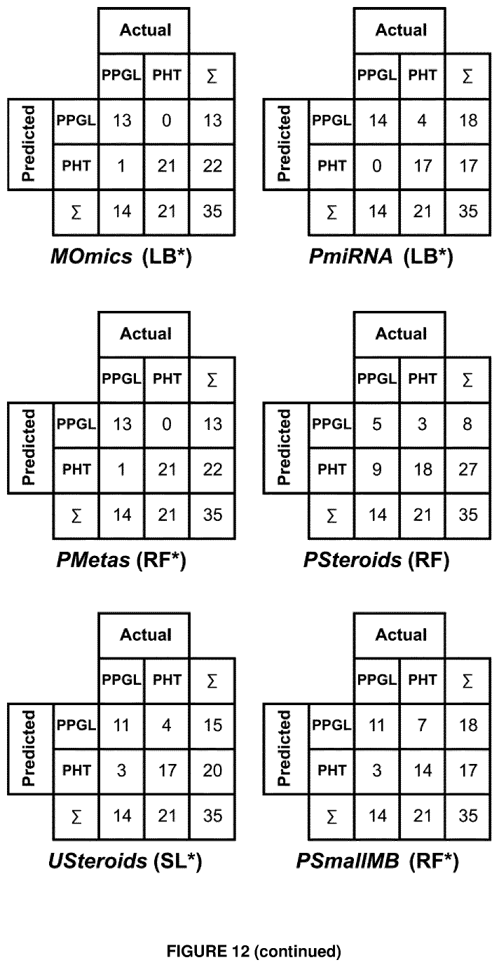

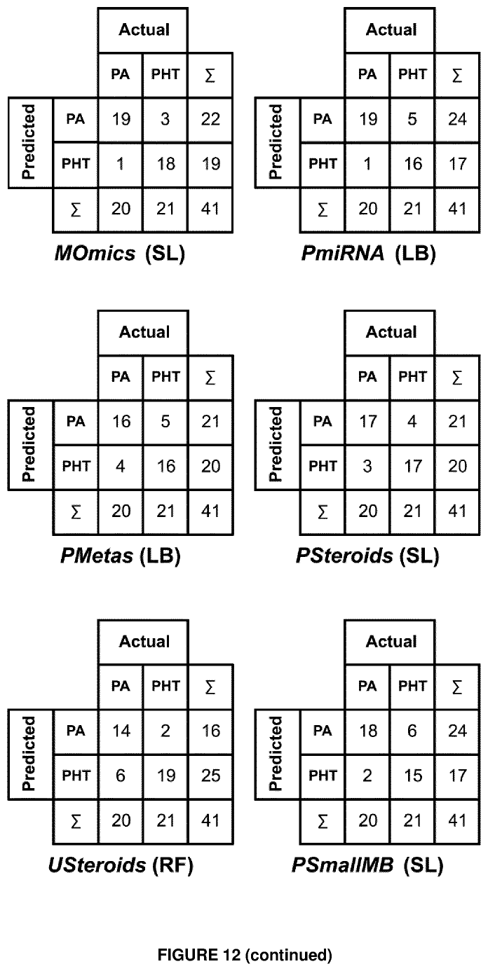

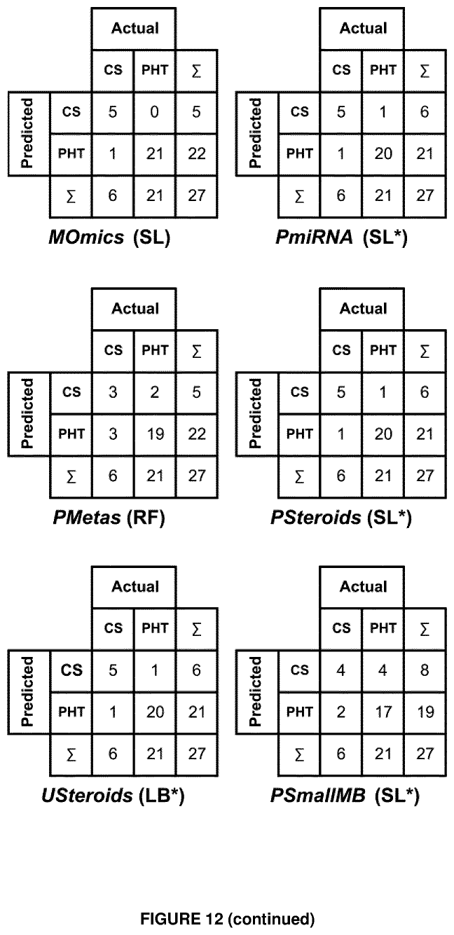

The MOmics classifier outperformed mono-omics classifiers when considering balanced accuracy, AUC (except CS-PHT), F1, and Kappa score (FIG. 10). Using MOmics data for ALL-ALL combination, RF classifier (with balanced dataset) provided better classification performance (˜92% balanced accuracy, 0.95 AUC with 88% sensitivity, and 96% specificity) when compared to other 5 mono-omics. The corresponding decision value (prediction probability) for each test sample was evaluated (FIG. 11). High decision values highlighted the confidence of the classifier in predicting the test sample. Many correctly classified samples had high decision values, which emphasize the fact that MOmics classifier provided better performance in comparison to others. For ALL-ALL, MOmics classifier had 7 incorrectly classified samples (FIG. 11). In contrast, the best performing (amongst 5 mono-omics) PSteroids-based RF classifier achieved ˜81% balanced accuracy, 0.88 AUC with ˜72% sensitivity, and ˜90% specificity. The corresponding decision values showed low confidence with 18 incorrectly classified samples. These results were also evaluated as confusion matrices (FIG. 12).

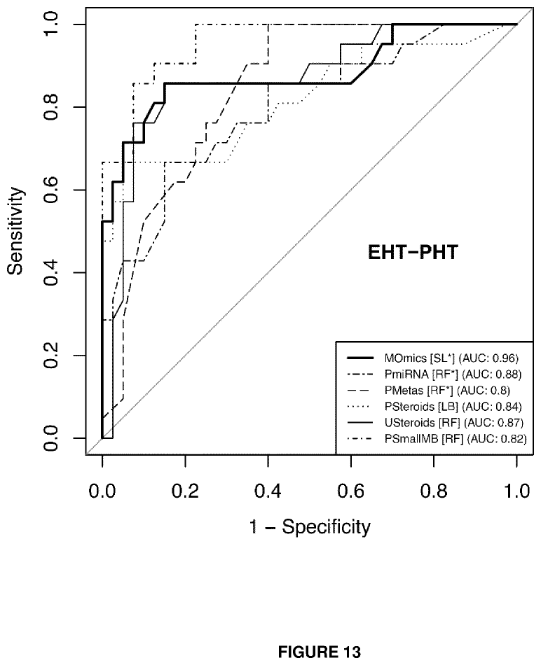

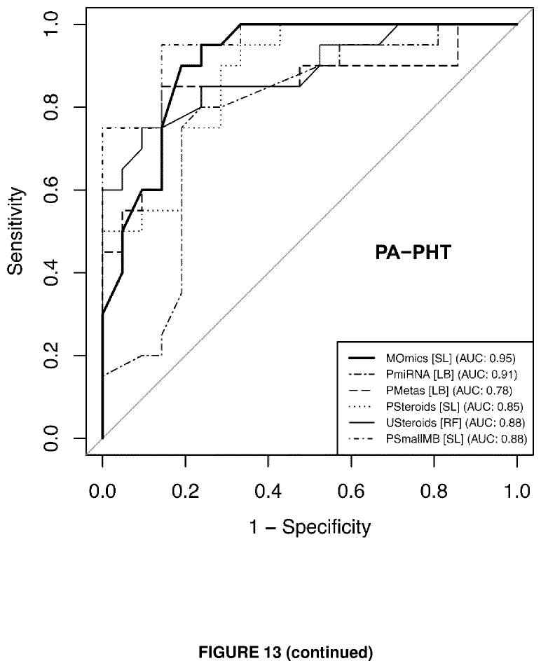

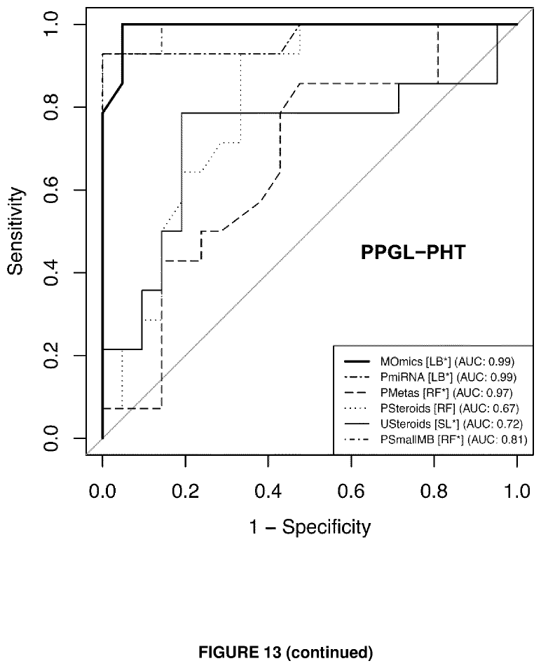

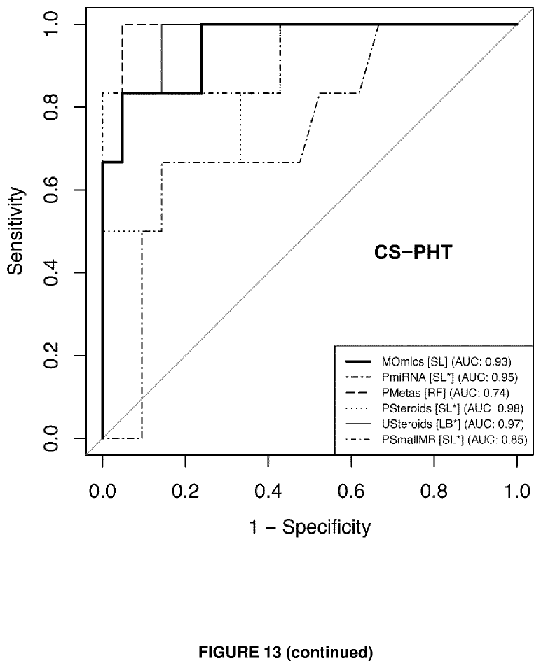

For the EHT-PHT combination, the SL classifier with MOmics (using balanced data) provided 0.96 AUC (FIG. 13) with 90% sensitivity, and ˜86% specificity (FIG. 10). High decision confidence was observed for most of the correctly classified samples (FIG. 11). Although PmiRNA and PMetas-based RF classifier achieved ˜86% specificity (same as MOmics), their AUC was 0.88 and 0.80 respectively. Notably, both PSmallMB-based classifier provided the highest sensitivity of 95% however with a lower AUC of 8.2.

For the PA-PHT combination, the SL classifier using MOmics provided highest balanced accuracy and AUC (˜90% and 0.95 respectively) with 95% sensitivity and ˜86% specificity (FIG. 10 and FIG. 13). Although the PmiRNA-based LB classifier provided the highest AUC 0.91 (with 95% sensitivity) amongst mono-omics, USteroids achieved the highest specificity of ˜90%. The decision values highlighted the high confidence of the MOmics classifier in comparison to the others (FIG. 11). In the case of PPGL-PHT combination, the LB classifier using MOmics and RF classifier using PMetas achieved the same balanced accuracy of ˜96% with AUC of 0.99 and 0.97 respectively. The comparative performance of the decision values for these classifiers showed their high confidence (FIG. 11). Also, PmiRNA-based LB classifier provided 0.99 AUC with 81% specificity. Moreover, for the CS-PHT combination, the MOmics-based SL classifier provided 100% specificity and ˜92% balanced accuracy, but with a lower AUC of 0.93. In contrast, mono-omics based classifiers using PSteroids and USteroids achieved higher AUC of 0.98 and 0.97 respectively. The probability values for the test set showed the difference of confidence amongst classifiers (FIG. 11).

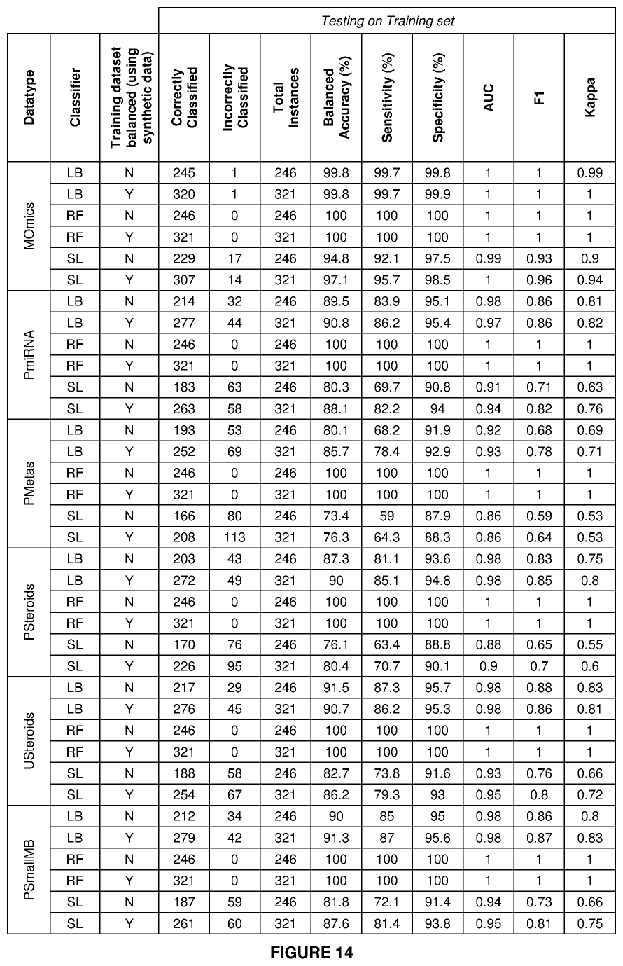

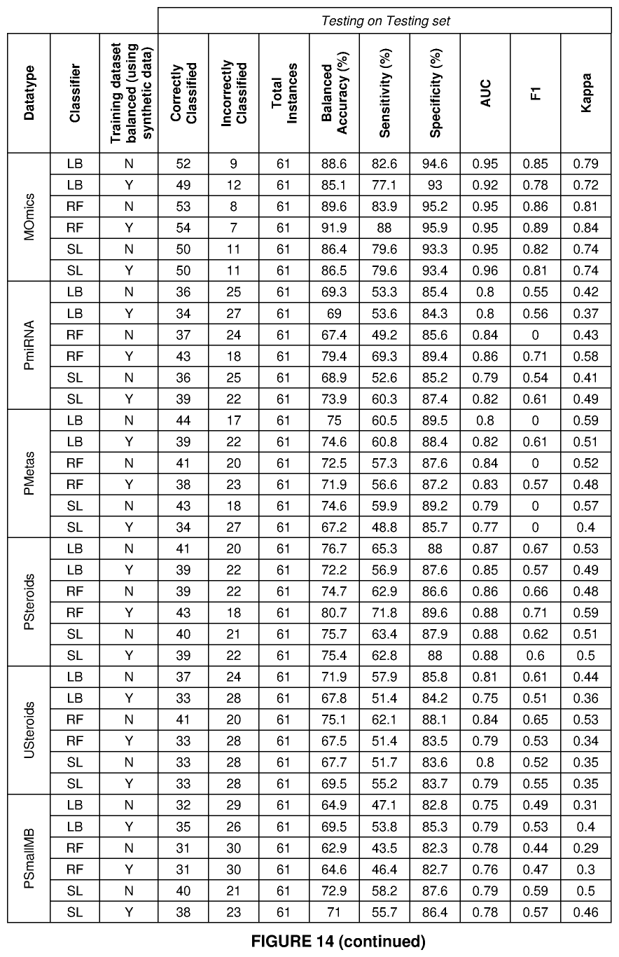

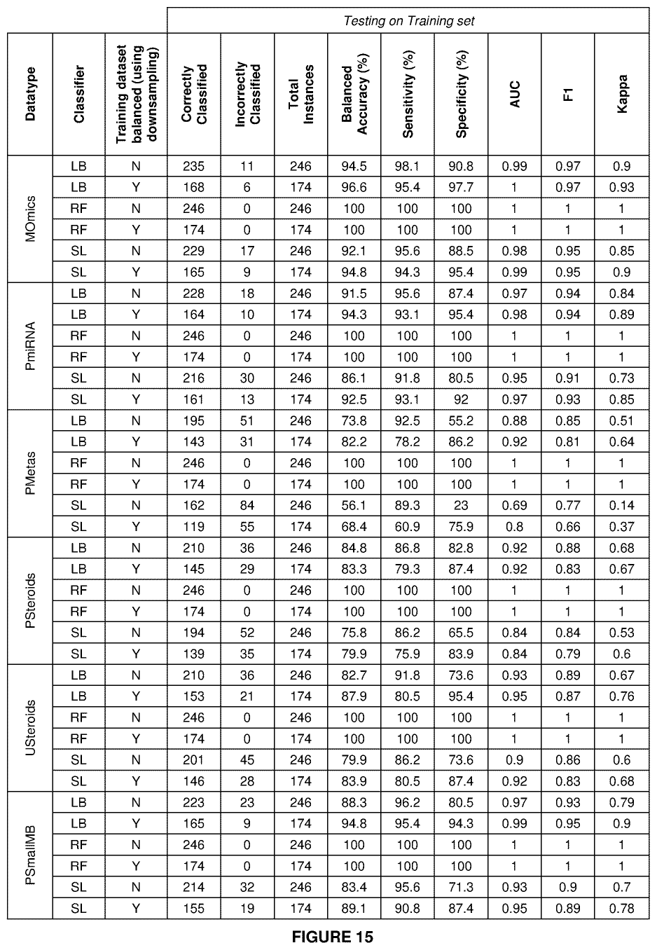

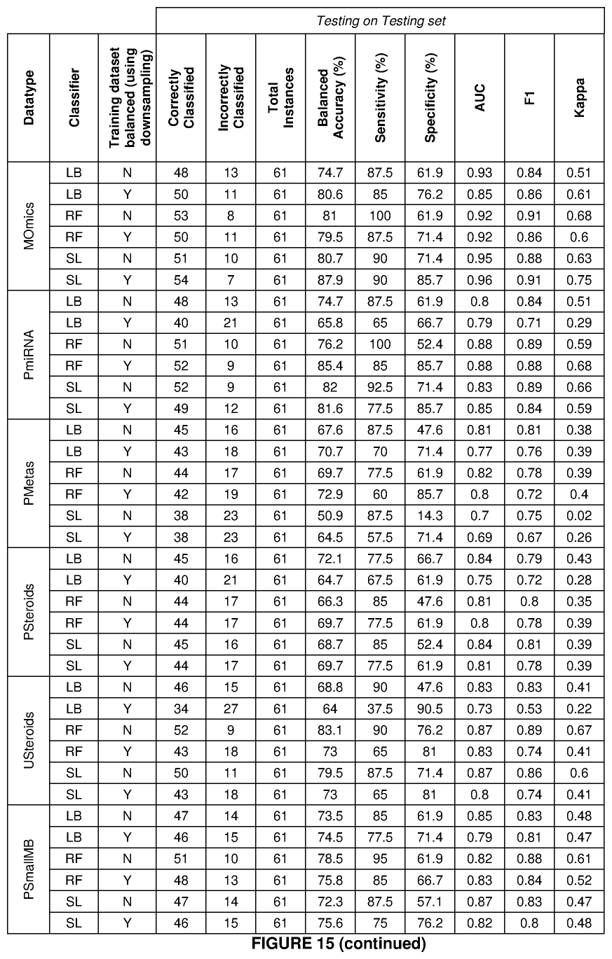

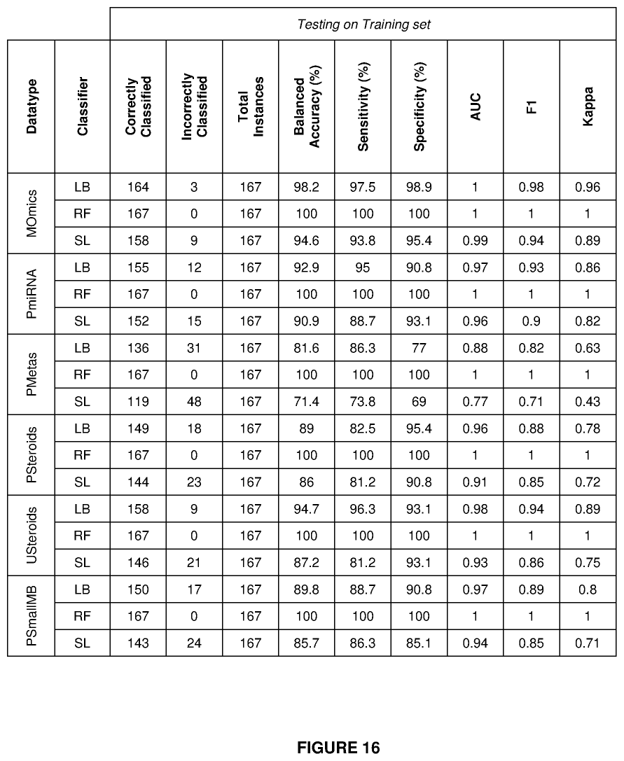

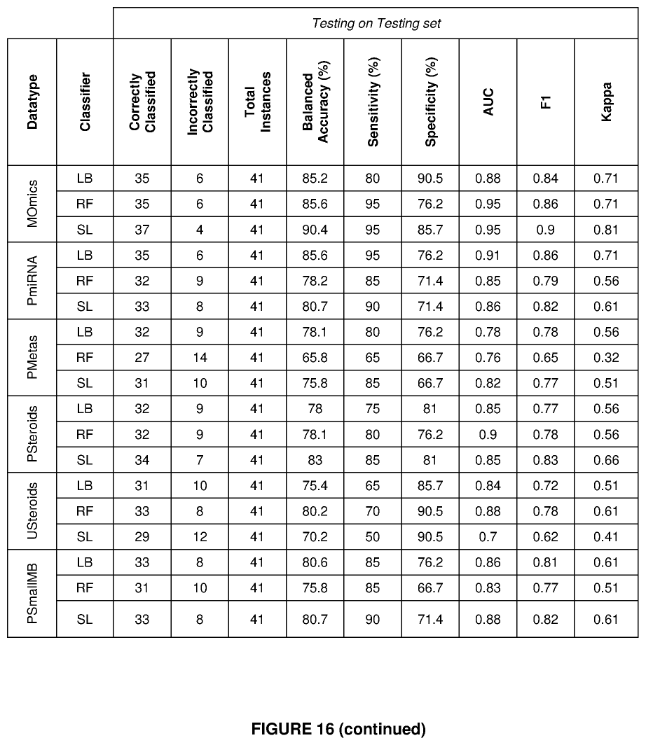

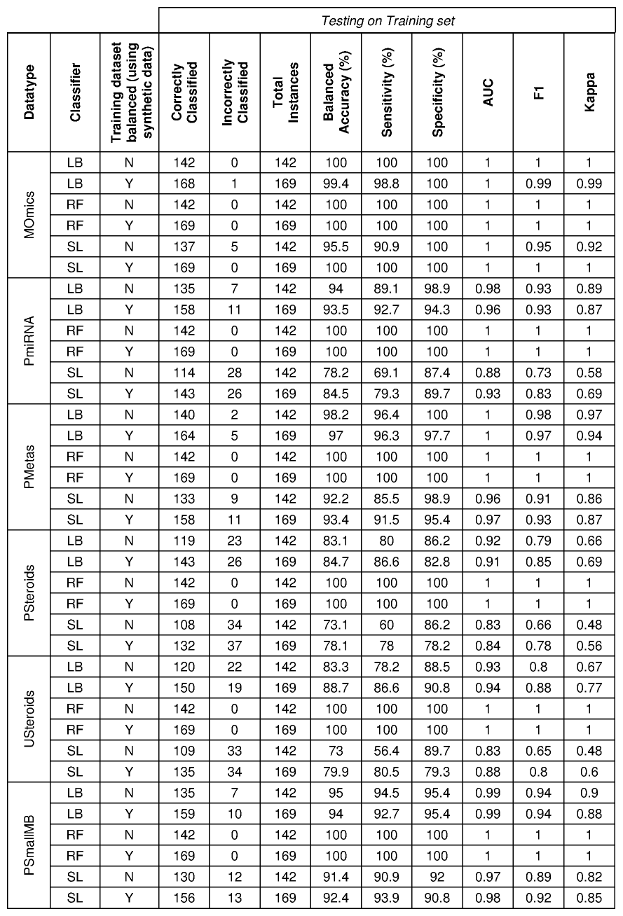

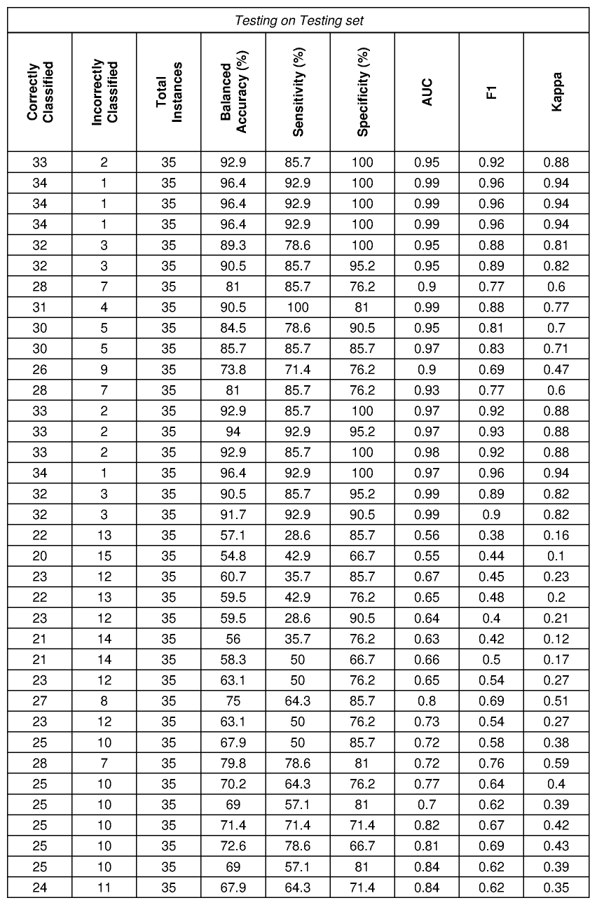

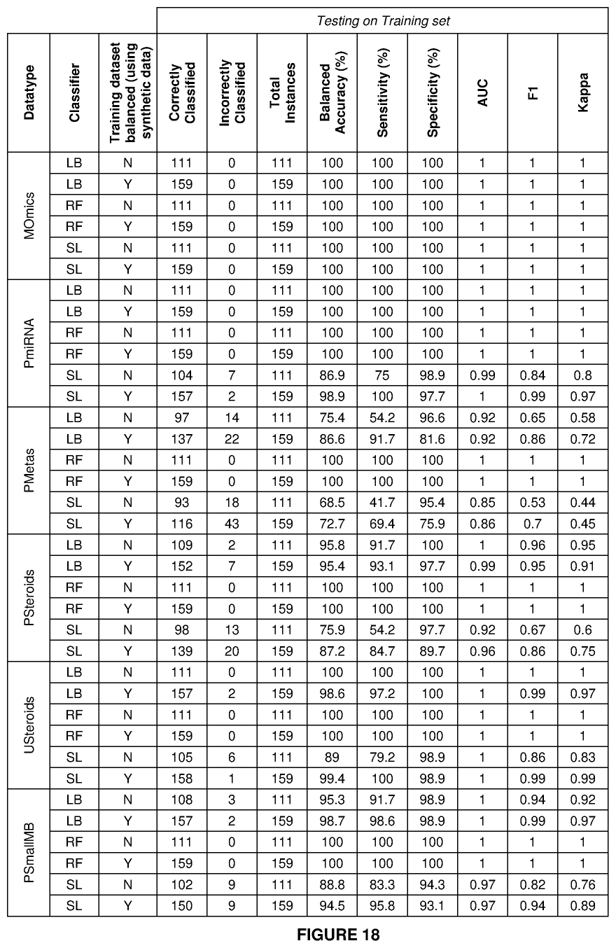

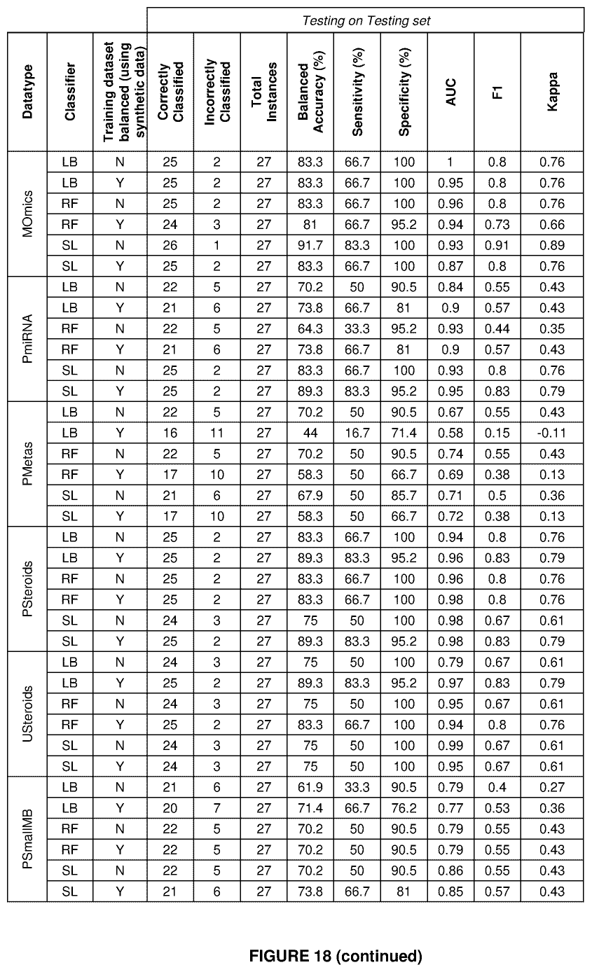

These classifiers were also tested on the training dataset to understand the effect of overfitting (FIG. 14-18). Amongst the three classifiers (LB, RF and SL), evidently RF provided superior classification results when tested on the training set in comparison to the testing set. This highlighted the overfitted training of the RF classifier irrespective of whether the training data was balanced or not. On the other hand, LB and SL classifiers were less overfitted and performed consistently for both training and testing set.

### Discriminating Features

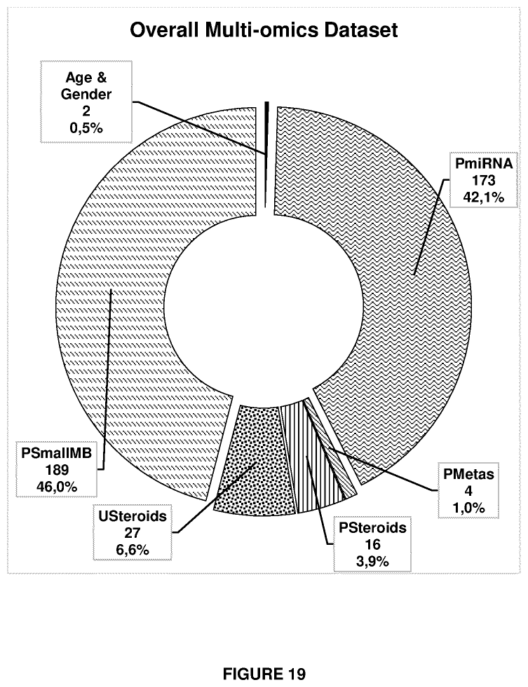

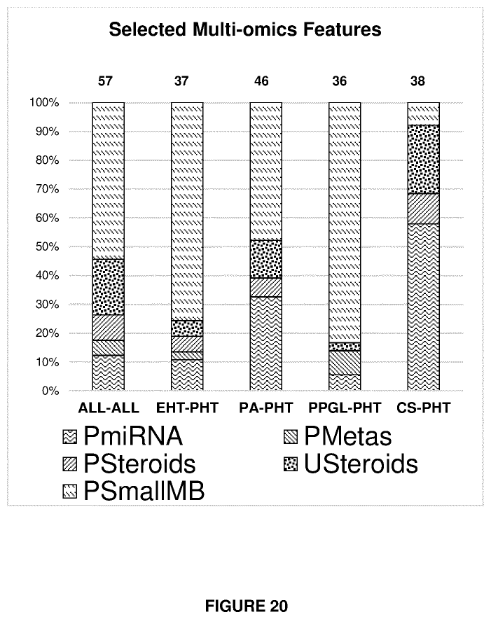

The final selected set of MOmics features used for classifier training comprised different omics features for each disease combination. The PmiRNA and PSmallMB features represent 88% of the whole MOmics dataset (FIG. 19), a similar share was observed within the final selected set of features (FIG. 20). For example, PSmallMB forms a considerable part of all the disease combinations except CS-PHT where a high number of PmiRNAs were found to be highly discriminating (˜58% of total selected features). In contrast, for PPGL-PHT, very few PmiRNAs (˜5.5% of total features) were selected.

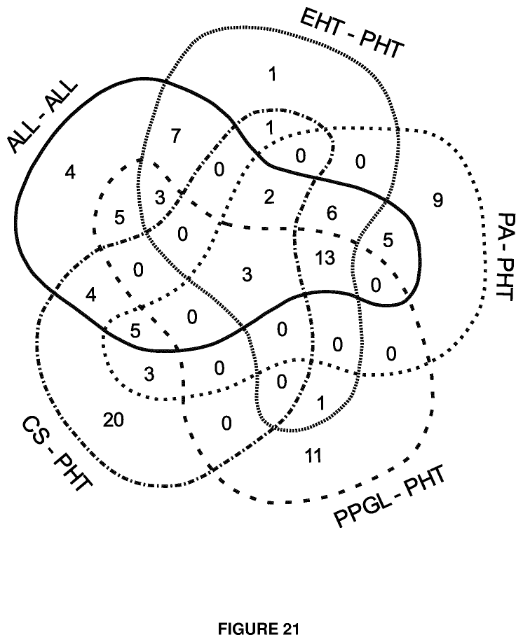

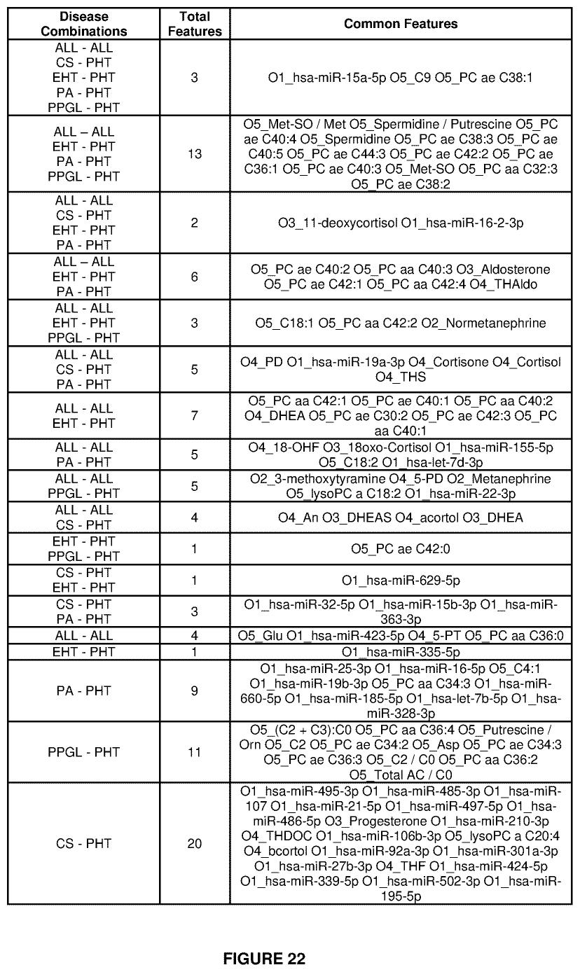

The commonality of selected MOmics features amongst different disease combinations was also investigated (FIG. 21). Two PSmallMB features (O5_PC ae C38:1 and O5_C9) and one PmiRNA (O1_hsa-miR-15a-5p) were present in all five disease combinations (FIG. 22). Similarly, thirteen PSmallMB features were common amongst 4 disease combinations (i.e. all except CS-PHT). Various unique features were selected for each disease combination. For example, twenty features (15 PmiRNAs, 1 PSteroids, 3 USteroids and 1 PSmallMB) were selected only for CS-PHT. Overall, ALL-ALL has more discriminating features (57 in total) than any of the other four disease combinations. Not unexpectedly, age and sex were not selected in any of the five disease combinations.

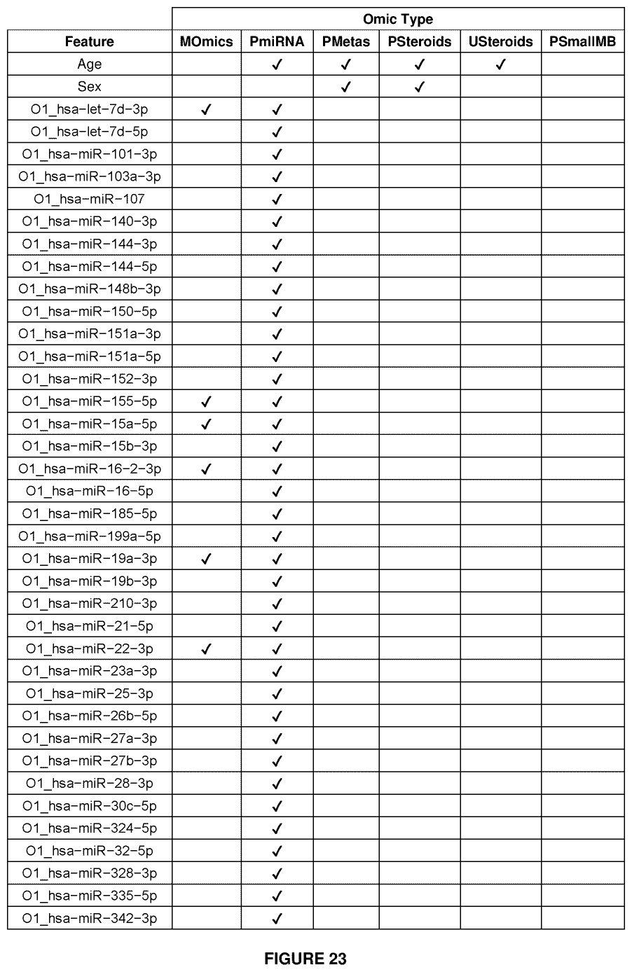

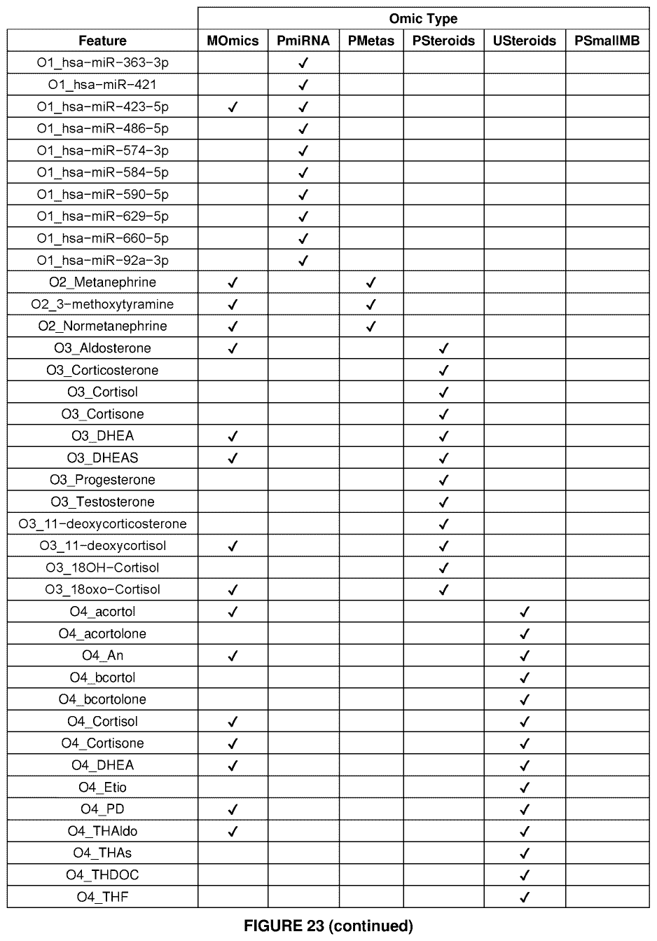

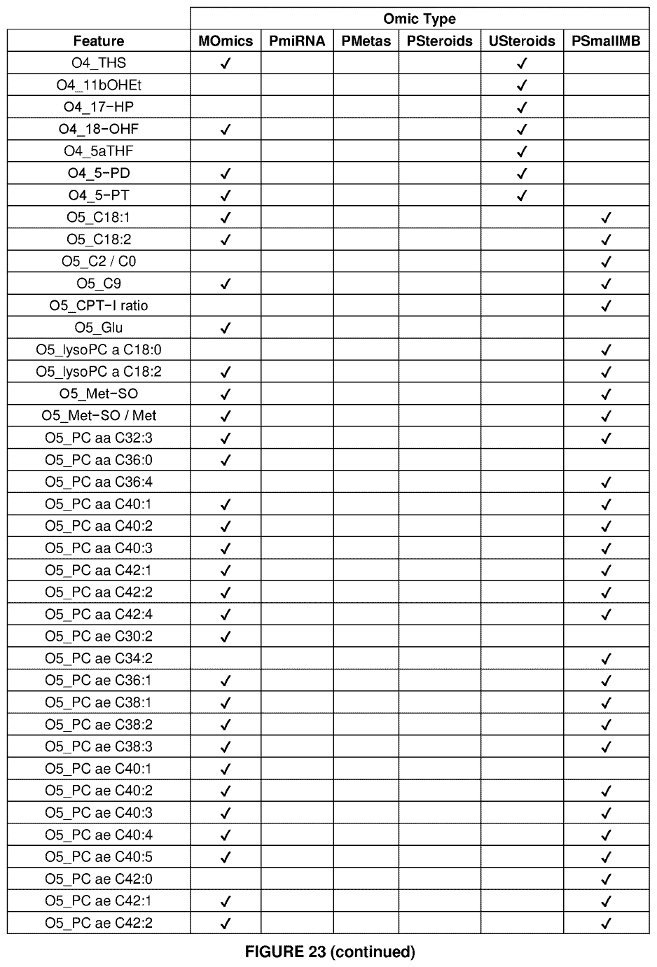

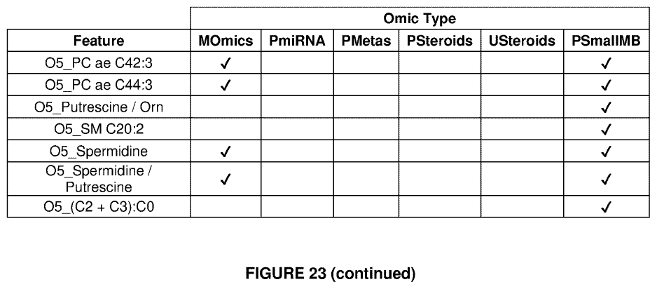

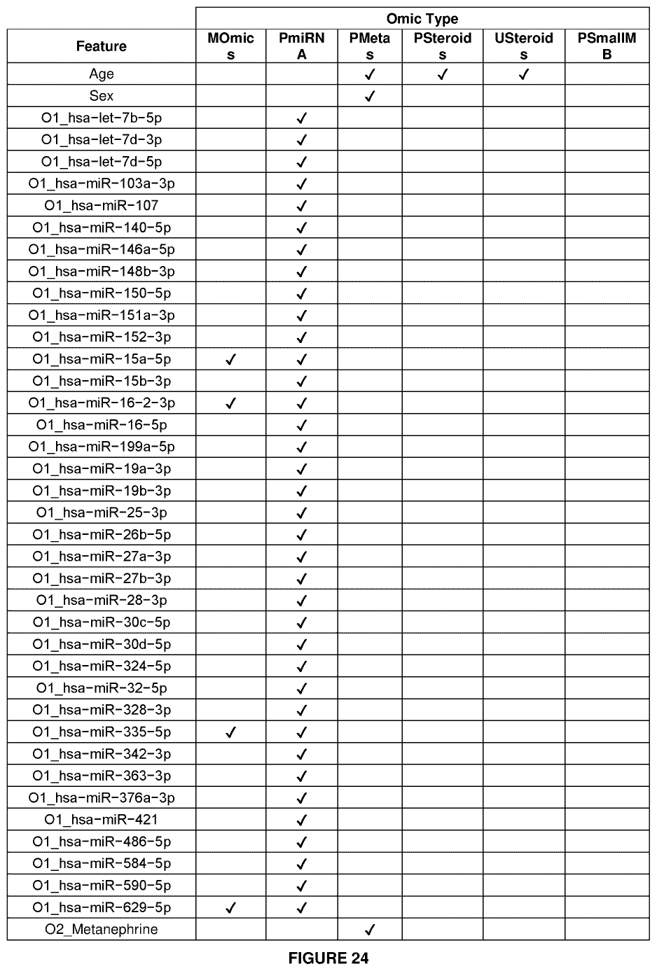

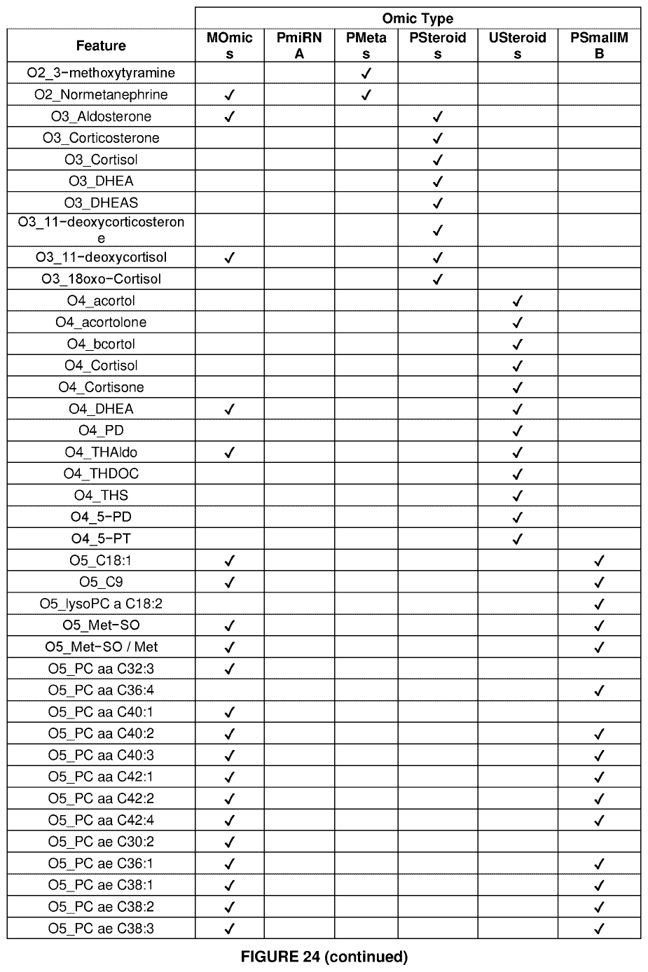

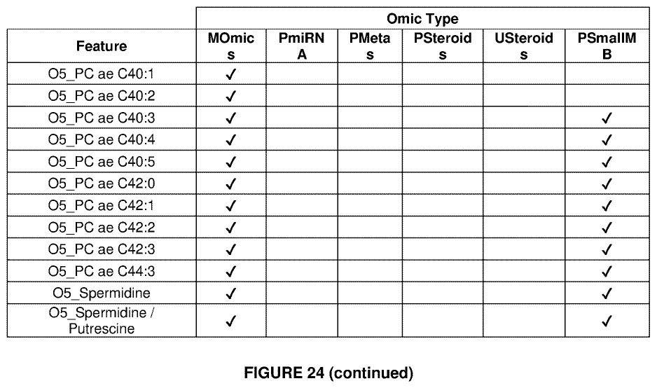

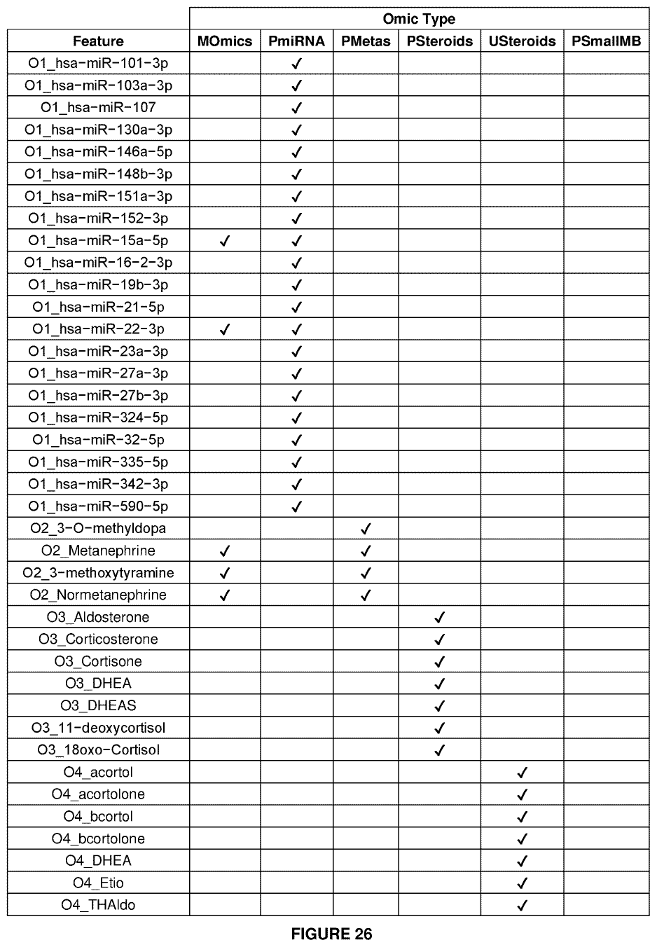

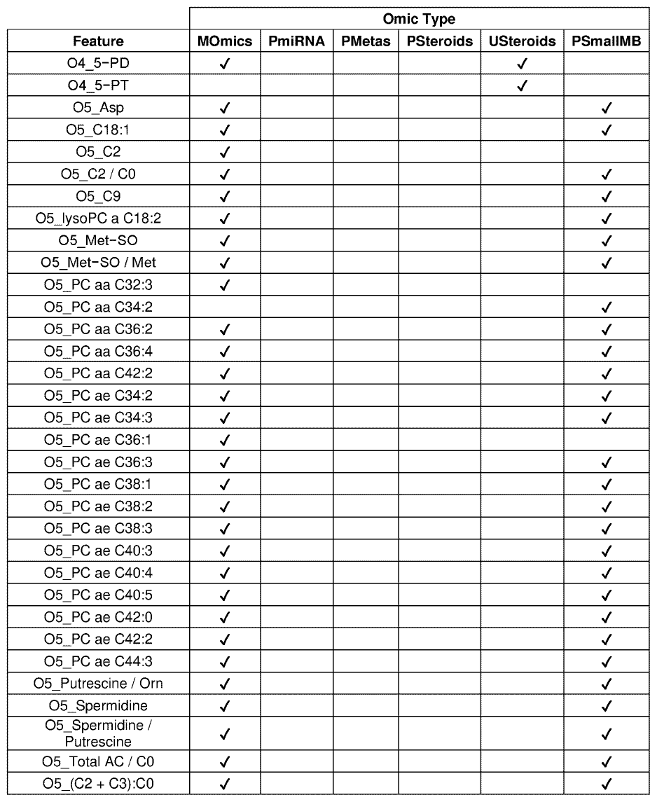

The discriminating features selected for mono-omics classifiers were also examined (FIGS. 23-27). Age and sex were selected in most of the mono-omics classifiers (except PSmallMB) in all disease combinations other than PPGL-PHT. A higher number of PmiRNAs were selected in the case of ALL-ALL and EHT-PHT in comparison to other disease combinations. On the contrary, for PSmallMB classifier only 9 features were selected in CS-PHT amongst all disease combinations.

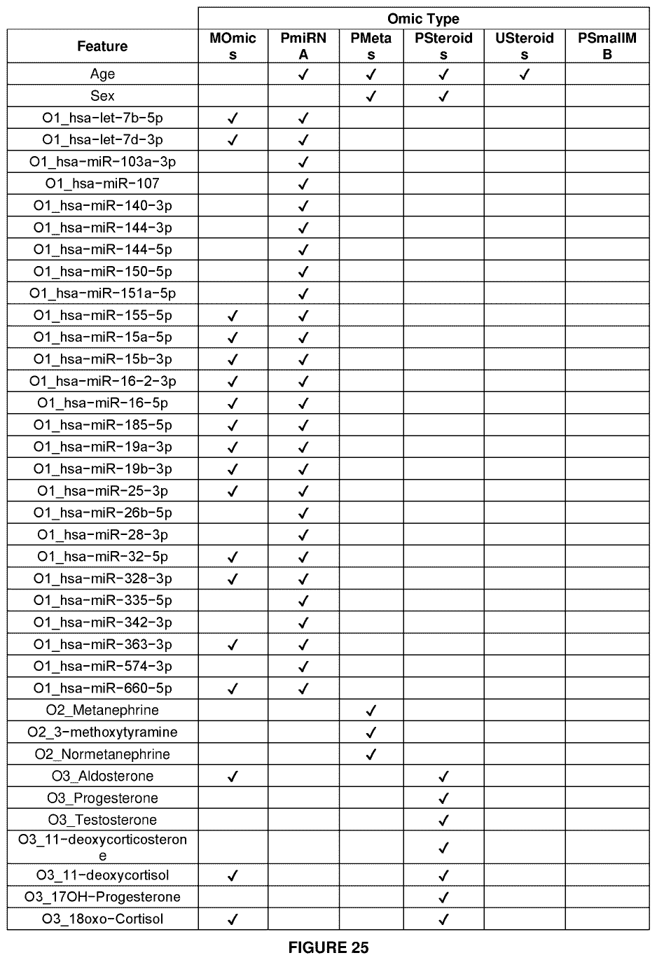

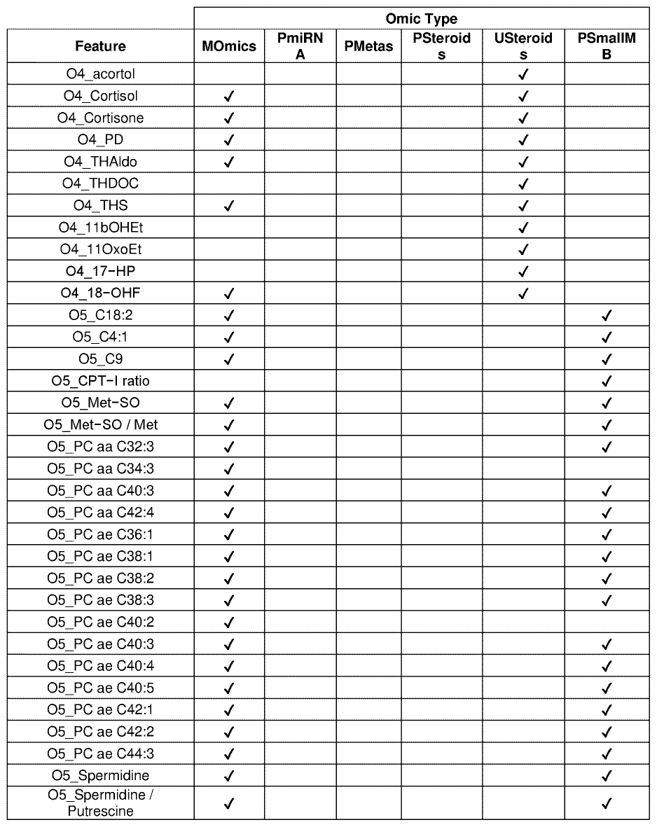

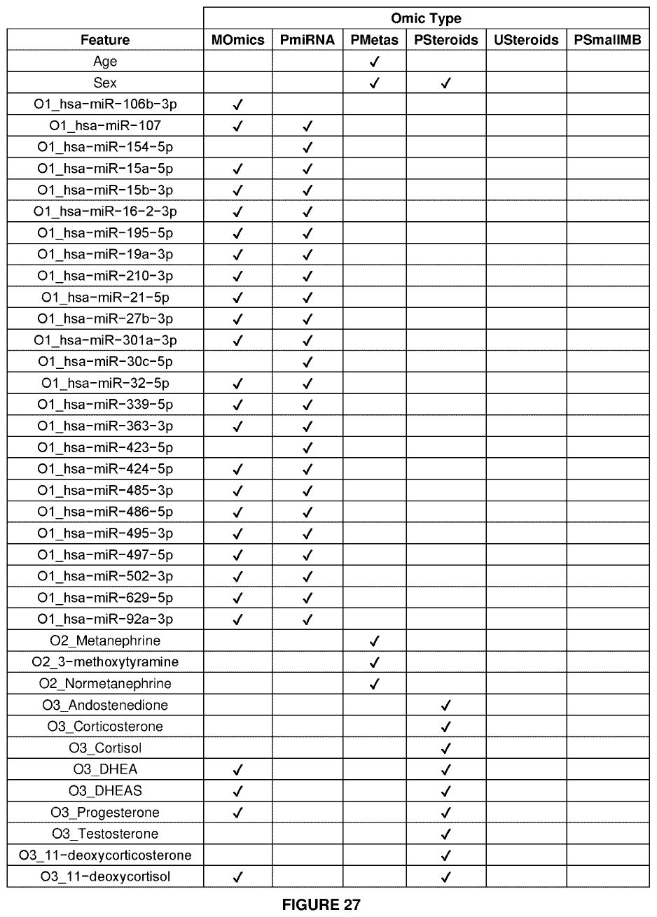

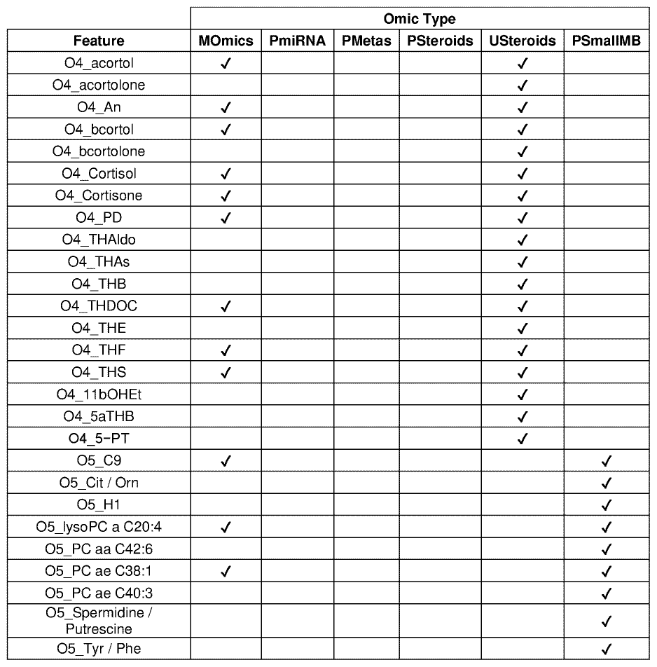

The contribution of each omic in the final selected MOmics features (with regard to the count in the whole dataset) was also analysed. For example, 3 out of 4 PMetas features (75%) were selected in ALL-ALL and PPGL-PHT disease combinations. None of the PSteroids features was selected in PPGL-PHT. No PMetas were selected in PA-PHT and CS-PHT combinations (FIG. 25 and FIG. 27).

The close examination of features amongst MOmics and mono-omics classifiers highlighted that all the features selected in MOmics were part of the individual omics classifiers, except PSmallMB and PmiRNA (FIGS. 23-27). In case of PSmallMB, some features were exclusively selected in MOmics classifiers (For example, O5_PC aa C34:3 and O5_PC ae C40:2 in PA-PHT). Similarly, in CS-PHT, PmiRNA O1_hsa-miR-106b-3p was exclusively selected for MOmics classifier.

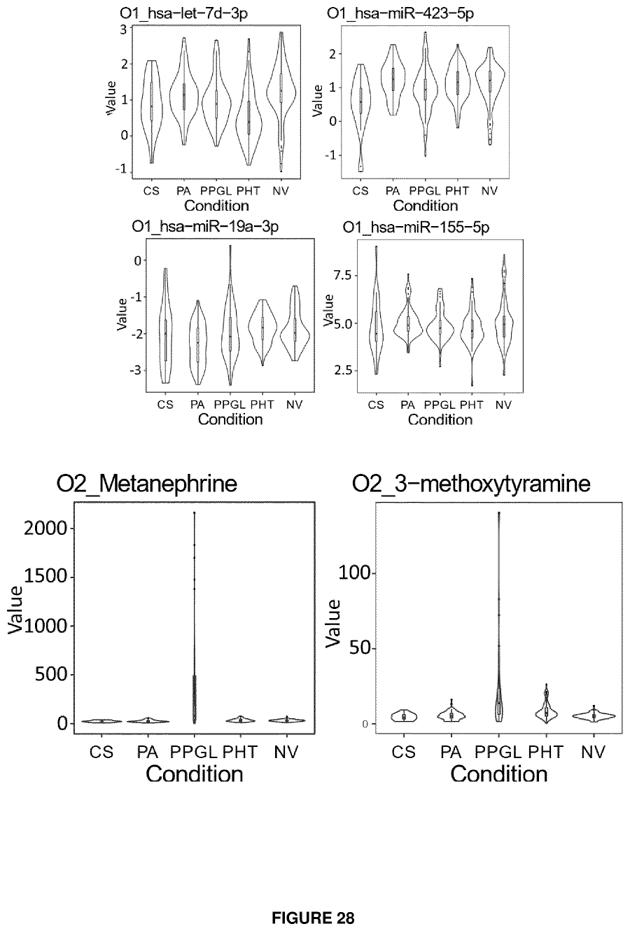

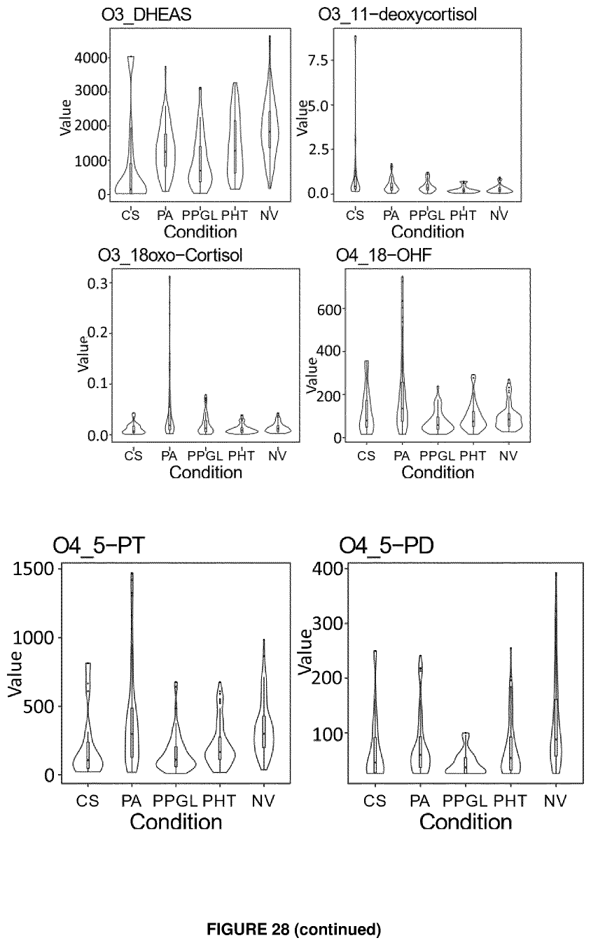

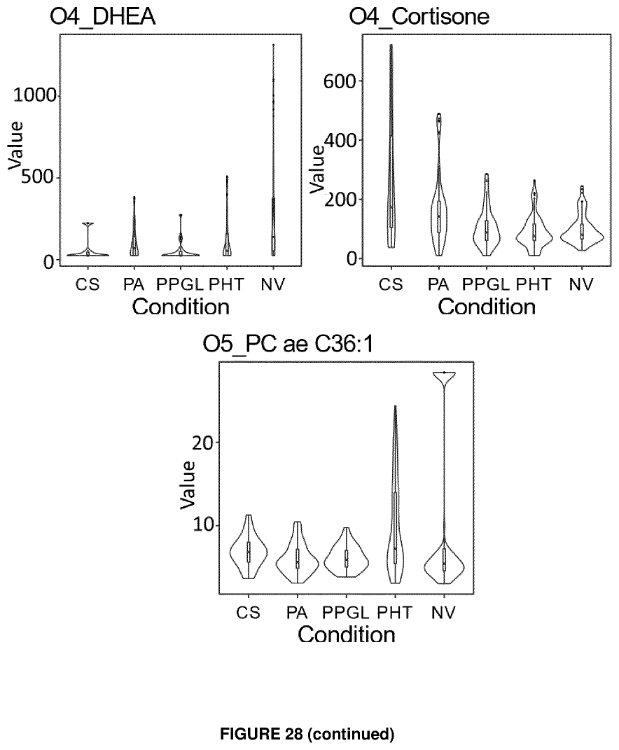

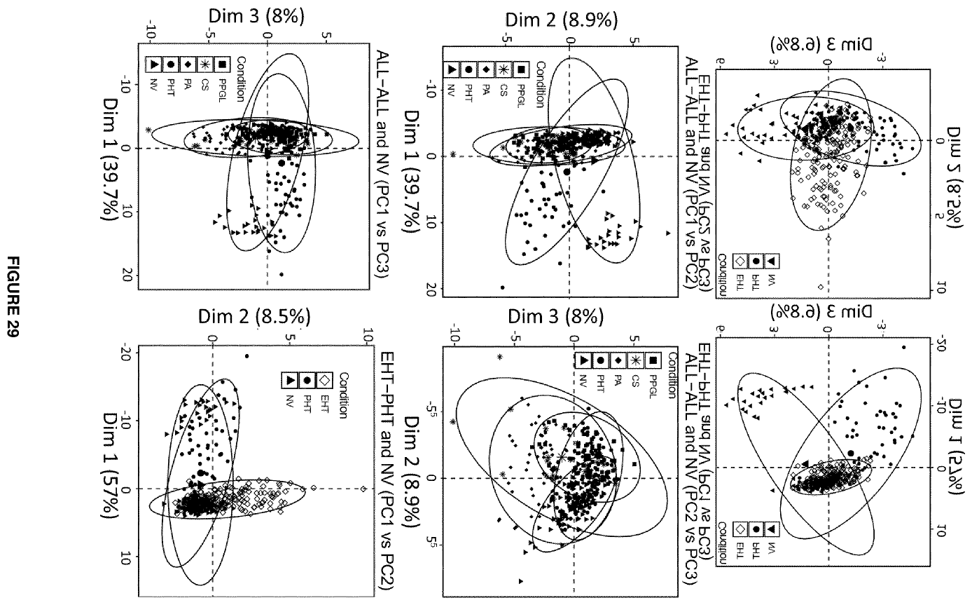

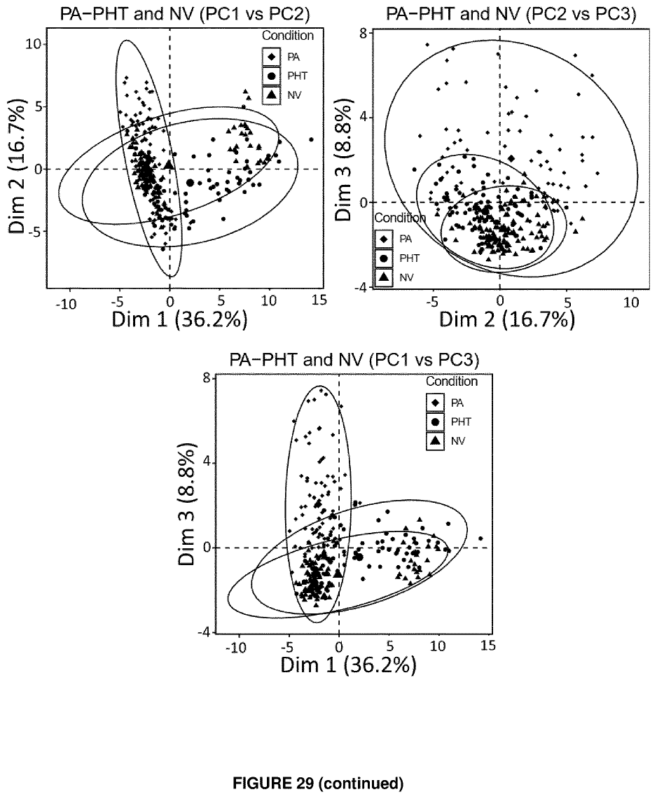

The MOmics features selected in ALL-ALL disease combination were compared with corresponding omic features for NV in the training set (FIG. 28). Also, PCA analysis was conducted for all five disease combinations alongside NV (FIG. 29). The first component of ALL-ALL and EHT-PHT accounted for ˜40% and 57% of the explained variance respectively.

### In-Depth Analysis of Primary and Endocrine Hypertension

The training set of the MOmics data was studied for a different set of scenarios (Table 1). These scenarios include the use of age and sex as features and understanding the effect of age and sex segregated subsets on feature selection in different disease combinations (See stage 2 in FIG. 7).

### Scenario 1: Including (Set A) Vs Excluding (Set B) Age and Sex as Features

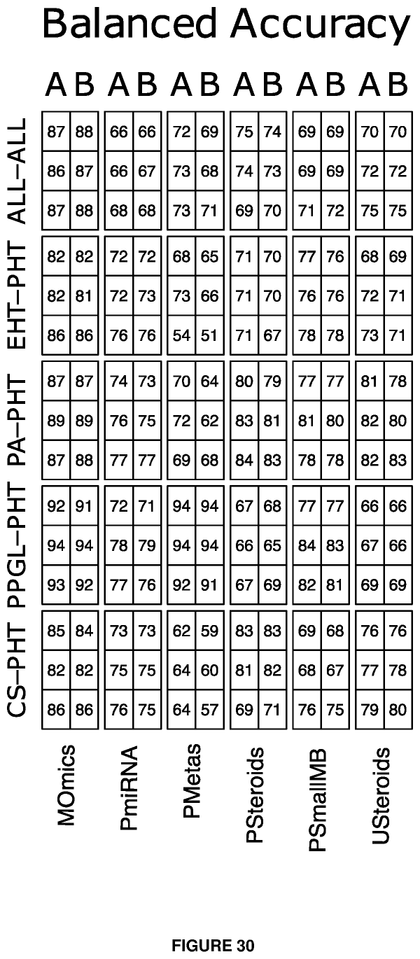

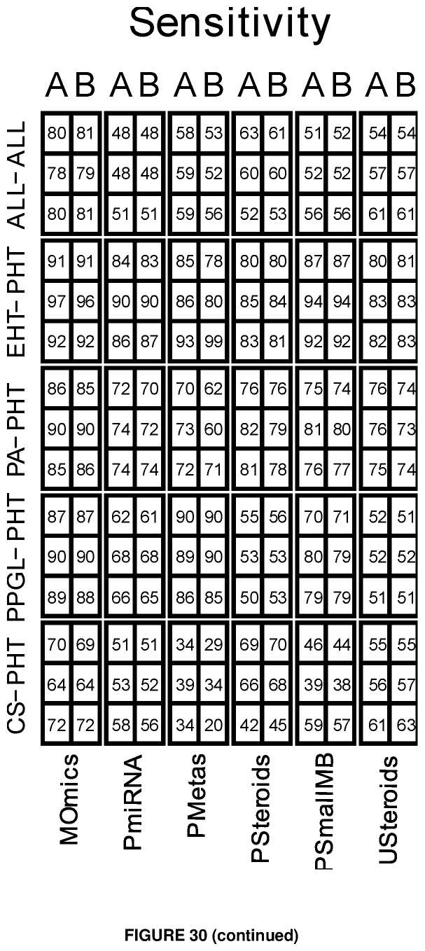

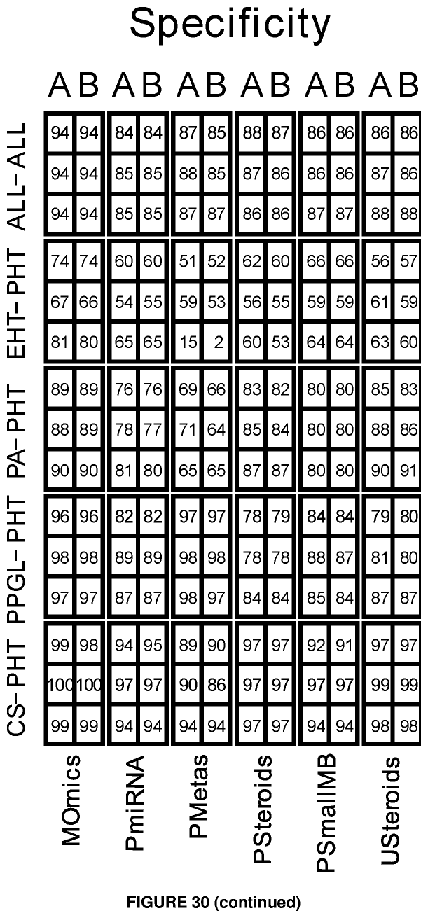

For Scenario 1, MOmics data provided better performance for all disease combinations (FIG. 30). In intra-set comparison, MOmics achieved similar performance in Sets A and B across all disease combinations. Hence, excluding age and sex (in Set B) as features did not materially alter the performance of the classification. However, for PMetas, balanced accuracy dropped when age and sex were excluded (Set B) from the feature set (except for PPGL-PHT). For example, balanced accuracy was down by 5% and 7% for ALL-ALL and EHT-PHT respectively.

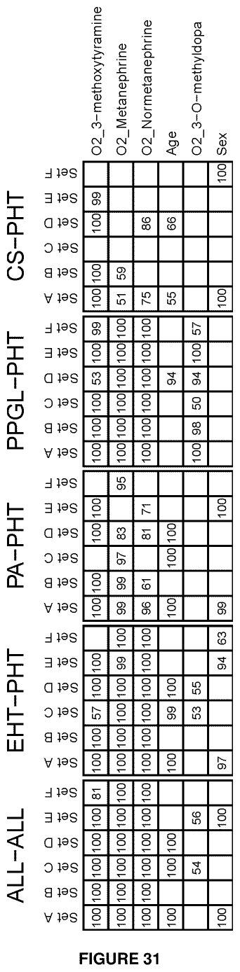

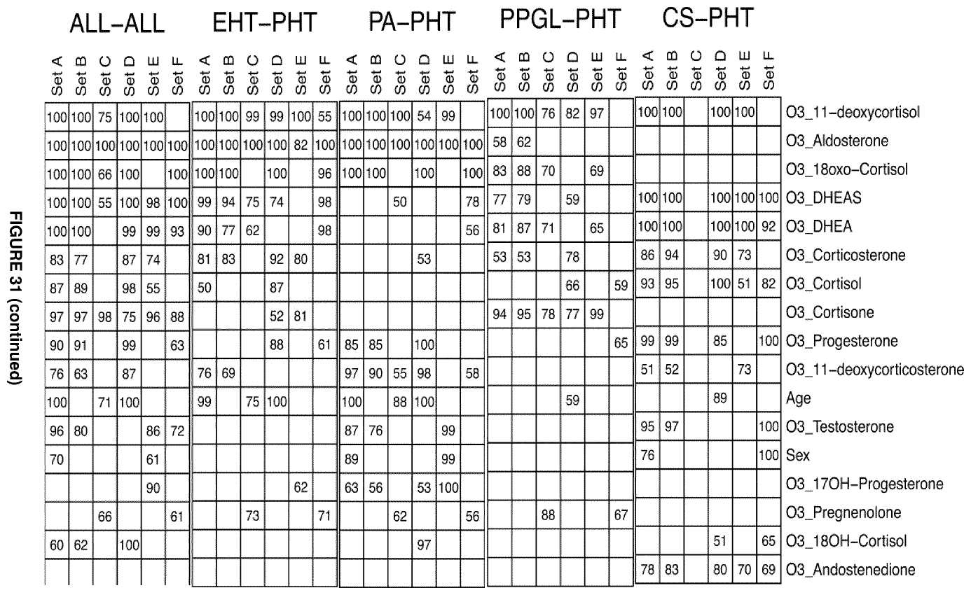

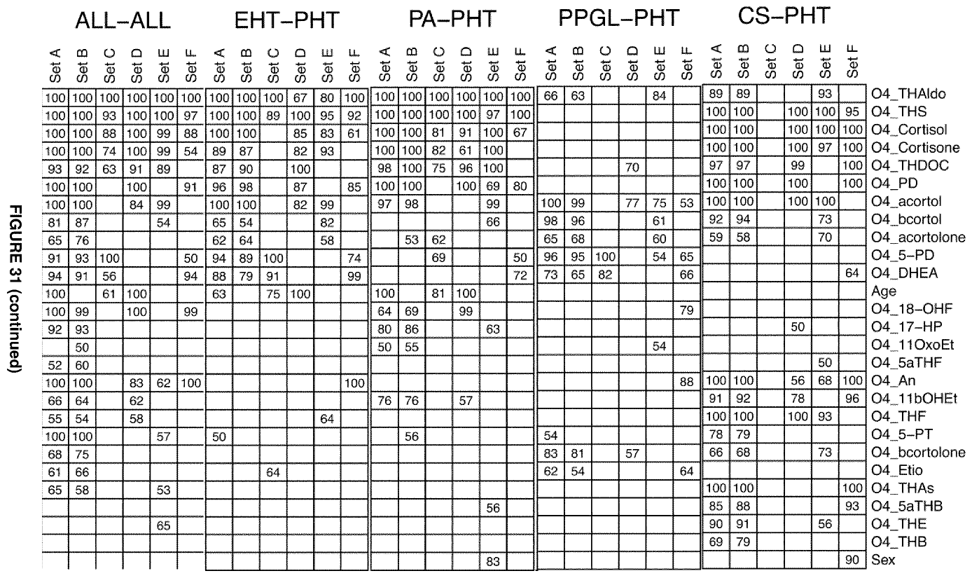

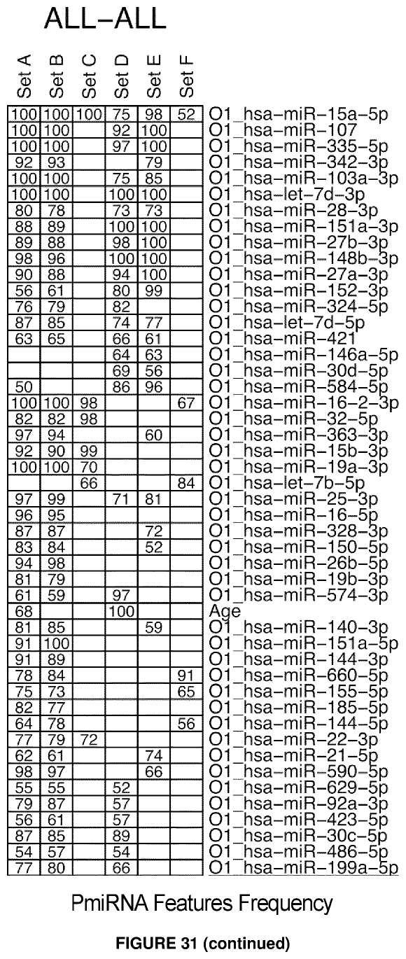

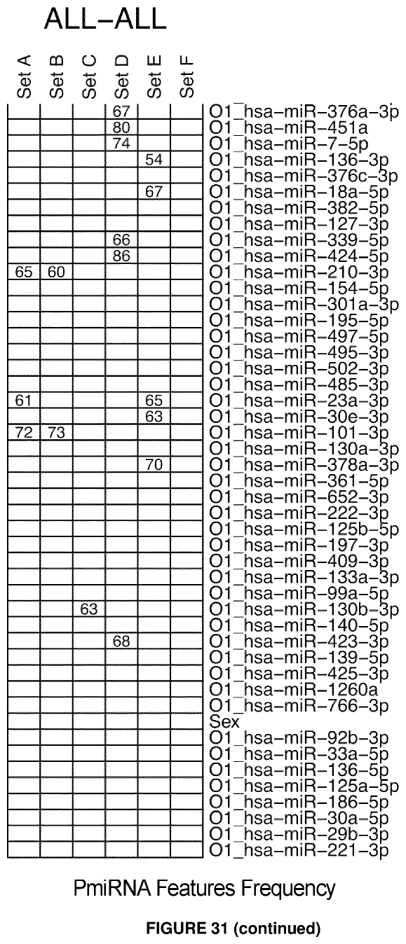

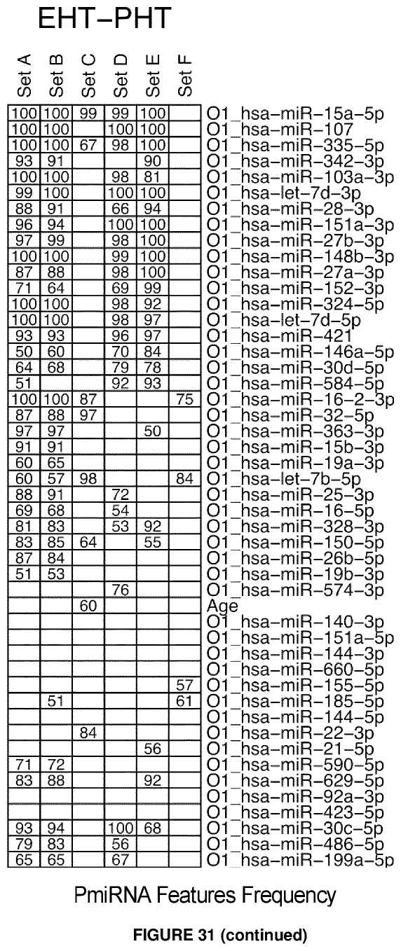

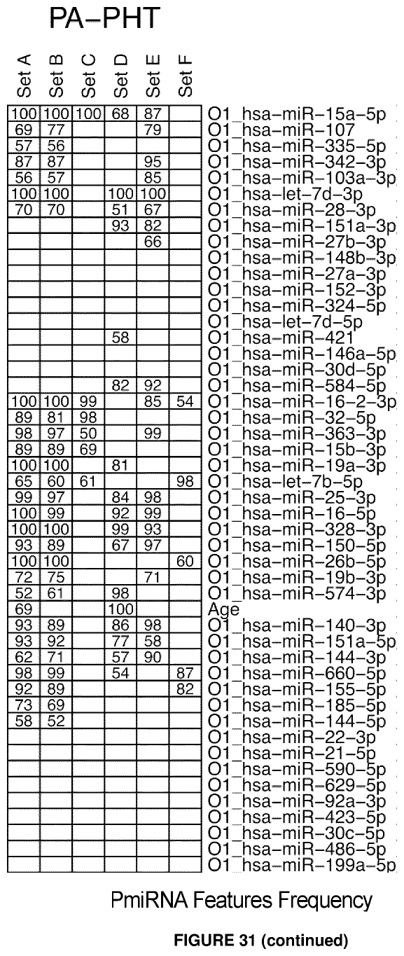

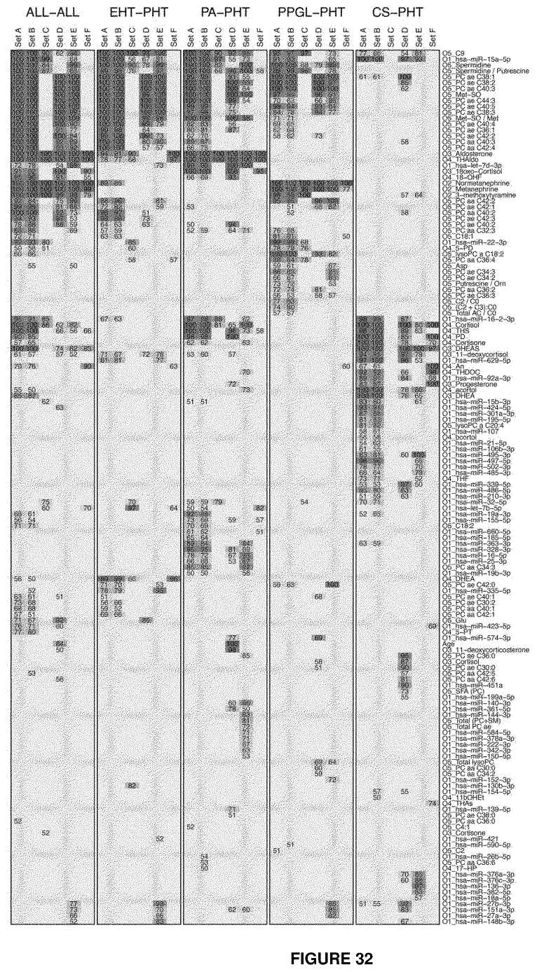

The remaining four mono-omics provided comparable performance irrespective of age and sex being used as features. For example, in the case of USteroids, the detailed summary of features selected during the 100 RR show that almost the same features are selected approximately equal number of times for Set A and B (FIG. 31). Similar trends were observed for other mono-omics (FIG. 31). Notably, for MOmics despite including age and sex as features (Set A), the selection frequencies were below the threshold in 100 RR and therefore they were not designated as top features (FIG. 32).

### Scenario 2: Males (Set C) Vs Females (Set D)

The classification performance of Sets C and D was not comparable since they used different numbers of samples for training and testing. However, it is noticeable that the female subset provided better accuracy for EHT-PHT and PA-PHT in comparison to male subset for MOmics data (FIG. 30). The intra-set comparison highlighted the superior performance of MOmics dataset for ALL-ALL and EHT-PHT disease combinations in comparison to mono-omics irrespective of classifier selection. For PPGL-PHT, PMetas outperformed the MOmics classification for both the Sets. However, in case of CS-PHT, Set C could not be run due to an insufficient number of male CS samples for classifier training (FIG. 8).

From the perspective of feature selection, in the MOmics dataset, different features were selected for Sets C and D (FIG. 32). For example, in ALL-ALL, O1_hsa-miR-15a-5p, O5_Spermidine and O5_Spermidine/Putrescine were only selected for male dataset. On the contrary, various other features such as O5_PC ae C38:1, O3_18oxo-Cortisol, and O4_18-OHF were only selected for female dataset. On close examination, it was evident that the union of Set C and Set D features approximately intersect with both Sets A and B. Similar trends were also observed across most of the disease combinations in MOmics and mono-omics datasets (FIG. 31-32).

### Scenario 3: Older (Set E) Vs Younger (Set F)

Overall, MOmics data provided better classification performance in comparison to mono-omics (except PPGL-PHT), irrespective of the cohort age (FIG. 30). When considering the inter-set comparison, Set E (age >=50) provided better results than Set F (age <50) for almost all disease combinations. A higher number of unique features were selected for both the cohorts for all disease combinations (FIG. 32). Similar trends were noticed for mono-omics datasets (FIG. 31).

FIG. 22 represents a diagram showing the number of unique biomarkers within different overlapping hypertension conditions comparisons.

### DISCUSSION

Here is implemented a MOmics ML integration approach for stratification of arterial hypertension. Our results show that the MOmics approach provided improved discriminatory power in comparison to single omics (mono-omics) data analysis and was able to correctly identify different forms of endocrine hypertension with high sensitivity and specificity, providing potential diagnostic biomarker combinations for diagnosing hypertension subtypes.

With the availability of recent high-throughput experimental and computational technologies, ML-based integration will facilitate the discovery of biomarkers for diagnosis and improve the understanding of complex diseases such as arterial hypertension. However, obtaining MOmics data can be logistically challenging when biosamples are sourced from multiple recruitment sites and require multi-centre omics measurements. This can lead to fewer samples with all available omics for integration. The ENS@T-HT study, by obtaining a complete set of omics for ˜84% of the total patients, provided a straightforward example that this challenge can be successfully addressed. Although a few mono-omics studies on identification of endocrine forms of hypertension have been published, (Eisenhofer et al., 2020; Erlic et al., 2021) to the best of our knowledge, no other study exists that collected and analysed MOmics data for hypertension stratification and predicting hypertension subtypes.

This study predicted EHT subtypes using a dedicated and customisable ML pipeline. The imbalance of classes is a well-known problem in ML which does not allow the classifier to learn from the minority class. This was corrected for CS and PPGL patients with the use of Synthetic Minority Over-sampling TEchnique (SMOTE).(Chawla et al., 2002) Evaluating classification performance was one of the key outcomes for this study. The method used also enabled the assessment of top discriminating features and comparison of these to the NV.

Despite the strong classification performance, the analysis had a few shortcomings. Firstly, since CS is a rare disease, samples for CS patients were limited. Secondly, advanced ML techniques such as deep learning could not be used for this analysis as they require a much larger number of samples than was available in this study. Finally, all the samples could not be used for MOmics integration because of limitations in sample volume or specific quality measures, which is a common problem for a study with multi-site biosamples and multi-centre omics measurement. However, a major strength of this study was to rely on unambiguous diagnosis of the major subtypes of EHT according to guidelines by expert centres. In addition, our analysis only explored the MOmics data using a ML based data-driven approach. The discovered top discriminating features need further investigation in terms of biological significance and pathway network analysis.

For future research, it will be helpful to include a wider population that is enrolled in a prospective manner. This would allow the classifier to become more robust and well-trained for a formal clinical deployment. The ENS@T-HT study is currently capturing such relevant prospective data with an aim to measure the most discriminating features of the new samples and to perform an independent validation. (ClinicalTrials.gov Identifier: NCT02772315). The refined algorithm could be deployed as a webserver-based prediction tool and utilized to screen patients at primary care to refer patients identified as being at risk of endocrine hypertension to centers with appropriate expertise for subsequent evaluation if required. The developed ML pipeline is fully customizable and can be deployed for other mono/MOmics data-based biomarker discovery and analysis studies. For example, it can be used to investigate MOmics signatures for other forms of secondary hypertension such as renal artery stenosis.

### Example 2-A: Materials and Methods

### Patient Details

The plasma and 24 h urine samples from prospective patients with primary aldosteronism (PA), pheochromocytoma/functional paraganglioma (PPGL), Cushing's syndrome (CS), and primary hypertension (PHT) were provided by 7 collaborating clinical centers participating to the ENS@T-HT Horizon2020 consortium (ENSAT-HT Project. ENSAT-HT Project http://www.ensat-ht.eu/.). EHT patients are grouping PA, PPGL and CS. The diagnosis was made following current guidelines (Funder, J. W. et al. The Management of Primary Aldosteronism: Case Detection, Diagnosis, and Treatment: An Endocrine Society Clinical Practice Guideline. J. Clin. Endocrinol. Metab. 101, 1889-1916 (2016); Mulatero, P. et al. Genetics, prevalence, screening and confirmation of primary aldosteronism: a position statement and consensus of the Working Group on Endocrine Hypertension of The European Society of Hypertension*. Journal of Hypertension 38, 1919-1928 (2020); Lenders, J. W. M. et al. Pheochromocytoma and Paraganglioma: An Endocrine Society Clinical Practice Guideline. J Clin Endocrinol Metab 99, 1915-1942 (2014)).

Plasma and 24 h urine samples were collected from 1093 male and female participants, who suffered from one of four hypertension subtypes (PA=513, PPGL=40, CS=47, PHT=493). The plasma and urine samples were stored at −80° C. before dispatch and analysis. All study protocols under which patients were recruited were approved by the local ethics committee of each participating center and all subjects provided written informed consent before participation in the study.

### Multi-Omics Data

Five omics sets were generated (See Table 4).

TABLE 4: The various omics and their feature count.

TABLE 4

| Omic | | | |
|---|---|---|---|
| Number | Omic Name | No of features | |
| 1 | Plasma miRNA | 55 | miRNAs |
| | (PmiRNA) | | |
| 2 | Plasma O-methylated | 4 | metabolites |
| | catecholamines | | |
| | (PMetas) | | |
| 3 | Plasma Steroids | 15 | steroids |
| | (PSteroids) | | |
| 4 | Urine Steroids | 27 | steroids |
| | (USteroids) | | |
| 5 | Plasma Small metabolites | 177 | Small metabolites |
| | (PSmallMB) | | |

All the omics sets were catalogued in the Research Data Management Platfrom (RDMP) (Nind, T. et al. The Research Data Management Platform (RDMP): A novel, process driven, open-source tool for the management of longitudinal cohorts of clinical data. GigaScience (2018) doi: 10.1093/gigascience/giy060) hosted at the Health Informatics Centre (HIC) Safe Haven for systematic access. Although biosamples for 1093 patients were available, after quality controls, omics measurements for all five omics were available for 961 (CS=37, PA=454, PPGL=32, and PHT=438) patients (See Table 5).

TABLE 5: Omics availability with respect to disease type

TABLE 5

| | Number | Omics Type | | | | | | | | | |
|---|---|---|---|---|---|---|---|---|---|---|---|
| S. | of Omics | 1. | 2. | 3. | 4. | 5. | Disease Type | | | | |
| no | Available | PmiRNA | PMetas | PSteroids | USteroids | PSmallMB | CS | PA | PPGL | PHT | Total |
| 1 | 5 | ✓ | ✓ | ✓ | ✓ | ✓ | 37 | 454 | 32 | 438 | 961 |
| 2 | 4 | | ✓ | ✓ | ✓ | ✓ | 3 | 35 | 1 | 20 | 59 |
| 3 | 4 | ✓ | | ✓ | ✓ | ✓ | | 1 | | 1 | 2 |
| 4 | 4 | ✓ | ✓ | ✓ | | ✓ | 2 | 12 | 5 | 14 | 33 |
| 5 | 4 | ✓ | ✓ | ✓ | ✓ | | | 1 | | 1 | 2 |
| 6 | 3 | | ✓ | ✓ | ✓ | | | | 1 | | 1 |
| 7 | 3 | | ✓ | ✓ | | ✓ | | | | 3 | 3 |
| 8 | 3 | ✓ | ✓ | ✓ | | | 1 | | | 2 | 3 |
| 9 | 3 | ✓ | | | ✓ | ✓ | | 1 | | | 1 |
| 10 | 2 | | ✓ | ✓ | | | | 8 | 1 | 13 | 22 |
| 11 | 1 | ✓ | | | | | 4 | 1 | | 1 | 6 |
| | | | | | | | | | | Grand | 1093 |
| | | | | | | | | | | Total: | |

In total 278 multi-omics features (PmiRNA: 55, PMetas: 4, PSteroids: 15, USteroids: 27, and PSmallMB: 177) along with age and sex were exported and used to conduct supervised Machine Learning (ML) experiments.

Table 6 provides a list of names of all omics features. The extreme outliers in omics features were carefully evaluated and capped. The names of features were prefixed with ‘O1_’, ‘O2_’, ‘O3_’, ‘O4_’, and ‘O5_’ for Plasma miRNA, Plasma O-methylated catecholamines (PMetas), Plasma Steroids, Urinary Steroids, and Plasma Small metabolites respectively.

TABLE 6: List of all omics features

TABLE 6

| | | | USteroids | PSmallMB |
|---|---|---|---|---|
| PmiRNA (55) | PMetas (4) | PSteroids (15) | (27) | (129) |
| hsa-let-7b-5p | 3-O-methyldopa | Aldosterone | αcortol | AAA |
| hsa-let-7d-3p | Metanephrine | Androstenedione | αcortolone | Ac-Orn |
| hsa-let-7d-5p | 3-methoxytyramine | Corticosterone | An | ADMA |
| hsa-let-7g-5p | Normetanephrine | Cortisol | βcortol | ADMA/Arg |
| hsa-miR-103a-3p | | Cortisone | βcortolone | Ala |
| hsa-miR-106b-3p | | DHEA | Cortisol | alpha-AAA |
| hsa-miR-107 | | DHEAS | Cortisone | Arg |
| hsa-miR-130a-3p | | 11-deoxycorticosterone | DHEA | Asn |
| hsa-miR-130b-3p | | 11-deoxycortisol | Etio | Asp |
| hsa-miR-140-5p | | 17OH-Progesterone | PD | BCAA |
| hsa-miR-144-3p | | 18OH-Cortisol | PT | C0 |
| hsa-miR-146a-5p | | 18oxo-Cortisol | THAIdo | C10 |
| hsa-miR-148b-3p | | 21-deoxycortisol | THAS | C10:1 |
| hsa-miR-150-5p | | 18OH-Corticosterone | THB | C10:2 |
| hsa-miR-151a-3p | | 11-dehydrocorticosterone | THDOC | C12 |
| hsa-miR-152-3p | | | THE | C12-DC |
| hsa-miR-155-5p | | | THF | C14 |
| hsa-miR-15a-5p | | | THS | C14:1 |
| hsa-miR-15b-3p | | | 11βOHAn | C14:2 |
| hsa-miR-16-2-3p | | | 11βOHEt | C16 |
| hsa-miR-16-5p | | | 11OxoEt | C16:2 |
| hsa-miR-181a-5p | | | 17-HP | C18 |
| hsa-miR-195-5p | | | 18-OHF | C18:1 |
| hsa-miR-199a-5p | | | 5αTHB | C18:1-OH |
| hsa-miR-19a-3p | | | 5αTHF | C18:2 |
| hsa-miR-19b-3p | | | 5-PD | C2 |
| hsa-miR-210-3p | | | 5-PT | C2/C0 |
| hsa-miR-222-3p | | | | C3 |
| hsa-miR-22-3p | | | | C3-DC (C4-OH) |
| hsa-miR-23a-3p | | | | C4 |
| hsa-miR-23b-3p | | | | C4:1 |
| hsa-miR-24-3p | | | | C5 |
| hsa-miR-25-3p | | | | C6:1 |
| hsa-miR-26b-5p | | | | C6 (C4:1-DC) |
| hsa-miR-27a-3p | | | | Cit |
| hsa-miR-27b-3p | | | | Cit/Arg |
| hsa-miR-301a-3p | | | | Cit/Orn |
| hsa-miR-30c-5p | | | | CPT-I ratio |
| hsa-miR-30d-5p | | | | Creatinine |
| hsa-miR-324-5p | | | | Essential AA |
| hsa-miR-32-5p | | | | Fisher ratio |
| hsa-miR-328-3p | | | | Gln |
| hsa-miR-335-5p | | | | Glu |
| hsa-miR-33a-5p | | | | Glucogenic AA |
| hsa-miR-342-3p | | | | Gly |
| hsa-miR-363-3p | | | | H1 |
| hsa-miR-423-5p | | | | His |
| hsa-miR-451a | | | | Ile |
| hsa-miR-485-3p | | | | Kynurenine |
| hsa-miR-486-5p | | | | Kynurenine/Trp |
| hsa-miR-495-3p | | | | Leu |
| hsa-miR-497-5p | | | | Lys |
| hsa-miR-502-3p | | | | lysoPC a C14:0 |
| hsa-miR-629-5p | | | | lysoPC a C16:0 |
| hsa-miR-92a-3p | | | | lysoPC a C16:1 |
| | | | | lysoPC a C17:0 |
| | | | | lysoPC a C18:0 |
| | | | | lysoPC a C18:1 |
| | | | | lysoPC a C18:2 |
| | | | | lysoPC a C20:3 |
| | | | | lysoPC a C20:4 |
| | | | | lysoPC a C28:0 |
| | | | | lysoPC a C28:1 |
| | | | | Met |
| | | | | Met-SO |
| | | | | Met-SO/Met |
| | | | | Non essential |
| | | | | AA |
| | | | | Orn |
| | | | | Orn/Arg |
| | | | | PC aa C24:0 |
| | | | | PC aa C26:0 |
| | | | | PC aa C28:1 |
| | | | | PC aa C30:0 |
| | | | | PC aa C32:0 |
| | | | | PC aa C32:1 |
| | | | | PC aa C32:2 |
| | | | | PC aa C32:3 |
| | | | | PC aa C34:1 |
| | | | | PC aa C34:2 |
| | | | | PC aa C34:3 |
| | | | | PC aa C34:4 |
| | | | | PC aa C36:0 |
| | | | | PC aa C36:1 |
| | | | | PC aa C36:2 |
| | | | | PC aa C36:3 |
| | | | | PC aa C36:4 |
| | | | | PC aa C36:5 |
| | | | | PC aa C36:6 |
| | | | | PC aa C38:0 |
| | | | | PC aa C38:1 |
| | | | | PC aa C38:3 |
| | | | | PC aa C38:4 |
| | | | | PC aa C38:5 |
| | | | | PC aa C38:6 |
| | | | | PC aa C40:2 |
| | | | | PC aa C40:3 |
| | | | | PC aa C40:4 |
| | | | | PC aa C40:5 |
| | | | | PC aa C40:6 |
| | | | | PC aa C42:0 |
| | | | | PC aa C42:1 |
| | | | | PC aa C42:2 |
| | | | | PC aa C42:4 |
| | | | | PC aa C42:5 |
| | | | | PC aa C42:6 |
| | | | | PC ae C30:0 |
| | | | | PC ae C32:1 |
| | | | | PC ae C32:2 |
| | | | | PC ae C34:0 |
| | | | | PC ae C34:1 |
| | | | | PC ae C34:2 |
| | | | | PC ae C34:3 |
| | | | | PC ae C36:0 |
| | | | | PC ae C36:1 |
| | | | | PC ae C36:2 |
| | | | | PC ae C36:3 |
| | | | | PC ae C36:4 |
| | | | | PC ae C36:5 |
| | | | | PC ae C38:0 |
| | | | | PC ae C38:1 |
| | | | | PC ae C38:2 |
| | | | | PC ae C38:3 |
| | | | | PC ae C38:4 |
| | | | | PC ae C38:5 |
| | | | | PC ae C38:6 |
| | | | | PC ae C40:1 |
| | | | | PC ae C40:2 |
| | | | | PC ae C40:3 |
| | | | | PC ae C40:4 |
| | | | | PC ae C40:5 |
| | | | | PC ae C40:6 |
| | | | | PC ae C42:0 |
| | | | | PC ae C42:1 |
| | | | | PC ae C42:2 |
| | | | | PC ae C42:3 |
| | | | | PC ae C42:4 |
| | | | | PC ae C42:5 |
| | | | | PC ae C44:3 |
| | | | | PC ae C44:4 |
| | | | | PC ae C44:5 |
| | | | | PC ae C44:6 |
| | | | | Phe |
| | | | | Pro |
| | | | | Putrescine |
| | | | | Putrescine/Orn |
| | | | | SDMA |
| | | | | SDMA/Arg |
| | | | | Ser |
| | | | | Serotonin |
| | | | | Serotonin/Trp |
| | | | | SM C16:0 |
| | | | | SM C16:1 |
| | | | | SM C18:0 |
| | | | | SM C18:1 |
| | | | | SM C20:2 |
| | | | | SM C24:0 |
| | | | | SM C24:1 |
| | | | | SM C26 C26:0 |
| | | | | SM C26 C26:1 |
| | | | | SM (OH) C14:1 |
| | | | | SM (OH) C16:1 |
| | | | | SM (OH) C22:1 |
| | | | | SM (OH) C22:2 |
| | | | | SM (OH) C24:1 |
| | | | | Spermidine |
| | | | | t4-OH-Pro |
| | | | | Taurine |
| | | | | Thr |
| | | | | Total AA |
| | | | | total DMA |
| | | | | Total DMA/Arg |
| | | | | Total SM-OH |
| | | | | Trp |
| | | | | Tyr |
| | | | | Tyr/Phe |
| | | | | Val |
| | | | | (C2 + C3):C0 |

### Supervised Machine Learning Pipeline

### Training of the Machine Learning (ML)

The machine learning (ML) pipeline developed for the molecular signature discovery is disclosed in PCT/EP2022/053142 filed on Feb. 9, 2022, the content of which is incorporated by reference.

The ML system was trained as disclosed in Example 1.

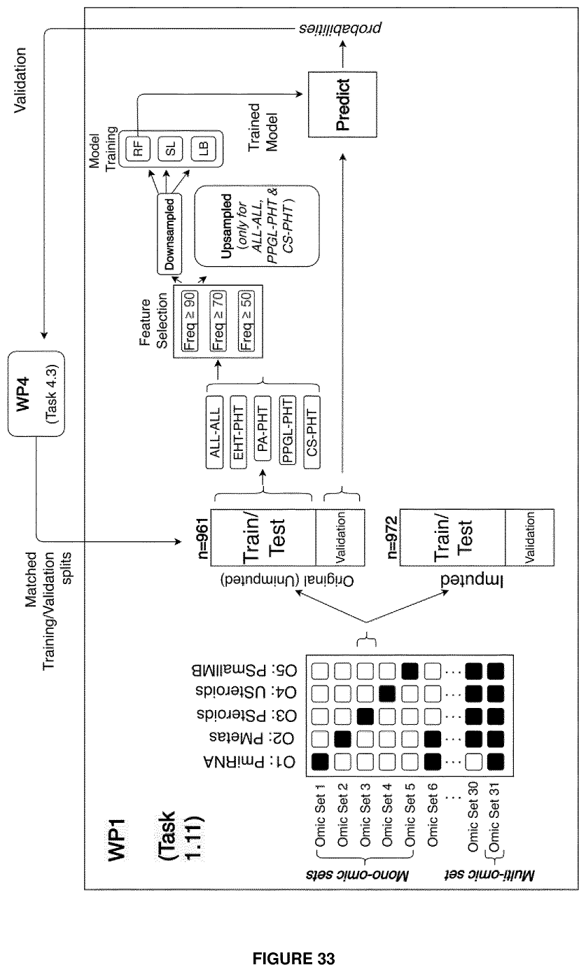

FIG. 33 shows the high-level schematic.

The following subsections highlight the improvements for retraining and refinement of molecular signature.

### Retraining of the Machine Learning (ML)

The following subsections highlight the improvements for retraining and refinement of molecular signature (combination of biomarkers).

### Disease Combinations

The five different disease combinations used for classification were: ALL-ALL (PPGL vs PA vs CS vs PHT), EHT (PPGL+PA+CS)-PHT, and each endocrine hypertension (i.e., PPGL/PA/CS)-PHT.

### Omic Combinations, Data Imputation and Sampling

In addition to single omics and complete multi-omics set other intermediate combinations were also investigated. Table 4 shows the list of all 31 omics combinations.

TABLE 1: Various omic combinations used during the analysis.

TABLE 4

| Omic | | | | | | |
|---|---|---|---|---|---|---|
| Combination | Omics | | | | | |
| No. | used | PmiRNA | PMetas | PSteroids | Usteroids | PSmallMB |
| 1 | 1 | ✓ | | | | |
| 2 | 1 | | ✓ | | | |
| 3 | 1 | | | ✓ | | |
| 4 | 1 | | | | ✓ | |
| 5 | 1 | | | | | ✓ |
| 6 | 2 | ✓ | ✓ | | | |
| 7 | 2 | ✓ | | ✓ | | |
| 8 | 2 | ✓ | | | ✓ | |
| 9 | 2 | ✓ | | | | ✓ |
| 10 | 2 | | ✓ | ✓ | | |
| 11 | 2 | | ✓ | | ✓ | |
| 12 | 2 | | ✓ | | | ✓ |
| 13 | 2 | | | ✓ | ✓ | |
| 14 | 2 | | | ✓ | | ✓ |
| 15 | 2 | | | | ✓ | ✓ |
| 16 | 3 | ✓ | ✓ | ✓ | | |
| 17 | 3 | ✓ | ✓ | | ✓ | |
| 18 | 3 | ✓ | ✓ | | | ✓ |
| 19 | 3 | ✓ | | ✓ | ✓ | |
| 20 | 3 | ✓ | | ✓ | | ✓ |
| 21 | 3 | ✓ | | | ✓ | ✓ |
| 22 | 3 | | ✓ | ✓ | ✓ | |
| 23 | 3 | | ✓ | ✓ | | ✓ |
| 24 | 3 | | ✓ | | ✓ | ✓ |
| 25 | 3 | | | ✓ | ✓ | ✓ |
| 26 | 4 | ✓ | ✓ | ✓ | ✓ | |
| 27 | 4 | ✓ | ✓ | ✓ | | ✓ |
| 28 | 4 | ✓ | ✓ | | ✓ | ✓ |
| 29 | 4 | ✓ | | ✓ | ✓ | ✓ |
| 30 | 4 | | ✓ | ✓ | ✓ | ✓ |
| 31 | 5 | ✓ | ✓ | ✓ | ✓ | ✓ |

Since very few CS and PPGL samples were available for the analysis, two approaches were incorporated to minimize the impact of the class imbalance problem during model training. Firstly, the 5 CS and 6 PPGL samples (See rows 2 and 4 of Table 2) were imputed for the fifth unavailable omic using DMwR27 package in R. These 11 samples were then concatenated with the 961 samples. The 961 and 972 sample sets were denoted as ‘Original’ and ‘Imputed’ respectively. Secondly, up-sampling and down-sampling were performed for disease combinations with class imbalance. All five disease combinations were down-sampled, while only three (CS-PHT, PPGL-PHT and ALL-ALL) were up-sampled. The up-sampling involved additional synthetic samples generation for CS and PPGL using SMOTE8. These sampling approaches were denoted as ‘up’ and ‘down’ respectively.

### Classifiers and Feature Selection

Three different machine learning classifiers were used: LogitBoost (LB) (Friedman, J., Hastie, T. & Tibshirani, R. Additive Logistic Regression: a Statistical View of Boosting. Annals of Statistics 28, 2000 (1998)), Simple Logistic (SL) (Sumner, M., Frank, E. & Hall, M. Speeding up logistic model tree induction. in Proceedings of the 9th European Conference on European Conference on Machine Learning and Principles and Practice of Knowledge Discovery in Databases 675-683 (Springer-Verlag, 2005). doi: 10.1007/11564126_72), and Random Forest (RF) (Breiman, L. Random Forests. Machine Learning 45, 5-32 (2001)). The classification was implemented using the caret (Kuhn, M. Building Predictive Models in R Using the caret Package. Journal of Statistical Software 28, 1-26 (2008)) and RWeka (Hornik, K., Buchta, C. & Zeileis, A. Open-source machine learning: R meets Weka. Comput Stat 24, 225-232 (2009)) in R (R Core Team. R: A Language and Environment for Statistical Computing. (R Foundation for Statistical Computing, 2013)).

For feature selection, the wrapper Boruta (Kursa, M. B. & Rudnicki, W. R. Feature Selection with the Boruta Package. Journal of Statistical Software 36, 1-13 (2010)) method was used. One of the key objectives of the analysis was to identify the list of most discriminating features for a given disease combination. The top features during feature selection were chosen across 100 repeats with a cut-off for the feature frequency of 50, 70 and 90, which were used for the final training. This value was chosen empirically as a trade-off for finding the optimal number of reduced features without impacting the classification performance.

### Evaluation Scenarios

As shown in FIG. 33, considering the various combinations, in total 446,400 iterations were evaluated that led to 4044 final models (a few models did not converge since no valuable top features were found). The iterations were computed as:

$$31 omics combinations \times 2 imputation sets ( original \& imputed ) \times [ 5 disease combinations ( down - sampling ) + 3 disease combinations ( up - sampling ) \times 3 classifiers \times 100 random repeats$$

### Training/Test—Validation Split and Performance Metrics

The omics sets were split into training and validation sets (80-20%) based on the patient IDs. These patient IDs were randomly drawn and matched for age and sex amongst the two sets. The validation set was kept apart throughout the ML model training pipeline. However, the training set was further split into training and test using Monte-Carlo based random repeats 100 times. The classifications results were evaluated over these random repeats using performance metrics: balanced accuracy (to adjust for the class imbalance problem), sensitivity, specificity, AUC, F1, and Kappa score. All the models were ranked w.r.t to balanced accuracy and top 5 models for each disease combination with 2 imputations (original & imputed) and sampling (up and down) types. Finally, the configuration from the top 5 models was used to train on the complete training set. The top-trained models were then used to predict the disease for validation set patients. These outcomes were delivered in the form of probabilities. The features selected for training the top models were then evaluated to understand the effect of various chosen parameters.

### Example 2-B: Results

The results of the retraining and refining molecular signature (combination of biomarkers) are described as follows:

### Pre-Processing

The pre-processing involved standard exploratory data analysis which included evaluation of descriptive statistics, principal component analysis (PCA) followed by outlier detection and removal. Next, the various datasets as per the training/validation splits were set up and the ML pipeline was run.

### Top ML Models and Discriminating Features

All 4044 trained models were ranked based on the balanced accuracy for each disease combination, patient (original, imputed) and sampling (up, down) type. The top 5 models for each setting were then further investigated (80 models). Table 7-11 show the top models and their details for ALL vs ALL, EHT-PHT, CS-PHT, PA-PHT, and PPGL-PHT respectively.

It shows the omics combination, count of chosen features and mean performance on the training set. The blank cell with black color highlights the omics from which no features was selected.

Table 7: Top models for All vs All disease combination. Black shaded cell highlights the omic that was included in the omics combination but none of their features was selected in the final model.

Table 8: Top models for EHT vs PHT disease combination. Grey shaded cell highlights the omic that was included in the omics combination but none of their features was selected in the final model.

Table 9: Top models for CS vs PHT disease combination. Grey shaded cell highlights the omic that was included in the omics combination but none of their features was selected in the final model.

Table 10: Top models for PA vs PHT disease combination. Grey shaded cell highlights the omic that was included in the omics combination but none of their features was selected in the final model.

Table 11: Top models for PPGL vs PHT disease combination. Grey shaded cell highlights the omic that was included in the omics combination but none of their features was selected in the final model.

Once the top models were identified and selected following the training phase, they were applied to the validation set for the first time and predictions made by each model for all the patients in the validation set. The outcome of prediction (in the form of probabilities) for validation set patients using these top 80 models was selected. Table 12 shows an example of the outcome probabilities for the ALL-ALL combination.

TABLE 12

| S. | | Probability outcome | | | |
|---|---|---|---|---|---|
| no | Patient ID | PPGL | CS | PA | PHT |
| 1 | Patient 1 | 3.03 × 10⁻⁰² | 1.14 × 10⁻⁰² | 1.53 × 10⁻⁰¹ | 8.06 × 10⁻⁰¹ |
| 2 | Patient 2 | 5.02 × 10⁻⁰⁸ | 3.50 × 10⁻⁰³ | 9.91 × 10⁻⁰¹ | 5.90 × 10⁻⁰³ |
| 3 | Patient 3 | 4.43 × 10⁻⁰⁵ | 3.32 × 10⁻⁰² | 2.69 × 10⁻⁰¹ | 6.98 × 10⁻⁰¹ |
| 4 | Patient 4 | 2.94 × 10⁻⁰⁵ | 2.88 × 10⁻⁰³ | 9.31 × 10⁻⁰¹ | 6.60 × 10⁻⁰² |
| 5 | Patient 5 | 5.09 × 10⁻⁰⁹ | 3.14 × 10⁻⁰³ | 9.93 × 10⁻⁰¹ | 3.99 × 10⁻⁰³ |
| 6 | Patient 6 | 1.05 × 10⁻⁰⁴ | 2.37 × 10⁻⁰² | 7.13 × 10⁻⁰¹ | 2.63 × 10⁻⁰¹ |
| 7 | Patient 7 | 2.49 × 10⁻⁰⁵ | 1.99 × 10⁻⁰³ | 9.65 × 10⁻⁰¹ | 3.32 × 10⁻⁰² |
| 8 | Patient 8 | 5.59 × 10⁻⁰² | 2.48 × 10⁻⁰² | 1.72 × 10⁻⁰¹ | 7.47 × 10⁻⁰¹ |
| 9 | Patient 9 | 5.78 × 10⁻⁰⁵ | 5.44 × 10⁻⁰² | 2.15 × 10⁻⁰¹ | 7.31 × 10⁻⁰¹ |
| 10 | Patient 10 | 1.42 × 10⁻⁰⁸ | 3.68 × 10⁻⁰⁶ | 9.99 × 10⁻⁰¹ | 7.42 × 10⁻⁰⁴ |

A heatmap of chosen features in top models across the various disease combinations was prepared (FIG. 34). Most of these features were common amongst ALL-ALL, EHT-PHT, and PA-PHT. However, in the case of CS-PHT unique features were observed. For example, one PSteroid (03_DHEA) and two PSmallMB (O5_Met-SO/Met and O5_Trp) were only selected for the CS-PHT combination. Also, no PmiRNA features were selected for PA-PHT and PPGL-PHT.

### CONCLUSION

This study reports that the retraining of the ML models and refinement of the molecular signature (combination of biomarkers) was performed successfully. The top-performing ML models and their corresponding discriminating features were evaluated. The prediction probabilities for the patients in the validation set were computed using the top models.

1. A combination of biomarkers comprising at least:
(i-a) one biomarker selected in each of the following group of biomarkers: Patient's age, Plasma steroids, and Urinary steroids, and at least one biomarker selected in at least one of the group of biomarkers: O-methylated catecholamines, Small metabolites, and miRNA;
(i-b) one biomarker selected in each of the following group of biomarkers: Plasma steroids, Urinary steroids, and Small metabolites, and at least one biomarker selected in at least one of the group of biomarkers: Patient's age, O-methylated catecholamines, and miRNA,
(i-c) one biomarker selected in each of the following group of biomarkers: Urinary steroids, and at least one biomarker selected in at least one of the group of biomarkers: Plasma steroids, Small metabolites, and miRNA,
(i-d) one biomarker selected in each of the following group of biomarkers: Patient's age, Plasma steroids, Urinary steroids, and Small metabolites, and at least one biomarker selected in at least one of the group of biomarkers: O-methylated catecholamines, and miRNA, or
(i-e) one biomarker selected in each of the following group of biomarkers: O-methylated catecholamines, and at least one biomarker selected in at least one of the group of biomarkers: Patient's age, Urinary steroids, and Small metabolites.

2. The combination of biomarkers according to claim 1, comprising at least:
(i-a) age, plasma 11-deoxycorticosterone, plasma 11-deoxycortisol, plasma 18OH-corticosterone, plasma 18OH-cortisol, plasma 18oxo-cortisol, plasma 21-deoxycortisol, plasma aldosterone, plasma corticosterone, plasma dehydroepiandrosterone sulfate (DHEAS), urinary 18-hydroxycortisol (18-OHF), urinary α-cortol (acortol), urinary pregnanediol (PD), urinary 3α,5β-tetrahydroaldosterone (THAldo), urinary tetrahydrodeoxycorticosterone (THDOC), and urinary tetrahydro-11-deoxycortisol (THS);
and at least one additional biomarker selected in the group comprising, or consisting in, plasma hsa-let-7g-5p, plasma hsa-miR-106b-3p, plasma hsa-miR-301a-3p, plasma hsa-miR-485-3p, plasma 3-methoxytyramine, plasma metanephrine, plasma normetanephrine, plasma 11-dehydrocorticosterone, plasma cortisol, plasma cortisone, urinary 11-β-hydroxy-androsterone (11-β-OHAn), urinary 17-OH-pregnanolone (17-HP), urinary 5-pregnanediol (PD), urinary 5-pregnenetriol (5-PT), urinary 5α-tetrahydrocortisol (5αTHF), urinary androsterone (An), urinary cortisol, urinary cortisone, urinary dehydroepiandrosterone (DHEA), urinary etiocholanolone (Etio), urinary pregnenetriol (PT), urinary tetrahydro-11-dehydrocorticosterone (THAs), urinary tetrahydrocorticosterone (THB), urinary tetrahydrocortisol (THF), urinary tetrahydrocortisone (THE), urinary α-cortolone (Acortolone), urinary β-cortol (Bcortol), urinary β-cortolone (Bcortolone), plasma acetylcarnitine (C2), plasma C6 (C4:1-DC) (hexanoylcarnitine), plasma creatinine, plasma decanoylcarnitine (C10), plasma dodecanoylcarnitine (C12), plasma glutamic acid, plasma H1 (sum of hexoses), plasma lysoPC a C16:0, plasma lysoPC a C17:0, plasma octadecadienylcarnitine (C18:2), plasma octadecenoylcarnitine (C18:1), plasma PC aa C32:1, plasma PC aa C32:2, plasma PC aa C32:3, plasma PC aa C34:1, plasma PC aa C34:2, plasma PC aa C34:3, plasma PC aa C34:4, plasma PC aa C36:1, plasma PC aa C36:2, plasma PC aa C36:3, plasma PC ae C36:3, plasma serotonin, plasma serotonin/tryptophan ratio (Serotonin/Trp), plasma spermidine, plasma tetradecenoylcarnitine (C14:1), plasma total dimethylarginine/arginine ratio (Total DMA/Arg), and combinations thereof; or
(i-b) plasma 11-deoxycorticosterone, plasma 11-deoxycortisol, plasma 18OH-corticosterone, plasma 18OH-cortisol, plasma 18oxo-cortisol, plasma 21-deoxycortisol, plasma aldosterone, urinary 18-hydroxycortisol (18-OHF), urinary 3α,5β-tetrahydroaldosterone (THAldo), urinary tetrahydrodeoxycorticosterone (THDOC), urinary tetrahydro-11-deoxycortisol (THS), plasma decanoylcarnitine (C10), plasma dodecanoylcarnitine (C12), plasma octadecadienylcarnitine (C18:2), plasma H1 (sum of hexoses), plasma PC aa C32:1, plasma PC aa C34:3, and plasma serotonin, plasma serotonin/tryptophan ratio (Serotonin/Trp),
and at least one additional biomarker selected in the group comprising, or consisting in, age, plasma hsa-let-7g-5p, plasma lysoPC a C16:0, plasma lysoPC a C17:0, plasma normetanephrine, plasma corticosterone, plasma dehydroepiandrosterone sulfate (DHEAS), urinary pregnanediol (PD), plasma acetylcarnitine (C2), plasma C6 (C4:1-DC) (hexanoylcarnitine (fumarylcarnitine)), plasma citrulline/arginine ratio (Cit/Arg), plasma creatinine, plasma glutamic acid (Glu), plasma octadecenoylcarnitine (C18:1), plasma PC aa C32:2, plasma PC aa C32:3, plasma PC aa C34:1, plasma PC aa C34:2, plasma PC aa C36:2, plasma PC aa C36:3, plasma PC ae C36:3, plasma taurine, plasma tetradecenoylcarnitine (C14:1), plasma total dimethylarginine/arginine ratio (Total DMA/Arg), and combinations thereof,
(i-c) urinary α-cortol (acortol), and urinary tetrahydro-11-deoxycortisol (THS),
and at least one of plasma 11-deoxycorticosterone, plasma 11-deoxycortisol, plasma dehydroepiandrosterone (DHEA), plasma dehydroepiandrosterone sulfate (DHEAS), plasma hsa-miR-19a-3p, plasma methioninesulfoxide/methionine ratio (Met-SO/Met), plasma tryptophan, urinary 5-pregnenetriol (5-PT), urinary androsterone (An), urinary cortisol, urinary etiocholanolone (Etio), and combinations thereof
(i-d) age, plasma 11-deoxycorticosterone, plasma 11-deoxycortisol, plasma 18OH-corticosterone, plasma 18OH-cortisol, plasma 18oxo-cortisol, plasma 21-deoxycortisol, plasma aldosterone, urinary 18-hydroxycortisol (18-OHF), urinary pregnanediol (PD), 3α,5β-tetrahydroaldosterone urinary (THAldo), urinary tetrahydrodeoxycorticosterone (THDOC), urinary tetrahydro-11-deoxycortisol (THS), plasma decanoylcarnitine (C10), plasma tetradecenoylcarnitine (C14:1), plasma octadecenoylcarnitine (C18:1), plasma octadecadienylcarnitine (C18:2), plasma acetylcarnitine (C2), plasma C6 (C4:1-DC) (hexanoylcarnitine), plasma lysoPC a C17:0, plasma PC aa C32:1, plasma PC aa C32:2, plasma PC aa C32:3, plasma PC aa C34:2, plasma PC aa C34:3, plasma PC aa C36:3, plasma serotonin, and plasma serotonin/tryptophan ratio (Serotonin/Trp),
and at least one of plasma creatinine, plasma dehydroepiandrosterone sulfate (DHEAS), plasma dodecanoylcarnitine (C12), plasma H1 (sum of hexoses), plasma lysoPC a C16:0, plasma PC aa C34:1, plasma PC aa C34:4, plasma PC aa C36:1, plasma PC aa C36:2, plasma taurine, urinary dehydroepiandrosterone (DHEA), and combinations thereof, or
(i-e) methoxytyramine, plasma metanephrine, and plasma normetanephrine,
and at least one of age, plasma acetylornithine (Ac-Orn), urinary androsterone (An), urinary etiocholanolone (Etio), and combinations thereof.

3. The combination of biomarkers according to claim 1, wherein
(ii-a) the at least one additional biomarker in (i-a) is selected from plasma metanephrine, plasma normetanephrine, urinary cortisol, urinary dehydroepiandrosterone (DHEA), urinary tetrahydro-11-dehydrocorticosterone (THAs), urinary tetrahydrocortisone (THE), urinary tetrahydrocortisol (THF), and combinations thereof, or
(ii-b) the at least one additional biomarker in (i-b) is plasma normetanephrine, or
(ii-c) the at least one additional biomarker in (i-c) is urinary androsterone (An).

4. The combination of biomarkers according to claim 1, wherein the at least one additional biomarker in (i-a) is a combination comprising at least, or consisting in, plasma metanephrine, plasma normetanephrine, urinary cortisol, urinary dehydroepiandrosterone (DHEA), urinary tetrahydro-11-dehydrocorticosterone (THAs), urinary tetrahydrocortisone (THE), and urinary tetrahydrocortisol (THF).

5. The combination of biomarkers according to claim 1, wherein the combination of biomarkers is:
(i-a) age, plasma metanephrine, plasma normetanephrine, plasma 11-deoxycorticosterone, plasma 11-deoxycortisol, plasma 18OH-corticosterone, plasma 18OH-cortisol, plasma 18oxo-cortisol, plasma 21-deoxycortisol, plasma aldosterone, plasma corticosterone, plasma dehydroepiandrosterone sulfate (DHEAS), urinary 18-hydroxycortisol (18-OHF), urinary α-cortol (acortol), urinary cortisol, urinary dehydroepiandrosterone (DHEA), urinary pregnanediol (PD), urinary 3α,5β-tetrahydroaldosterone (THAldo), urinary tetrahydro-11-dehydrocorticosterone (THAs), urinary tetrahydrodeoxycorticosterone (THDOC), urinary tetrahydrocortisone (THE), urinary tetrahydrocortisol (THF), and urinary tetrahydro-11-deoxycortisol (THS),
(i-b) plasma normetanephrine, plasma 11-deoxycorticosterone, plasma 11-deoxycortisol, plasma 18OH-corticosterone, plasma 18OH-cortisol, plasma 18oxo-cortisol, plasma 21-deoxycortisol, plasma aldosterone, urinary 18-hydroxycortisol (18-OHF), urinary 3α,5β-tetrahydroaldosterone (THAldo), urinary tetrahydrodeoxycorticosterone (THDOC), urinary tetrahydro-11-deoxycortisol (THS), plasma decanoylcarnitine (C10), plasma dodecanoylcarnitine (C12), plasma octadecadienylcarnitine (C18:2), plasma H1 (sum of hexoses), plasma PC aa C32:1, plasma PC aa C34:3, plasma serotonin, plasma serotonin/tryptophan ratio (Serotonin/Trp),
(i-c) urinary α-cortol (acortol), urinary androsterone (An), and urinary tetrahydro-11-deoxycortisol (THS),
(i-d) age, plasma 11-deoxycorticosterone, plasma 11-deoxycortisol, plasma 18OH-corticosterone, plasma 18OH-cortisol, plasma 18oxo-cortisol, plasma 21-deoxycortisol, plasma aldosterone, urinary 18-hydroxycortisol (18-OHF), urinary pregnanediol (PD), urinary 3α,5β-tetrahydroaldosterone (THAldo), urinary tetrahydrodeoxycorticosterone (THDOC), urinary tetrahydro-11-deoxycortisol (THS), plasma decanoylcarnitine (C10), plasma tetradecenoylcarnitine (C14:1), plasma octadecenoylcarnitine (C18:1), plasma octadecadienylcarnitine (C18:2), plasma acetylcarnitine (C2), plasma C6 (C4:1-DC) (hexanoylcarnitine), plasma lysoPC a C17:0, plasma PC aa C32:1, plasma PC aa C32:2, plasma PC aa C32:3, plasma PC aa C34:2, plasma PC aa C34:3, plasma PC aa C36:3, plasma serotonin, and plasma serotonin/tryptophan ratio (Serotonin/Trp), or
(i-e) plasma 3-methoxytyramine, plasma metanephrine, and plasma normetanephrine.

6. A method of using a combination of biomarkers according to claim 1 for stratifying a hypertensive patient among a plurality of hypertensive diseases,
(i-a) the plurality of hypertensive diseases comprising Primary Aldosteronism (PA), Pheochromocytoma/Functional Paraganglioma (PPGL), Cushing's Syndrome (CS) and Primary Hypertension (PHT), and the combination of biomarkers being a combination of biomarkers (i-a), or
(i-b) the plurality of hypertensive diseases comprising Endocrine Hypertension (EHT) and Primary Hypertension (PHT), and the combination of biomarkers being a combination of biomarkers (i-b), or
(i-c) the plurality of hypertensive diseases comprising Cushing's Syndrome (CS) and Primary Hypertension (PHT), and the combination of biomarkers being a combination of biomarkers (i-c), or
(i-d) the plurality of hypertensive diseases comprising Primary Aldosteronism (PA) and Primary Hypertension (PHT), and the combination of biomarkers being a combination of biomarkers (i-d), or
(i-e) the plurality of hypertensive diseases comprising Pheochromocytoma/Functional Paraganglioma (PPGL) and Primary Hypertension (PHT), and the combination of biomarkers being a combination of biomarkers (i-e),
wherein the use comprises:
measuring, ex vivo, said combination of biomarkers, and
operating a trained classifier on said measured combination of biomarkers for obtaining a probability, for each hypertensive disease, of associating the hypertensive patient with a hypertensive disease in order to stratify said hypertensive patient among said plurality of types of hypertensive diseases according to (i-a) or according to (i-b) or according to (i-c) or according to (i-d) or according to (i-e),
said trained classifier being a classifier trained beforehand to learn a plurality of combinations of biomarkers previously selected based on at least one computed evaluation parameter and for several comparisons of at least two types of hypertensive patients.

7. A method for stratifying a hypertensive patient among a plurality of types of hypertensive diseases, using the combination of biomarkers of claim 1
(i-a) the plurality of types of hypertensive diseases comprising Primary Aldosteronism (PA), Pheochromocytoma/Functional Paraganglioma (PPGL), Cushing's Syndrome (CS) and Primary Hypertension (PHT), or
(i-b) the plurality of types of hypertensive diseases comprising Endocrine Hypertension (EHT) and Primary Hypertension (PHT), or
(i-c) the plurality of hypertensive diseases comprising Cushing's Syndrome (CS) and Primary Hypertension (PHT), or
(i-d) the plurality of hypertensive diseases comprising Primary Aldosteronism (PA) and Primary Hypertension (PHT), or
(i-e) the plurality of hypertensive diseases comprising Pheochromocytoma/Functional Paraganglioma (PPGL) and Primary Hypertension (PHT),
the method using at least one classifier trained beforehand to learn a plurality of combinations of biomarkers previously selected based on at least one computed evaluation parameter and for several comparisons of at least two types of hypertensive patients, and
the method comprising at least the steps of:
a) measuring, ex vivo, a combination of biomarkers,
wherein for the plurality of hypertensive diseases according to (i-a), the combination of biomarkers is a combination of biomarkers (i-a), or
wherein for the plurality of hypertensive diseases according to (i-b), the combination of biomarkers is a combination of biomarkers (i-b), or
wherein for the plurality of hypertensive diseases according to (i-c), the combination of biomarkers is a combination of biomarkers (i-c), or
wherein for the plurality of hypertensive diseases according to (i-d), the combination of biomarkers is a combination of biomarkers (i-d),
wherein for the plurality of hypertensive diseases according to (i-e), the combination of biomarkers is a combination of biomarkers (i-e),
b) operating said trained classifier on the combination of biomarkers obtained at step a) from said hypertensive patient for obtaining a probability, for each hypertensive disease, of associating the hypertensive patient with a hypertensive disease in order to stratify said hypertensive patient among said plurality of types of hypertensive diseases according to (i-a) or according to (i-b), or according to (i-c), or according to (i-d), or according to (i-e).

8. The method according to claim 6, wherein the trained classifier is selected from Decision Trees (J48), Naïve Bayes (NB), K-nearest neighbours (IBk), LogitBoost (LB), support vector machine (SVM), Logistic Model Tree (LMT), Bagging, Simple Logistic (SL), Random Forest (RF) and Sequential Minimal Optimisation (SMO).

9. The method according to claim 7, wherein the trained classifier is selected from LogitBoost (LB), Simple Logistic (SL), and Random Forest (RF).

10. The method according to claim 6, wherein the classifier has been trained with at least one predefined input dataset according to a method comprising at least the steps of:
a) for said at least one predefined input dataset and for at least one given comparison between at least a first and a second hypertensive patient, each patient having a first and a second type of hypertensive disease selected in said group of hypertensive diseases, the first and second type hypertensive disease being different, using said classifier to rank several combinations of biomarkers based on a computation of at least one evaluation parameter, and
b) based on said computed evaluation parameter(s), selecting a combination of biomarkers in order to stratify a hypertensive patient among said plurality of types of hypertensive diseases.

11. The method according to claim 6, wherein said evaluation parameter is chosen among accuracy, sensitivity, specificity, AUC, F1, Kappa score, and combinations thereof.

12. Kit for stratifying a hypertensive patient among a plurality of hypertensive diseases using a combination of biomarkers according to claim 1,
(i-a) the plurality of hypertensive diseases comprising Primary Aldosteronism (PA), Pheochromocytoma/Functional Paraganglioma (PPGL), Cushing's Syndrome (CS) and Primary Hypertension (PHT), and the kit comprising at least means for measuring a combination of biomarkers (i-a), or
(i-b) the plurality of hypertensive diseases comprising Endocrine Hypertension (EHT) and Primary Hypertension (PHT), and the kit comprising at least means for measuring a combination of biomarkers (i-b), or
(i-c) the plurality of hypertensive diseases comprising Cushing's Syndrome (CS) and Primary Hypertension (PHT), and the kit comprising at least means for measuring a combination of biomarkers (i-e), or
(i-d) the plurality of hypertensive diseases comprising Primary Aldosteronism (PA) and Primary Hypertension (PHT), and the kit comprising at least means for measuring a combination of biomarkers (i-d), or
(i-e) the plurality of hypertensive diseases comprising Pheochromocytoma/Functional Paraganglioma (PPGL) and Primary Hypertension (PHT), and the kit comprising at least means for measuring a combination of biomarkers (i-e).

13. Computer program product for stratifying a hypertensive patient among a plurality of hypertensive diseases based on the biomarkers according to claim 1,
(i-a) the plurality of hypertensive diseases comprising Primary Aldosteronism (PA), Pheochromocytoma/Functional Paraganglioma (PPGL), Cushing's Syndrome (CS) and Primary Hypertension (PHT), or
(i-b) the plurality of hypertensive diseases comprising Endocrine Hypertension (EHT) and Primary Hypertension (PHT), or
(i-c) the plurality of hypertensive diseases comprising Cushing's Syndrome (CS) and Primary Hypertension (PHT), or
(i-d) the plurality of hypertensive diseases comprising Primary Aldosteronism (PA) and Primary Hypertension (PHT), or
(i-e) the plurality of hypertensive diseases comprising Pheochromocytoma/Functional Paraganglioma (PPGL) and Primary Hypertension (PHT),
the computer program using
(1) at least one classifier trained beforehand to learn a plurality of combinations of biomarkers previously selected based on at least one computed evaluation parameter and for several comparisons of at least a first and a second type of hypertensive disease selected in a group (i-a), (i-b), (i-c), (i-d) and (i-e), of hypertensive diseases comprising
(i-a) Primary Aldosteronism (PA), Pheochromocytoma/Functional Paraganglioma (PPGL), Cushing's Syndrome (CS) and Primary Hypertension (PHT),
(i-b) Endocrine Hypertension (EHT) and Primary Hypertension (PHT),
(i-c) Cushing's Syndrome (CS) and Primary Hypertension (PHT),
(i-d) Primary Aldosteronism (PA) and Primary Hypertension (PHT), or
(i-e) Pheochromocytoma/Functional Paraganglioma (PPGL) and Primary Hypertension (PHT),
the first and second type hypertensive disease being different, wherein said classifier has been trained with at least one predefined input dataset according to a method comprising at least the steps of:
a) for said at least one predefined input dataset and for at least one given comparison between at least a first and a second hypertensive patient, each patient having a first and a second type of hypertensive disease selected in said group of hypertensive diseases (i-a), (i-b), (i-c), (i-d) and (i-e), the first and second type hypertensive disease being different, using said classifier to rank several combinations of biomarkers based on a computation of at least one evaluation parameter, and
b) based on said computed evaluation parameter(s), selecting a combination of biomarkers in order to stratify a hypertensive patient among said plurality of hypertensive diseases (i-a), (i-b), (i-c), (i-d) and (i-e),
(2) with at least one input of measured biomarkers, said input of measured biomarkers being obtained by measuring, in suitable biological samples previously isolated from said patient, a combination of biomarkers (i-a) wherein the plurality of hypertensive diseases is according to (i-a) or a combination of biomarkers (i-b wherein the plurality of hypertensive diseases is according to (i-b), or a combination of biomarkers (i-c) wherein the plurality of hypertensive diseases is according to (i-c), or a combination of biomarkers (i-d) wherein the plurality of hypertensive diseases is according to (i-d), or a combination of biomarkers (i-e) wherein the plurality of hypertensive diseases is according to (i-e),
the computer program product comprising instructions which, when the program is executed by a computer, cause the computer to, for said at least one input of measured biomarkers, use said trained classifier for associating each hypertensive disease of said plurality of hypertensive diseases (i-a), (i-b), (i-c), (i-d) or (i-e), with a probability of associating the hypertensive patient with said hypertensive disease in order to stratify said hypertensive patient among said plurality of hypertensive diseases (i-a), (i-b), (i-c), (i-d) and (i-e).

14. A combination of biomarkers according to claim 1 for use in a method for treating a hypertensive disease in a patient in need thereof,
the hypertensive disease being selected among
(i-a) Primary Aldosteronism (PA), Pheochromocytoma/Functional Paraganglioma (PPGL), Cushing's Syndrome (CS) and Primary Hypertension (PHT), and the combination of biomarkers being a combination of biomarkers (i-a), or
(i-b) Endocrine Hypertension (EHT) and Primary Hypertension (PHT), and the combination of biomarkers being a combination of biomarkers (i-b), or
(i-c) the plurality of hypertensive diseases comprising Cushing's Syndrome (CS) and Primary Hypertension (PHT), and the combination of biomarkers being a combination of biomarkers (i-c), or
(i-d) the plurality of hypertensive diseases comprising Primary Aldosteronism (PA) and Primary Hypertension (PHT), and the combination of biomarkers being a combination of biomarkers (i-d), or
(i-e) the plurality of hypertensive diseases comprising Pheochromocytoma/Functional Paraganglioma (PPGL) and Primary Hypertension (PHT), and the combination of biomarkers being a combination of biomarkers (i-e),
the method of treating comprising a step of stratifying the hypertensive patient among the hypertensive diseases of (i-a), (i-b), (i-c), (i-d) or (i-e), said step of stratifying comprising
measuring, ex vivo, said combination of biomarkers, and
operating a trained classifier on said measured combination of biomarkers for obtaining a probability, for each hypertensive disease, of associating the hypertensive patient with a hypertensive disease in order to stratify said hypertensive patient among said plurality of types of hypertensive diseases according to (i-a) or according to (i-b) or according to (i-c) or according to (i-d) or according to (i-e),
said trained classifier being a classifier trained beforehand to learn a plurality of combinations of biomarkers previously selected based on at least one computed evaluation parameter and for several comparisons of at least two types of hypertensive patients.

15. A method for treating a hypertensive patient using a combination of biomarkers according to claim 1, said method comprising:
a) stratifying said hypertensive patient among a plurality of hypertensive diseases,
(i-a) the plurality of hypertensive diseases comprising Primary Aldosteronism (PA), Pheochromocytoma/Functional Paraganglioma (PPGL), Cushing's Syndrome (CS) and Primary Hypertension (PHT), and the combination of biomarkers being a combination of biomarkers (i-a), or
(i-b) the plurality of hypertensive diseases comprising Endocrine Hypertension (EHT) and Primary Hypertension (PHT), and the combination of biomarkers being a combination of biomarkers (i-b), or
(i-c) the plurality of hypertensive diseases comprising Cushing's Syndrome (CS) and Primary Hypertension (PHT), and the combination of biomarkers being a combination of biomarkers (i-c), or
(i-d) the plurality of hypertensive diseases comprising Primary Aldosteronism (PA) and Primary Hypertension (PHT), and the combination of biomarkers being a combination of biomarkers (i-d), or
(i-e) the plurality of hypertensive diseases comprising Pheochromocytoma/Functional Paraganglioma (PPGL) and Primary Hypertension (PHT), and the combination of biomarkers being a combination of biomarkers (i-e),
The stratification of said hypertensive patient among said plurality of hypertensive diseases (i-a), (i-b), (i-c), (i-d) and (i-e), being carried for associating a hypertensive disease to said patient,
b) selecting a therapeutic treatment presumed to treat or to relieve at least one symptom of the hypertensive disease associated to said patient, and
c) treating said patient by administering to said patient said therapeutic treatment selected at step b).
